(19) United States
(12) Patent Application Publication
(10) Pub. No.: US 2024/0384293 A1
(43) Pub. Date: Nov. 21, 2024

(54) VIRAL VECTOR PRODUCTION SYSTEM

(71) Applicant: Novartis AG, Basel (CH)

(72) Inventors: Astrid Bosse, Birsfelden (CH); Benoit Bossuge, Sierentz (FR); Laurence Croute, Village Neuf (FR); Lars Ellenrieder, Freiburg (DE); Laurence Guianvarch, Mulhouse (FR); David Schmitt, Hirsingue (FR); Eleonora Toffoli, Basel (CH)

(21) Appl. No.: 18/288,345

(22) PCT Filed: Apr. 27, 2022
(86) PCT No.: PCT/IB2022/053880

§ 371 Date: Oct. 25, 2023

Related U.S. Application Data
(60) Provisional application No. 63/180,423, filed on Apr. 27, 2021.

(51) Int. Cl. C12N 15/86

(52) U.S. Cl. CPC C12N 15/86; C12N 2740/15043; C12N 2740/15052; C12N 2750/14143; C12N 2760/20222

(57) ABSTRACT

The disclosure provides, at least in part, to a method for producing high titer lentiviral vectors, and for producing lentiviral particles carrying a transgene of interest and under satisfactory safety conditions. The disclosure also provides at least in part, methods of purification of such lentiviral particle, e.g., from a cell culture. The disclosure also provides a formulation to lentiviral preparations that maintain structural integrity of the viral vector during purification, storage, and gene transfer events.

### RELATED APPLICATIONS

This application claims priority to U.S. Provisional Application 63/180,423 filed on Apr. 27, 2021, the entire contents of which are hereby incorporated by reference.

### BACKGROUND

Viruses are highly efficient at nucleic acid delivery to specific cell types, while often avoiding detection by the infected host immune system. These features make certain viruses attractive candidates as gene-delivery vehicles for use in gene therapies.

Among the viral vectors available for gene therapy applications are lentiviral vectors. Such vectors include reconstructed viral vector systems derived from human immunodeficiency virus-1 (HIV-1) and are capable of introducing a gene of interest into animal and human primary cells or cell lines. Lentiviral vector-mediated gene expression can be used to achieve continuous and stable protein production, because the gene of interest has been integrated into a host cell's genome and is thus replicated upon division of the cell. Lentiviral vectors can effectively transduce non-dividing cells as well as those actively progressing through the cell cycle. Tissues and cells in which lentiviral vector-mediated chronic expression of a gene of interest can occur include the brain, liver, muscle cells, retina, hematopoietic stem cells, marrow mesenchymal stem cells, and macrophages, among others.

The large-scale production of lentiviral vectors has been hindered by several challenges, such as low titer of the viral yield and low stability of the vector. Additionally, Lentiviral vectors are susceptible to inactivation during purification process which can contribute to diminished final quality and efficacy of the vector preparation further creating another hurdle for production of large scale of purified lentiviral vector. Thus, there remains a need for a method for large-scale production of lentiviral vectors with high titer and a large-scale purification process and formulation buffers that preserve vector stability.

### SUMMARY

The disclosure provides, at least in part, to a method for producing high titer lentiviral vectors, carrying a transgene of interest under satisfactory safety conditions. The disclosure also provides at least in part, methods of purification of such lentiviral particles, e.g., from a cell culture. The disclosure also provides a formulation for lentiviral preparations that maintain structural integrity of the viral vector during purification, storage, and gene transfer events, e.g., ex vivo gene transfer.

In some aspects, the present disclosure provides a method for manufacturing a lentiviral vector, the method comprising:
a) providing a plurality of mammalian (e.g., human) cells,
b) contacting the plurality of mammalian cells with:
   i) FectoVIR®-AAV transfection reagent, and
   ii) nucleic acid encoding a therapeutic effector, e.g., a therapeutic protein (e.g., a CAR) and sufficient LTR sequence for packaging into a viral particle, and optionally nucleic acid encoding a lentiviral packaging protein, a lentiviral envelope protein, and,
under conditions that allow the nucleic acid to be introduced into at least a subset of the cells; and
c) culturing the cell under conditions suitable for production of the lentiviral vector.

In certain embodiments, when the plurality of mammalian cells is in a 50 L culture yields a number of transducing units per ml culture that is no less than 50%, 60%, 70%, or 80% the number of transducing units per ml culture in an otherwise similar 100 ml culture.

In certain embodiments, the method yields at least 1×10⁷ or 3×10⁷ or at least 1×10⁸ transducing units when used under conditions described in Example 5.

In certain embodiments, the method yields a ratio of equal to or less than 1188:1, 953:1, and 1800:1 PP (physical particles): IP (infectious particles).

In certain embodiments, the mammalian cells are 293 cells, e.g., Expi293F cells.

In certain embodiments, the FectoVIR®-AAV is used at a concentration of 0.3-0.6 μl FectoVIR®-AAV/million cells, e.g., about 0.4 μl/million cells.

In certain embodiments, the nucleic acid is used at a concentration of 0.3-0.6 μg of nucleic acid/million cells, e.g., about 0.4 μg/million cells.

In certain embodiments, the ratio of FectoVIR®-AAV: DNA for transfection 1:0.5 to 1:2, e.g., about 1:1 (wherein optionally the DNA for transfection comprises DNA encoding the therapeutic effector, DNA encoding one or more retroviral packaging protein and DNA encoding a retroviral envelope protein).

In certain embodiments, the FectoVIR®-AAV transfection reagent is complexed with the nucleic acid.

In some embodiments, the method further comprises admixing the FectoVIR®-AAV transfection reagent with the nucleic acid before step b).

In certain embodiments, complexation volume of the transfection reagent and the nucleic acid is between about 1% and about 15%, e.g., about 1% and about 10% (e.g., about 5-7.5% or 7.5-10%).

In some embodiments, the complexation volume is 3-7%, 4-6%, or about 5%.

In certain embodiments, the FectoVIR®-AAV transfection reagent and the nucleic acid are incubated for sufficient time to allow complexation to occur, e.g., about 10-90 minutes, e.g., 15-60, e.g., 15-30, 30-45, or 45-60 minutes.

In some aspects, the present disclosure provides a method of manufacturing a lentiviral vector, the method comprising:
a) culturing a plurality of mammalian (e.g., human) cells at a pH of above about 6.9 or about 6.9-7.3, e.g., about 7.0-7.1;
b) subsequently to step a), adjusting the pH of the culture to about 6.0-6.8, e.g., 6.6-6.8, e.g., about 6.7;
c) subsequently to step b), contacting the culture with a transfection reagent and DNA.

In certain embodiments, the transfection reagent comprises FectoVIR®-AAV transfection reagent.

In some embodiments, the DNA encodes one or more retroviral packaging protein, a retroviral envelope protein, and a therapeutic effector, e.g., a therapeutic protein (e.g., a CAR).

In some embodiments, a) comprises culturing the cells for about 2-4 days, e.g., about 3 days.

In certain embodiments, the method further comprises an additional step of culturing the cells between steps b) and c).

In some embodiments, the method further comprises an additional step of culturing the cells after step c).

In some embodiments, step b) comprises lowering the pH by about 0.1, 0.2, 0.3, 0.4, 0.5, 0.6, 0.7, 0.8, 0.9, or 1.

In certain embodiments, prior to step a), the plurality of mammalian cells are inoculated at between 0.1×10⁶ cells/mL—and 0.3×10⁶ cells/mL (e.g., about 0.15×10⁶ cells/mL or about 0.2×10⁶ cells/mL) in culture medium (e.g., FreeStyle™ medium) at a final volume.

In some embodiments, the plurality of mammalian cells are inoculated between 50 and 80 hours (e.g., about 55 hours, about 60 hours, about 65 hours, about 70 hours, about 72 hours, about 75 hours, or about 80 hours) prior to step a).

In certain embodiments, the plurality of mammalian cells are cultured under conditions suitable to allow for cell growth and amplification to a suitable cell density at transfection (e.g., between about 1.0×10⁶ cells/mL and about 3.0×10⁶ cells/mL (e.g., between 1.5×10⁶ cells/mL and 2.5×10⁶ cells/mL).

In some aspects, the present disclosure provides a method of manufacturing a lentiviral vector, the method comprising:
a) providing a composition comprising the lentiviral vector and at least one impurity (e.g., wherein the composition comprises a clarified cell harvest or a filtrate), and
b) contacting the composition with arginine or a salt thereof.

In certain embodiments, one or more of:
i) the arginine is at a concentration of about 25-50 mM (about 50 mM), 50-100 mM (e.g., about 75 mM), 100-200 mM (e.g., about 150 mM), or 200-400 (e.g., about 300) mM arginine); or
ii) the arginine is at a concentration sufficient to increase level of transducing units of the lentiviral vector by about 10%-300%, 20%-180%, 30%-160%, 50%-150%, 75%-125% or about 100% compared to an otherwise similar composition, e.g., in an assay according to Example 7;
iii) after step b) the composition shows a total particle concentration per ml of less than 400,000, 300,000, 200,000, or 100,000, as measured by micro-flow imaging, e.g., in an assay described in Example 10, wherein optionally the particles comprise aggregated lentivirus;
iv) after step b) the composition shows a concentration of particles that are ≥10 μm per ml of less than about 5,000, 4,500, 4,000, 3,500, 3,000, or 2,500, as measured by micro-flow imaging, e.g., in an assay described in Example 10 wherein optionally the particles comprise aggregated lentivirus;
v) after step b) the composition shows a concentration of particles that are ≥25 μm per ml of less than about 500, 400, 300, or 200, as measured by micro-flow imaging, e.g., in an assay described in Example 10 wherein optionally the particles comprise aggregated lentivirus;
vi) after step b), the composition shows reduced aggregation of the lentiviral vector compared to an otherwise similar filtrate without addition of the arginine or salt thereof;
vii) recovery of transducing units of the lentiviral vector is greater than an otherwise similar control without arginine added, e.g., by at least about 10%, 20%, 50%, 100%, or 200%, e.g., as measured in an assay according to Example 7.

In some embodiments, b) comprises contacting the composition with a solution comprising the arginine and a buffer, wherein optionally the buffer is PIPES, wherein optionally the PIPES is at a concentration of from about 10 mM to about 50 mM, e.g., about of 20 mM in the solution. In certain embodiments, the solution has a pH of about 6.0 to about 7.0, e.g., about 6.5.

In some embodiments, the solution further comprises a salt, wherein optionally the salt is selected from the group consisting of sodium chloride, magnesium chloride, and calcium chloride, e.g., sodium chloride.

In some embodiments, the salt is present in the solution at a concentration of from about 25-150 mM, e.g., 50-100 mM, e.g., about 75 mM.

In certain embodiments, the concentration of the salt in the solution has a pH of about 6.5.

In some embodiments, the solution further comprises a carbohydrate, e.g., a non-reducing carbohydrate, e.g., sucrose or trehalose.

In certain embodiments, the carbohydrate is present in the solution at a concentration of from about 1% to about 10% by weight per volume of said solution, e.g., about 2% to about 5% by weight per volume of the solution, about 2.5% by weight per volume of the solution.

In certain embodiments, the carbohydrate is present in the solution at a concentration of about 30-150 mM (about 73 mM), or 150-300 (e.g., about 220) mM.

In some embodiments, the solution further comprises one or both of NaCl (e.g., about 25-150 mM, e.g., 50-100 mM, e.g., about 75 mM), and sucrose (e.g., about 30-150 mM, e.g., about 73 mM, or e.g., about 150-300 mM, e.g., about 220 mM, or about 2.5% by weight per volume) of the solution.

In some embodiments, the solution comprises 20 mM PIPES, 75 mM sodium chloride, and 2.5% sucrose by weight per volume of the solution, and wherein the solution has a pH of about 6.5. In certain embodiments, the solution comprises about 20 mM PIPES, about 75 mM sodium chloride, and about 2.5% sucrose by weight per volume of the solution, and wherein the solution has a pH of about 6.5.

In certain embodiments, the solution comprises 20 mM PIPES, 75 mM sodium chloride, and 73 mM sucrose and wherein the solution has a pH of about 6.5. In certain embodiments, the solution comprises about 20 mM PIPES, about 75 mM sodium chloride, and about 73 mM sucrose and wherein the solution has a pH of about 6.5.

In some embodiments, the solution comprises 20 mM PIPES, 75 mM sodium chloride, and 220 mM sucrose and wherein the solution has a pH of about 6.5. In some embodiments, the solution comprises about 20 mM PIPES, about 75 mM sodium chloride, and about 220 mM sucrose and wherein the solution has a pH of about 6.5.

In some embodiments, the solution further comprises 20 mM PIPES, 75 mM arginine, e.g., arginine-HCl, and wherein the solution has a pH of about 6.5. In some embodiments, the solution further comprises about 20 mM PIPES, about 75 mM arginine, e.g., arginine-HCl, and wherein the solution has a pH of about 6.5.

In some embodiments, the osmolality of said solution is from about 270 mOsm/kg to about 330 mOsm/kg, e.g., about 275 mOsm/kg to about 300 mOsm/k, e.g., about 285 mOsm/kg.

In certain embodiments, the method further comprises: c) performing a purification step, e.g., a filtration step, on the composition of b), thereby producing a semi-purified composition comprising the lentiviral vector.

In certain embodiments, the method further comprises, after step c), contacting the semi-purified composition with arginine or a salt thereof.

In some embodiments, the arginine encapsulates the lentiviral vector.

In certain embodiments, the arginine stabilizes the lentiviral vector.

In some embodiments, the impurity comprises a protein (e.g., a host cell protein), a nucleic acid (e.g., a host cell nucleic acid), a carbohydrate (e.g., a host cell carbohydrate), a lipid, an enzyme, a salt, a buffer, or any combination thereof.

In certain embodiments, the cell density at transfection is between about 1.0×10⁶ cells/mL and about 3.0×10⁶ cells/mL (e.g., between 1.5×10⁶ cells/mL and 2.5×10⁶ cells/mL).

In some embodiments, the viability of the cells is, or is assessed to be, at least 90% (e.g., 91%, 92%, 93%, 94%, 95%, 96%, 97%, 98%, 99%, or 100%) at the time of transfection.

In certain embodiments, the viability of the cells is measured at or around the time of transfection (e.g., within 30 minutes prior to transfection).

In some embodiments, the method is used for a process with two or more nucleic acids (e.g., two or more plasmids, e.g., two plasmids, three plasmids, four plasmids, or five plasmids).

In some aspects, the present disclosure provides a method of manufacturing a lentiviral vector, the method comprising:
a) providing a population of human cells (e.g., 293 cells);
b) introducing into the cells nucleic acid encoding a retroviral packaging protein, a retroviral envelope protein, and a therapeutic effector, e.g., a therapeutic protein (e.g., a CAR),
c) contacting the cells with benzonase at a time about 6-40, 10-40, 10-30, or about 20 hours after step b); and
d) culturing the cells under conditions suitable for production of the lentiviral vector.

In some aspects, the present disclosure provides a method of manufacturing a lentiviral vector, the method comprising:
a) providing a population of human cells (e.g., 293 cells);
b) introducing into the cells nucleic acid encoding a retroviral packaging protein, a retroviral envelope protein, and a therapeutic effector, e.g., a therapeutic protein (e.g., a CAR),
c) contacting the cells with benzonase;
d) culturing the cells under conditions suitable for production of the lentiviral vector;
e) harvesting the lentiviral vectors from cells 6-10 hours, 10-20 hours, 20-30 hours, 30-40 hours, or 40-50 hours after step c).

In some aspects, the present disclosure provides a method of manufacturing a lentiviral vector, the method comprising:
a) providing a plurality of mammalian (e.g., human) cells, wherein the plurality of cells (e.g., wherein the cell is a fibroblast cell, e.g., an embryonic kidney fibroblast cell, e.g., an Expi293F cell), wherein the cell comprises a nucleic acid (e.g., DNA) encoding one or more retroviral packaging protein, a retroviral envelope protein, and a therapeutic effector, e.g., a therapeutic protein (e.g., a CAR),
b) culturing the cell under conditions suitable for production of the lentiviral vector.

In some aspects, the present disclosure provides, an aqueous composition comprising a lentiviral vector, arginine, a 1,4-piperazinediethanesulfonic acid (PIPES) buffer, and a salt.

In certain embodiments, the arginine in the aqueous composition is at a concentration of about 25-50 mM (about 50 mM), 50-100 mM (e.g., about 75 mM), 100-200 mM (e.g., about 150 mM), or 200-400 (e.g., about 300) mM arginine), wherein optionally the PIPES aqueous composition is at a concentration of from about 10 mM to about 50 mM, e.g., about, e.g., 20 mM.

In some embodiments, the aqueous composition has a pH of about 6.0 to about 7.0, e.g., about 6.5.

In certain embodiments, the aqueous composition further comprises a salt, wherein optionally the salt is selected from the group consisting of sodium chloride, magnesium chloride, and calcium chloride.

In some embodiments, the salt is sodium chloride (NaCl).

In certain embodiments, the salt in the aqueous composition is from about 25 mM to about 150 mM, e.g., about 50 mM to about 75 mM.

In some embodiments, the aqueous composition comprises 20 mM PIPES and 75 mM sodium chloride, and wherein the aqueous composition has a pH of about 6.5.

In certain embodiments, the aqueous composition further comprises a carbohydrate, e.g., a non-reducing carbohydrate, e.g., sucrose or trehalose.

In some embodiments, the carbohydrate is present in the aqueous composition at a concentration of from about 1% to about 10% by weight per volume of said solution, e.g., about 2% to about 5% by weight per volume of the aqueous composition, about 2.5% by weight per volume of the aqueous composition.

In one embodiment, the carbohydrate is present in the aqueous composition at a concentration of from about 30-150 mM (about 73 mM), or 150-300 (e.g., about 220) mM.

In some embodiments, the aqueous composition comprises one or both of NaCl (e.g., about 25-150 mM, e.g., 50-100 mM, e.g., about 75 mM), and sucrose (e.g., about 30-150 mM, e.g., about 73 mM, or e.g., about 150-300 mM, e.g., about 220 mM, or about 2.5% by weight per volume) of the aqueous composition.

In one embodiment, the aqueous composition comprises 20 mM PIPES, 75 mM sodium chloride, and 2.5% sucrose by weight per volume of the aqueous composition and wherein the aqueous composition has a pH of about 6.5.

In certain embodiments, the aqueous composition comprises 20 mM PIPES, 75 mM sodium chloride and 73 mM sucrose and wherein the aqueous composition has a pH of about 6.5.

In one embodiment, the aqueous composition comprises 20 mM PIPES, 75 mM sodium chloride and 220 mM sucrose and wherein the aqueous composition has a pH of about 6.5.

In certain embodiments, the osmolality of said aqueous composition is from about 270 mOsm/kg to about 330 mOsm/kg, e.g., about 275 mOsm/kg to about 300 mOsm/k, e.g., about 285 mOsm/kg.

In one embodiment, the lentiviral vector of any preceding claims is present at a concentration of from about 3×10⁸ TU/mL to about 5×10⁸ TU/mL.

In certain embodiments, the aqueous composition is free of one or more proteins selected from the group consisting of human serum albumin (HSA), recombinant human serum albumin (rHSA), bovine serum albumin (BSA), and a lipoprotein.

In one embodiment, lentiviral vector comprises a transgene, e.g., a transgene encoding a protein, e.g., a protein comprising a chimeric antigen receptor (CAR).

In certain embodiments, said CAR comprises, in an N-terminal to C-terminal direction, an antigen binding domain, a transmembrane domain, and one or more signaling domains.

In some embodiments, said signaling domain comprises one or more primary signaling domains and/or one or more costimulatory signaling domains.

In certain embodiments, one of said one or more primary signaling domains comprises a CD3-zeta stimulatory domain.

In some embodiments, one or more of said costimulatory signaling domains comprises an intracellular domain selected from a costimulatory protein selected from the group consisting of OX40, CD27, CD28, CDS, ICAM-1, LFA-1 (CD11a/CD18), ICOS(CD278), 4-1BB (CD137), CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CDIId, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, e.g., a 4-1 BB (CD137) costimulatory domain or a CD28 costimulatory domain.

In some embodiments, one or more of said costimulatory signaling domains comprises an intracellular domain selected from a costimulatory protein selected from the group consisting ofCD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CDIIc, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, or NKG2D.

In certain embodiments, said antigen binding domain is an scFv.

In some embodiments, said antigen binding domain binds to an antigen selected from the group consisting of CD19; CD123; CD22; CD30; CD171; CS-1; C-type lectin-like molecule-1, CD33; epidermal growth factor receptor variant III (EGFRvlll); ganglioside G2 (GD2); ganglioside GD3; TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD1 17); lnterleukin-13 receptor subunit alpha-2; mesothelin; Interleukin 1 1 receptor alpha (IL-1 1 Ra); prostate stem cell antigen (PSCA); Protease Serine 21; vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3; transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51 E2 (OR51 E2); TCR Gamma Alternate Reading Frame Protein (TARP); Wilms tumor protein (WT1); Cancer/testis antigen 1 (NY-ESO-1); Cancer/testis antigen 2 (LAGE-1 a); Melanoma-associated antigen 1 (MAGE-A1); ETS translocation-variant gene 6, located on chromosome 12p (ETV6-AML); sperm protein 17 (SPA17); X Antigen Family, Member 1 A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1, melanoma antigen recognized by T cells 1; Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1 B1 (CYP1 B1); CCCTC-Binding Factor (Zinc Finger Protein)-Like, Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1), e.g., to CD19, CD22, mesothelin, or CD123.

In certain embodiments, said CAR comprises an anti-CD19 antibody or a fragment thereof, a 4-1 BB (CD137) transmembrane domain, and a CD3-zeta signaling domain.

In some embodiments, the lentiviral vector comprises a second transgene, e.g., a second transgene encoding a second protein, e.g., a second protein comprising a second chimeric antigen receptor (CAR).

In one aspect, the present disclosure provides a method of manufacturing a lentiviral vector, comprising:
a) providing a population of human cells (e.g., 293 cells);
b) introducing into the cells nucleic acid encoding a retroviral packaging protein, a retroviral envelope protein, and a therapeutic effector, e.g., a therapeutic protein (e.g., a CAR),
c) contacting the cells with benzonase at a time about 2-6 (e.g., about 3), 4-10 (e.g., about 6), 6-40, 10-40, 10-30 (e.g., about 24), or about 20 hours after step b); and
d) culturing the cells under conditions suitable for production of the lentiviral vector.

In certain embodiments, Benzonase is added 6-10 hours, 10-20 hours, 20-30 hours, 30-40 hours, or 40-50 hours, before harvest of lentiviral vector from the cells.

In one aspect, the present disclosure provides a method of manufacturing a lentiviral vector, comprising:
a) providing a population of human cells (e.g., 293 cells);
b) introducing into the cells nucleic acid encoding a retroviral packaging protein, a retroviral envelope protein, and a therapeutic effector, e.g., a therapeutic protein (e.g., a CAR),
c) contacting the cells with benzonase (e.g., 3-24 hours after step b);
d) culturing the cells under conditions suitable for production of the lentiviral vector;
e) harvesting the lentiviral vectors from cells 6-10 hours, 10-20 hours, 20-30 hours, 30-40 hours, or 40-50 hours after step c).

In some embodiments, benzonase is at a concentration of about 10-40 U/mL, e.g., 20-30 U/mL, e.g., about 25 U/mL.

In certain embodiments, benzonase is at a concentration of about 3-60 U/mL, 3-10 U/mL, 3-7 U/mL, 4-6 U/mL, or about 5 U/mL.

In one embodiment, the benzonase is at a concentration of 5-50, 5-15, 15-25, or 25-50 U/mL.

In certain embodiments, the method further comprises, before step c), contacting the benzonase with MgCl₂, e.g., at about 1-5 mM, 1-3 mM, or about 2 mM.

In one aspect, the present disclosure provides a method of manufacturing a lentiviral vector, comprising:
a) providing a plurality of mammalian (e.g., human) cells, wherein the plurality of mammalian cells do not comprise SV40 large T antigen (e.g., wherein the cell is a fibroblast cell, e.g., an embryonic kidney fibroblast cell, e.g., an Expi293F cell), wherein the plurality of mammalian cells comprise a nucleic acid (e.g., DNA) encoding one or more retroviral packaging protein, a retroviral envelope protein, and a therapeutic effector, e.g., a therapeutic protein (e.g., a CAR),
b) culturing the cell under conditions suitable for production of the lentiviral vector.

In certain embodiments, a) comprises introducing the nucleic acid into the plurality of mammalian cells.

In some embodiments, the method further comprises at least partially separating the lentiviral vector from the plurality of mammalian cells.

In one embodiment, the one or more retroviral packaging proteins comprises a lentiviral gag, a lentiviral pol, or a lentiviral rev, or any combination thereof.

In certain embodiments, the retroviral envelope protein comprises a VSV-G.

In some aspects the present disclosure provides a preparation of lentiviral vector, the preparation comprising:
a plurality of lentiviral vector that comprise:
a) a lentivirus genome encoding a therapeutic effector, e.g., a therapeutic protein (e.g., a CAR), and
b) an envelope enclosing the lentivirus genome (wherein optionally the envelope comprises VSV-G);
wherein the preparation comprises at least 5×10⁷, 1×10⁸, 1×10⁹, or 1×10¹⁰, transducing units;
wherein the preparation comprises less than 90% of SV40 large T antigen or less than 10 μg/ml, 1 μg/ml of nucleic acid (e.g., DNA) encoding SV40 large T antigen.

In some embodiments, the plurality of lentiviral vectors comprises at least 1×10⁹, 2×10⁹, 5×10⁹, or 1×10¹⁰, 2×10¹⁰, 5×10¹⁰, 1×10¹¹, 2×10¹¹, 5×10¹, or 1×10¹² of the cells.

In certain embodiments, the plurality of mammalian cells are in a culture volume of at least 5, 10, 20, 50, 100, 200, or 500 L.

In one embodiment, comprises culturing the plurality of mammalian cells in serum-free medium.

In certain embodiments, the plurality of mammalian cells are grown in suspension.

In some embodiments, the CAR comprises a CD19 CAR (e.g., a humanized CD19 CAR, e.g., as described in WO2014153270A1.

In certain embodiments, the CAR comprises a dual CAR (e.g., a humanized CD19-CD22 CAR, e.g., as described in WO2016164731A2.

In some embodiments, the nucleic acid encoding a CAR further encodes a shRNA, e.g., as described in WO2017049166A.

In one embodiment, the lentiviral vector is produced in cells cultured in the absence of serum.

In certain embodiments, the lentiviral vector is characterized by a hydrodynamic radius of 100±25 nm as measured by dynamic light scattering (DLS).

In certain embodiments, the lentiviral vector maintains said hydrodynamic radius of 100±25 nm within a temperature range of from 25° C. to 55° C. (e.g., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., or 55° C.).

In some embodiments, the lentiviral vector is characterized by a polydispersity of from 10% to 25% (e.g., 10%, 11%, 12%, 13%, 14%, 15%, 16%, 17%, 18%, 19%, 20%, 21%, 22%, 23%, 24% or 25%).

In one embodiment, the lentiviral vector maintains said polydispersity of from 10% to 25% (e.g., 10%, 11%, 12%, 13%, 14%, 15%, 16%, 17%, 18%, 19%, 20%, 21%, 22%, 23%, 24% or 25%) within a temperature range of from 25° C. to 55° C. (e.g., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., or 55° C.).

In certain embodiments, the lentiviral vector maintains a concentration after 3 freeze/thaw cycles of from about 70% to about 100% (e.g., about 70%, about 75%, about 80%, about 85%, about 90%, about 95%, about 97%, or about 100%) relative to the concentration of said lentiviral vector in said aqueous composition prior to said freeze/thaw cycles, wherein each of said freeze/thaw cycles comprises freezing said aqueous composition and subsequently allowing said aqueous composition to thaw at room temperature.

In some embodiments, the lentiviral vector maintains said concentration of from about 70% to about 100% (e.g, about 70%, about 75%, about 80%, about 85%, about 90%, about 95%, about 97%, or about 100%) after 6-10 of said freeze/thaw cycles, e.g., after 6-9 of said freeze/thaw cycles.

In some aspects, the present disclosure provides an aqueous composition comprising a lentiviral vector, a buffer selected from the group consisting of a phosphate buffer, a sodium citrate buffer, a 2-(N-morpholino) ethanesulfonic acid (MES) buffer, a 3-morpholinopropane-1-sulfonic acid (MOPS) buffer, and a salt.

In some embodiments, said salt is selected from the group consisting of sodium chloride, magnesium chloride, and calcium chloride.

In one embodiment, said aqueous composition further comprises a non-reducing carbohydrate selected from the group consisting of sucrose and trehalose.

In some aspects, the present disclosure provides scalable processes for the production of large quantities of viral vectors (e.g., lentiviral vectors), e.g., for prophylactic, diagnostic, immunotherapeutic or therapeutic use. The processes may be performed using suspension cells (e.g., HEK293 cells, e.g., Expi293F cells). In some embodiments, substantially all of the suspension cells do not express a large T antigen, e.g., SV40 T antigen. In some embodiments, the process may be performed using a bioreactor.

In some aspects, the present disclosure provides highly reproducible efficient scalable processes for the production of large quantities of viral vectors (e.g., lentiviral vectors) having one or both of a high viral titer or high viral yield.

In some aspect, the present disclosure provides highly reproducible efficient scalable processes for the purification of large quantities of viral vector (e.g., lentiviral) having one or both of a high viral titer or high viral yield.

In another aspect, the present disclosure provides compositions and methods for stabilizing viral vectors, e.g., lentiviral vectors during a purification process.

### BRIEF DESCRIPTION OF THE FIGURES

FIG. 1A shows a LV productivity of ˜1.5E7 TU/mL with Expi293F cells and a LV productivity of ˜3.9E7 TU/mL with HEK293T/17 cells, and the PP/IP ratio obtained with Expi293F cells is about 1900, compared to a PP/IP ratio of about 1000 achieved with HEK293T/17 cells.

FIG. 1B shows the cell densities observed at each passage are comparable between both cell lines (˜3×10⁶ cells/mL).

FIG. 1C shows both Expi293F and HEK293T/17 cells show high viability in culture (>90%).

FIG. 2A shows the transfection reagent FectoVIR®-AAV increases significantly the LV productivity of Expi293F cells, from 1.9-fold to 2.8-fold depending on the gene of interest.

FIG. 2B shows a consistent and robust increase in LV productivity of Expi293F cells when FectoVIR®-AAV is used as transfection reagent in different culture volumes.

FIG. 3 shows the amount of lentivirus obtained using different amount of DNA for transfection. The highest viral production and lowest PP/IP in this experiment was obtained with 0.4 μg DNA/1E6 cells.

FIG. 4 shows the amount of lentivirus obtained with two different lentiviral vectors, where a ˜3-fold increase in productivity was induced by the shift of pH to 6.7 before transfection. A 2.5 L scale bioreactor was used.

FIG. 5 shows the comparative lentiviral productivity using different CAR constructs in two production systems: (i) Expi293F cells using FectoVIR®-AAV as a transfection reagent and (ii) HEK293T cells using PEIpro® as a transfection reagent.

FIG. 6 shows that, in the presence of arginine, the filtration process time was reduced from 244 min to 145 min compared to the control run when samples were subjected to ultrafiltration.

FIG. 7 shows the addition of arginine prior to TFF improved the vector recovery of the subsequent process from about 40% to over 80%.

FIG. 8 shows the vector recovery increased further when arginine spike was implemented prior to both filtration steps.

FIG. 9 shows addition of arginine reduces the particle count and size in a concentration dependent manner.

FIG. 10 is a bar graph showing productivity of infectious LVV (TU/mL—TU assay) and ratio PP/IP (Physical Particles/Infectious Particles) at harvest with different concentrations of benzonase and different times of addition.

FIG. 11 is a bar graph showing quantity of DNA (ng/1E+7 TU) at harvest with different concentrations of benzonase and different times of addition.

FIG. 12 is a bar graph showing quantity of DNA (ng/mL) at harvest with different concentrations of benzonase and different times of addition.

FIG. 13 is a bar graph showing productivity of infectious LVV (TU/mL—TU assay) and ratio PP/IP (Physical Particles/Infectious Particles) at harvest with different incubation times and complexation volumes.

FIG. 14 is a bar graph showing productivity of infectious LVV (TU/mL—TU assay) and ratio PP/IP (Physical Particles/Infectious Particles) at harvest with different incubation times.

FIG. 15 is a bar graph showing cell density and viability at 50 L scale (n=4) during the LVV process.

FIG. 16 is a bar graph showing productivity of infectious LVV (TU/mL—TU assay) at different scales with 2 different products (C1 and I1).

### DETAILED DESCRIPTION

This disclosure is based, at least in part, on a method for producing high titer lentiviral vectors, carrying a transgene of interest and under satisfactory safety conditions. The disclosure also provides at least in part, methods of purification of such lentiviral particle, e.g., from a cell culture. The disclosure also provides a formulation to lentiviral preparations that maintain structural integrity of the viral vector during purification, storage, and gene transfer events ex vivo.

### Definitions

Unless stated otherwise, the following terms and phrases as used herein are intended to have the following meanings.

As used herein, the singular form “a” or “an” includes plural references unless indicated otherwise.

The term “or” is used herein to mean, and is used interchangeably with, the term “and/or” unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The term “amino acid” refers to naturally occurring, synthetic, and unnatural amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

As used herein, the term “buffer” refers to a mixture of a weak acid and its conjugate base or a weak base and its conjugate acid. For instance, as used herein, a “1,4-piperazinediethanesulfonic acid buffer” refers to a mixture that includes 1,4-piperazinediethanesulfonic acid and the 1,4-piperazinediethanesulfonate anion (e.g., sodium 1,4-piperazinediethanesulfonate). Likewise, a “sodium citrate buffer” as used herein refers to a mixture that includes sodium citrate, as well as its conjugate acid, citric acid. Due to the chemical equilibrium that is established between a weak acid and its conjugate base, a solution containing a buffer resists abrupt changes in pH upon the addition of small quantities of acid or base to the solution.

As used herein, the term “binding domain” or “antibody molecule” refers to a protein, e.g., an immunoglobulin chain or fragment thereof, comprising at least one immunoglobulin variable domain sequence. The term “binding domain” or “antibody molecule” encompasses antibodies and antibody fragments. In some embodiments, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In some embodiments, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

The term “antigen binding fragment”, as used herein, refers to one or more portions of an antibody that retain the ability to specifically interact with (e.g., by binding, steric hindrance, stabilizing/destabilizing, spatial distribution) an epitope of an antigen. Examples of binding fragments include, but are not limited to, single-chain Fvs (scFv), camelid antibodies, disulfide-linked Fvs (sdFv), Fab fragments, F(ab′) fragments, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., Nature 341:544-546, 1989), which consists of a VH domain; and an isolated complementarity determining region (CDR), or other epitope-binding fragments of an antibody.

The portion of the CAR described comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). In some aspects, the antigen binding domain of a CAR composition comprises an antibody fragment. In a further aspect, the CAR comprises an antibody fragment that comprises a scFv. The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme), or a combination thereof.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (“scFv”); see, e.g., Bird et al., Science 242:423-426, 1988; and Huston et al., Proc. Natl. Acad. Sci. 85:5879-5883, 1988). Such single chain antibodies are also intended to be encompassed within the term “antigen binding fragment.” These antigen binding fragments are obtained using conventional techniques known to those of skill in the art, and the fragments are screened for utility in the same manner as are intact antibodies.

The term “Chimeric Antigen Receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains, e.g., as provided in an RCAR.

In some aspects, the cytoplasmic signaling domain comprises a primary signaling domain (e.g., a primary signaling domain of CD3-zeta). In some aspects, the cytoplasmic signaling domain further comprises one or more functional signaling domains derived from at least one costimulatory molecule as defined below. In some aspects, the costimulatory molecule is chosen from 41BB (i.e., CD137), CD27, ICOS, and/or CD28. In some aspects, the CAR comprises a chimeric fusion protein comprising an extracellular antigen recognition domain, a transmembrane domain and an intracellular signaling domain comprising a functional signaling domain derived from a stimulatory molecule. In some aspects, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In some aspects, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In some aspects, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In some aspects the CAR comprises an optional leader sequence at the amino-terminus (N-term) of the CAR fusion protein. In some aspects, the CAR further comprises a leader sequence at the N-terminus of the extracellular antigen recognition domain, wherein the leader sequence is optionally cleaved from the antigen recognition domain (e.g., an scFv) during cellular processing and localization of the CAR to the cellular membrane.

A CAR that comprises an antigen binding domain (e.g., an scFv, a single domain antibody, or TCR (e.g., a TCR alpha binding domain or TCR beta binding domain)) that targets a specific tumor marker X, wherein X can be a tumor marker as described herein, is also referred to as XCAR. For example, a CAR that comprises an antigen binding domain that targets BCMA is referred to as BCMA CAR. The CAR can be expressed in any cell, e.g., an immune effector cell as described herein (e.g., a T cell or an NK cell).

The term “signaling domain” refers to the functional portion of a protein which acts by transmitting information within the cell to regulate cellular activity via defined signaling pathways by generating second messengers or functioning as effectors by responding to such messengers. The term “stimulatory molecule,” refers to a molecule expressed by an immune cell (e.g., T cell, NK cell, B cell) that provides the cytoplasmic signaling sequence(s) that regulate activation of the immune cell in a stimulatory way for at least some aspect of the immune cell signaling pathway. In some aspects, the signal is a primary signal that is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immuno-receptor tyrosine-based activation motif or ITAM. Examples of an ITAM containing-cytoplasmic signaling sequence that is of particular use in the invention include, but are not limited to, those derived from CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12. In certain CARs, the intracellular signaling domain in any one or more CARs of the invention comprises an intracellular signaling sequence, e.g., a primary signaling sequence of CD3-zeta. In a specific CAR of the invention, the primary signaling sequence of CD3-zeta is a human sequence, or the equivalent residues from a non-human species, e.g., mouse, rodent, monkey, ape and the like.

An “intracellular signaling domain,” as the term is used herein, refers to an intracellular portion of a molecule. The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines.

In some embodiments, the intracellular signaling domain can comprise a primary intracellular signaling domain. Exemplary primary intracellular signaling domains include those derived from the molecules responsible for primary stimulation, or antigen dependent simulation. In some embodiments, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immuno-receptor tyrosine-based activation motif or ITAM. Examples of ITAM containing primary cytoplasmic signaling sequences include, but are not limited to, those derived from CD3 zeta, common FcR gamma (FCERIG), Fc gamma RIIa, FcR beta (Fc Epsilon Rib), CD3 gamma, CD3 delta, CD3 epsilon, CD79a, CD79b, DAP10, and DAP12.

The term “zeta” or alternatively “zeta chain”, “CD3-zeta” or “TCR-zeta” refers to CD247. Swiss-Prot accession number P20963 provides exemplary human CD3 zeta amino acid sequences. A “zeta stimulatory domain” or alternatively a “CD3-zeta stimulatory domain” or a “TCR-zeta stimulatory domain” refers to a stimulatory domain of CD3-zeta or a variant thereof (e.g., a molecule having mutations, e.g., point mutations, fragments, insertions, or deletions). In some embodiments, the cytoplasmic domain of zeta comprises residues 52 through 164 of GenBank Acc. No. BAG36664.1 or a variant thereof (e.g., a molecule having mutations, e.g., point mutations, fragments, insertions, or deletions). Alternatively or in addition, the term “zeta” or alternatively “zeta chain”, “CD3-zeta” (or “CD3zeta, CD3 zeta or CD3z) or “TCR-zeta” is defined as the protein provided as GenBank Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain, or functional derivatives thereof, that are sufficient to functionally transmit an initial signal necessary for T cell activation. In some aspects, the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof.

The term a “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12 contribute to an efficient immune response. Costimulatory molecules include, but are not limited to, an MHC class I molecule, BTLA and a Toll ligand receptor, as well as OX40, CD27, CD28, CDS, ICAM-1, LFA-1 (CD11a/CD18), ICOS(CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, ICAM-1, lymphocyte function-associated antigen-1 (LFA-1), CD2, CDS, CD7, CD287, LIGHT, NKG2C, NKG2D, SLAMF7, NKp80, NKp30, NKp44, NKp46, CD160, B7-H3, and a ligand that specifically binds with CD83, and the like.

The intracellular signaling domain can comprise the entire intracellular portion, or the entire native intracellular signaling domain, of the molecule from which it is derived, or a functional fragment or derivative thereof.

“Complementarity-determining domains” or “complementary-determining regions (“CDRs”) interchangeably refer to the hypervariable regions of VL and VH. The CDRs are the target protein-binding site of the antibody chains that harbors specificity for such target protein. There are three CDRs (CDR1-3, numbered sequentially from the N-terminus) in each human VL or VH, constituting about 15-20% of the variable domains. The CDRs are structurally complementary to the epitope of the target protein and are thus directly responsible for the binding specificity. The remaining stretches of the VL or VH, the so-called framework regions, exhibit less variation in amino acid sequence (Kuby, Immunology, 4th ed., Chapter 4. W.H. Freeman & Co., New York, 2000).

The positions of the CDRs and framework regions can be determined using various well known definitions in the art, e.g., Kabat, Chothia, international ImMunoGeneTics database (IMGT) (on the worldwide web at www.imgt.org/), and AbM (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); A1-Lazikani et al., J. Mol. Biol., 273:927-748 (1997)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996).

As used herein, the term “contaminating polynucleotide” refers to a polynucleotide not derived from a lentiviral vector. Contaminating polynucleotides may include, e.g., non-lentiviral polynucleotides derived from a cell in which the lentiviral vector was produced, such as chromosomal mammalian DNA (e.g., human DNA) that is not included within a transgene or other component of a lentiviral vector.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that it has the required function, namely, the ability to generate a signal under the appropriate conditions. It does not connotate or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

As used herein, the term “freeze/thaw cycle” refers to exposure of a liquid mixture, such as an aqueous solution or suspension, to a temperature at or less than its freezing point until the mixture is frozen, followed by thawing the mixture at a temperature greater than its freezing point. The freezing step can be performed, e.g., by placing the mixture in an environment in which the temperature is from about −80° C. to about −20° C. The mixture can remain frozen, e.g., for a period of one or more days, weeks, months, or years prior to thawing. The thawing step can be performed by exposing the mixture to conditions in which the temperature is from about 2° C. to about 8° C., or by storing the mixture at room temperature (e.g., the ambient temperature of a laboratory, or about 25° C.). Alternatively, thawing can take place by use of a water bath (e.g., at 37° C.).

As used herein, the term “hydrodynamic radius” refers to the apparent radius (Rh in nm) of a particle in a solution as inferred from the diffusional characteristics of the particle. The hydrodynamic radius of a viral particle is one factor that dictates the rate of diffusion of the viral particle in aqueous solution, as well as the ability of the particle to migrate in gels of macromolecules. The hydrodynamic radius of a viral particle is determined in part by the mass and molecular structure of each of the components of the particle, as well as its hydration state. Methods for determining the hydrodynamic radius of a viral particle are well known in the art and include the use of dynamic light scattering and size exclusion chromatography.

As used herein, the term “non-reducing carbohydrate” refers to a carbohydrate that does not exist in a state of chemical equilibrium with an aldehyde, and thus lacks the ability to be oxidized to a carboxylic acid by transition metal cations, such as silver (Ag+) and copper (Cu2+). Exemplary non-reducing carbohydrates include, without limitation, disaccharides such as sucrose, trehalose, and palatinitol, trisaccharides such as raffinose and melezitose, as well as tetrasaccharides such as stachyose. Non-reducing carbohydrates additionally include monosaccharide derivatives such as sorbitol, mannitol, erythritol, and xylitol, disaccharide derivatives such as lacitol and maltitol, aldonic acids and their lactones such as gluconic acid, gluconic acid γ-lactone, aldaric acids and their lactones such as ribaraic acid, arabinaric acid, and galactaric acid, uronic acids such as glucuronic acid, galaccuronic acid, and itiannuronic acid, ester derivatives such as trehalose octaacetate, sucrose octaacetate, and cellobiose octaacetate, and ether derivatives in which hydroxyl groups are O-alkylated. Non-reducing carbohydrates include those that have a D or L stereochemical orientation.

As used herein, the term “osmolality” refers to a measure of the osmotic pressure of dissolved solute particles in an aqueous solution. The solute particles include both ions as well as non-ionized molecules. Osmolality is expressed as the concentration of osmotically active particles (i.e., osmoles) dissolved in 1 kg of solvent (i.e., water). Osmolality is expressed herein in units of milliosmoles per 1 kg of water (mOsm/kg).

As used herein, the term “percent by weight per volume” or “% w/v” denotes the percentage weight (in grams) of a single component relative to the total volume of the mixture that contains the component. For instance, 500 mg of a component in a total volume of 8 ml is 6.25% w/v, and 500 mg of a component in a total volume of 5 ml is 10% w/v.

As used herein, the term “polydispersity” refers to the degree of homogeneity of the sizes of particles, such as lentiviral particles, within a sample. A higher polydispersity indicates less homogeneity and a lower polydispersity indicates a higher level of homogeneity. For instance, when the level of homogeneity is high, lentiviral particles can be considered to be approaching identical sizes and are thus monodisperse. As will be understood by one of ordinary skill in the art, as the polydispersity decreases, the level of homogeneity increases. As such, a lower polydispersity indicates a higher level of homogeneity. For example, a formulation with 15% polydispersity has less homogeneity than a formulation with 10% polydispersity. When the level of homogeneity is low, the particle population can be considered to contain significantly different sizes and thus be polydisperse.

As used herein, the term “prevent”, “preventing,” or “prevention” of any disease or disorder refers to the prophylactic treatment of the disease or disorder; or delaying the onset or progression of the disease or disorder.

The term “recognize” as used herein refers to an antibody or antigen binding fragment thereof that finds and interacts (e.g., binds) with its epitope, whether that epitope is linear or conformational. The term “epitope” refers to a site on an antigen to which an antibody or antigen binding fragment of the disclosure specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include techniques in the art, for example, x-ray crystallography and 2-dimensional nuclear magnetic resonance (see, e.g., Epitope Mapping Protocols in Methods in Molecular Biology, Vol. 66, G. E. Morris, Ed. (1996)).

As used herein, the term “retroviral packaging protein” refers to a protein derived from a retrovirus, or a variant thereof, that assists with packaging of a nucleic acid (e.g., a viral genome) into an envelope. Exemplary retroviral packaging proteins include gag, pol, and rev, e.g., lentiviral gag, pol, and rev, e.g., the wild-type proteins or variant thereof, e.g., sequences having at least 80%, 90%, or 95% sequence identity thereto. In some embodiments, one or more retroviral packaging protein is provided as a polyprotein.

As used herein, the term “retroviral envelope protein” refers to a protein derived from a retrovirus, or a variant thereof, that can be assembled into an envelope around a nucleic acid (e.g., a viral genome). An exemplary retroviral envelope protein is env, e.g., wild-type or a variant thereof. In some embodiments, the retroviral envelope protein is a lentiviral envelope protein, e.g., wild-type or a variant thereof. In some embodiments, the retroviral envelope protein is VSV-G, e.g., wild-type or variant thereof. In some embodiments, the retroviral envelop protein is pseudotyped. In some embodiments, the retroviral envelope protein is from a different virus than one or more of the retroviral packaging protein or LTRs of the nucleic acid to be packaged.

As used herein, the phrases “specifically binds” and “binds” refer to a binding reaction which is determinative of the presence of a particular protein in a heterogeneous population of proteins and other biological molecules that is recognized, e.g., by a ligand with particularity. A ligand (e.g., a protein, proteoglycan, or glycosaminoglycan) that specifically binds to a protein will bind to the protein with a KD of less than 500 nM. For example, a ligand that specifically binds to a protein will bind to the protein with a KD of up to 500 nM (e.g., between 1 pM and 500 nM). A ligand that does not exhibit specific binding to a protein or a domain thereof will exhibit a KD of greater than 500 nM (e.g., greater than 600 nm, 700 nM, 800 nM, 900 nM, 1 μM, 100 μM, 500 μM, or 1 mM) for that particular protein or domain thereof. A variety of assay formats may be used to determine the affinity of a ligand for a specific protein. For example, solid-phase ELISA assays are routinely used to identify ligands that specifically bind a target protein. See, e.g., Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of assay formats and conditions that can be used to determine specific protein binding.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The term “therapeutic effector”, as used herein, refers to a molecule (e.g., an RNA or polypeptide) that, at an effective level, can exert a therapeutic effect on a subject.

The term “therapeutically acceptable amount” or “therapeutically effective dose” interchangeably refers to an amount sufficient to effect the desired result (i.e., a reduction in tumor size, inhibition of tumor growth, prevention of metastasis, inhibition or prevention of viral, bacterial, fungal or parasitic infection). In some embodiments, a therapeutically acceptable amount does not induce or cause undesirable side effects. In some embodiments, a therapeutically acceptable amount induces or causes side effects but only those that are acceptable by the healthcare providers in view of a patient's condition. A therapeutically acceptable amount can be determined by first administering a low dose, and then incrementally increasing that dose until the desired effect is achieved. A “prophylactically effective dosage,” and a “therapeutically effective dosage,” can, in some embodiments, prevent the onset of, or result in a decrease in severity of, respectively, disease symptoms, including symptoms associated with cancer.

The term “transfection” as used herein refers to the introduction of DNA into a eukaryotic cell. Transfection may be accomplished by a variety of means including but not limited to calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

As used herein, the terms “treat,” “treating,” or “treatment” of any disease or disorder refers in one embodiment, to ameliorating the disease or disorder (i.e., slowing or arresting or reducing the development of the disease or at least one of the clinical symptoms thereof). In another embodiment, “treat,” “treating,” or “treatment” refers to alleviating or ameliorating at least one physical parameter including those which may not be discernible by the patient. In yet another embodiment, “treat,” “treating,” or “treatment” refers to modulating the disease or disorder, either physically, (e.g., stabilization of a discernible symptom), physiologically, (e.g., stabilization of a physical parameter), or both.

As used herein, the term “viral titer” refers to the number of infectious vector particles, or “transducing units,” that result in the transfer of a given nucleic acid sequence from the particles into a target cell. Viral titer can be measured by a functional assay, such as an assay described in Xiao et al., Exp. Neurobiol. 144:1 13-124, 1997, or Fisher et al., J. Virol. 70:520-532, 1996, the disclosures of both of which are incorporated by reference in their entirety. Alternatively, viral titer can be measured by determining the quantity of viral DNA that has integrated into a host cell genome, e.g., using polymerase chain reaction (PCR) techniques known in the art.

As used herein, the term “viral vector” refers to a viral particle which has a capability of introducing a nucleic acid molecule into a host. A viral vector carrying an exogenous gene(s) is typically packaged into an infectious virus particle via virus packaging with the aid of packaging plasmids using specific cell-lines. The infectious virus particle infects a cell to achieve expression of the exogenous gene. A “recombinant” viral vector refers to a viral vector constructed by gene recombinant technologies. A recombinant viral vector can be constructed using any suitable method, such as by transducing or transfecting a packaging cell-line with a nucleic acid encoding the viral genome and subsequently isolating newly packaged viral particles. It is understood that the recombinant technologies may be performed at a stage upstream of production of the viral vector itself. For example, recombinant technologies may be used to produce a plasmid, and the plasmid may then be produced at a larger scale, and finally the plasmid may be introduced into a cell line for packaging to produce the viral vector.

The term “lentiviral vector” refers to a vector derived from at least a portion of a lentivirus genome, including, for example, a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

### Methods for Producing Lentivirus

This disclosure provides, inter alia, improved methods for manufacturing lentiviral vectors. The following general steps may be used. First, host cells can be cultured. Exemplary types of host cells, such as human cells lacking the large T antigen, are described in more detail in the section entitled “Host cells” herein. As described in Example 1 herein, host cells lacking the large T antigen can lead to manufacturing advantages compared to host cells comprising the large T antigen.

In some embodiments, in order to produce large quantities of the cells, the host cells are cultured in sequentially larger vessels (e.g., bioreactors) until sufficiently large numbers of cells are produced.

Once sufficient numbers of host cells are obtained, the desired nucleic acids can be introduced into the host cells. The nucleic acids may be introduced by transfection, e.g., using the FectoVIR®-AAV transfection reagent, e.g., as described in the section entitled “Transfection” herein. Benefits of FectoVIR®-AAV transfection reagent are described in Examples 2 and 3 herein. The transfected nucleic acids may include a viral genome to be packaged, wherein the viral genome includes a therapeutic gene of interest and sufficient LTR sequence for packaging into a viral particle. Additional nucleic acids that may be introduced into the host cell include plasmids that promote packaging, e.g., plasmids encoding viral gag, pol, env, and rev. In some embodiments, the pH of the culture medium may be shifted downwards before transfection, e.g., from about 7.1 to about 6.7, e.g., as described in the section herein entitled “Culture conditions and transfection conditions” and in Example 4 herein. The cells then begin to produce lentivirus.

After transfection, a nuclease such as benzonase may be added to the culture media, e.g., as described in the section entitled “Culture media” and in Example 5 herein. Without wishing to be bound by theory, in some embodiments, the cell culture medium is a source of contaminating nucleic acids to the final lentiviral preparation, e.g., the culture medium may contain host cell DNA from lysed host cells. Accordingly, addition of benzonase to the cell culture medium may degrade the contaminating nucleic acids, allowing for improved purification of the lentivirus.

Next, lentivirus can be harvested from the host cell culture to begin purification of the lentivirus. In some embodiments, harvesting of lentivirus comprises separating the supernatant or cell culture media from the cell. In some embodiments, the cell is not lysed before clarification. In some embodiments, the cells may be lysed, and the lysate may be clarified.

Purification of the lentivirus from the cell culture media or cell lysate typically involves several sequential purification steps. Purification steps may include filtration (e.g., ultrafiltration) and chromatography steps. In some embodiments, arginine can be added during the purification process, e.g., before or after a filtration step or a chromatography step. Addition of arginine is described, e.g., in the section entitled “Purification” and in Examples 8-12 herein. Without wishing to be bound by theory, in some embodiments, the arginine stabilizes the lentiviral vectors and/or reduces their aggregation.

The purified lentivirus can be used for a variety of applications. For example, the lentivirus can be used to deliver a gene to cells ex vivo, e.g., to generate CART cells from immune effector cells from an apheresis sample. As another example, the lentivirus may be administered to a subject, to deliver a gene to cells of the subject in situ. For instance, the lentivirus may be used for in vivo CART. In some embodiments, the lentivirus is suitable for administration in a human subject, e.g., a lentivirus encoding a CAR maybe administered to a subject allowing for introduction of the CAR encoding nucleic acid into immune effector cells in the subject's body.

Naturally occurring lentiviruses are a genus of viruses of the Retroviridae family, characterized by a long incubation period. Lentiviruses can typically deliver a significant amount of genetic information into the DNA of the host cell. Examples of lentiviruses include HIV (human immunodeficiency virus; including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates. Diseases caused by these viruses are characterized by a long incubation period and protracted course. Usually, the viruses latently infect monocytes and macrophages, from which they spread to other cells. HIV, FIV, and SIV also readily infect T lymphocytes (i.e., T-cells).

### Transgene

In some embodiments, the lentivirus or lentiviral vector disclosed herein, may include a nucleic acid, e.g., a transgene, such as a protein-encoding transgene. The nucleic acid may comprise a transgene, e.g., as described in the section herein entitled “Transgene”. The transgene may be operably linked to a promoter sequence. The nucleic acid may also comprise one or more (e.g., two) LTR sequences. Without wishing to be bound by theory, the LTRs may promote insertion of the transgene and promoter into a host cell genome. The LTR sequences may comprise wild-type lentiviral LTR sequences or variants thereof. For instance, the 3′ LTR may comprise a deletion that renders the virus self-inactivating after integration. In addition, the 5′ LTR may be a chimeric LTR. In some embodiments, the transgene can be integrated into the chromosomal DNA of a target cell.

Exemplary transgenes include those that encode a chimeric antigen receptor (CAR). The CAR may include several domains, such as an antigen binding domain, a transmembrane domain, and one or more signaling domains. In these cases, the signaling domains may contain one or more primary signaling domains (such as a CD3-zeta stimulatory domain) and/or one or more costimulatory signaling domains (such as CD27, CD28, 4-1 BB (CD137), OX40, GITR, CD30, CD40, ICOS, BAFFR, HVEM, ICAM-1, lymphocyte function-associated antigen-1 (LFA-1), CD2, CDS, CD7, CD287, LIGHT, NKG2C, NKG2D, SLAMF7, NKp80, NKp30, NKp44, NKp46, CD160, B7-H3, or a ligand that specifically binds with CD83.

In some embodiments, the transgene, e.g., a transgene including a CAR, may encode an antigen-binding domain (such as a scFv) that binds a particular target protein or carbohydrate. Exemplary antigens include CD19, CD123, CD22, CD30, CD171, CS-1, C-type lectin-like molecule-1, CD33, epidermal growth factor receptor variant III (EGFRvlll), ganglioside G2 (GD2), ganglioside GD3, TNF receptor family member B cell maturation (BCMA), Tn antigen ((Tn Ag) or (GalNAca-Ser/Thr)), prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG 72), CD38, CD44v6, Carcinoembryonic antigen (CEA), Epithelial cell adhesion molecule (EPCAM), B7H3 (CD276), KIT (CD1 17), lnterleukin-13 receptor subunit alpha-2, mesothelin, Interleukin 1 1 receptor alpha (IL-1 1 Ra), prostate stem cell antigen (PSCA), Protease Serine 21, vascular endothelial growth factor receptor 2 (VEGFR2), Lewis(Y) antigen, CD24, Platelet-derived growth factor receptor beta (PDGFR-beta), Stage-specific embryonic antigen-4 (SSEA-4), CD20, Folate receptor alpha, Receptor tyrosine-protein kinase ERBB2 (Her2/neu), Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit, Beta Type, 9 (LMP2), glycoprotein 1 00 (gp100), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRC5D), chromosome X open reading frame 61 (CXORF61), CD97, CD179a, anaplastic lymphoma kinase (ALK), Polysialic acid, placenta-specific 1 (PLAC1), hexasaccharide portion of globoH glycoceramide (GloboH), mammary gland differentiation antigen (NY-BR-1), uroplakin 2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 complex, locus K 9 (LY6K), Olfactory receptor 51 E2 (OR51 E2), TCR Gamma Alternate Reading Frame Protein (TARP), Wilms tumor protein (WT1), Cancer/testis antigen 1 (NY-ESO-1), Cancer/testis antigen 2 (LAGE-1 a), Melanoma-associated antigen 1 (MAGE-A1), ETS translocation-variant gene 6, located on chromosome 12p (ETV6-AML), sperm protein 17 (SPA17), X Antigen Family, Member 1 A (XAGE1), angiopoietin-binding cell surface receptor 2 (Tie 2), melanoma cancer testis antigen-1 (MAD-CT-1), melanoma cancer testis antigen-2 (MAD-CT-2), Fos-related antigen 1, tumor protein p53 (p53), p53 mutant, prostein, surviving, telomerase, prostate carcinoma tumor antigen-1, melanoma antigen recognized by T cells 1, Rat sarcoma (Ras) mutant, human Telomerase reverse transcriptase (hTERT), sarcoma translocation breakpoints, melanoma inhibitor of apoptosis (ML-IAP), ERG (transmembrane protease, serine 2 (TMPRSS2) ETS fusion gene), N-Acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), Tyrosinase-related protein 2 (TRP-2), Cytochrome P450 1 B1 (CYP1 B1), CCCTC-Binding Factor (Zinc Finger Protein)-Like, Squamous Cell Carcinoma Antigen Recognized By T Cells 3 (SART3), Paired box protein Pax-5 (PAX5), proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), renal ubiquitous 2 (RU2), legumain, human papilloma virus E6 (HPV E6), human papilloma virus E7 (HPV E7), intestinal carboxyl esterase, heat shock protein 70-2 mutated (mut hsp70-2), CD79a, CD79b, CD72, Leukocyte-associated immunoglobulin-like receptor 1 (LAIR1), Fc fragment of IgA receptor (FCAR or CD89), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), and immunoglobulin lambda-like polypeptide 1 (IGLL1).

In some embodiments, a lentiviral vector described herein comprises more than one transgene, e.g., a first transgene encoding a first CAR, e.g., a CD19 CAR and a second transgene encoding a second CAR, e.g., a CD22 CAR.

In some embodiments, a dual CAR lentiviral vector described herein encodes two different CARs, e.g., a CD19 CAR and a CD22 CAR. In some embodiments, the two CARs are part of a single open reading frame and are separated by a protease cleavage site, e.g., a self-cleavage site, e.g., a P2A site. In some embodiments, the open reading frame encodes, from N-terminal to C-terminal, a first leader sequence, a first scFv (e.g., that binds CD22), optionally a first hinge domain, a first transmembrane domain, a first costimulatory domain (e.g., 4-1BB), a first primary signaling domain (e.g., CD3-zeta), a protease cleavage site (e.g., P2A), a second leader sequence, a second scFv (e.g., that binds CD19), optionally a second hinge domain, a second transmembrane domain, a second costimulatory domain (e.g., 4-1BB), and a second primary signaling domain (e.g., CD3-zeta). In some embodiments, the first and second leader sequences have the same sequence. In some embodiments, the first and second hinge domains have the same sequence. In some embodiments, the first and second transmembrane domains have the same sequence. In some embodiments, the first and second costimulatory domains have the same sequence. In some embodiments, the first and second primary signaling domains have the same sequence.

Additional CARs that can be encoded by transgene described herein are provided, e.g., in the section herein entitled “CAR targets”.

In some embodiments, a lentiviral vector described herein encodes a siRNA or shRNA that targets a nucleic acid in an immune effector cell. For instance, the siRNA or shRNA may target a nucleic acid encoding a TCR and/or HLA, and/or an inhibitory molecule (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a T cell. Expression systems for siRNA and shRNAs, and exemplary shRNAs, are described, e.g., in paragraphs 649 and 650 of International Application WO2015/142675, filed Mar. 13, 2015, which is incorporated by reference in its entirety. These nucleic acids can also be targeted, for example, using a CRISPER system, Zinc finger nucleases, or TALENs. The immune effector cell may be autologous or allogeneic to the subject to be treated.

In some embodiments, a lentiviral vector described herein comprises or encodes one or more inhibitor of a methylcytosine dioxygenase gene (e.g., Tet1, Tet2, or Tet3). Uses of such compositions and methods for increasing the functional activities of engineered cells (e.g., gene-modified antigen-specific T cells, such as CAR T cells) are also contemplated. While not to be bound by the theory, disruption of a single allele of a Tet gene (e.g., a Tet1, Tet2, or Tet3) leads to decreased total levels of 5-hydroxymethylcytosine in association with enhanced proliferation, regulation of effector cytokine production and degranulation, and thereby increases CAR T cell proliferation and/or function. In some embodiments, the expression and/or function of Tet2 in said cell has been reduced or eliminated.

In some embodiments, the inhibitor of Tet1, Tet2 and/or Tet3, is an siRNA or shRNA specific for Tet1, Tet2, Tet3, or nucleic acid encoding said siRNA or shRNA. In some embodiments, the siRNA or shRNA comprises a sequence complementary to a sequence of a Tet2 mRNA, e.g., comprises a target sequence of shRNA listed in Table 4 of WO2017/049166, which application is herein incorporated by reference in its entirety, including Table 4. In some embodiments, the inhibitor of Tet1, Tet2 and/or Tet3, is (1) a gene editing system targeted to one or more sites within the gene encoding Tet1, Tet2 and/or Tet3, or its regulatory elements, e.g., Tet2, or its regulatory elements; (2) nucleic acid encoding one or more components of said gene editing system; or (3) combinations thereof. In some embodiments, the gene editing system is selected from the group consisting of: a CRISPR/Cas9 system, a zinc finger nuclease system, a TALEN system and a meganuclease system.

In some embodiments, a lentiviral vector described here comprises a transgene, e.g., a transgene encoding a chimeric antigen receptor (CAR) and further comprises a siRNA or shRNA that targets a nucleic acid in an immune effector cell.

### Characteristics of Lentiviral Vectors

In some embodiments, the lentiviral vectors are characterized by a hydrodynamic radius of 100±25 nm as measured by dynamic light scattering (DLS). For example, the lentiviral vectors may maintain a hydrodynamic radius of 100±25 nm within a temperature range of from 25° C. to 55° C.

In some embodiments, the lentiviral vectors are characterized by a polydispersity of from 10% to 25%. For example, the lentiviral vectors may maintain a polydispersity of from 10% to 25% within a temperature range of from 25° C. to 55° C.

In some embodiments, the lentiviral vectors maintains a concentration after 3, 6, or 9 freeze/thaw cycles of from about 70% to about 100% relative to the concentration of the lentiviral vector in the aqueous composition prior to the freeze/thaw cycles, wherein each of the freeze/thaw cycles includes freezing the aqueous composition and subsequently allowing the aqueous composition to thaw at room temperature.

In some embodiments, a lentivirus prepared, purified or stored using any of the methods or formulations disclosed herein may have lower vector copy number (VCN), e.g., at least 5%, at least 10%, at least 20%, at least 30%, at least 50%, at least 60% lower VCN compared to a lentivirus not produced, purified or stored by the methods or in formulations as described herein, e.g., when tested at MOI of 1.

### Lentiviral Vector Packaging System

A packaging system can be used to package a nucleic acid, e.g., an RNA encoding a transgene into a lentiviral vector. Accordingly, the systems and methods described herein may comprise, e.g., a lentiviral packaging system comprising at least one plasmid adapted for the production of a lentiviral vector, e.g., a lentiviral vector optionally comprising a transgene. Various lentiviral components useful for the production of a lentiviral vector are known in the art. See for example Zufferey et al., 1997, Nat. Biotechnol. 15:871-875 and Dull et al, 1998, J. Virol. 72(11):8463-8471. The different functions suitable for the production of a lentiviral vector can be provided to the host cells in a lentiviral packaging system comprising one or more nucleic acids (e.g., plasmids), e.g., at least one, two, three, or four plasmids, wherein one plasmid encodes a retroviral envelope protein (Env plasmid), one plasmid encodes one or more retroviral packaging proteins, e.g., Gag and Pol proteins (packaging plasmid or Gag-Pol plasmid), one plasmid encodes a lentiviral Rev protein (Rev plasmid) and one or more plasmids comprising at least one transgene of interest (TOI) expression cassette. In some embodiments, the lentiviral packaging system further comprises, or a method described herein comprises use of, at least one, two, three, or four plasmids. In some embodiments, the lentiviral packaging system further comprises, or a method described herein comprises use of, a fifth plasmid. In certain embodiments, a method described herein comprises transfecting five plasmids into the host cell, wherein the fifth plasmid does not encode a protein of the lentiviral vector packaging system. In some embodiments, the lentiviral packaging system comprises one or more nucleic acids (e.g., plasmids), e.g., five plasmids, wherein one plasmid encodes an expression vector, one plasmid encodes a Tat (e.g., pcDNATat), one plasmid encodes a Rev protein (e.g., pHCMV-Rev), one plasmid encodes a gagpol (e.g., pHCMV-gagpol), and one plasmid encodes VSV-G (e.g., pVSVG), e.g., as described in Rout-Pitt et al., J Biol. Methods 5(2): 1-9, 2018). In some embodiments, a plasmid may comprise a dual gene expression cassette, e.g., a bicistronic cassette, e.g., a bicistronic construct encoding two transgenes of interest. In some embodiments, the first transgene of interest encodes a first CAR, e.g., a CD19 CAR, and the second transgene of interest encodes a second CAR, e.g., a CD22 CAR. In some embodiments the retroviral packaging proteins are derived from a lentivirus, e.g., lentiviral packaging proteins, e.g., lentiviral gag and pol proteins. In some embodiments, the lentiviral gag protein is a wild-type lentiviral gag protein, and in other embodiments it has one or more sequence modifications relative to the wild-type sequence. In some embodiments, the lentiviral pol protein is a wild-type lentiviral pol protein, and in other embodiments it has one or more sequence modifications relative to the wild-type sequence. In some embodiments, the rev protein is a wild-type rev protein, and in other embodiments it has one or more sequence modifications relative to the wild-type sequence. In some embodiments, the lentiviral vector may be a pseudotyped vector, comprising a modified envelope protein, e.g., an envelope protein derived from a different virus or a chimeric envelope protein, e.g., the Env plasmid may encode a VSV-G Env protein, e.g., a wild type VSV-G protein or a modified variant.

In some embodiments, a lentiviral vector is generated using a packaging system comprising pMDLgpRRE, pRSV-Rev and pMD.G plasmids (Dull et al., supra), but using a kanamycin resistance marker, e.g., a marker that confers resistance to both kanamycin and neomycin, e.g., neomycin phosphotransferase II instead of an ampicillin gene.

In some embodiments, a system described herein comprises a transfer vector comprising a kanamycin resistance marker, e.g., a marker that confers resistance to both kanamycin and neomycin, e.g., neomycin phosphotransferase II, e.g., instead of an ampicillin gene. In some embodiments, the transfer vector comprises sequence from, e.g., a pELPS construct as disclosed in WO2017087861A or Milone et al., Mol. Ther. 17(8):1453-1464, 2009, each of which is incorporated by reference herein in its entirety. In some embodiments, the therapeutic protein is encoded on a self-inactivating transfer vector that comprises one or more of, e.g., all of, lentiviral 5′ LTR (e.g., a truncated lentiviral 5′ LTR), lentiviral 3′ LTR, cPPT, and WPRE. In some embodiments, the transfer vector lacks one or more of, e.g., all of: a promoter active in bacteria (e.g., lacking all of a T7 promoter, a T3 promoter, and a lac promoter), M13 primer binding site (e.g., lacking both an M13 forward primer binding site and an M13 reverse primer binding site), a phage origin (e.g., f1 ori), and a fluorescent protein-encoding gene (e.g., a GFP, e.g., EGFP). In some embodiments, the transfer vector lacks both of a CAP binding site and lac operator. In some embodiments, the transfer vector comprises pELPS construct as disclosed in WO2017087861, except that the transfer vector lacks a T7 promoter, an M13 forward primer binding site, an f1 ori, a CAP binding site, an IPTG inducible promoter, a lac operator, an M13 reverse primer binding site, a T3 promoter, and EGFP wherein optionally the transfer vector encodes a therapeutic protein, e.g., a CAR. In some embodiments, the transfer vector has one or more of the following properties: (a) is more stable than an otherwise similar control transfer vector, (b) results in lower cell toxicity than an otherwise similar control transfer vector, or (c) results in a lower vector copy number (VCN) when integrated into target cells, e.g., as described herein. In some embodiments, the control transfer vector comprises a T7 promoter, an M13 forward primer binding site, an f1 ori, a CAP binding site, an IPTG inducible promoter, a lac operator, an M13 reverse primer binding site, and a T3 promoter.

In some embodiments, the gene expression cassette encodes a protein, e.g., a chimeric antigen receptor (CAR). In some embodiments, the gene expression cassette encodes two proteins, e.g., a first CAR and a second CAR. Exemplary transgenes suitable for a gene expression cassette are described in the current disclosure.

### Transfection

In some embodiments, the different functions for production of a lentiviral vector are provided to a plurality of host cells, e.g., mammalian cells, e.g., HEK293 cells, e.g., Expi293F cells (e.g., plurality of Expi293F cells growing in suspension under serum-free conditions) by transfection, e.g., transient or stable transfection, of a lentiviral packaging system adapted for producing lentiviral vectors. In some embodiments, at least 25%, at least 30%, at least 35%, at least 40%, at least 50%, at least 60%, at least 70%, at least 80%, at least 90%, at least 95% of host cells, e.g., HEK293 cells, e.g., Expi293F cells are transfected. Methods for transfection or infection are well known by those of skill in the art. In some embodiments, at least 0.3 μg, at least 0.4 μg, at least 0.5 μg, at least 0.6 μg, at least 0.7 μg, at least 0.8 μg cells, at least 0.9 μg, or at least 1.0 μg of lentiviral packaging system is provided per million cells for transfection. In some embodiments, a transfection reagent is used for transfecting the host cells, e.g., mammalian cells, e.g., HEK293 cells, e.g., Expi293F cells. In some embodiments, a transfection reagent is used. Transfection reagents are well known in the art and are available from commercial suppliers. Examples of transfection reagents include but are not limited to, Lipofectamine™ (Invitrogen), Polifectamine, LentiTran (Origene), PEIpro® (Polyplus), FectoVIR®-AAV (Polyplus), and ProFection® (Promega). In some embodiments, the transfection reagent, e.g., FectoVIR®-AAV is used at a level of 0.1 μl, 02. μl, 0.3 μl, 0.4 μl, 0.5 μl, 0.6 μl, 0.7 μl, 0.8 μl, 0.9 μl, or 1.0 μl per million cells. In some embodiments the packaging system and the transfection reagent, e.g., FectoVIR®-AAV are used at ratio of about 1:0.5, 1:0.75, 1:1, 1:1.5, or 1:2, or any range therebetween, for transfection.

In some embodiments, the transfection reagent comprises FectoVIR®-AAV transfection reagent. FectoVIR®-AAV can be obtained, e.g., from Polyplus (850 bd Sebastien Brant, 67400 Illkirch, FRANCE; 1251 Ave of the Americas; 3rd Fl, New York; NY 10020 USA). FectoVIR®-AAV is a chemical-based, animal-free transfection reagent.

In some embodiments, at the time of transfection the cells (e.g., Expi293F cells) are at a density of about 0.5×10⁶ cells/mL-1×10⁷ cells/mL, 1×10⁶ cells/mL-6×10⁶ cells/mL, 1×10⁶ cells/mL-5×10⁶ cells/mL, 1.50×10⁶ cells/mL-2.50×10⁶ cells/mL, 2.0×10⁶ cells/mL-3.0×10⁶ cells/mL, 2.0×10⁶ cells/mL-2.5×10⁶ cells/mL. In some embodiments, at the time of transfection the cell population has a viability of at least about 80%, 90%, or 95%.

In some embodiments, the PP/IP (physical particle/infectious particle) ratio is less than 500, 700, 900, 1000, 1100, 1200, 1300, 1400, 1500, 1600, 1700, 1800, 1900, 2000 after transfection.

### Host Cells

Any host cells suitable for expression of viral vectors, e.g., lentiviral vectors e.g., lentiviral vectors as disclosed herein may be used to carry out the methods disclosed herein. In some embodiments, a suitable host cell is a eukaryotic cell, e.g., a mammalian cell. In some embodiments, the mammalian cells may be genetically modified mammalian cells for expressing a virus, e.g., a lentivirus, e.g., a lentiviral vector or a lentivirus of interest. A number of mammalian cell lines are suitable host cells for recombinant expression of viruses. Mammalian host cell lines include, for example, COS, PER.C6, TM4, VERO, MDCK, BRL-3A, W138, Hep G2, MMT, MRC 5, FS4, CHO, 293T, A431, 3T3, CV-1, C3H10T1/2, Colo205, HEK293, HeLa, L cells, BHK, HL-60, FRhL-2, U937, HaK, Jurkat cells, Rat2, BaF3, 32D, FDCP-1, PC12, Mix, murine myelomas (e.g., SP2/0 and NSO) and C2C12 cells, as well as transformed primate cell lines, hybridomas, normal diploid cells, and cell strains derived from in vitro culture of primary tissue and primary explants. In some embodiments the host cell is a HEK293 cell, including a cell derived from HEK293 cells, e.g., 293F cells, e.g., Expi293F cells. In some embodiments, at least 80%, at least 85%, at least 90%, at least 90%, at least 95% of host cells in a culture express a large T antigen, e.g., a polyomaviral large T antigen, e.g., a SV40 large T antigen, e.g., a mutant SV40 large T antigen. In some embodiments, at least 99%, at least 98%, at least 97%, at least 96%, at least 95% of the host cells in a culture do not express a large T cell antigen. In some embodiments, the host cell is suitable for growing in suspension.

### Culture Process

The cell lines described herein can be cultured under conditions that allow for the production of lentiviral vector particles, with high titer. Eukaryotic cells, e.g., mammalian cells, e.g., HEK293 cells, e.g., Expi293F cells may be cultured as non-anchorage dependent cells growing freely in suspension throughout the bulk of the culture; or as anchorage-dependent cells requiring attachment to a solid substrate for their propagation (e.g., as a monolayer).

In some embodiments, a microcarrier system may be used to accommodate cell growth. In some embodiments the microcarrier system may comprise a suspension culture, e.g., a large-scale suspension culture. The suspension culture may be operated in open or closed systems, e.g., batch or fed-batch closed systems. In some embodiments, nutrients are not added, and waste products are not removed through the duration of culture. In some embodiments, nutrients are continuously fed into the system to prolong the growth cycle although cells, products, by products, and waste products, including toxic metabolites, are not removed. In some embodiments, the culture system may be an open, e.g., a continuous system, e.g., a perfusion system or a chemostat system. In some embodiments, the system may comprise one or more cell retention device. Cell retention devices may include, for example, microcarriers, fine mesh spin filters, hollow fibers, flat plate membrane filters, settling tubes, ultrasonic cell retention devices, and the like. In some embodiments, the concentration of cells in the bioreactor are higher than the concentration of cells present the supernatant harvested from the bioreactor. In some embodiments, the concentration of cells in the bioreactor are substantially identical than the supernatant harvested from the bioreactor.

In continuous fermentation process a defined media often is continuously added to a bioreactor while an equal amount of culture volume is removed simultaneously for product recovery. Continuous cultures generally maintain cells in the log phase of growth at a constant cell density. Continuous or semi-continuous culture methods permit the modulation of one factor or any number of factors that affect cell growth or end product concentration. For example, an approach may limit the carbon source and allow all other parameters to moderate metabolism. In some systems, a number of factors affecting growth may be altered continuously while the cell concentration, measured by media turbidity, is kept constant. Continuous systems often maintain steady state growth and thus the cell growth rate often is balanced against cell loss due to media being drawn off the culture. Methods of modulating nutrients and growth factors for continuous culture processes are known and a variety of methods are known in the art.

In some embodiments, a culture of suspension cells comprises only cells that are in suspension. In some embodiments, a culture of suspension cells may comprise a small number (e.g., less than 1%) of cells that adhere, e.g., transiently, to a surface.

“Cell culture” may refer to any in vitro culture of cells. Included within this term are continuous cell lines (e.g., with an immortal phenotype), primary cell cultures, finite cell lines (e.g., non-transformed cells), and any other cell population maintained in vitro.

In some embodiments, a system or method described herein makes uses of packaging cells or a packaging cell line for production of a viral vector. The cell line may be stably transfected with elements for production of the lentiviral vector, for example retroviral packaging proteins and retroviral envelope protein. Typically, such packaging cells contain one or more expression cassettes which are capable of expressing viral proteins (such as gag, pol and env) but the expression cassettes do not contain a packaging signal. A packaging cell may be a cell cultured in vitro. A packaging cell line may be utilized to create producer cell lines for production of the lentiviral particles, e.g., by providing at least one plasmid comprising at least one transgene of interest (TOI) expression cassette. In some embodiments, a producer cell transiently expresses a plasmid (e.g., a transfer plasmid) encoding a therapeutic effector and comprising sufficient LTR sequence to allow for packaging of RNA comprising the LTR(s) into a viral vector. In some embodiments, a producer cell line stably expresses an expression cassette encoding a therapeutic effector and comprising sufficient LTR sequence to allow for packaging of RNA comprising the LTR(s) into a viral vector.

### Culture Media

The methods of the current disclosure may be carried out using any media suitable (e.g., supports cell growth and maintenance under the conditions of the current disclosure) for culturing eukaryotic cells, e.g., mammalian cells, e.g., HEK293 cells, e.g., Expi293F cells. The terms “cell culture medium” and “culture medium” (or simply “medium”) refer to a nutrient solution used for growing eukaryote cells e.g., mammalian cells, e.g., HEK293 cells, e.g., Expi293F cells, that typically provides at least one component from one or more of the following categories: (1) salts (e.g., sodium, potassium, magnesium, calcium, etc.) contributing to the osmolality of the medium; (2) an energy source, usually in the form of a carbohydrate such as glucose; (3) all essential amino acids, and usually the basic set of twenty amino acids; (4) vitamins and/or other organic compounds required at low concentrations; and (5) trace elements, where trace elements are defined as inorganic compounds that are typically required at very low concentrations, usually in the micromolar range. Compositions of such media are known in the art (see, e.g., Mather, J. P., et al. (1999) “Culture media, animal cells, large scale production,” Encyclopedia of Bioprocess Technology: Fermentation, Biocatalysis, and Bioseparation, Vol. 2:777-785, hereby incorporated herein by reference in their entirety.) The nutrient solution may optionally be supplemented with one or more of the components from any of the following categories: (a) animal serum; (b) hormones and other growth factors such as, for example, insulin, transferrin, and epidermal growth factor; and (c) hydrolysates of plant, yeast, and/or tissues, including protein hydrolysates thereof.

In some embodiments, the culture media may comprise serum, e.g., fetal bovine serum (FBS). In some embodiments, the culture media is serum free. In some embodiments, the culture media is chemically defined, e.g., medium lacking animal-derived components. As used herein, “animal-derived” components are any components that are produced in an intact animal (such as, e.g., proteins isolated and purified from serum), or produced using components produced in an intact animal (such as, e.g., an amino acid made by using an enzyme isolated and purified from an animal to hydrolyze a plant source material). By contrast, a protein which has the sequence of an animal protein (i.e., has a genomic origin in an animal) but which is produced in vitro in cell culture (such as, e.g., in a recombinant yeast or bacterial cells or in an established continuous eukaryote cell line, recombinant or not), using media lacking components produced in, or isolated and purified from, an intact animal is not an “animal-derived” component.

Chemically defined media are media in which all components have a known chemical structure. Chemically-defined medium are available from commercial suppliers, such as, for example, Sigma, ThermoFisher, Invitrogen, JRH Biosciences, and Gibco. In some embodiments, the media is FreeStyle™ 293 Expression Medium. In some embodiments, a concentrated serum may be used, e.g., medium that contains higher concentration of nutrients than is normally necessary and normally provided to a growing culture. In some embodiments, the medium may contain an amino acid(s) derived from any source or method known in the art.

In some embodiments, an enzyme, e.g., a nuclease, e.g., an endonuclease, e.g., a recombinant endonuclease, e.g., a Benzonase® may be added in the culture media. In some embodiments, at least 2 U/ml, at least 5 U/ml, at least 7 U/ml, at least 10 U/ml, at least 15 U/ml, at least 20 U/ml, at least 25 U/ml, at least 25 U/ml, at least 30 U/ml, at least 35 U/ml, at least 40 U/ml, at least 45 U/ml, at least 50 U/ml, at least 55 U/ml, or at least 60 U/ml of Benzonase® is added. In some embodiments, between 2 U/mL and 10 U/mL, between 10 U/mL and 20 U/mL, between 20 U/mL and 30 U/mL, between 30 U/mL and 40 U/mL, between 40 U/mL and 50 U/mL, or between 50 U/mL and 60 U/mL of Benzonase® is added. In some embodiments, the Benzonase® is added after at a time about 5-40, 10-40, 10-30, 20-30, or about 20 hours or about 24 hours after transfecting the host cells, e.g., Expi293F cells. In some embodiments, the benzonase is added at a concentration of 3-7 U/mL (e.g., about 5 U/mL) at 20-30 hours (e.g., about 24 hours) after transfecting the host cells. In some embodiments, the benzonase is added at a concentration of 3-7 U/mL (e.g. about 5 U/mL) at 1-5 hours (e.g., about 3 hours) after transfecting the host cells. In some embodiments, the benzonase is added at a concentration of 3-7 U/mL (e.g. about 5 U/mL) at 4-8 hours (e.g., about 6 hours) after transfecting the host cells. In some embodiments, the benzonase is added at a concentration of 12-18 U/mL (e.g. about 15 U/mL) at 1-5 hours (e.g., about 3 hours) after transfecting the host cells. In some embodiments, the benzonase is added at a concentration of 12-18 U/mL (e.g. about 15 U/mL) at 4-8 hours (e.g., about 6 hours) after transfecting the host cells. In some embodiments, the benzonase is added at a concentration of 12-18 U/mL (e.g. about 15 U/mL) at 20-30 hours (e.g., about 24 hours) after transfecting the host cells. In some embodiments, the benzonase is added at a concentration of 20-30 U/mL (e.g. about 25 U/mL) at 1-5 hours (e.g., about 3 hours) after transfecting the host cells. In some embodiments, the benzonase is added at a concentration of 20-30 U/mL (e.g. about 25 U/mL) at 4-8 hours (e.g., about 6 hours) after transfecting the host cells. In some embodiments, the benzonase is added at a concentration of 20-30 U/mL (e.g. about 25 U/mL) at 20-30 hours (e.g., about 24 hours) after transfecting the host cells. In some embodiments, the benzonase is added at a concentration of 40-60 U/mL (e.g. about 50 U/mL) at 1-5 hours (e.g., about 3 hours) after transfecting the host cells. In some embodiments, the benzonase is added at a concentration of 40-60 U/mL (e.g. about 50 U/mL) at 4-8 hours (e.g., about 6 hours) after transfecting the host cells. In some embodiments, the benzonase is added at a concentration of 40-60 U/mL (e.g. about 50 U/mL) at 20-30 hours (e.g., about 24 hours) after transfecting the host cells. In some embodiments, the benzonase is added at a concentration of 5 U/mL at about 3 hours after transfecting the host cells. In some embodiments, the benzonase is added at a concentration of 15 U/mL at about 3 hours after transfecting the host cells. In some embodiments, the benzonase is added at a concentration of 25 U/mL at about 3 hours after transfecting the host cells. In some embodiments, the benzonase is added at a concentration of 50 U/mL at about 3 hours after transfecting the host cells. In some embodiments, the benzonase is added at a concentration of 5 U/mL at about 6 hours after transfecting the host cells. In some embodiments, the benzonase is added at a concentration of 15 U/mL at about 6 hours after transfecting the host cells. In some embodiments, the benzonase is added at a concentration of 25 U/mL at about 6 hours after transfecting the host cells. In some embodiments, the benzonase is added at a concentration of 50 U/mL at about 6 hours after transfecting the host cells. In some embodiments, the benzonase is added at a concentration of 5 U/mL at about 24 hours after transfecting the host cells. In some embodiments, the benzonase is added at a concentration of 15 U/mL at about 24 hours after transfecting the host cells. In some embodiments, the benzonase is added at a concentration of 25 U/mL at about 24 hours after transfecting the host cells. In some embodiments, the benzonase is added at a concentration of 50 U/mL at about 24 hours after transfecting the host cells. In some embodiments, a salt, e.g., MgCl₂ is added to the Benzonase®, e.g., in a concentration at about 1-5 mM, 1-3 mM, or about 2 mM. In some embodiments, the methods disclosed herein may comprise addition of Benzonase® in production and/or purification process.

In some embodiments, a chemical compound may be added to the media to influence culture growth, e.g., inhibition of proliferation, induction of differentiation and induction or repression of gene expression. In some embodiments, the chemical compound is sodium butyrate. In some embodiments, a cell culture medium described herein comprises sodium butyrate.

### Culture Conditions and Transfection Conditions

Culture conditions can include any culture conditions suitable for maintaining a cell (e.g., in a static or proliferative state). For example, culture conditions can include several parameters, including without limitation, temperature, oxygen content, nutrient content (e.g., glucose content), pH (e.g., increasing or decreasing pH), agitation level (e.g., rotations per minute), gas flow rate (e.g., air, oxygen, nitrogen gas), redox potential, cell density (e.g., optical density), cell viability and the like. A change in culture conditions can comprise an alteration, modification or shift of one or more culture parameters. For example, one can change culture conditions by increasing or decreasing temperature, increasing or decreasing pH (e.g., adding or removing an acid, a base or carbon dioxide), increasing or decreasing oxygen content (e.g., introducing air, oxygen, carbon dioxide, nitrogen), increasing or decreasing air pressure (e.g., by introducing air, oxygen, carbon dioxide, nitrogen), increasing or decreasing agitation, and/or adding or removing a nutrient (e.g., one or more sugars or sources of sugar, biomass, vitamin and the like), increasing or decreasing the ratio of culture and flask volume, or combinations of the foregoing. In some embodiments, a change in culture condition, e.g., increasing or decreasing pH is introduced at a certain time during the culture, e.g., before transfection. In some embodiments, the pH is modified, e.g., adjusted to about 6.0-6.8, e.g., 6.2-6.8, e.g., 6.4-6.8, e.g., 6.7-6.75 before transfection with a lentiviral packaging system.

### Culture Volume and Culture Unit

The methods of the disclosure may be carried out in a small cell culture, e.g., in a laboratory scale, or in a large-scale culture, e.g., in industrial scale. The methods may be carried out in an appropriate culture unit, e.g., a culture flask or a bioreactor. The bioreactor can be of any size as long as it is useful for culturing cells, e.g., mammalian cells. In some embodiments, the methods of this disclosure are highly scalable, e.g., the plurality of mammalian cells is in a scaled culture (e.g., at least 1 L, at least 2 L, at least 5 L, at least 10 L, at least 15 L, at least 20 L yields a number of transducing units per ml culture that is no less than 30%, 40%, 50%, 60%, 70%, or 80% the number of transducing units per ml culture in an otherwise similar small-scale culture, e.g., 100 ml, 200 ml, e.g., 300 ml, 400 ml, 500 ml. In some embodiments, the scale culturing (i.e., with culture volumes greater than 50 L) and may be particularly amenable to scaling up from small, laboratory scale cultures (e.g., 10 L) to production scale cultures (e.g., 50 L and greater) with minimal modification of culture conditions. The internal conditions of the culture unit, including but not limited to pH, PO₂, and temperature, are typically controlled during the culturing period. A production culture unit refers to the final culture unit used in the production of the polypeptide, virus, and/or any other product of interest. The volume of a large-scale production culture unit is generally greater than about 50 liters, and may be about 100, about 200, about 300, about 500, about 800, about 1000, about 2500, about 5000, about 8000, about 10,000, about 12,0000 L or more, or any intermediate volume. A suitable culture unit or production culture unit may be composed of (i.e., constructed of) any material that is suitable for holding cell cultures suspended in media under the culture conditions contemplated herein, and one that is conducive to mammalian cell, e.g., HEK293 cells, e.g., Expi293F cell growth and viability. Examples of suitable materials include, without limitation, glass, plastic, and/or metal. In some embodiments, the material(s) do not interfere, or do not significantly or do not substantially interfere, with expression and/or stability of the desired product, e.g., the lentiviral vector.

In some embodiments, the cell culture process is operated in more than one distinct culture units, such as using one or more seed culture unit(s) followed by use of the production culture unit. In some embodiments, the process involves transferring the propagated seed culture from one or more seed culture unit to a large production unit. In some embodiments, expansion of the cells to the production culture unit and the production phase may be accomplished in one physical culture unit, e.g., the cells may be expanded to a final production scale and the process switched to production conditions. The spent medium is harvested at the end of culture period for down-stream processing the lentivirus or lentiviral vector. In some embodiments, harvest may be collected after 24 hours, after 48 hours, after 72 hours, after 96 hours, or after 120 hours post-transfection.

In some embodiments, down-stream processing comprises purification, formulation and/or long-term storage of the lentivirus. In some embodiments, the viral harvest collected at the end of culture period comprises lentivirus, at a concentration of, e.g., from about 5×10⁶ transducing units per milliliter (TU/mL) to about 7×10⁷ TU/mL (e.g., 5×10⁶ TU/mL, 5.5×10⁶ TU/mL, 6×10⁶ TU/mL, 6.5×10⁶ TU/mL, 7×10⁶ TU/mL, 7.5×10⁶ TU/mL, 8×10⁶ TU/mL, 8.5×10⁶ TU/mL, 9×10⁶ TU/mL, 9.5×10⁶ TU/mL, 1×10⁷ TU/mL, 1.5×10⁷ TU/mL, 2×10⁷ TU/mL, 2.5×10⁷ TU/mL, 3×10⁷ TU/mL, 3.5×10⁷ TU/mL, 4×10⁷ TU/mL, 4.5×10⁷ TU/mL, 5×10⁷ TU/mL, 5.5×10⁷ TU/mL, 6×10⁷ TU/mL, 6.5×10⁷ TU/mL, or 7×10⁷ TU/mL). In some embodiments, the viral harvest collected at the end of culture period comprises lentivirus, at a concentration of at least 5×10⁶ TU/mL, 5.5×10⁶ TU/mL, 6×10⁶ TU/mL, 6.5×10⁶ TU/mL, 7×10⁶ TU/mL, 7.5×10⁶ TU/mL, 8×10⁶ TU/mL, 8.5×10⁶ TU/mL, 9×10⁶ TU/mL, 9.5×10⁶ TU/mL, 1×10⁷ TU/mL, 1.5×10⁷ TU/mL, 2×10⁷ TU/mL, 2.5×10⁷ TU/mL, 3×10⁷ TU/mL, 3.5×10⁷ TU/mL, 4×10⁷ TU/mL, 4.5×10⁷ TU/mL, 5×10⁷ TU/mL, 5.5×10⁷ TU/mL, 6×10⁷ TU/mL, 6.5×10⁷ TU/mL, or 7×10⁷ TU/mL. In some embodiments, the viral harvest collected at the end of culture period comprises lentivirus, at a concentration of 5×10⁶ TU/mL-6×10⁶ TU/mL, 6×10⁶ TU/mL-7×10⁶ TU/mL, 7×10⁶ TU/mL-8×10⁶ TU/mL, 8×10⁶ TU/mL-9×10⁶ TU/mL, 9×10⁶ TU/mL-1×10⁷ TU/mL, 1×10⁷ TU/mL-2×10⁷ TU/mL, 2×10⁷ TU/mL-3×10⁷ TU/mL, 3×10⁷ TU/mL-4×10⁷ TU/mL, 4×10⁷ TU/mL-5×10⁷ TU/mL, 5×10⁷ TU/mL-6×10⁷ TU/mL, 6×10⁷ TU/mL-7×10⁷ TU/mL.

### Purification Methods Including Filtration and Chromatography

In some aspects, the disclosure provides processes for purifying lentiviral vectors with improved efficiency, e.g., such that higher quantities of lentiviral vector are recovered. In some embodiments, at least one step in the purification process comprises adding an agent, e.g., an amino acid or a salt thereof, e.g., an arginine or a salt thereof, e.g., arginine-HCl to the purification intermediate composition (an intermediate composition comprising a buffer before completion of purification) before further purification, e.g., centrifugation, filtration, or chromatography, to improve the purification process. In some embodiments, filtration may refer to but are not limited to flow filtration, depth filtration, tangential flow filtration. In some embodiments, chromatography may include but are not limited to Size Exclusion Chromatography, Affinity Chromatography, Hydrophobic Interaction Chromatography, Ion Exchange Chromatography.

In some embodiments, a lentiviral vector produced according to a method described herein has one or more of the following properties: complies with GMP guidelines, is sterile, is substantially free of contaminants, is suitable for pharmaceutical use, is suitable for administration to a human subject, or is suitable for ex vivo treatment of human cells.

In some embodiments of the methods described herein, a solution or a suspension is subjected to a semi-permeable membrane (filtration) that retains larger particles e.g., viral particles, while allowing solvent and small solute molecules to pass through. In some embodiments, a method described herein uses a filter to remove and exchange salts, sugars, and non-aqueous solvents, to separate free from bound species, to remove low molecular-weight material, and/or to cause the rapid change of ionic and/or pH environments. A filtration step may be used to increase the concentration of vectors in a solution or suspension. In some embodiments, a filtration step is used to increase the concentration of a lentiviral particle in harvest. In some embodiments, a method described herein makes use of a process, technique or combination of techniques comprises a filtration step (e.g., one or more of microfiltration, ultrafiltration, nanofiltration, and diafiltration) either sequentially or simultaneously. In some embodiments, filtration is performed using a flat-sheet membrane or a hollow fiber. In some embodiments, the filtration is performed using an average transmembrane pressure of about 0.1-0.5 bar (e.g., about 0.1, 0.2, 0.3, 0.4, or 0.5 bar). In some embodiments, filtration is performed using a load of 4-100 L/m², e.g., about 4-10, 10-20, 20-30, 30-40, 40-50, 50-60, 60-70, 70-80, or 80-90. In certain embodiments of the present disclosure, a filtration step is employed to exchange the various buffers used in connection with the instant disclosure, optionally in combination with chromatography or other purification steps, and optionally also to remove impurities from viral yield.

Filtration techniques, such as those described above and known in the art, can be used so as to produce lentiviral preparations that are substantially free of microorganisms and cells (e.g., mammalian cells, e.g., HEK293 cells, e.g., Expi293F cells) from which the lentiviral vector is prepared. Additionally, or alternatively, lentiviral vector preparations of the disclosure may be treated with nucleases so as to produce a preparation that is substantially free of contaminating polynucleotides (e.g., non-lentiviral polynucleotides derived from the cell in which the lentiviral vector was produced, such as chromosomal mammalian DNA, human DNA, RNA, or other polynucleotides that are not included within the lentiviral transgene).

### Buffers, e.g., for Use in Purification

Various buffers, e.g., an aqueous composition comprising buffering agents comprising buffering agents used for viral vector purification are known in the arts and may include but not limited to sulfonic based acid buffer, e.g., 1,4-piperazinediethanesulfonic acid (PIPES) based buffer (PIPES buffer), polyol-based buffer, tris buffer, phosphate buffer, acetate buffer, citrate buffer. In some embodiments, the buffer used in relation to the purification process disclosed herein is a sulfonic acid-based buffer, e.g., PIPES buffer. In some embodiments, a PIPES buffer may comprise, a buffering agent, e.g., PIPES at a concentration of from about 10 mM to about 50 mM, from about 15 mM to about 40 mM, from about 20 mM to about 30 mM, e.g., about 20 mM.

In some embodiments, a buffer may further comprise a salt, e.g., Sodium Chloride (NaCl), Magnesium Chloride (MgCl₂), or Calcium Chloride (CaCl₂), or any combination thereof. The salt may be present, e.g., at a concentration of from about 1 mM to about 1 M in the aqueous lentiviral preparation (e.g., 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 100 mM, 125 mM, 150 mM, 175 mM, 200 mM, 225 mM, 250 mM, 275 mM, 300 mM, 325 mM, 350 mM, 375 mM, 400 mM, 450 mM, 475 mM, 500 mM, 525 mM, 575 mM, 600 mM, 625 mM, 650 mM, 675 mM, 700 mM, 725 mM, 750 mM, 775 mM, 800 mM, 825 mM, 850 mM, 875 mM, 900 mM, 925 mM, 950 mM, 957 mM, or 1 M). In some embodiments, the concentration of salt is from about 25 mM to about 250 mM, about 50 mM to about 75 mM, about 50 mM to about 200 mM, or about 100 mM to about 150 mM (e.g., 25 mM, 30 mM, 35 mM, 40 mM, 45 mM, 50 mM, 55 mM, 60 mM, 65 mM, 70 mM, 75 mM, 80 mM, 85 mM, 90 mM, 100 mM, 125 mM, or 150 mM). In some embodiments, the concentration of salt may be 50 mM or 75 mM, as desired.

In some embodiments, the buffer may also comprise a carbohydrate, e.g., a non-reducing carbohydrate, e.g., sucrose or trehalose. In some embodiments, the carbohydrate, e.g., sucrose, is present at a concentration of about 30 mM to about 300 mM, from about 40 mM to about 275 mM, from about 50 mM to about 250 mM, from about 60 mM to about 240 mM, from about 70 mM to about 220 mM, from about 30 mM to 150 mm, or from about 150-300 mM. In some embodiments the buffer, e.g., the PIPES buffer, e.g., the filtration buffer, the exchange buffer comprises sucrose at a concentration from about 50 mM to about 80 mM, e.g., about 73 mM. In some embodiments, the buffer, e.g., the PIPES buffer, e.g., the formulation buffer, the storage buffer comprises sucrose at a concentration of from about 200 mM to 250 mM, e.g., about 220 mM.

In some embodiments, a carbohydrate may be present at a concentration of, e.g., from about 1% to about 10%, from about 2.5% to about 10%, or from about 2.5% to about 5% by weight per volume (w/v) of the aqueous lentiviral preparation. For instance, a carbohydrate, such as a non-reducing carbohydrate described herein, can be present within an aqueous lentiviral preparation at a concentration of 1% w/v, 1.5% w/v, 2% w/v, 2.5% w/v, 3% w/v, 3.5% w/v, 4% w/v, 4.5% w/v, 5% w/v, 5.5% w/v, 6% w/v, 6.5% w/v, 7% w/v, 7.5% w/v, 8% w/v, 8.5% w/v, 9% w/v, 9.5% w/v, or 10% w/v. In some embodiments, a carbohydrate, such as a non-reducing carbohydrate described herein, can be present within an aqueous lentiviral preparation at a concentration of at least 1% w/v, 1.5% w/v, 2% w/v, 2.5% w/v, 3% w/v, 3.5% w/v, 4% w/v, 4.5% w/v, 5% w/v, 5.5% w/v, 6% w/v, 6.5% w/v, 7% w/v, 7.5% w/v, 8% w/v, 8.5% w/v, 9% w/v, 9.5% w/v, or 10% w/v. In some embodiments, a carbohydrate, such as a non-reducing carbohydrate described herein, can be present within an aqueous lentiviral preparation at a concentration of 1% w/v-2% w/v, 2% w/v-3% w/v, 3% w/v-4% w/v, 4% w/v-5% w/v, 5% w/v-6% w/v, 6% w/v-7% w/v, 7% w/v-8% w/v, 8% w/v-9% w/v, 9% w/v-10% w/v.

In some embodiments, the buffer further comprises e.g., arginine or a salt thereof, e.g., arginine-HCl. In some embodiments, the agent, e.g., arginine or a salt thereof, e.g., arginine monohydrochloride (arginine-HCl) is added at a concentration of about 25-50 mM (e.g., about 50 mM), 50-100 mM (e.g., about 75 mM), 100-200 mM (e.g., about 150 mM), or 200-400 (e.g., about 300) mM. In some embodiments, at least one of buffers, e.g., PIPES buffer used for viral purification (e.g., lentiviral purification using a process disclosed herein) comprises arginine, e.g., arginine-HCl.

In some embodiments, the pH of the buffers used in the purification process disclosed herein is from about 5.0 to about 8.0, e.g., 6.0 to about 7.0 (e.g., 6.0, 6.1, 6.2, 6.3, 6.4, 6.5, 6.6, 6.7, 6.8, 6.9, or 7.0), e.g., about 6.5.

In some embodiments, the PIPES buffer may be used as one or more of exchange buffer, filtration buffer, formulation buffer, and/or storage buffer. In some embodiments, the ratio of concentration of PIPES, NaCl, and sucrose are different in PIPES filtration buffer, PIPES exchange buffer, PIPES formulation buffer, and PIPES storage buffer. In some embodiments, the ratio of concentration of PIPES, NaCl, and sucrose are identical in PIPES filtration buffer, PIPES exchange buffer, PIPES formulation buffer, and PIPES storage buffer. In some embodiments the ratio of concentration of PIPES, NaCl, and sucrose are identical in PIPES exchange buffer and PIPES filtration buffer. In some embodiments the ratio of concentration of PIPES, NaCl, sucrose, and optionally arginine, e.g., arginine-HCl are identical in PIPES formulation buffer and PIPES storage buffer.

### Arginine Spike

In some embodiments, arginine, e.g., arginine-HCl is added to cell culture harvest during purification. In some embodiments, arginine, e.g., arginine-HCl is added to the purification intermediate composition comprising a buffer, e.g., a PIPES buffer or PIPES buffer during purification. In some embodiments, arginine, e.g., arginine-HCl is added to a PIPES buffer that does not comprise arginine. In some embodiments, arginine, e.g., arginine-HCl is added to a PIPES buffer that comprises arginine. In some embodiments, the agent, e.g., arginine or a salt thereof, e.g., arginine monochloride (arginine-HCl) is added at a concentration of about 25-50 mM (e.g., about 50 mM), 50-100 mM (e.g., about 75 mM), 100-200 mM (e.g., about 150 mM), or 200-400 (e.g., about 300) mM.

In some embodiments the vector recovery, e.g., the amount of transducing units of the lentivirus increases in a purification process which comprises a purification step comprising adding arginine to the purification intermediate composition by about 10%-300%, 20%-180%, 30%-160%, 50%-150%, 75%-125% or about 100% higher relative to a purification process which does not comprise a purification step comprising adding arginine to the purification intermediate composition. In some embodiments, addition of arginine decreases the process time of purification. In some embodiments, when purification comprises addition of e.g., an arginine or a salt thereof, e.g., arginine-HCl, to the purification intermediate composition, the process time of the purification is improved by at least 5%, at least 10%, at least 15%, at least 20%, at least 25%, at least 30%, at least 35%, at least 40%, at least 45%, or by at least 50% compared an otherwise similar purification process which does not comprise adding arginine to the purification intermediate composition. In some embodiments, the purification intermediate composition after addition of arginine or a salt thereof, e.g., arginine-HCl and subsequent purification step shows a total particle concentration per ml of less than 400,000, 300,000, 200,000, or 100,000, as measured by micro-flow imaging. Without wishing to be bound by theory, in some embodiments, the micro-flow imaging does not substantially detect individual lentiviral particles (e.g., infectious viral particles), but detects larger particles comprising aggregates, e.g., aggregates of non-functional virus. In some embodiments, the purification intermediate composition after addition of arginine or a salt thereof, e.g., arginine-HCl and subsequent purification step show a concentration of particles that are ≥10 μm per ml of less than about 5,000, 4,500, 4,000, 3,500, 3,000, or 2,500, as measured by micro-flow imaging. In some embodiments, the purification intermediate composition after addition of arginine or a salt thereof, e.g., arginine-HCl and subsequent purification step show a concentration≥25 μm per ml of less than about 500, 400, 300, or 200, as measured by micro-flow imaging. In some embodiments, the reduction of aggregates reduces blockage of filtration membrane at a given time point. In some embodiments, the arginine stabilizes the lentiviral particles.

In some embodiments, the purified lentiviral composition comprises a lentiviral vector at a concentration of, e.g., from about 1×10⁷ transducing units per milliliter (TU/mL) to about 7×10⁷ TU/mL (e.g., 1×10⁷ TU/mL, 1.5×10⁷ TU/mL, 2×10⁷ TU/mL, 2.5×10⁷ TU/mL, 3×10⁷ TU/mL, 3.5×10⁷ TU/mL, 4×10⁷ TU/mL, 4.5×10⁷ TU/mL, 5×10⁷ TU/mL, 5.5×10⁷ TU/mL, 6×10⁷ TU/mL, 6.5×10⁷ TU/mL, or 7×10⁷ TU/mL).

### Aqueous Compositions for Lentiviral Storage

In some embodiments, the disclosure provides a preparation, e.g., an aqueous mixture, e.g., an aqueous solution or a suspension e.g., an aqueous composition comprising a lentiviral vector disclosed herein and a buffer, e.g., a formulation buffer or a storage buffer, e.g., a PIPES buffer, e.g., a PIPES buffer comprising arginine, e.g., arginine-HCl. In some embodiments, lentiviral preparations comprising a formulation buffer or a storage buffer, e.g., a PIPES buffer, e.g., a PIPES buffer comprising arginine, e.g., arginine-HCl exhibit improved biological properties relative to lentiviral preparations containing a conventional lentiviral formulation buffer, such as HEPES. These improved biological characteristics include elevated resistance to aggregation across a range of temperatures and salt concentrations as disclosed in WO2017087861A1. In some embodiments, the PIPES buffer shows an improved transduction capacity at physiological and at elevated temperatures (such as 42° C. and 50° C.), and greater resistance to loss of infectivity during multiple freeze/thaw cycles. Other buffers useful in conjunction with lentiviral preparations of the disclosure include histidine buffers, phosphate buffers, sodium citrate buffers, MES buffers, and MOPS buffers. Lentiviral preparations of the disclosure may optionally include a salt, such as sodium chloride, and may optionally contain a carbohydrate, such as a non-reducing carbohydrate.

In some embodiments, a PIPES formulation buffer and/or storage buffer may comprise, a buffering agent, e.g., PIPES at a concentration of from about 10 mM to about 50 mM, from about 15 mM to about 40 mM, from about 20 mM to about 30 mM, e.g., about 20 mM. Lentiviral vector preparations can optionally include a salt, such as sodium chloride, magnesium chloride, or calcium chloride. The salt may be present, e.g., at a concentration of from about 1 mM to about 1 M in the aqueous lentiviral preparation (e.g., 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 100 mM, 125 mM, 150 mM, 175 mM, 200 mM, 225 mM, 250 mM, 275 mM, 300 mM, 325 mM, 350 mM, 375 mM, 400 mM, 450 mM, 475 mM, 500 mM, 525 mM, 575 mM, 600 mM, 625 mM, 650 mM, 675 mM, 700 mM, 725 mM, 750 mM, 775 mM, 800 mM, 825 mM, 850 mM, 875 mM, 900 mM, 925 mM, 950 mM, 957 mM, or 1 M). In some embodiments, the concentration of salt is at least about 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 100 mM, 125 mM, 150 mM, 175 mM, 200 mM, 225 mM, 250 mM, 275 mM, 300 mM, 325 mM, 350 mM, 375 mM, 400 mM, 450 mM, 475 mM, 500 mM, 525 mM, 575 mM, 600 mM, 625 mM, 650 mM, 675 mM, 700 mM, 725 mM, 750 mM, 775 mM, 800 mM, 825 mM, 850 mM, 875 mM, 900 mM, 925 mM, 950 mM, 957 mM, or 1 M. In some embodiments, the concentration of salt is 1-2 mM, 2-3 mM, 3-4 mM, 4-5 mM, 5-6 mM, 6-7 mM, 7-8 mM, 8-9 mM, 9-10 mM, 10-15 mM, 15-20 mM, 20-25 mM, 25-30 mM, 30-35 mM, 35-40 mM, 40-45 mM, 45-50 mM, 50-55 mM, 55-60 mM, 60-65 mM, 65-70 mM, 70-75 mM, 75-80 mM, 80-85 mM, 85-90 mM, 90-100 mM, 100-125 mM, 125-150 mM, 150-175 mM, 175-200 mM, 200-225 mM, 225-250 mM, 250-275 mM, 275-300 mM, 300-325 mM, 325-350 mM, 350-375 mM, 375-400 mM, 400-450 mM, 450-475 mM, 475-500 mM, 500-525 mM, 525-575 mM, 575-600 mM, 600-625 mM, 625-650 mM, 650-675 mM, 675-700 mM, 700-725 mM, 725-750 mM, 750-775 mM, 775-800 mM, 800-825 mM, 825-850 mM, 850-875 mM, 875-900 mM, 900-925 mM, 925-950 mM, 950-975 mM, or 957 mM-1 M. In some embodiments, the concentration of salt is from about 25 mM to about 250 mM, about 50 mM to about 75 mM, about 50 mM to about 200 mM, or about 100 mM to about 150 mM (e.g., 25 mM, 30 mM, 35 mM, 40 mM, 45 mM, 50 mM, 55 mM, 60 mM, 65 mM, 70 mM, 75 mM, 80 mM, 85 mM, 90 mM, 100 mM, 125 mM, or 150 mM). In some embodiments, the concentration of salt may be 50 mM or 75 mM, as desired.

A lentiviral vector preparation of the disclosure may optionally contain a carbohydrate, such as a non-reducing carbohydrate as described herein. Exemplary non-reducing carbohydrates include sucrose and trehalose, among others. When included in a lentiviral vector preparation, a carbohydrate may be present at a concentration of, e.g., from about 1% to about 10%, from about 2.5% to about 10%, or from about 2.5% to about 5% by weight per volume (w/v) of the aqueous lentiviral preparation. For instance, a carbohydrate, such as a non-reducing carbohydrate described herein, can be present within an aqueous lentiviral preparation at a concentration of 1% w/v, 1.5% w/v, 2% w/v, 2.5% w/v, 3% w/v, 3.5% w/v, 4% w/v, 4.5% w/v, 5% w/v, 5.5% w/v, 6% w/v, 6.5% w/v, 7% w/v, 7.5% w/v, 8% w/v, 8.5% w/v, 9% w/v, 9.5% w/v, or 10% w/v.

A lentiviral vector preparation of the disclosure may comprise an amino acid or a salt thereof; such as alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine or a salt thereof. When included in a lentiviral vector preparation, an amino acid, e.g., arginine-HCl may be present at a concentration of, about 25-50 mM (e.g., about 50 mM), 50-100 mM (e.g., about 75 mM), 100-200 mM (e.g., about 150 mM), or 200-400 (e.g., about 300) mM. In some embodiments, a lentiviral vector preparation as disclosed herein may comprise more than one amino acid or salt thereof, e.g., an arginine or salt thereof and an histidine or salt thereof.

Lentiviral vector preparations described herein may exhibit a pH, e.g., of from about 5.0 to about 8.0, e.g., 6.0 to about 7.0 (e.g., 6.0, 6.1, 6.2, 6.3, 6.4, 6.5, 6.6, 6.7, 6.8, 6.9, or 7.0). In some embodiments, the pH of the lentiviral vector preparation is 6.5.

In some embodiments, PIPES formulation buffer and PIPES storage buffer comprises identical composition, e.g., identical concentration of PIPES, NaCl, sucrose, and optionally arginine, e.g., arginine-HCl. In some embodiments, PIPES formulation buffer and PIPES storage buffer comprises different composition, e.g., different concentration of PIPES, NaCl, sucrose, and optionally arginine, e.g., arginine-HCl.

A lentiviral vector may be present within a lentiviral preparation of the disclosure within a range of concentrations. For instance, a lentiviral vector may be present within a lentiviral preparation at a concentration of, e.g., from about 1×10⁷ transducing units per milliliter (TU/mL) to about 1×10⁹ TU/mL (e.g., 1×10⁷ TU/mL, 2×10⁷ TU/mL, 3×10⁷ TU/mL, 4×10⁷ TU/mL, 5×10⁷ TU/mL, 6×10⁷ TU/mL, 7×10⁷ TU/mL, 8×10⁷ TU/mL, 9×10⁷ TU/mL, 1×10⁸ TU/mL, 1.5×10⁸ TU/mL, 2×10⁸ TU/mL, 2.5×10⁸ TU/mL, 3×10⁸ TU/mL, 3.5×10⁸ TU/mL, 4×10⁸ TU/mL, 4.5×10⁸ TU/mL 5×10⁸ TU/mL, 5.5×10⁸ TU/mL, 6×10⁸ TU/mL, 6.5×10⁸ TU/mL, 7×10⁸ TU/mL, 7.5×10⁸ TU/mL, 8×10⁸ TU/mL, 8.5×10⁸ TU/mL, 9×10⁸ TU/mL, 9.5×10⁸ TU/mL, or 1×10⁹ TU/mL). When desirable, a lentiviral preparation may contain a lentiviral vector at a concentration of from about 3×10⁸ TU/mL to about 5×10⁸ TU/mL (e.g., 3×10⁸ TU/mL, 3.5×10⁸ TU/mL, 4×10⁸ TU/mL, 4.5×10⁸ TU/mL, or 5×10⁸ TU/mL).

The disclosure also provides aqueous compositions that each include a lentiviral vector, a buffer selected from the group consisting of a phosphate buffer, a sodium citrate buffer, a 2-(N-morpholino)ethanesulfonic acid (MES) buffer, a 3-morpholinopropane-1-sulfonic acid (MOPS) buffer, and a salt (e.g., sodium chloride, magnesium chloride, or calcium chloride). These compositions can further include a carbohydrate, for example, a non-reducing carbohydrate (e.g., sucrose or trehalose).

In some embodiments, the aqueous composition, e.g., an aqueous composition comprising a lentiviral vector described herein may be stored at low temperatures, e.g., at 10° C., at 6° C., at 4° C., at 0° C., at −10° C., at −20° C., at −30° C., at −40° C., at −50° C., at −60° C., at −70° C., at −80° C., or at −90° C. for a period of time, e.g., for about 20 minutes, 40 minutes, 60 minutes, 1.5 hour, 2 hours, 5 hours, 10 hours, 15 hours, 20 hours, 1 day, 2 days, 3 days, 4 days, 5 days, 7 days, 10 days, 12 days, 14 days, 16 days, 18 days, 20 days, 21 days, or 25 days. In some embodiments, the aqueous composition is stored at less than 10° C., 6° C., 4° C., 0° C., −10° C., −20° C., −30° C., −40° C., −50° C., −60° C., −70° C., −80° C., or −90° C. In some embodiments, a purified lentiviral sample stored in a PIPES storage buffer is stored at −80° C. immediately after purification in a frozen condition. In some embodiments, the lentiviral preparation thus stored may be thawed prior to use and refrozen (e.g., a freeze-thaw cycle). In some embodiments, a lentiviral preparation prepared and stored as disclosed herein may undergo at least 1, at least 2, at least 3, at least 4, at least 5, at least 6, at least 7, at least 8, at least 9 freeze-thaw cycles without any significant loss of stability and/or infectivity. In some embodiments, the preparation displays no more than 0.5%, more than 1%, more than 2%, more than 3%, more than 4%, more than 5%, more than 10%, more than 15%, more than 20%, more than 25%, more than 30%, more than 40%, more than 50%, more than 60%, more than 70%, or more than 80% loss of stability and/or infectivity compared to a lentiviral preparation that never underwent a freeze-thaw cycle.

In some embodiments, a lentivirus preparation as disclosed herein may be stored at a chilled condition at 4° C. for a period of time, e.g., for about 20 minutes, 40 minutes, 60 minutes, 1.5 hour, 2 hours, 5 hours, 10 hours, 15 hours, 20 hours, 1 day, 2 days, 3 days, 4 days, 5 days, 7 days, 10 days, 12 days, 14 days, 16 days, 18 days, 20 days, 21 days, or 25 days. In some embodiments, a lentiviral preparation as disclosed herein may be stored in a frozen condition at −80° C. for a period of time, e.g., for about 20 minutes, 40 minutes, 60 minutes, 1.5 hour, 2 hours, 5 hours, 10 hours, 15 hours, 20 hours, 1 day, 2 days, 3 days, 4 days, 5 days, 7 days, 10 days, 12 days, 14 days, 16 days, 18 days, 20 days, 21 days, or 25 days. In some embodiments, a lentivirus preparation stored as disclosed (e.g., stored in a frozen condition) herein, displays at least 30%, at least 40%, at least 50%, at least 60%, at least 70%, at least 75%, at least 80%, at least 85%, at least 90%, at least 92%, at least 95%, at least 98%, at least 99% or 100% infectivity compared to a lentivirus that was never frozen. In some embodiments, the lentivirus preparation does not lose more than 0.5%, more than 1%, more than 2%, more than 5%, more than 7%, more than 10%, more than 15%, more than 20%, more than 25%, more than 30%, more than 40%, more than 50%, more than 60%, more than 70%, more than 80% loss of infectivity after undergoing more than 1, (e.g., 2, 3, 4, 5, 6, 7, 8, or 9) freeze-thaw cycles. In some embodiments, a lentivirus preparation stored as disclosed (e.g., stored in a frozen condition) herein, is at least 30%, at least 40%, at least 50%, at least 60%, at least 70%, at least 75%, at least 80%, at least 85%, at least 90%, at least 92%, at least 95%, at least 98%, at least 99% or 100% stable compared to a lentivirus that was never frozen. In some embodiments, a lentivirus preparation is used after freezing for at least 5 hours, at least 12 hours, at least 18 hours, at least 1 days, at least 2 days, at least 3 days, at least 5 days, at least 7 days for improved vector integration.

The disclosure further includes dried or lyophilized compositions, which are prepared by drying or lyophilizing the aqueous compositions described herein, as well as aqueous compositions that are prepared by reconstituting such dried or lyophilized compositions in a buffer described herein (or another, standard vehicle for administration).

### CAR Targets

Described herein are viral vectors to transduce immune effector cells (e.g., T cells, NK cells) that are engineered to contain one or more chimeric antigen receptors (CAR)s that direct the immune effector cells to undesired cells (e.g., cancer cells). This is achieved through an antigen binding domain on the CAR that is specific for a cancer associated antigen. Two classes of cancer associated antigens (tumor antigens) that can be targeted by CARs are: (1) cancer associated antigens that are expressed on the surface of cancer cells; and (2) cancer associated antigens that itself is intracellular, however, a fragment of such antigen (peptide) is presented on the surface of the cancer cells by MHC (major histocompatibility complex).

In some embodiments, the tumor antigen is chosen from one or more of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcu-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation End products (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

A CAR described herein can comprise an antigen binding domain (e.g., antibody or antibody fragment, TCR or TCR fragment) that binds to a tumor-supporting antigen (e.g., a tumor-supporting antigen as described herein). In some embodiments, the tumor-supporting antigen is an antigen present on a stromal cell or a myeloid-derived suppressor cell (MDSC). Stromal cells can secrete growth factors to promote cell division in the microenvironment. MDSC cells can inhibit T cell proliferation and activation. Without wishing to be bound by theory, in some embodiments, the CAR-expressing cells destroy the tumor-supporting cells, thereby indirectly inhibiting tumor growth or survival.

In embodiments, the stromal cell antigen is chosen from one or more of: bone marrow stromal cell antigen 2 (BST2), fibroblast activation protein (FAP) and tenascin. In an embodiment, the FAP-specific antibody is, competes for binding with, or has the same CDRs as, sibrotuzumab. In embodiments, the MDSC antigen is chosen from one or more of: CD33, CD11b, C14, CD15, and CD66b. Accordingly, in some embodiments, the tumor-supporting antigen is chosen from one or more of: bone marrow stromal cell antigen 2 (BST2), fibroblast activation protein (FAP) or tenascin, CD33, CD11b, C14, CD15, and CD66b.

### CD19

An non-limiting exemplary tumor antigen is CD19. CARs that bind to CD19 are known in the art. For example, those disclosed in WO2012/079000 and WO2014/153270 may be used in accordance with the present disclosure. Any known CD19 CAR, for example, the CD19 antigen binding domain of any known CD19 CAR, in the art can be used in accordance with the present disclosure. For example, LG-740; CD19 CAR described in the U.S. Pat. Nos. 8,399,645; 7,446,190; Xu et al., Leuk Lymphoma. 2013 54(2):255-260(2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25):4129-39(2013); and 16th Annu Meet Am Soc Gen Cell Ther (ASGCT) (May 15-18, Salt Lake City) 2013, Abst 10.

Non-limiting exemplary CD19 CARs include CD19 CARs described herein or an anti-CD19 CAR described in Xu et al. Blood 123.24(2014):3750-9; Kochenderfer et al. Blood 122.25(2013):4129-39, Cruz et al. Blood 122.17(2013):2965-73, NCT00586391, NCT01087294, NCT02456350, NCT00840853, NCT02659943, NCT02650999, NCT02640209, NCT01747486, NCT02546739, NCT02656147, NCT02772198, NCT00709033, NCT02081937, NCT00924326, NCT02735083, NCT02794246, NCT02746952, NCT01593696, NCT02134262, NCT01853631, NCT02443831, NCT02277522, NCT02348216, NCT02614066, NCT02030834, NCT02624258, NCT02625480, NCT02030847, NCT02644655, NCT02349698, NCT02813837, NCT02050347, NCT01683279, NCT02529813, NCT02537977, NCT02799550, NCT02672501, NCT02819583, NCT02028455, NCT01840566, NCT01318317, NCT01864889, NCT02706405, NCT01475058, NCT01430390, NCT02146924, NCT02051257, NCT02431988, NCT01815749, NCT02153580, NCT01865617, NCT02208362, NCT02685670, NCT02535364, NCT02631044, NCT02728882, NCT02735291, NCT01860937, NCT02822326, NCT02737085, NCT02465983, NCT02132624, NCT02782351, NCT01493453, NCT02652910, NCT02247609, NCT01029366, NCT01626495, NCT02721407, NCT01044069, NCT00422383, NCT01680991, NCT02794961, or NCT02456207, each of which is incorporated herein by reference in its entirety.

In some embodiments, the antigen binding domain binds to CD19 and has the same or a similar binding specificity as the FMC63 scFv fragment described in Nicholson et al. Mol. Immun. 34 (16-17): 1157-1165 (1997). In some embodiments, the antigen binding domain binds to CD19 and includes the scFv fragment described in Nicholson et al. Mol. Immun. 34 (16-17): 1157-1165 (1997).

In some embodiments, the antigen binding domain (for example, a humanized antigen binding domain) binds to CD19 and comprises a sequence from Table 3 of WO2014/153270, incorporated herein by reference. WO2014/153270 also describes methods of assaying the binding and efficacy of various CAR constructs.

Humanization of murine CD19 antibody is desired for the clinical setting, where the mouse-specific residues may induce a human-anti-mouse antigen (HAMA) response in patients who receive CART19 treatment, i.e., treatment with T cells transduced with the CAR19 construct. The production, characterization, and efficacy of humanized CD19 CAR sequences is described in International Application WO2014/153270 which is herein incorporated by reference in its entirety, including Examples 1-5 (p. 115-159).

In some embodiments, the antigen binding domain comprises the parental murine scFv sequence of the CAR19 construct provided in WO2012/079000 (incorporated herein by reference). In some embodiments, the antigen binding domain binds CD19 and comprises a scFv described in WO2012/079000.

In some embodiments, the CD19 CAR comprises the fusion polypeptide sequence provided as SEQ ID NO: 12 in WO2012/079000, which provides an scFv fragment of murine origin that specifically binds to human CD19.

In some embodiments, the CD19 CAR comprises an amino acid sequence provided as SEQ ID NO: 12 in WO2012/079000.

In some embodiments, the CD19 CAR comprises the amino acid sequence: digmtqttsslsaslgdrvtiscrasqdiskylnwyqqkpdgtvklliyhtsrlhsgvpsrfsgsgsgtdysltisnlegediatyfcqqgntlpytfg ggtkleitggggsggggsggggsevklgesgpglvapsgslsvtctvsgvslpdygvswirqpprkglewlgviwgsettyynsalksrltiik dnsksqvflkmnslqtddtaiyycakhyyyggsyamdywgqgtsvtvsstttpaprpptpaptiasqplslrpeacrpaaggavhtrgldfac diyiwaplagtcgvlllslvitlyckrgrkkllyifkqpfmrpvqttgeedgcscrfpeeeeggcelrvkfsrsadapaykqggnglynelnlgrr eeydvldkrrgrdpemggkprrknpgeglynelqkdkmaeayseigmkgerrrgkghdglygglstatkdtydalhmgalppr (SEQ ID NO: 757), or a sequence substantially homologous thereto.

In some embodiments, the CD19 CAR comprises the amino acid sequence: eivmtqspatlslspgeratlscrasqdiskylnwyqqkpgqaprlliyhtsrlhsgiparfsgsgsgtdytltisslqpedfavyfcqqgntlpytf gqgtkleikggggsggggsggggsqvqlqesgpglvkpsetlsltctvsgvslpdygvswirqppgkglewigviwgsettyyqsslksrvtis kdnsknqvslklssvtaadtavyycakhyyyggsyamdywgqgtlvtvss (SEQ ID NO: 758)

In some embodiments, the CD19 CAR is a humanized CD19 CAR comprising the amino acid sequence: eivmtqspatlslspgeratlscrasqdiskylnwyqqkpgqaprlliyhtsrlhsgiparfsgsgsgtdytltisslqpedfavyfcqqgntlpytf gqgtkleikggggsggggsggggsqvqlqesgpglvkpsetlsltctvsgvslpdygvswirqppgkglewigviwgsettyyqsslksrvtis kdnsknqvslklssvtaadtavyycakhyyyggsyamdywgqgtlvtvsstttpaprpptpaptiasqplslrpeacrpaaggavhtrgldfac diyiwaplagtcgvlllslvitlyckrgrkkllyifkqpfmrpvqttgeedgcscrfpeeeeggcelrvkfsrsadapaykqggnglynelnlgrr eeydvldkrrgrdpemggkprrknpqeglynelqkdkmaeayseigmkgerrrgkghdglyqglstatkdtydalhmqalppr (SEQ ID NO: 759)

In some embodiments, CD19 CARs comprise a sequence, for example, a CDR, VH, VL, scFv, or full-CAR sequence, disclosed in Table 1 below, or a sequence having at least 80%, 85%, 90%, 95%, or 99% identity thereto.

TABLE 1
Amino acid and nucleic acid sequences of exemplary anti-CD19 molecules

| SEQ ID NO | Region | Sequence |
|---|---|---|
| CTL019 | | |
| 760 | HCDR1 | DYGVS |
| | (Kabat) | |
| 761 | HCDR2 | VIWGSETTYYNSALKS |
| | (Kabat) | |
| 762 | HCDR3 | HYYYGGSYAMDY |
| | (Kabat) | |
| 763 | LCDR1 | RASQDISKYLN |
| | (Kabat) | |
| 764 | LCDR2 | HTSRLHS |
| | (Kabat) | |
| 765 | LCDR3 | QQGNTLPYT |
| | (Kabat) | |
| 766 | CTL019 | MALPVTALLLPLALLLHAARPdiqmtqttsslsaslgdrvtiscrasqdisky1 |
| | Full amino | nwyqqkpdgtvklliyhtsrlhsgvpsrfsgsgsgtdysltisnleqediatyfcqqgntlpytf |
| | acid | gggtkleitggggsggggsggggsevklqesgpglvapsqslsvtctvsgvslpdygvswir |
| | sequence | qpprkglewlgviwgsettyynsalksrltiikdnsksqvflkmnslqtddtaiyycakhyy |
| | | yggsyamdywgqgtsvtvsstttpaprpptpaptiasqplslrpeacrpaaggavhtrgldfa |
| | | cdiyiwaplagtcgvlllslvitlyckrgrkkllyifkqpfmrpvqttqeedgcscrfpeeeeg |
| | | gcelrvkfsrsadapaykqgqnqlynelnlgrreeydvldkrrgrdpemggkprrknpqeg |
| | | lynelqkdkmaeayseigmkgerrrgkghdglyqglstatkdtydalhmqalppr |
| 685 | CTL019 | atggccttaccagtgaccgccttgctcctgccgctggccttgctgctccacgccgccaggccg |
| | Full | gacatccagatgacacagactacatcctccctgtctgcctctctgggagacagagtcaccatca |
| | nucleotide | gttgcagggcaagtcaggacattagtaaatatttaaattggtatcagcagaaaccagatggaact |
| | sequence | gttaaactcctgatctaccatacatcaagattacactcaggagtcccatcaaggttcagtggcagt |
| | | gggtctggaacagattattctctcaccattagcaacctggagcaagaagatattgccacttactttt |
| | | gccaacagggtaatacgcttccgtacacgttcggaggggggaccaagctggagatcacaggt |
| | | ggcggtggctcgggcggtggtgggtcgggtggcggcggatctgaggtgaaactgcaggagt |
| | | caggacctggcctggtggcgccctcacagagcctgtccgtcacatgcactgtctcaggggtct |
| | | cattacccgactatggtgtaagctggattcgccagcctccacgaaagggtctggagtggctgg |
| | | gagtaatatggggtagtgaaaccacatactataattcagctctcaaatccagactgaccatcatc |
| | | aaggacaactccaagagccaagttttcttaaaaatgaacagtctgcaaactgatgacacagcca |
| | | tttactactgtgccaaacattattactacggtggtagctatgctatggactactggggccaaggaa |
| | | cctcagtcaccgtctcctcaaccacgacgccagcgccgcgaccaccaacaccggcgcccac |
| | | catcgcgtcgcagcccctgtccctgcgcccagaggcgtgccggccagcggggggggcgc |
| | | agtgcacacgagggggctggacttcgcctgtgatatctacatctgggcgcccttggccgggac |
| | | ttgtggggtccttctcctgtcactggttatcaccctttactgcaaacggggcagaaagaaactcct |
| | | gtatatattcaaacaaccatttatgagaccagtacaaactactcaagaggaagatggctgtagct |
| | | gccgatttccagaagaagaagaaggaggatgtgaactgagagtgaagttcagcaggagcgc |
| | | agacgcccccgcgtacaagcagggccagaaccagctctataacgagctcaatctaggacgaa |
| | | gagaggagtacgatgttttggacaagagacgtggccgggaccctgagatggggggaaagcc |
| | | gagaaggaagaaccctcaggaaggcctgtacaatgaactgcagaaagataagatggcggag |
| | | gcctacagtgagattgggatgaaaggcgagcgccggaggggcaaggggcacgatggccttt |
| | | accagggtctcagtacagccaccaaggacacctacgacgcccttcacatgcaggccctgccc |
| | | cctcgc |
| 686 | CTL019 | Diqmtqttsslsaslgdrvtiscrasqdiskylnwyqqkpdgtvklliyhtsrlhsgvpsrfsg |
| | scFv domain | sgsgtdysltisnleqediatyfcqqgntlpytfgggtkleitggggsggggsggggsevklqe |
| | | sgpglvapsqslsvtctvsgvslpdygvswirqpprkglewlgviwgsettyynsalksrltii |
| | | kdnsksqvflkmnslqtddtaiyycakhyyyggsyamdywgqgtsvtvss |
| Humanized | | |
| CAR2 | | |
| 760 | HCDR1 | DYGVS |
| | (Kabat) | |
| 687 | HCDR2 | VIWGSETTYYQSSLKS |
| | (Kabat) | |
| 762 | HCDR3 | HYYYGGSYAMDY |
| | (Kabat) | |
| 763 | LCDR1 | RASQDISKYLN |
| | (Kabat) | |
| 764 | LCDR2 | HTSRLHS |
| | (Kabat) | |
| 76- | LCDR3 | QQGNTLPYT |
| | (Kabat) | |
| 758 | CAR2 scFv | EIVMTQSPATLSLSPGERATLSCRASQDISKYLNWYQQKPG |
| | domain-aa | QAPRLLIYHTSRLHSGIPARFSGSGSGTDYTLTISSLQPEDFA |
| | (Linker is | VYFCQQGNTLPYTFGQGTKLEIKGGGGSGGGGSGGGGSQV |
| | underlined) | QLQESGPGLVKPSETLSLTCTVSGVSLPDYGVSWIRQPPGKG |
| | | LEWIGVIWGSETTYYQSSLKSRVTISKDNSKNQVSLKLSSVT |
| | | AADTAV-YCAKHYYYGGSYAMDYWGQGTLVTVSS |
| 688 | CAR2 scFv | atggccctccctgtcaccgccctgctgcttccgctggctcttctgctccacgccgctcggcccga |
| | domain-nt | aattgtgatgacccagtcacccgccactcttagcctttcacccggtgagcgcgcaaccctgtctt |
| | | gcagagcctcccaagacatctcaaaataccttaattggtatcaacagaagcccggacaggctc |
| | | ctcgccttctgatctaccacaccagccggctccattctggaatccctgccaggttcagcggtagc |
| | | ggatctgggaccgactacaccctcactatcagctcactgcagccagaggacttcgctgtctattt |
| | | ctgtcagcaagggaacaccctgccctacacctttggacagggcaccaagctcgagattaaagg |
| | | tggaggtggcagcggaggaggtgggtccggcggtggaggaagccaggtccaactccaaga |
| | | aagcggaccgggtcttgtgaagccatcagaaactctttcactgacttgtactgtgagcggagtgt |
| | | ctctccccgattacggggtgtcttggatcagacagccaccggggaagggtctggaatggattg |
| | | gagtgatttggggctctgagactacttactaccaatcatccctcaagtcacgcgtcaccatctcaa |
| | | aggacaactctaagaatcaggtgtcactgaaactgtcatctgtgaccgcagccgacaccgccg |
| | | tgtactattgcgctaagcattactattatggcgggagctacgcaatggattactggggacagggt |
| | | actctggtcaccgt-tccagccaccaccatcatcaccatcaccat |
| 689 | CAR 2- | MALPVTALLLPLALLLHAARPEIVMTQSPATLSLSPGERATL |
| | Full-aa | SCRASQDISKYLNWYQQKPGQAPRLLIYHTSRLHSGIPARFS |
| | | GSGSGTDYTLTISSLQPEDFAVYFCQQGNTLPYTFGQGTKLE |
| | | IKGGGGSGGGGSGGGGSQVQLQESGPGLVKPSETLSLTCTV |
| | | SGVSLPDYGVSWIRQPPGKGLEWIGVIWGSETTYYQSSLKS |
| | | RVTISKDNSKNQVSLKLSSVTAADTAVYYCAKHYYYGGSY |
| | | AMDYWGQGTLVTVSSTTTPAPRPPTPAPTIASQPLSLRPEAC |
| | | RPAAGGAVHTRGLDFACDIYIWAPLAGTCGVLLLSLVITLY |
| | | CKRGRKKLLYIFKQPFMRPVQTTQEEDGCSCRFPEEEEGGC |
| | | ELRVKFSRSADAPAYKQGQNQLYNELNLGRREEYDVLDKR |
| | | RGRDPEMGGKPRRKNPQEGLYNELQKDKMAEAYSEIGMK |
| | | GERR-GKGHDGLYQGLSTATKDTYDALHMQALPPR |
| 690 | CAR 2- | atggccctccctgtcaccgccctgctgcttccgctggctcttctgctccacgccgctcggcccga |
| | Full-nt | aattgtgatgacccagtcacccgccactcttagcctttcacccggtgagcgcgcaaccctgtctt |
| | | gcagagcctcccaagacatctcaaaataccttaattggtatcaacagaagcccggacaggctc |
| | | ctcgccttctgatctaccacaccagccggctccattctggaatccctgccaggttcagcggtagc |
| | | ggatctgggaccgactacaccctcactatcagctcactgcagccagaggacttcgctgtctattt |
| | | ctgtcagcaagggaacaccctgccctacacctttggacagggcaccaagctcgagattaaagg |
| | | tggaggtggcagcggaggaggtgggtccggcggtggaggaagccaggtccaactccaaga |
| | | aagcggaccgggtcttgtgaagccatcagaaactctttcactgacttgtactgtgagcggagtgt |
| | | ctctccccgattacggggtgtcttggatcagacagccaccggggaagggtctggaatggattg |
| | | gagtgatttggggctctgagactacttactaccaatcatccctcaagtcacgcgtcaccatctcaa |
| | | aggacaactctaagaatcaggtgtcactgaaactgtcatctgtgaccgcagccgacaccgccg |
| | | tgtactattgcgctaagcattactattatggcgggagctacgcaatggattactggggacagggt |
| | | actctggtcaccgtgtccagcaccactaccccagcaccgaggccacccaccccggctcctac |
| | | catcgcctcccagcctctgtccctgcgtccggaggcatgtagacccgcagctggtggggccgt |
| | | gcatacccggggtcttgacttcgcctgcgatatctacatttgggcccctctggctggtacttgcgg |
| | | ggtcctgctgctttcactcgtgatcactctttactgtaagcgcggtcggaagaagctgctgtacat |
| | | ctttaagcaacccttcatgaggcctgtgcagactactcaagaggaggacggctgttcatgccgg |
| | | ttcccagaggaggaggaaggcggctgcgaactgcgcgtgaaattcagccgcagcgcagatg |
| | | ctccagcctacaagcaggggcagaaccagctctacaacgaactcaatcttggtcggagagag |
| | | gagtacgacgtgctggacaagcggagaggacgggacccagaaatgggcgggaagccgcg |
| | | cagaaagaatccccaagagggcctgtacaacgagctccaaaaggataagatggcagaagcc |
| | | tatagcgagattggtatgaaaggggaacgcagaagaggcaaaggccacgacggactgtacc |
| | | agggactcagcaccgccaccaaggacacctatgacgctcttcacatgcaggccctgccgcct |
| | | cgg |
| 349 | CAR 2A- | MALPVTALLLPLALLLHAARPEIVMTQSPATLSLSPGERATL |
| | Full amino | SCRASQDISKYLNWYQQKPGQAPRLLIYHTSRLHSGIPARFS |
| | acid | GSGSGTDYTLTISSLQPEDFAVYFCQQGNTLPYTFGQGTKLE |
| | sequence; | IKGGGGSGGGGSGGGGSQVQLQESGPGLVKPSETLSLTCTV |
| | signal | SGVSLPDYGVSWIRQPPGKGLEWIGVIWGSETTYYQSSLKS |
| | peptide | RVTISKDNSKNQVSLKLSSVTAADTAVYYCAKHYYYGGSY |
| | underlined | AMDYWGQGTLVTVSSTTTPAPRPPTPAPTIASQPLSLRPEAC |
| | | RPAAGGAVHTRGLDFACDIYIWAPLAGTCGVLLLSLVITLY |
| | | CKRGRKKLLYIFKQPFMRPVQTTQEEDGCCRFPEEEEGGC |
| | | ELRVKFSRSADAPAYQQGQNQLYNELNLGRREEYDVLDKR |
| | | RGRDPEMGGKPRRKNPQEGLYNELQKDKMAEAYSEIGMK |
| | | GERRRGKGHDGLYQGLSTATKDTYDALHMQALPPR |
| 225 | CAR 2A- | EIVMTQSPATLSLSPGERATLSCRASQDISKYLNWYQQKPG |
| | amino acid | QAPRLLIYHTSRLHSGIPARFSGSGSGTDYTLTISSLQPEDFA |
| | sequence; | VYFCQQGNTLPYTFGQGTKLEIKGGGGSGGGGSGGGGSQV |
| | no signal | QLQESGPGLVKPSETLSLTCTVSGVSLPDYGVSWIRQPPGKG |
| | peptide | LEWIGVIWGSETTYYQSSLKSRVTISKDNSKNQVSLKLSSVT |
| | | AADTAVYYCAKHYYYGGSYAMDYWGQGTLVTVSSTTTPA |
| | | PRPPTPAPTIASQPLSLRPEACRPAAGGAVHTRGLDFACDIYI |
| | | WAPLAGTCGVLLLSLVITLYCKRGRKKLLYIFKQPFMRPVQ |
| | | TTQEEDGCSCRFPEEEEGGCELRVKFSRSADAPAYQQGQNQ |
| | | LYNELNLGRREEYDVLDKRRGRDPEMGGKPRRKNPQEGLY |
| | | NELQKDKMAEAYSEIGMKGERRRGKGHDGLYQGLSTATK |
| | | DTYDALHMQALPPR |
| 354 | CAR 2A | atggccctccctgtcaccgccctgctgcttccgctggctcttctgctccacgccgctcggcccga |
| | full nucleic | aattgtgatgacccagtcacccgccactcttagcctttcacccggtgagcgcgcaaccctgtctt |
| | acid | gcagagcctcccaagacatctcaaaataccttaattggtatcaacagaagcccggacaggctc |
| | sequence; | ctcgccttctgatctaccacaccagccggctccattctggaatccctgccaggttcagcggtagc |
| | signal | ggatctgggaccgactacaccctcactatcagctcactgcagccagaggacttcgctgtctattt |
| | peptide and | ctgtcagcaagggaacaccctgccctacacctttggacagggcaccaagctcgagattaaagg |
| | stop codon | tggaggtggcagcggaggaggtgggtccggcggtggaggaagccaggtccaactccaaga |
| | underlined | aagcggaccgggtcttgtgaagccatcagaaactctttcactgacttgtactgtgagcggagtgt |
| | | ctctccccgattacggggtgtcttggatcagacagccaccggggaagggtctggaatggattg |
| | | gagtgatttggggctctgagactacttactaccaatcatccctcaagtcacgcgtcaccatctcaa |
| | | aggacaactctaagaatcaggtgtcactgaaactgtcatctgtgaccgcagccgacaccgccg |
| | | tgtactattgcgctaagcattactattatggcgggagctacgcaatggattactggggacagggt |
| | | actctggtcaccgtgtccagcaccactaccccagcaccgaggccacccaccccggctcctac |
| | | catcgcctcccagcctctgtccctgcgtccggaggcatgtagacccgcagctggtggggccgt |
| | | gcatacccggggtcttgacttcgcctgcgatatctacatttgggcccctctggctggtacttgcgg |
| | | ggtcctgctgctttcactcgtgatcactctttactgtaagcgcggtcggaagaagctgctgtacat |
| | | ctttaagcaacccttcatgaggcctgtgcagactactcaagaggaggacggctgttcatgccgg |
| | | ttcccagaggaggaggaaggcggctgcgaactgcgcgtgaaattcagccgcagcgcagatg |
| | | ctccagcctaccagcaggggcagaaccagctctacaacgaactcaatcttggtcggagagag |
| | | gagtacgacgtgctggacaagcggagaggacgggacccagaaatgggcgggaagccgcg |
| | | cagaaagaatccccaagagggcctgtacaacgagctccaaaaggataagatggcagaagcc |
| | | tatagcgagattggtatgaaaggggaacgcagaagaggcaaaggccacgacggactgtacc |
| | | agggactcagcaccgccaccaaggacacctatgacgctcttcacatgcaggccctgccgcct |
| | | cggtaa |
| 355 | CAR 2A | atggccctccctgtcaccgccctgctgcttccgctggctcttctgctccacgccgctcggcccga |
| | nucleic acid | aattgtgatgacccagtcacccgccactcttagcctttcacccggtgagcgcgcaaccctgtctt |
| | sequence; | gcagagcctcccaagacatctcaaaataccttaattggtatcaacagaagcccggacaggctc |
| | signal | ctcgccttctgatctaccacaccagccggctccattctggaatccctgccaggttcagcggtagc |
| | peptide | ggatctgggaccgactacaccctcactatcagctcactgcagccagaggacttcgctgtctattt |
| | underlined; | ctgtcagcaagggaacaccctgccctacacctttggacagggcaccaagctcgagattaaagg |
| | no stop | tggaggtggcagcggaggaggtgggtccggcggtggaggaagccaggtccaactccaaga |
| | codon | aagcggaccgggtcttgtgaagccatcagaaactctttcactgacttgtactgtgagcggagtgt |
| | | ctctccccgattacggggtgtcttggatcagacagccaccggggaagggtctggaatggattg |
| | | gagtgatttggggctctgagactacttactaccaatcatccctcaagtcacgcgtcaccatctcaa |
| | | aggacaactctaagaatcaggtgtcactgaaactgtcatctgtgaccgcagccgacaccgccg |
| | | tgtactattgcgctaagcattactattatggcgggagctacgcaatggattactggggacagggt |
| | | actctggtcaccgtgtccagcaccactaccccagcaccgaggccacccaccccggctcctac |
| | | catcgcctcccagcctctgtccctgcgtccggaggcatgtagacccgcagctggtggggccgt |
| | | gcatacccggggtcttgacttcgcctgcgatatctacatttgggcccctctggctggtacttgcgg |
| | | ggtcctgctgctttcactcgtgatcactctttactgtaagcgcggtcggaagaagctgctgtacat |
| | | ctttaagcaacccttcatgaggcctgtgcagactactcaagaggaggacggctgttcatgccgg |
| | | ttcccagaggaggaggaaggcggctgcgaactgcgcgtgaaattcagccgcagcgcagatg |
| | | ctccagcctaccagcaggggcagaaccagctctacaacgaactcaatcttggtcggagagag |
| | | gagtacgacgtgctggacaagcggagaggacgggacccagaaatgggcgggaagccgcg |
| | | cagaaagaatccccaagagggcctgtacaacgagctccaaaaggataagatggcagaagcc |
| | | tatagcgagattggtatgaaaggggaacgcagaagaggcaaaggccacgacggactgtacc |
| | | agggactcagcaccgccaccaaggacacctatgacgctcttcacatgcaggccctgccgcct |
| | | cgg |
| 356 | CAR 2A | gaaattgtgatgacccagtcacccgccactcttagcctttcacccggtgagcgcgcaaccctgt |
| | nucleic acid | cttgcagagcctcccaagacatctcaaaataccttaattggtatcaacagaagcccggacaggc |
| | sequence; | tcctcgccttctgatctaccacaccagccggctccattctggaatccctgccaggttcagcggta |
| | no signal | gcggatctgggaccgactacaccctcactatcagctcactgcagccagaggacttcgctgtcta |
| | peptide; stop | tttctgtcagcaagggaacaccctgccctacacctttggacagggcaccaagctcgagattaaa |
| | codon | ggtggaggtggcagcggaggaggtgggtccggcggtggaggaagccaggtccaactccaa |
| | underlined | gaaagcggaccgggtcttgtgaagccatcagaaactctttcactgacttgtactgtgagcggag |
| | | tgtctctccccgattacggggtgtcttggatcagacagccaccggggaagggtctggaatggat |
| | | tggagtgatttggggctctgagactacttactaccaatcatccctcaagtcacgcgtcaccatctc |
| | | aaaggacaactctaagaatcaggtgtcactgaaactgtcatctgtgaccgcagccgacaccgc |
| | | cgtgtactattgcgctaagcattactattatgggggagctacgcaatggattactggggacagg |
| | | gtactctggtcaccgtgtccagcaccactaccccagcaccgaggccacccaccccggctccta |
| | | ccatcgcctcccagcctctgtccctgcgtccggaggcatgtagacccgcagctggtggggcc |
| | | gtgcatacccggggtcttgacttcgcctgcgatatctacatttgggcccctctggctggtacttgc |
| | | ggggtcctgctgctttcactcgtgatcactctttactgtaagcgcggtcggaagaagctgctgtac |
| | | atctttaagcaacccttcatgaggcctgtgcagactactcaagaggaggacggctgttcatgcc |
| | | ggttcccagaggaggaggaaggcggctgcgaactgcgcgtgaaattcagccgcagcgcag |
| | | atgctccagcctaccagcaggggcagaaccagctctacaacgaactcaatcttggtcggagag |
| | | aggagtacgacgtgctggacaagcggagaggacgggacccagaaatgggcgggaagccg |
| | | cgcagaaagaatccccaagagggcctgtacaacgagctccaaaaggataagatggcagaag |
| | | cctatagcgagattggtatgaaaggggaacgcagaagaggcaaaggccacgacggactgta |
| | | ccagggactcagcaccgccaccaaggacacctatgacgctcttcacatgcaggccctgccgc |
| | | ctcggtaa |
| 417 | CAR 2A | gaaattgtgatgacccagtcacccgccactcttagcctttcacccggtgagcgcgcaaccctgt |
| | nucleic acid | cttgcagagcctcccaagacatctcaaaataccttaattggtatcaacagaagcccggacaggc |
| | sequence; | tcctcgccttctgatctaccacaccagccggctccattctggaatccctgccaggttcagcggta |
| | no signal | gcggatctgggaccgactacaccctcactatcagctcactgcagccagaggacttcgctgtcta |
| | peptide; no | tttctgtcagcaagggaacaccctgccctacacctttggacagggcaccaagctcgagattaaa |
| | stop codon | ggtggaggtggcagcggaggaggtgggtccggcggtggaggaagccaggtccaactccaa |
| | | gaaagcggaccgggtcttgtgaagccatcagaaactctttcactgacttgtactgtgagcggag |
| | | tgtctctccccgattacggggtgtcttggatcagacagccaccggggaagggtctggaatggat |
| | | tggagtgatttggggctctgagactacttactaccaatcatccctcaagtcacgcgtcaccatctc |
| | | aaaggacaactctaagaatcaggtgtcactgaaactgtcatctgtgaccgcagccgacaccgc |
| | | cgtgtactattgcgctaagcattactattatggcgggagctacgcaatggattactggggacagg |
| | | gtactctggtcaccgtgtccagcaccactaccccagcaccgaggccacccaccccggctccta |
| | | ccatcgcctcccagcctctgtccctgcgtccggaggcatgtagacccgcagctggtggggcc |
| | | gtgcatacccggggtcttgacttcgcctgcgatatctacatttgggcccctctggctggtacttgc |
| | | ggggtcctgctgctttcactcgtgatcactctttactgtaagcgcggtcggaagaagctgctgtac |
| | | atctttaagcaacccttcatgaggcctgtgcagactactcaagaggaggacggctgttcatgcc |
| | | ggttcccagaggaggaggaaggcggctgcgaactgcgcgtgaaattcagccgcagcgcag |
| | | atgctccagcctaccagcaggggcagaaccagctctacaacgaactcaatcttggtcggagag |
| | | aggagtacgacgtgctggacaagcggagaggacgggacccagaaatgggcgggaagccg |
| | | cgcagaaagaatccccaagagggcctgtacaacgagctccaaaaggataagatggcagaag |
| | | cctatagcgagattggtatgaaaggggaacgcagaagaggcaaaggccacgacggactgta |
| | | ccagggactcagcaccgccaccaaggacacctatgacgctcttcacatgcaggccctgccgc |
| | | ctcgg |
| 250 | Anti-CD19 | QVQLQESGPGLVKPSETLSLTCTVSGVSLPDYGVSWIRQPPG |
| | VH | KGLEWIGVIWGSETTYYQSSLKSRVTISKDNSKNQVSLKLSS |
| | | VTAADTAVYYCAKHYYYGGSYAMDYWGQGTL VTVSS |
| 251 | Anti-CD19 | EIVMTQSPATLSLSPGERATLSCRASQDISKYLNWYQQKPG |
| | VL | QAPRLLIYHTSRLHSGIPARFSGSGSGTDYTLTISSLQPEDFA |
| | | VYFCQQGNTLPYTFGQGTKLEIK |
| 331 | VH | QVQLQESGPGLVKPSETLSLTCTVSGVSLPDYGVSWIRQPPG |
| | | KCLEWIGVIWGSETTYYQSSLKSRVTISKDNSKNQVSLKLSS |
| | | VTAADTAVYYCAKHYYYGGSYAMDYWGQGTL VTVSS |
| 332 | VL | EIVMTQSPATLSLSPGERATLSCRASQDISKYLNWYQQKPG |
| | | QAPRLLIYHTSRLHSGIPARFSGSGSGTDYTLTISSLQPEDFA |
| | | VYFCQQGNTLPYTFGCGTKLEIK |

### Exemplary CD19 CAR A

In some embodiments, the CD19 CAR is a comprises a binding domain of the FMC63 monoclonal antibody-derived single-chain variable fragment (scFv), IgG4 hinge region, CD28 transmembrane domain, 4-1BB (CD137) costimulatory domain, and CD3 zeta activation domain. In some embodiments, the CD19 CAR is encoded by a nucleotide sequence of Table 25, or a nucleotide sequence having at least 80%, 85%, 90%, 95%, 97%, or 100% or no more than 20, 15, 10, 8, 5, 4, 3, 2, or 1 variations thereto. In some embodiments, the CD19 CAR comprises a polypeptide encoded by a nucleotide sequence of Table 25, or a nucleotide sequence having at least 80%, 85%, 90%, 95%, 97%, or 100% or no more than 20, 15, 10, 8, 5, 4, 3, 2, or 1 variations thereto. In some embodiments, the CD19 CAR comprises a polypeptide sequence of Table 25, or a polypeptide sequence having at least 80%, 85%, 90%, 95%, 97%, or 100% or no more than 20, 15, 10, 8, 5, 4, 3, 2, or 1 variations thereto. In some embodiments, the CD19 CAR comprises a heavy chain CDR1-3 and a light chain CDR1-3, of a sequence of Table 25. In some embodiments, the CD19 CAR comprises a heavy chain CDR1-3 and a light chain CDR1-3, of a sequence of Table 25 according to Kabat. In some embodiments, the CD19 CAR comprises a heavy chain CDR1-3 and a light chain CDR1-3, of a sequence of Table 25 according to Chothia. In some embodiments, the CD19 CAR comprises a heavy chain CDR1-3 of a sequence of Table 25. In some embodiments, the CD19 CAR comprises a heavy chain CDR1-3 of a sequence of Table 25 according to Kabat. In some embodiments, the CD19 CAR comprises a heavy chain CDR1-3 of a sequence of Table 25 according to Chothia. In some embodiments, the CD19 CAR comprises a light chain CDR1-3 of a sequence of Table 25. In some embodiments, the CD19 CAR comprises a light chain CDR1-3 of a sequence of Table 25 according to Kabat. In some embodiments, the CD19 CAR comprises a light chain CDR1-3 of a sequence of Table 25 according to Chothia.

TABLE 25
Amino acid and nucleic acid sequences of an exemplary CD19 CAR

| SEQ ID NO: | Description | Sequence |
|---|---|---|
| 357 | CAR A full | atgcttctcctggtgacaagccttctgctctgtgagttaccacaccca |
| | nucleotide | gcattcctcctgatcccagacatccagatgacacagactacatcctcc |
| | sequence; | ctgtctgcctctctgggagacagagtcaccatcagttgcagggcaagt |
| | with leader | caggacattagtaaatatttaaattggtatcagcagaaaccagatgga |
| | | actgttaaactcctgatctaccatacatcaagattacactcaggagtc |
| | | ccatcaaggttcagtggcagtgggtctggaacagattattctctcacc |
| | | attagcaacctggagcaagaagatattgccacttacttttgccaacag |
| | | ggtaatacgcttccgtacacgttcggaggggggactaagttggaaata |
| | | acaggctccacctctggatccggcaagcccggatctggcgagggatcc |
| | | accaagggcgaggtgaaactgcaggagtcaggacctggcctggtggcg |
| | | ccctcacagagcctgtccgtcacatgcactgtctcaggggtctcatta |
| | | cccgactatggtgtaagctggattcgccagcctccacgaaagggtctg |
| | | gagtggctgggagtaatatggggtagtgaaaccacatactataattca |
| | | gctctcaaatccagactgaccatcatcaaggacaactccaagagccaa |
| | | gttttcttaaaaatgaacagtctgcaaactgatgacacagccatttac |
| | | tactgtgccaaacattattactacggtggtagctatgctatggactac |
| | | tggggtcaaggaacctcagtcaccgtctcctcagcggccgcaattgaa |
| | | gttatgtatcctcctccttacctagacaatgagaagagcaatggaacc |
| | | attatccatgtgaaagggaaacacctttgtccaagtcccctatttccc |
| | | ggaccttctaagcccttttgggtgctggtggtggttgggggagtcctg |
| | | gcttgctatagcttgctagtaacagtggcctttattattttctgggtg |
| | | aggagtaagaggagcaggctcctgcacagtgactacatgaacatgact |
| | | ccccgccgccccgggcccacccgcaagcattaccagccctatgcccca |
| | | ccacgcgacttcgcagcctatcgctccagagtgaagttcagcaggagc |
| | | gcagacgcccccgcgtaccagcagggccagaaccagctctataacgag |
| | | ctcaatctaggacgaagagaggagtacgatgttttggacaagagacgt |
| | | ggccgggaccctgagatggggggaaagccgagaaggaagaaccctcag |
| | | gaaggcctgtacaatgaactgcagaaagataagatggcggaggcctac |
| | | agtgagattgggatgaaaggcgagcgccggaggggcaaggggcacgat |
| | | ggcctttaccagggtctcagtacagccaccaaggacacctacgacgcc |
| | | cttcacatgcaggccctgccccctcgc |
| 358 | CAR A-full | MLLLVTSLLLCELPHPAFLLIPDIQMTQTTSSLSASLGDRVTISCRAS |
| | amino acid | QDISKYLNWYQQKPDGTVKLLIYHTSRLHSGVPSRFSGSGSGTDYSLT |
| | transgene | ISNLEQEDIATYFCQQGNTLPYTFGGGTKLEITGSTSGSGKPGSGEGS |
| | sequence; | TKGEVKLQESGPGLVAPSQSLSVTCTVSGVSLPDYGVSWIRQPPRKGL |
| | with leader | EWLGVIWGSETTYYNSALKSRLTIIKDNSKSQVFLKMNSLQTDDTAIY |
| | | YCAKHYYYGGSYAMDYWGQGTSVTVSSAAAIEVMYPPPYLDNEKSNGT |
| | | IIHVKGKHLCPSPLFPGPSKPFWVLVVVGGVLACYSLLVTVAFIIFWV |
| | | RSKRSRLLHSDYMNMTPRRPGPTRKHYQPYAPPRDFAAYRSRVKFSRS |
| | | ADAPAYQQGQNQLYNELNLGRREEYDVLDKRRGRDPEMGGKPRRKNPQ |
| | | EGLYNELQKDKMAEAYSEIGMKGERRRGKGHDGLYQGLSTATKDTYDA |
| | | LHMQALPPR |
| 359 | CAR A- | atgcttctcctggtgacaagccttctgctctgtgagttaccacaccca |
| | CD19 scFv | gcattcctcctgatcccagacatccagatgacacagactacatcctcc |
| | nucleotide | ctgtctgcctctctgggagacagagtcaccatcagttgcagggcaagt |
| | sequence | caggacattagtaaatatttaaattggtatcagcagaaaccagatgga |
| | with leader | actgttaaactcctgatctaccatacatcaagattacactcaggagtc |
| | | ccatcaaggttcagtggcagtgggtctggaacagattattctctcacc |
| | | attagcaacctggagcaagaagatattgccacttacttttgccaacag |
| | | ggtaatacgcttccgtacacgttcggaggggggactaagttggaaata |
| | | acaggctccacctctggatccggcaagcccggatctggcgagggatcc |
| | | accaagggcgaggtgaaactgcaggagtcaggacctggcctggtggcg |
| | | ccctcacagagcctgtccgtcacatgcactgtctcaggggtctcatta |
| | | cccgactatggtgtaagctggattcgccagcctccacgaaagggtctg |
| | | gagtggctgggagtaatatggggtagtgaaaccacatactataattca |
| | | gctctcaaatccagactgaccatcatcaaggacaactccaagagccaa |
| | | gttttcttaaaaatgaacagtctgcaaactgatgacacagccatttac |
| | | tactgtgccaaacattattactacggtggtagctatgctatggactac |
| | | tggggtcaaggaacctcagtcaccgtctcctca |
| 360 | CAR A- | MLLLVTSLLLCELPHPAFLLIPDIQMTQTTSSLSASLGDRVTISCRAS |
| | CD19 scFv | QDISKYLNWYQQKPDGTVKLLIYHTSRLHSGVPSRFSGSGSGTDYSLT |
| | amino acid | ISNLEQEDIATYFCQQGNTLPYTFGGGTKLEITGSTSGSGKPGSGEGS |
| | sequence; | TKGEVKLQESGPGLVAPSQSLSVTCTVSGVSLPDYGVSWIRQPPRKGL |
| | with leader | EWLGVIWGSETTYYNSALKSRLTIIKDNSKSQVFLKMNSLQTDDTAIY |
| | | YCAKHYYYGGSYAMDYWGQGTSVTVS |
| 361 | CAR A-full | gacatccagatgacacagactacatcctccctgtctgcctctctggga |
| | nucleotide | gacagagtcaccatcagttgcagggcaagtcaggacattagtaaatat |
| | sequence; | ttaaattggtatcagcagaaaccagatggaactgttaaactcctgatc |
| | no leader | taccatacatcaagattacactcaggagtcccatcaaggttcagtggc |
| | | agtgggtctggaacagattattctctcaccattagcaacctggagcaa |
| | | gaagatattgccacttacttttgccaacagggtaatacgcttccgtac |
| | | acgttcggaggggggactaagttggaaataacaggctccacctctgga |
| | | tccggcaagcccggatctggcgagggatccaccaagggcgaggtgaaa |
| | | ctgcaggagtcaggacctggcctggtggcgccctcacagagcctgtcc |
| | | gtcacatgcactgtctcaggggtctcattacccgactatggtgtaagc |
| | | tggattcgccagcctccacgaaagggtctggagtggctgggagtaata |
| | | tggggtagtgaaaccacatactataattcagctctcaaatccagactg |
| | | accatcatcaaggacaactccaagagccaagttttcttaaaaatgaac |
| | | agtctgcaaactgatgacacagccatttactactgtgccaaacattat |
| | | tactacggtggtagctatgctatggactactggggtcaaggaacctca |
| | | gtcaccgtctcctcagcggccgcaattgaagttatgtatcctcctcct |
| | | tacctagacaatgagaagagcaatggaaccattatccatgtgaaaggg |
| | | aaacacctttgtccaagtcccctatttcccggaccttctaagcccttt |
| | | tgggtgctggtggtggttgggggagtcctggcttgctatagcttgcta |
| | | gtaacagtggcctttattattttctgggtgaggagtaagaggagcagg |
| | | ctcctgcacagtgactacatgaacatgactccccgccgccccgggccc |
| | | acccgcaagcattaccagccctatgccccaccacgcgacttcgcagcc |
| | | tatcgctccagagtgaagttcagcaggagcgcagacgcccccgcgtac |
| | | cagcagggccagaaccagctctataacgagctcaatctaggacgaaga |
| | | gaggagtacgatgttttggacaagagacgtggccgggaccctgagatg |
| | | gggggaaagccgagaaggaagaaccctcaggaaggcctgtacaatgaa |
| | | ctgcagaaagataagatggcggaggcctacagtgagattgggatgaaa |
| | | ggcgagcgccggaggggcaaggggcacgatggcctttaccagggtctc |
| | | agtacagccaccaaggacacctacgacgcccttcacatgcaggccctg |
| | | ccccctcgc |
| 362 | CAR A-full | DIQMTQTTSSLSASLGDRVTISCRASQDISKYLNWYQQKPDGTVKLLI |
| | amino acid | YHTSRLHSGVPSRFSGSGSGTDYSLTISNLEQEDIATYFCQQGNTLPY |
| | transgene | TFGGGTKLEITGSTSGSGKPGSGEGSTKGEVKLQESGPGLVAPSQSLS |
| | sequence; | VTCTVSGVSLPDYGVSWIRQPPRKGLEWLGVIWGSETTYYNSALKSRL |
| | no leader | TIIKDNSKSQVFLKMNSLQTDDTAIYYCAKHYYYGGSYAMDYWGQGTS |
| | | VTVSSAAAIEVMYPPPYLDNEKSNGTIIHVKGKHLCPSPLFPGPSKPF |
| | | WVLVVVGGVLACYSLLVTVAFIIFWVRSKRSRLLHSDYMNMTPRRPGP |
| | | TRKHYQPYAPPRDFAAYRSRVKFSRSADAPAYQQGQNQLYNELNLGRR |
| | | EEYDVLDKRRGRDPEMGGKPRRKNPQEGLYNELQKDKMAEAYSEIGMK |
| | | GERRRGKGHDGLYQGLSTATKDTYDALHMQALPPR |
| 363 | CAR A- | gacatccagatgacacagactacatcctccctgtctgcctctctggga |
| | CD19 scFv | gacagagtcaccatcagttgcagggcaagtcaggacattagtaaatat |
| | nucleotide; | ttaaattggtatcagcagaaaccagatggaactgttaaactcctgatc |
| | no leader | taccatacatcaagattacactcaggagtcccatcaaggttcagtggc |
| | | agtgggtctggaacagattattctctcaccattagcaacctggagcaa |
| | | gaagatattgccacttacttttgccaacagggtaatacgcttccgtac |
| | | acgttcggaggggggactaagttggaaataacaggctccacctctgga |
| | | tccggcaagcccggatctggcgagggatccaccaagggcgaggtgaaa |
| | | ctgcaggagtcaggacctggcctggtggcgccctcacagagcctgtcc |
| | | gtcacatgcactgtctcaggggtctcattacccgactatggtgtaagc |
| | | tggattcgccagcctccacgaaagggtctggagtggctgggagtaata |
| | | tggggtagtgaaaccacatactataattcagctctcaaatccagactg |
| | | accatcatcaaggacaactccaagagccaagttttcttaaaaatgaac |
| | | agtctgcaaactgatgacacagccatttactactgtgccaaacattat |
| | | tactacggtggtagctatgctatggactactggggtcaaggaacctca |
| | | gtcaccgtctcctca |
| 364 | CAR A- | DIQMTQTTSSLSASLGDRVTISCRASQDISKYLNWYQQKPDGTVKLLI |
| | CD19 scFv | YHTSRLHSGVPSRFSGSGSGTDYSLTISNLEQEDIATYFCQQGNTLPY |
| | amino acid | TFGGGTKLEITGSTSGSGKPGSGEGSTKGEVKLQESGPGLVAPSQSLS |
| | sequence; | VTCTVSGVSLPDYGVSWIRQPPRKGLEWLGVIWGSETTYYNSALKSRL |
| | no leader | TIIKDNSKSQVFLKMNSLQTDDTAIYYCAKHYYYGGSYAMDYWGQGTS |
| | | VTVSS |

### Exemplary CD19 CAR B

In some embodiments, the CD19 CAR comprises a murine anti-CD19 single-chain variable fragment (scFv) linked to CD28 and CD3-zeta co-stimulatory domains. In certain embodiments, the anti-CD19 single-chain variable fragment comprises the FMC63 antibody (e.g., the antibody described in Nicholson et al., Molecular Immunology, 34(16-17):1157-1165, 1997; the entire contents of which are incorporated herein by reference). In some embodiments, the CD19 CAR is encoded by a nucleotide sequence of Table 26, or a nucleotide sequence having at least 80%, 85%, 90%, 95%, 97%, or 100% or no more than 20, 15, 10, 8, 5, 4, 3, 2, or 1 variations thereto. In some embodiments, the CD19 CAR comprises a polypeptide encoded by a nucleotide sequence of Table 26, or a nucleotide sequence having at least 80%, 85%, 90%, 95%, 97%, or 100% or no more than 20, 15, 10, 8, 5, 4, 3, 2, or 1 variations thereto. In some embodiments, the CD19 CAR comprises a polypeptide sequence of Table 26, or a polypeptide sequence having at least 80%, 85%, 90%, 95%, 97%, or 100% or no more than 20, 15, 10, 8, 5, 4, 3, 2, or 1 variations thereto. In some embodiments, the CD19 CAR comprises a heavy chain CDR1-3 and a light chain CDR1-3, of a sequence of Table 26. In some embodiments, the CD19 CAR comprises a heavy chain CDR1-3 and a light chain CDR1-3, of a sequence of Table 26 according to Kabat. In some embodiments, the CD19 CAR comprises a heavy chain CDR1-3 and a light chain CDR1-3, of a sequence of Table 26 according to Chothia. In some embodiments, the CD19 CAR comprises a heavy chain CDR1-3 of a sequence of Table 26. In some embodiments, the CD19 CAR comprises a heavy chain CDR1-3 of a sequence of Table 26 according to Kabat. In some embodiments, the CD19 CAR comprises a heavy chain CDR1-3 of a sequence of Table 26 according to Chothia. In some embodiments, the CD19 CAR comprises a light chain CDR1-3 of a sequence of Table 26. In some embodiments, the CD19 CAR comprises a light chain CDR1-3 of a sequence of Table 26 according to Kabat. In some embodiments, the CD19 CAR comprises a light chain CDR1-3 of a sequence of Table 26 according to Chothia.

TABLE 26
Amino acid and nucleic acid sequences of an exemplary CD19 CAR

| SEQ ID NO: | Description | Sequence |
|---|---|---|
| 365 | Exemplary | MLLLVTSLLLCELPHPAFLLIPDIQMTQTTSSLSASLGDRVTISCR |
| | CD19 CAR | ASQDISKYLNWYQQKPDGTVKLLIYHTSRLHSGVPSRFSGSGSGTD |
| | Protein | YSLTISNLEQEDIATYFCQQGNTLPYTFGGGTKLEITGSTSGSGKP |
| | Sequence | GSGEGSTKGEVKLQESGPGLVAPSQSLSVTCTVSGVSLPDYGVSWI |
| | | RQPPRKGLEWLGVIWGSETTYYNSALKSRLTIIKDNSKSQVFLKMN |
| | | SLQTDDTAIYYCAKHYYYGGSYAMDYWGQGTSVTVSSFVPVFLPAK |
| | | PTTTPAPRPPTPAPTIASQPLSLRPEACRPAAGGAVHTRGLDFACD |
| | | IYIWAPLAGTCGVLLLSLVITLYCNHRNRSKRSRLLHSDYMNMTPR |
| | | RPGPTRKHYQPYAPPRDFAAYRSRVKFSRSADAPAYQQGQNQLYNE |
| | | LNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| 366 | Exemplary | ATGCTTCTCCTGGTGACAAGCCTTCTGCTCTGTGAGTTACCA |
| | CD19 CAR | CACCCAGCATTCCTCCTGATCCCAGACATCCAGATGACACAG |
| | Nucleic | ACTACATCCTCCCTGTCTGCCTCTCTGGGAGACAGAGTCACC |
| | Acid | ATCAGTTGCAGGGCAAGTCAGGACATTAGTAAATATTTAAAT |
| | Sequence | TGGTATCAGCAGAAACCAGATGGAACTGTTAAACTCCTGATC |
| | | TACCATACATCAAGATTACACTCAGGAGTCCCATCAAGGTTC |
| | | AGTGGCAGTGGGTCTGGAACAGATTATTCTCTCACCATTAGC |
| | | AACCTGGAGCAAGAAGATATTGCCACTTACTTTTGCCAACAG |
| | | GGTAATACGCTTCCGTACACGTTCGGAGGGGGGACTAAGTTG |
| | | GAAATAACAGGCTCCACCTCTGGATCCGGCAAGCCCGGATCT |
| | | GGCGAGGGATCCACCAAGGGCGAGGTGAAACTGCAGGAGTCA |
| | | GGACCTGGCCTGGTGGCGCCCTCACAGAGCCTGTCCGTCACA |
| | | TGCACTGTCTCAGGAGGGTCTGGAGGTCTCATTACCCGACTA |
| | | TGGTGTAAGCTGGATTCGCCAGCCTCCACGAAGTGGCTGGGA |
| | | GTAATATGGGGTAGTGAAACCACATACTATAATTCAGCTCTC |
| | | AAATCCAGACTGACCATCATCAAGGACAACTCCAAGAGCCAA |
| | | GTTTTCTTAAAAATGAACAGTCTGCAAACTGATGACACAGCC |
| | | ATTTACTACTGTGCCAAACATTATTACTACGGTGGTAGCTAT |
| | | GCTATGGACTACTGGGGTCAAGGAACCTCAGTCACCGTCTCC |
| | | TCAGCGGCCGCAATTGAAGTTATGTATCCTCCTCCTTACCTA |
| | | GACAATGAGAAGAGCAATGGAACCATTATCCATGTGAAAGGG |
| | | AAACACCTTTGTCCAAGTCCCCTATTTCCCGGACCTTCTAAG |
| | | CCCTTTTGGGTGCTGGTGGTGGTTGGGGGAGTCCTGGCTTGC |
| | | TATAGCTTGCTAGTAACAGTGGCCTTTATTATTTTCTGGGTG |
| | | AGGAGTAAGAGGAGCAGGCTCCTGCACAGTGACTACATGAAC |
| | | ATGACTCCCCGCCGCCCCGGGCCCACCCGCAAGCATTACCAG |
| | | CCCTATGCCCCACCACGCGACTTCGCAGCCTATCGCTCCAGA |
| | | GTGAAGTTCAGCAGGAGCGCAGACGCCCCCGCGTACCAGCAG |
| | | GGCCAGAACCAGCTCTATAACGAGCTCAATCTAGGACGAAGA |
| | | GAGGAGTACGATGTTTTGGACAAGAGACGTGGCCGGGACCCT |
| | | GAGATGGGGGGAAAGCCGAGAAGGAAGAACCCTCAGGAAGGC |
| | | CTGTACAATGAACTGCAGAAAGATAAGATGGCGGAGGCCTAC |
| | | AGTGAGATTGGGATGAAAGGCGAGCGCCGGAGGGGCAAGGGG |
| | | CACGATGGCCTTTACCAGGGTCTCAGTACAGCCACCAAGGAC |
| | | ACCTACGACGCCCTTCACATGCAGGCCCTGCCCCCTCGC |

### Exemplary CD19 CAR C

In some embodiments, the CD19 CAR comprises a murine anti-CD19 single chain variable fragment (scFv) linked to CD28 and CD3-zeta co-stimulatory domains. In some embodiments, the CD19 CAR is encoded by a nucleotide sequence of Table 27, or a nucleotide sequence having at least 80%, 85%, 90%, 95%, 97%, or 100% or no more than 20, 15, 10, 8, 5, 4, 3, 2, or 1 variations thereto. In some embodiments, the CD19 CAR comprises a polypeptide encoded by a nucleotide sequence of Table 27, or a nucleotide sequence having at least 80%, 85%, 90%, 95%, 97%, or 100% or no more than 20, 15, 10, 8, 5, 4, 3, 2, or 1 variations thereto. In some embodiments, the CD19 CAR comprises a polypeptide sequence of Table 27, or a polypeptide sequence having at least 80%, 85%, 90%, 95%, 97%, or 100% or no more than 20, 15, 10, 8, 5, 4, 3, 2, or 1 variations thereto. In some embodiments, the CD19 CAR comprises a heavy chain CDR1-3 and a light chain CDR1-3, of a sequence of Table 27. In some embodiments, the CD19 CAR comprises a heavy chain CDR1-3 and a light chain CDR1-3, of a sequence of Table 27 according to Kabat. In some embodiments, the CD19 CAR comprises a heavy chain CDR1-3 and a light chain CDR1-3, of a sequence of Table 27 according to Chothia. In some embodiments, the CD19 CAR comprises a heavy chain CDR1-3 of a sequence of Table 27. In some embodiments, the CD19 CAR comprises a heavy chain CDR1-3 of a sequence of Table 27 according to Kabat. In some embodiments, the CD19 CAR comprises a heavy chain CDR1-3 of a sequence of Table 27 according to Chothia. In some embodiments, the CD19 CAR comprises a light chain CDR1-3 of a sequence of Table 27. In some embodiments, the CD19 CAR comprises a light chain CDR1-3 of a sequence of Table 27 according to Kabat. In some embodiments, the CD19 CAR comprises a light chain CDR1-3 of a sequence of Table 27 according to Chothia.

TABLE 27
Amino acid and nucleic acid sequences of an exemplary CD19 CAR

| SEQ ID NO: | Description | Sequence |
|---|---|---|
| 367 | CAR C-full | ATGCTGCTGCTGGTGACCAGCCTGCTGCTGTGCGAGCTGCCCCACCCCGC |
| | nucleotide | CTTTCTGCTGATCCCCGACATCCAGATGACCCAGACCACCTCCAGCCTGA |
| | sequence; | GCGCCAGCCTGGGCGACCGGGTGACCATCAGCTGCCGGGCCAGCCAGGAC |
| | with leader | ATCAGCAAGTACCTGAACTGGTATCAGCAGAAGCCCGACGGCACCGTCAA |
| | | GCTGCTGATCTACCACACCAGCCGGCTGCACAGCGGCGTGCCCAGCCGGT |
| | | TTAGCGGCAGCGGCTCCGGCACCGACTACAGCCTGACCATCTCCAACCTG |
| | | GAACAGGAAGATATCGCCACCTACTTTTGCCAGCAGGGCAACACACTGCC |
| | | CTACACCTTTGGCGGCGGAACAAAGCTGGAAATCACCGGCAGCACCTCCG |
| | | GCAGCGGCAAGCCTGGCAGCGGCGAGGGCAGCACCAAGGGCGAGGTGAAG |
| | | CTGCAGGAAAGCGGCCCTGGCCTGGTGGCCCCCAGCCAGAGCCTGAGCGT |
| | | GACCTGCACCGTGAGCGGCGTGAGCCTGCCCGACTACGGCGTGAGCTGGA |
| | | TCCGGCAGCCCCCCAGGAAGGGCCTGGAATGGCTGGGCGTGATCTGGGGC |
| | | AGCGAGACCACCTACTACAACAGCGCCCTGAAGAGCCGGCTGACCATCAT |
| | | CAAGGACAACAGCAAGAGCCAGGTGTTCCTGAAGATGAACAGCCTGCAGA |
| | | CCGACGACACCGCCATCTACTACTGCGCCAAGCACTACTACTACGGCGGC |
| | | AGCTACGCCATGGACTACTGGGGCCAGGGCACCAGCGTGACCGTGAGCAG |
| | | CGAATCTAAGTACGGACCGCCCTGCCCCCCTTGCCCTATGTTCTGGGTGC |
| | | TGGTGGTGGTCGGAGGCGTGCTGGCCTGCTACAGCCTGCTGGTCACCGTG |
| | | GCCTTCATCATCTTTTGGGTGAAACGGGGCAGAAAGAAACTCCTGTATAT |
| | | ATTCAAACAACCATTTATGAGACCAGTACAAACTACTCAAGAGGAAGATG |
| | | GCTGTAGCTGCCGATTTCCAGAAGAAGAAGAAGGAGGATGTGAACTGCGG |
| | | GTGAAGTTCAGCAGAAGCGCCGACGCCCCTGCCTACCAGCAGGGCCAGAA |
| | | TCAGCTGTACAACGAGCTGAACCTGGGCAGAAGGGAAGAGTACGACGTCC |
| | | TGGATAAGCGGAGAGGCCGGGACCCTGAGATGGGCGGCAAGCCTCGGCGG |
| | | AAGAACCCCCAGGAAGGCCTGTATAACGAACTGCAGAAAGACAAGATGGC |
| | | CGAGGCCTACAGCGAGATCGGCATGAAGGGCGAGCGGAGGCGGGGCAAGG |
| | | GCCACGACGGCCTGTATCAGGGCCTGTCCACCGCCACCAAGGATACCTAC |
| | | GACGCCCTGCACATGCAGGCCCTGCCCCCAAGG |
| 368 | CAR C-full | MLLLVTSLLLCELPHPAFLLIPDIQMTQTTSSLSASLGDRVTISCRASQD |
| | transgene | ISKYLNWYQQKPDGTVKLLIYHTSRLHSGVPSRFSGSGSGTDYSLTISNL |
| | amino acid | EQEDIATYFCQQGNTLPYTFGGGTKLEITGSTSGSGKPGSGEGSTKGEVK |
| | sequence; | LQESGPGLVAPSQSLSVTCTVSGVSLPDYGVSWIRQPPRKGLEWLGVIWG |
| | with leader | SETTYYNSALKSRLTIIKDNSKSQVFLKMNSLQTDDTAIYYCAKHYYYGG |
| | | SYAMDYWGQGTSVTVSSESKYGPPCPPCPMFWVLVVVGGVLACYSLLVTV |
| | | AFIIFWVKRGRKKLLYIFKQPFMRPVQTTQEEDGCCRFPEEEEGGCELR |
| | | VKFSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATKDTY |
| | | DALHMQALPPR |
| 369 | CAR C- | ATGCTGCTGCTGGTGACCAGCCTGCTGCTGTGCGAGCTGCCCCACCCCGC |
| | CD 19 scFv | CTTTCTGCTGATCCCCGACATCCAGATGACCCAGACCACCTCCAGCCTGA |
| | nucleotide | GCGCCAGCCTGGGCGACCGGGTGACCATCAGCTGCCGGGCCAGCCAGGAC |
| | sequence; | ATCAGCAAGTACCTGAACTGGTATCAGCAGAAGCCCGACGGCACCGTCAA |
| | with leader | GCTGCTGATCTACCACACCAGCCGGCTGCACAGCGGCGTGCCCAGCCGGT |
| | | TTAGCGGCAGCGGCTCCGGCACCGACTACAGCCTGACCATCTCCAACCTG |
| | | GAACAGGAAGATATCGCCACCTACTTTTGCCAGCAGGGCAACACACTGCC |
| | | CTACACCTTTGGCGGCGGAACAAAGCTGGAAATCACCGGCAGCACCTCCG |
| | | GCAGCGGCAAGCCTGGCAGCGGCGAGGGCAGCACCAAGGGCGAGGTGAAG |
| | | CTGCAGGAAAGCGGCCCTGGCCTGGTGGCCCCCAGCCAGAGCCTGAGCGT |
| | | GACCTGCACCGTGAGCGGCGTGAGCCTGCCCGACTACGGCGTGAGCTGGA |
| | | TCCGGCAGCCCCCCAGGAAGGGCCTGGAATGGCTGGGCGTGATCTGGGGC |
| | | AGCGAGACCACCTACTACAACAGCGCCCTGAAGAGCCGGCTGACCATCAT |
| | | CAAGGACAACAGCAAGAGCCAGGTGTTCCTGAAGATGAACAGCCTGCAGA |
| | | CCGACGACACCGCCATCTACTACTGCGCCAAGCACTACTACTACGGCGGC |
| | | AGCTACGCCATGGACTACTGGGGCCAGGGCACCAGCGTGACCGTGAGCAG |
| | | C |
| 370 | CAR C- | MLLLVTSLLLCELPHPAFLLIPDIQMTQTTSSLSASLGDRVTISCRASQD |
| | CD19 scFv | ISKYLNWYQQKPDGTVKLLIYHTSRLHSGVPSRFSGSGSGTDYSLTISNL |
| | amino acid | EQEDIATYFCQQGNTLPYTFGGGTKLEITGSTSGSGKPGSGEGSTKGEVK |
| | sequence; | LQESGPGLVAPSQSLSVTCTVSGVSLPDYGVSWIRQPPRKGLEWLGVIWG |
| | with leader | SETTYYNSALKSRLTIIKDNSKSQVFLKMNSLQTDDTAIYYCAKHYYYGG |
| | | SYAMDYWGQGTSVTVSS |
| 371 | CAR C-full | GACATCCAGATGACCCAGACCACCTCCAGCCTGAGCGCCAGCCTGGGCGA |
| | nucleotide | CCGGGTGACCATCAGCTGCCGGGCCAGCCAGGACATCAGCAAGTACCTGA |
| | sequence; | ACTGGTATCAGCAGAAGCCCGACGGCACCGTCAAGCTGCTGATCTACCAC |
| | no leader | ACCAGCCGGCTGCACAGCGGCGTGCCCAGCCGGTTTAGCGGCAGCGGCTC |
| | | CGGCACCGACTACAGCCTGACCATCTCCAACCTGGAACAGGAAGATATCG |
| | | CCACCTACTTTTGCCAGCAGGGCAACACACTGCCCTACACCTTTGGCGGC |
| | | GGAACAAAGCTGGAAATCACCGGCAGCACCTCCGGCAGCGGCAAGCCTGG |
| | | CAGCGGCGAGGGCAGCACCAAGGGCGAGGTGAAGCTGCAGGAAAGCGGCC |
| | | CTGGCCTGGTGGCCCCCAGCCAGAGCCTGAGCGTGACCTGCACCGTGAGC |
| | | GGCGTGAGCCTGCCCGACTACGGCGTGAGCTGGATCCGGCAGCCCCCCAG |
| | | GAAGGGCCTGGAATGGCTGGGCGTGATCTGGGGCAGCGAGACCACCTACT |
| | | ACAACAGCGCCCTGAAGAGCCGGCTGACCATCATCAAGGACAACAGCAAG |
| | | AGCCAGGTGTTCCTGAAGATGAACAGCCTGCAGACCGACGACACCGCCAT |
| | | CTACTACTGCGCCAAGCACTACTACTACGGCGGCAGCTACGCCATGGACT |
| | | ACTGGGGCCAGGGCACCAGCGTGACCGTGAGCAGCGAATCTAAGTACGGA |
| | | CCGCCCTGCCCCCCTTGCCCTATGTTCTGGGTGCTGGTGGTGGTCGGAGG |
| | | CGTGCTGGCCTGCTACAGCCTGCTGGTCACCGTGGCCTTCATCATCTTTT |
| | | GGGTGAAACGGGGCAGAAAGAAACTCCTGTATATATTCAAACAACCATTT |
| | | ATGAGACCAGTACAAACTACTCAAGAGGAAGATGGCTGTAGCTGCCGATT |
| | | TCCAGAAGAAGAAGAAGGAGGATGTGAACTGCGGGTGAAGTTCAGCAGAA |
| | | GCGCCGACGCCCCTGCCTACCAGCAGGGCCAGAATCAGCTGTACAACGAG |
| | | CTGAACCTGGGCAGAAGGGAAGAGTACGACGTCCTGGATAAGCGGAGAGG |
| | | CCGGGACCCTGAGATGGGCGGCAAGCCTCGGCGGAAGAACCCCCAGGAAG |
| | | GCCTGTATAACGAACTGCAGAAAGACAAGATGGCCGAGGCCTACAGCGAG |
| | | ATCGGCATGAAGGGCGAGCGGAGGCGGGGCAAGGGCCACGACGGCCTGTA |
| | | TCAGGGCCTGTCCACCGCCACCAAGGATACCTACGACGCCCTGCACATGC |
| | | AGGCCCTGCCCCCAAGG |
| 372 | CAR C-full | DIQMTQTTSSLSASLGDRVTISCRASQDISKYLNWYQQKPDGTVKLLIYH |
| | amino acid | TSRLHSGVPSRFSGSGSGTDYSLTISNLEQEDIATYFCQQGNTLPYTFGG |
| | transgene | GTKLEITGSTSGSGKPGSGEGSTKGEVKLQESGPGLVAPSQSLSVTCTVS |
| | sequence; | GVSLPDYGVSWIRQPPRKGLEWLGVIWGSETTYYNSALKSRLTIIKDNSK |
| | no leader | SQVFLKMNSLQTDDTAIYYCAKHYYYGGSYAMDYWGQGTSVTVSSESKYG |
| | | PPCPPCPMFWVLVVVGGVLACYSLLVTVAFIIFWVKRGRKKLLYIFKQPF |
| | | MRPVQTTQEEDGCSCRFPEEEEGGCELRVKFSRSADAPAYQQGQNQLYNE |
| | | LNLGRREEYDVLDKRRGRDPEMGGKPRRKNPQEGLYNELQKDKMAEAYSE |
| | | IGMKGERRRGKGHDGLYQGLSTATKDTYDALHMQALPPR |
| 373 | CAR C- | GACATCCAGATGACCCAGACCACCTCCAGCCTGAGCGCCAGCCTGGGCGA |
| | CD19 scFv | CCGGGTGACCATCAGCTGCCGGGCCAGCCAGGACATCAGCAAGTACCTGA |
| | sequence; | ACTGGTATCAGCAGAAGCCCGACGGCACCGTCAAGCTGCTGATCTACCAC |
| | no leader | ACCAGCCGGCTGCACAGCGGCGTGCCCAGCCGGTTTAGCGGCAGCGGCTC |
| | | CGGCACCGACTACAGCCTGACCATCTCCAACCTGGAACAGGAAGATATCG |
| | | CCACCTACTTTTGCCAGCAGGGCAACACACTGCCCTACACCTTTGGCGGC |
| | | GGAACAAAGCTGGAAATCACCGGCAGCACCTCCGGCAGCGGCAAGCCTGG |
| | | CAGCGGCGAGGGCAGCACCAAGGGCGAGGTGAAGCTGCAGGAAAGCGGCC |
| | | CTGGCCTGGTGGCCCCCAGCCAGAGCCTGAGCGTGACCTGCACCGTGAGC |
| | | GGCGTGAGCCTGCCCGACTACGGCGTGAGCTGGATCCGGCAGCCCCCCAG |
| | | GAAGGGCCTGGAATGGCTGGGCGTGATCTGGGGCAGCGAGACCACCTACT |
| | | ACAACAGCGCCCTGAAGAGCCGGCTGACCATCATCAAGGACAACAGCAAG |
| | | AGCCAGGTGTTCCTGAAGATGAACAGCCTGCAGACCGACGACACCGCCAT |
| | | CTACTACTGCGCCAAGCACTACTACTACGGCGGCAGCTACGCCATGGACT |
| | | ACTGGGGCCAGGGCACCAGCGTGACCGTGAGCAGC |
| 374 | CAR C- | DIQMTQTTSSLSASLGDRVTISCRASQDISKYLNWYQQKPDGTVKLLIYH |
| | CD19 scFv | TSRLHSGVPSRFSGSGSGTDYSLTISNLEQEDIATYFCQQGNTLPYTFGG |
| | sequence; | GTKLEITGSTSGSGKPGSGEGSTKGEVKLQESGPGLVAPSQSLSVTCTVS |
| | no leader | GVSLPDYGVSWIRQPPRKGLEWLGVIWGSETTYYNSALKSRLTIIKDNSK |
| | | SQVFLKMNSLQTDDTAIYYCAKHYYYGGSYAMDYWGQGTSVTVSS |

### Exemplary CD19 CAR F

In some embodiments, the CD19 CAR is encoded by a nucleotide sequence of Table 27, or a nucleotide sequence having at least 80%, 85%, 90%, 95%, 97%, or 100% or no more than 20, 15, 10, 8, 5, 4, 3, 2, or 1 variations thereto. In some embodiments, the CD19 CAR comprises a polypeptide encoded by a nucleotide sequence of Table 34, or a nucleotide sequence having at least 80%, 85%, 90%, 95%, 97%, or 100% or no more than 20, 15, 10, 8, 5, 4, 3, 2, or 1 variations thereto. In some embodiments, the CD19 CAR comprises a polypeptide sequence of Table 34, or a polypeptide sequence having at least 80%, 85%, 90%, 95%, 97%, or 100% or no more than 20, 15, 10, 8, 5, 4, 3, 2, or 1 variations thereto. In some embodiments, the CD19 CAR comprises a heavy chain CDR1-3 and a light chain CDR1-3, of a sequence of Table 34. In some embodiments, the CD19 CAR comprises a heavy chain CDR1-3 and a light chain CDR1-3, of a sequence of Table 34 according to Kabat. In some embodiments, the CD19 CAR comprises a heavy chain CDR1-3 and a light chain CDR1-3, of a sequence of Table 34 according to Chothia. In some embodiments, the CD19 CAR comprises a heavy chain CDR1-3 of a sequence of Table 34. In some embodiments, the CD19 CAR comprises a heavy chain CDR1-3 of a sequence of Table 34 according to Kabat. In some embodiments, the CD19 CAR comprises a heavy chain CDR1-3 of a sequence of Table 34 according to Chothia. In some embodiments, the CD19 CAR comprises a light chain CDR1-3 of a sequence of Table 34. In some embodiments, the CD19 CAR comprises a light chain CDR1-3 of a sequence of Table 34 according to Kabat. In some embodiments, the CD19 CAR comprises a light chain CDR1-3 of a sequence of Table 34 according to Chothia.

TABLE 34
Amino acid and nucleic acid sequences of an exemplary CD19 CAR

| SEQ ID NO: | Description | Sequence |
|---|---|---|
| 387 | CAR19 F | MGTSLLCWMALCLLGADHADAQVQLQQSGPELVKPGASVKISCKAS |
| | polypeptide | GYAFSSSWMNWVKQRPGKGLEWIGRIYPGDEDTNYSGKFKDKATLT |
| | Sequence | ADKSSTTAYMQLSSLTSEDSAVYFCARSLLYGDYLDYWGQGTTLTV |
| | | SSGGGGSGGGGSGGGGSQIVLTQSPAIMSASPGEKVTMTCSASSSV |
| | | SYMHWYQQKSGTSPKRWIYDTSKLASGVPDRFSGSGSGTSYFLTIN |
| | | NMEAEDAATYYCQQWNINPLTFGAGTKLELKRSDPTTTPAPRPPTP |
| | | APTIASQPLSLRPEACRPAAGGAVHTRGLDFACDIYIWAPLAGTCG |
| | | VLLLSLVITLYCKRGRKKLLYIFKQPFMRPVQTTQEEDGCSCRFPE |
| | | EEEGGCELRVKFSRSADAPAYQQGQNQLYNELNLGRREEYDVLDKR |
| | | RGRDPEMGGKPRRKNPQEGLYNELQKDKMAEAYSEIGMKGERRRGK |
| | | GHDGLYQGLSTATKDTYDALHMQALPPR |
| 388 | CAR19 F | ATGGG CACCAGCCTG CTGTGCTGGA TGGCCCTGTG |
| | Nucleotide | CCTGCTGGGA GCCGACCACG CCGACGCCCA GGTGCAGCTG |
| | Sequence | CAGCAGAGCG GACCCGAGCT GGTGAAGCCC GGAGCCAGCG |
| | | TGAAGATCAG CTGCAAGGCC AGCGGCTACG CCTTCAGCAG |
| | | CAGCTGGATG AACTGGGTGA AGCAGCGGCC TGGCAAGGGC |
| | | CTGGAGTGGA TCGGCCGGAT CTACCCCGGC GACGAGGACA |
| | | CCAACTACAG CGGCAAGTTC AAGGACAAGG CCACCCTGAC |
| | | CGCCGACAAG AGCAGCACCA CCGCCTACAT GCAGCTGAGC |
| | | AGCCTGACCA GCGAGGACAG CGCCGTGTAC TTCTGCGCCC |
| | | GGAGCCTGCT GTACGGCGAC TACCTGGACT ACTGGGGCCA |
| | | GGGCACCACC CTGACCGTGA GCTCTGGCGG TGGCGGCTCT |
| | | GGCGGAGGTG GCTCTGGCGG AGGCGGCAGC CAGATCGTGC |
| | | TGACCCAGAG CCCTGCCATC ATGAGCGCCA GCCCTGGCGA |
| | | GAAGGTGACC ATGACCTGCA GCGCCAGCAG CAGCGTGAGC |
| | | TACATGCACT GGTACCAGCA GAAGAGCGGC ACCAGCCCTA |
| | | AGCGGTGGAT CTACGACACC AGCAAGCTGG CCAGCGGCGT |
| | | GCCCGACCGC TTCAGCGGCA GCGGCAGCGG CACCAGCTAC |
| | | TTCCTGACCA TCAACAACAT GGAGGCCGAG GACGCCGCCA |
| | | CCTACTACTG CCAGCAGTGG AACATCAATC CTCTGACCTT |
| | | CGGCGCCGGC ACCAAGCTGG AGCTGAAGCG GTCGGATCCC |
| | | ACCACCACCC CAGCCCCACG GCCACCTACC CCTGCCCCAA |
| | | CCATCGCCAG CCAGCCCCTG AGCCTGCGGC CTGAAGCCTG |
| | | CAGGCCTGCC GCCGGAGGAG CCGTGCACAC AAGGGGCCTG |
| | | GACTTCGCCT GCGACATCTA TATCTGGGCC CCCCTGGCCG |
| | | GGACATGCGG GGTGCTGCTG CTGTCCCTGG TGATTACACT |
| | | GTATTGCAAA CGGGGCCGGA AGAAGCTGCT GTACATCTTC |
| | | AAGCAGCCCT TCATGCGGCC CGTGCAGACC ACCCAGGAGG |
| | | AGGACGGCTG CAGCTGCCGG TTCCCCGAGG AAGAGGAAGG |
| | | CGGCTGCGAG CTGCGGGTGA AGTTCAGCCG GAGCGCCGAC |
| | | GCCCCAGCCT ACCAGCAGGG CCAGAACCAG CTGTACAACG |
| | | AGCTGAACCT GGGACGGCGG GAGGAGTACG ACGTGCTGGA |
| | | CAAGCGGCGG GGACGGGACC CCGAGATGGG CGGCAAGCCT |
| | | CGCCGGAAGA ATCCCCAGGA GGGCCTGTAC AACGAGCTGC |
| | | AGAAGGACAA GATGGCCGAG GCCTACAGCG AGATCGGCAT |
| | | GAAGGGCGAG CGGCGCCGGG GCAAGGGCCA CGACGGCCTG |
| | | TACCAGGGCC TGAGCACCGC CACCAAGGAC ACCTACGACG |
| | | CCCTGCACAT GCAGGCCCTG CCACCCCGGT GA |

### Exemplary CD19-CD20 CAR G

In some embodiments, the CD19 CAR is a bispecific CAR. In certain embodiments, the CD19 bispecific CAR comprises a light chain variable domain targeting CD19 and a heavy chain variable domain targeting a different target (e.g., CD20). In some embodiments, the bispecific car is an anti-CD19 and anti-CD20 CAR. In some embodiments, the bispecific CAR is encoded by a nucleotide sequence of Table 35, or a nucleotide sequence having at least 80%, 85%, 90%, 95%, 97%, or 100% or no more than 20, 15, 10, 8, 5, 4, 3, 2, or 1 variations thereto. In some embodiments, the bispecific CAR comprises a polypeptide encoded by a nucleotide sequence of Table 35, or a nucleotide sequence having at least 80%, 85%, 90%, 95%, 97%, or 100% or no more than 20, 15, 10, 8, 5, 4, 3, 2, or 1 variations thereto. In some embodiments, the bispecific CAR comprises a polypeptide sequence of Table 35, or a polypeptide sequence having at least 80%, 85%, 90%, 95%, 97%, or 100% or no more than 20, 15, 10, 8, 5, 4, 3, 2, or 1 variations thereto. In some embodiments, the bispecific CAR comprises a heavy chain CDR1-3 and a light chain CDR1-3, of a sequence of Table 35. In some embodiments, the bispecific CAR comprises a heavy chain CDR1-3 and a light chain CDR1-3, of a sequence of Table 35 according to Kabat. In some embodiments, the bispecific CAR comprises a heavy chain CDR1-3 and a light chain CDR1-3, of a sequence of Table 35 according to Chothia. In some embodiments, the bispecific CAR comprises a heavy chain CDR1-3 of a sequence of Table 35. In some embodiments, the bispecific CAR comprises a heavy chain CDR1-3 of a sequence of Table 35 according to Kabat. In some embodiments, the bispecific CAR comprises a heavy chain CDR1-3 of a sequence of Table 35 according to Chothia. In some embodiments, the bispecific CAR comprises a light chain CDR1-3 of a sequence of Table 35. In some embodiments, the bispecific CAR comprises a light chain CDR1-3 of a sequence of Table 35 according to Kabat. In some embodiments, the bispecific CAR comprises a light chain CDR1-3 of a sequence of Table 35 according to Chothia.

TABLE 35
Amino acid and nucleic acid sequences of an exemplary CD19 CAR

| SEQ ID NO: | Description | Sequence |
|---|---|---|
| 389 | Anti- | LLLVTSLLLCELPHPAFLLIPEVQLQQSGAELVKPGASVKMSCKASGY |
| | CD19/CD20 | TFTSYNMHWVKQTPGQGLEWIGAIYPGNGDTSYNQKFKGKATLTADKS |
| | CAR | SSTAYMQLSSLTSEDSADYYCARSNYYGSSYWFFDVWGAGTTVTVSSG |
| | polypeptide | GGGSGGGGSGGGGSDIVLTQSPAILSASPGEKVTMTCRASSSVNYMDW |
| | sequence | YQKKPGSSPKPWIYATSNLASGVPARFSGSGSGTSYSLTISRVEAEDA |
| | with leader | ATYYCQQWSFNPPTFGGGTKLEIKGGGGSGGGGSGGGGSGGGGSGGGG |
| | sequence | SDIQMTQTTSSLSASLGDRVTISCRASQDISKYLNWYQQKPDGTVKLL |
| | | IYHTSRLHSGVPSRFSGSGSGTDYSLTISNLEQEDIATYFCQQGNTLP |
| | | YTFGGGTKLEITGSTSGSGKPGSGEGSTKGEVKLQESGPGLVAPSQSL |
| | | SVTCTVSGVSLPDYGVSWIRQPPRKGLEWLGVIWGSETTYYNSALKSR |
| | | LTIIKDNSKSQVFLKMNSLQTDDTAIYYCAKHYYYGGSYAMDYWGQGT |
| | | SVTVSSAAATTTPAPRPPTPAPTIASQPLSLRPEACRPAAGGAVHTRG |
| | | LDFACDIYIWAPLAGTCGVLLLSLVITLYCKRGRKKLLYIFKQPFMRP |
| | | VQTTQEEDGCSCRFPEEEEGGCELRVKFSRSADAPAYQQGQNQLYNEL |
| | | NLGRREEYDVLDKRRGRDPEMGGKPRRKNPQEGLYNELQKDKMAEAYS |
| | | EIGMKGERRRGKGHDGLYQGLSTATKDTYDALHMQALPPR |
| 390 | Anti- | MSCKASGYTFTSYNMHWVKQTPGQGLEWIGAIYPGNGDTSYNQKFKGK |
| | CD19/CD20 | ATLTADKSSSTAYMQLSSLTSEDSADYYCARSNYYGSSYWFFDVWGAG |
| | CAR | TTVTVSSGGGGSGGGGSGGGGSDIVLTQSPAILSASPGEKVTMTCRAS |
| | polypeptide | SSVNYMDWYQKKPGSSPKPWIYATSNLASGVPARFSGSGSGTSYSLTI |
| | sequence | SRVEAEDAATYYCQQWSFNPPTFGGGTKLEIKGGGGSGGGGSGGGGSG |
| | | GGGSGGGGSDIQMTQTTSSLSASLGDRVTISCRASQDISKYLNWYQQK |
| | | PDGTVKLLIYHTSRLHSGVPSRFSGSGSGTDYSLTISNLEQEDIATYF |
| | | CQQGNTLPYTFGGGTKLEITGSTSGSGKPGSGEGSTKGEVKLQESGPG |
| | | LVAPSQSLSVTCTVSGVSLPDYGVSWIRQPPRKGLEWLGVIWGSETTY |
| | | YNSALKSRLTIIKDNSKSQVFLKMNSLQTDDTAIYYCAKHYYYGGSYA |
| | | MDYWGQGTSVTVSSAAATTTPAPRPPTPAPTIASQPLSLRPEACRPAA |
| | | GGAVHTRGLDFACDIYIWAPLAGTCGVLLLSLVITLYCKRGRKKLLYI |
| | | FKQPFMRPVQTTQEEDGCSCRFPEEEEGGCELRVKFSRSADAPAYQQG |
| | | QNQLYNELNLGRREEYDVLDKRRGRDPEMGGKPRRKNPQEGLYNELQK |
| | | DKMAEAYSEIGMKGERRRGKGHDGLYQGLSTATKDTYDALHMQALPPR |
| 391 | Anti- | GAGGTGCAGTTGCAACAGTCAGGAGCTGAACTGGTCAAGCCAGGAGCC |
| | CD19/CD20 | AGCGTGAAGATGAGCTGCAAGGCCTCCGGTTACACCTTCACCTCCTAC |
| | CAR | AACATGCACTGGGTGAAACAGACCCCGGGACAAGGGCTCGAATGGATT |
| | nucleotide | GGCGCCATCTACCCCGGGAATGGCGATACTTCGTACAACCAGAAGTTC |
| | sequence | AAGGGAAAGGCCACCCTGACCGCCGACAAGAGCTCCTCCACCGCGTAT |
| | | ATGCAGTTGAGCTCCCTGACCTCCGAGGACTCCGCCGACTACTACTGC |
| | | GCACGGTCCAACTACTATGGAAGCTCGTACTGGTTCTTCGATGTCTGG |
| | | GGGGCCGGCACCACTGTGACCGTCAGCTCCGGGGGCGGAGGATCCGGT |
| | | GGAGGCGGAAGCGGGGGTGGAGGATCCGACATTGTGCTGACTCAGTCC |
| | | CCGGCAATCCTGTCGGCCTCACCGGGCGAAAAGGTCACGATGACTTGT |
| | | AGAGCGTCGTCCAGCGTGAACTACATGGATTGGTACCAAAAGAAGCCT |
| | | GGATCGTCACCCAAGCCTTGGATCTACGCTACATCTAACCTGGCCTCC |
| | | GGCGTGCCAGCGCGGTTCAGCGGGTCCGGCTCGGGCACCTCATACTCG |
| | | CTGACCATCTCCCGCGTGGAGGCTGAGGACGCCGCGACCTACTACTGC |
| | | CAGCAGTGGTCCTTCAACCCGCCGACTTTTGGAGGCGGTACTAAGCTG |
| | | GAGATCAAAGGAGGCGGCGGCAGCGGCGGGGGAGGGTCCGGAGGGGGT |
| | | GGTTCTGGTGGAGGAGGATCGGGAGGCGGTGGCAGCGACATTCAGATG |
| | | ACTCAGACCACCTCCTCCCTGTCCGCCTCCCTGGGCGACCGCGTGACC |
| | | ATCTCATGCCGCGCCAGCCAGGACATCTCGAAGTACCTCAACTGGTAC |
| | | CAGCAGAAGCCCGACGGAACCGTGAAGCTCCTGATCTACCACACCTCC |
| | | CGGCTGCACAGCGGAGTGCCGTCTAGATTCTCGGGTTCGGGGTCGGGA |
| | | ACTGACTACTCCCTTACTATTTCCAACCTGGAGCAGGAGGATATTGCC |
| | | ACCTACTTCTGCCAACAAGGAAACACCCTGCCGTACACTTTTGGCGGG |
| | | GGAACCAAGCTGGAAATCACTGGCAGCACATCCGGTTCCGGGAAGCCC |
| | | GGCTCCGGAGAGGGCAGCACCAAGGGGGAAGTCAAGCTGCAGGAATCA |
| | | GGACCTGGCCTGGTGGCCCCGAGCCAGTCACTGTCCGTGACTTGTACT |
| | | GTGTCCGGAGTGTCGCTCCCGGATTACGGAGTGTCCTGGATCAGGCAG |
| | | CCACCTCGGAAAGGATTGGAATGGCTCGGAGTCATCTGGGGTTCCGAA |
| | | ACCACCTATTACAACTCGGCACTGAAATCCAGGCTCACCATTATCAAG |
| | | GATAACTCCAAGTCACAAGTGTTCCTGAAGATGAATAGCCTGCAGACT |
| | | GACGACACGGCGATCTACTATTGCGCCAAGCACTACTACTACGGCGGA |
| | | TCCTACGCTATGGACTACTGGGGCCAGGGGACCAGCGTGACCGTGTCA |
| | | TCCGCGGCCGCAACTACCACCCCTGCCCCTCGGCCGCCGACTCCGGCC |
| | | CCAACCATCGCAAGCCAACCCCTCTCCTTGCGCCCCGAAGCTTGCCGC |
| | | CCGGCCGCGGGTGGAGCCGTGCATACCCGGGGGCTGGACTTTGCCTGC |
| | | GATATCTACATTTGGGCCCCGCTGGCCGGCACTTGCGGCGTGCTCCTG |
| | | CTGTCGCTGGTCATCACCCTTTACTGCAAGAGGGGCCGGAAGAAGCTG |
| | | CTTTACATCTTCAAGCAGCCGTTCATGCGGCCCGTGCAGACGACTCAG |
| | | GAAGAGGACGGATGCTCGTGCAGATTCCCTGAGGAGGAAGAGGGGGGA |
| | | TGCGAACTGCGCGTCAAGTTCTCACGGTCCGCCGACGCCCCCGCATAT |
| | | CAACAGGGCCAGAATCAGCTCTACAACGAGCTGAACCTGGGAAGGAGA |
| | | GAGGAGTACGACGTGCTGGACAAGCGACGCGGACGCGACCCGGAGATG |
| | | GGGGGGAAACCACGGCGGAAAAACCCTCAGGAAGGACTGTACAACGAA |
| | | CTCCAGAAAGACAAGATGGCGGAAGCCTACTCAGAAATCGGGATGAAG |
| | | GGAGAGCGGAGGAGGGGAAAGGGTCACGACGGGCTGTACCAGGGACTG |
| | | AGCACCGCCACTAAGGATACCTACGATGCCTTGCATATGCAAGCACTC |
| | | CCACCCCGG |

### BCMA

A non-limiting exemplary tumor antigen is BCMA. CARs that bind to BCMA are known in the art. For example, those disclosed WO2016/014565 or WO2019/241426 can be used in accordance with the present disclosure. Any known BCMA CAR, for example, the BCMA antigen binding domain of any known BCMA CAR, in the art can be used in accordance with the present disclosure. For example, BCMA-1, BCMA-2, BCMA-3, BCMA-4, BCMA-5, BCMA-6, BCMA-7, BCMA-8, BCMA-9, BCMA-10, BCMA-11, BCMA-12, BCMA-13, BCMA-14, BCMA-15, 149362, 149363, 149364, 149365, 149366, 149367, 149368, 149369, BCMA_EBB-C1978-A4, BCMA_EBB-C1978-G1, BCMA_EBB-C1979-C1, BCMA_EBB-C1978-C7, BCMA_EBB-C1978-D10, BCMA_EBB-C1979-C12, BCMA_EBB-C1980-G4, BCMA_EBB-C1980-D2, BCMA_EBB-C1978-A10, BCMA_EBB-C1978-D4, BCMA_EBB-C1980-A2, BCMA_EBB-C1981-C3, BCMA_EBB-C1978-G4, A7D12.2, C11D5.3, C12A3.2, or C13F12.1, disclosed in WO2016/014565.

In some embodiments, the BCMA CAR comprises one or more CDRs, VH, VL, scFv, or full-length sequences of BCMA-1, BCMA-2, BCMA-3, BCMA-4, BCMA-5, BCMA-6, BCMA-7, BCMA-8, BCMA-9, BCMA-10, BCMA-11, BCMA-12, BCMA-13, BCMA-14, BCMA-15, 149362, 149363, 149364, 149365, 149366, 149367, 149368, 149369, BCMA_EBB-C1978-A4, BCMA_EBB-C1978-G1, BCMA_EBB-C1979-C1, BCMA_EBB-C1978-C7, BCMA_EBB-C1978-D10, BCMA_EBB-C1979-C12, BCMA_EBB-C1980-G4, BCMA_EBB-C1980-D2, BCMA_EBB-C1978-A10, BCMA_EBB-C1978-D4, BCMA_EBB-C1980-A2, BCMA_EBB-C1981-C3, BCMA_EBB-C1978-G4, A7D12.2, C11D5.3, C12A3.2, or C13F12.1 disclosed in WO2016/014565, or a sequence substantially (for example, 95-99%) identical thereto.

Exemplary antigen binding domains that bind BCMA are disclosed in WO2012/0163805, WO 2017/021450, WO 2017/011804, WO 2017/025038, WO 2016/090327, WO 2016/130598, WO 2016/210293, WO 2016/090320, WO 2016/014789, WO 2016/094304, WO 2016/154055, WO 2015/166073, WO 2015/188119, WO 2015/158671, U.S. Pat. Nos. 9,243,058, 8,920,776, 9,273,141, 7,083,785, 9,034,324, US 2007/0049735, US 2015/0284467, US 2015/0051266, US 2015/0344844, US 2016/0131655, US 2016/0297884, US 2016/0297885, US 2017/0051308, US 2017/0051252, WO 2016/020332, WO 2016/087531, WO 2016/079177, WO 2015/172800, WO 2017/008169, U.S. Pat. No. 9,340,621, US 2013/0273055, US 2016/0176973, US 2015/0368351, US 2017/0051068, US 2016/0368988, and US 2015/0232557, herein incorporated by reference in their entirety. In some embodiments, the antigen binding domain of one or more of the BCMA antigen binding domains disclosed therein.

In some embodiments, the antigen binding domain comprises a human antibody or a human antibody fragment that binds BCMA. In some embodiments, the antigen binding domain comprises one or more (for example, all three) LC CDR1, LC CDR2, and LC CDR3 of a human anti-BCMA binding domain described herein (for example, in Tables 2-14), and/or one or more (for example, all three) HC CDR1, HC CDR2, and HC CDR3 of a human anti-BCMA binding domain described herein (for example, in Tables 2-14). In some embodiments, the human anti-BCMA binding domain comprises a human VL described herein (for example, in Tables 2, 6, and 10) and/or a human VH described herein (for example, in Tables 2, 6, and 10). In some embodiments, the antigen binding domain is a scFv comprising a VL and a VH of an amino acid sequence of Tables 2, 6, and 10. In some embodiments, the antigen binding domain (for example, an scFv) comprises: a VL comprising an amino acid sequence having at least one, two or three modifications (for example, substitutions, for example, conservative substitutions) but not more than 30, 20 or 10 modifications (for example, substitutions, for example, conservative substitutions) of an amino acid sequence provided in Tables 2, 6, and 10, or a sequence with 95-99% identity with an amino acid sequence of Tables 2, 6, and 10; and/or a VH comprising an amino acid sequence having at least one, two or three modifications (for example, substitutions, for example, conservative substitutions) but not more than 30, 20 or 10 modifications (for example, substitutions, for example, conservative substitutions) of an amino acid sequence provided in Tables 2, 6, and 10, or a sequence with 95-99% identity to an amino acid sequence of Tables 2, 6, and 10.

In certain embodiments, the antigen binding domain described herein includes: (1) one, two, or three light chain (LC) CDRs chosen from:
(i) a LC CDR1 of SEQ ID NO: 54, LC CDR2 of SEQ ID NO: 55 and LC CDR3 of SEQ ID NO: 56; and/or
(2) one, two, or three heavy chain (HC) CDRs from one of the following: (i) a HC CDR1 of SEQ ID NO: 44, HC CDR2 of SEQ ID NO: 45 and HC CDR3 of SEQ ID NO: 84; (ii) a HC CDR1 of SEQ ID NO: 44, HC CDR2 of SEQ ID NO: 45 and HC CDR3 of SEQ ID NO: 46; (iii) a HC CDR1 of SEQ ID NO: 44, HC CDR2 of SEQ ID NO: 45 and HC CDR3 of SEQ ID NO: 68; or (iv) a HC CDR1 of SEQ ID NO: 44, HC CDR2 of SEQ ID NO: 45 and HC CDR3 of SEQ ID NO: 76.

In certain embodiments, the antigen binding domain described herein includes:
(1) one, two, or three light chain (LC) CDRs from one of the following:
(i) a LC CDR1 of SEQ ID NO: 95, LC CDR2 of SEQ ID NO: 131 and LC CDR3 of SEQ ID NO: 132; (ii) a LC CDR1 of SEQ ID NO: 95, LC CDR2 of SEQ ID NO: 96 and LC CDR3 of SEQ ID NO: 97; (iii) a LC CDR1 of SEQ ID NO: 95, LC CDR2 of SEQ ID NO: 114 and LC CDR3 of SEQ ID NO: 115; or (iv) a LC CDR1 of SEQ ID NO: 95, LC CDR2 of SEQ ID NO: 114 and LC CDR3 of SEQ ID NO: 97; and/or
(2) one, two, or three heavy chain (HC) CDRs from one of the following:
(i) a HC CDR1 of SEQ ID NO: 86, HC CDR2 of SEQ ID NO: 130 and HC CDR3 of SEQ ID NO: 88; (ii) a HC CDR1 of SEQ ID NO: 86, HC CDR2 of SEQ ID NO: 87 and HC CDR3 of SEQ ID NO: 88; or (iii) a HC CDR1 of SEQ ID NO: 86, HC CDR2 of SEQ ID NO: 109 and HC CDR3 of SEQ ID NO: 88.

In certain embodiments, the antigen binding domain described herein includes:
(1) one, two, or three light chain (LC) CDRs from one of the following:
(i) a LC CDR1 of SEQ ID NO: 147, LC CDR2 of SEQ ID NO: 182 and LC CDR3 of SEQ ID NO: 183; (ii) a LC CDR1 of SEQ ID NO: 147, LC CDR2 of SEQ ID NO: 148 and LC CDR3 of SEQ ID NO: 149; or (iii) a LC CDR1 of SEQ ID NO: 147, LC CDR2 of SEQ ID NO: 170 and LC CDR3 of SEQ ID NO: 171; and/or
(2) one, two, or three heavy chain (HC) CDRs from one of the following:
(i) a HC CDR1 of SEQ ID NO: 179, HC CDR2 of SEQ ID NO: 180 and HC CDR3 of SEQ ID NO: 181; (ii) a HC CDR1 of SEQ ID NO: 137, HC CDR2 of SEQ ID NO: 138 and HC CDR3 of SEQ ID NO: 139; or (iii) a HC CDR1 of SEQ ID NO: 160, HC CDR2 of SEQ ID NO: 161 and HC CDR3 of SEQ ID NO: 162.

In some embodiments, the HC CDR1, HC CDR2, HC CDR3, LC CDR1, LC CDR2, and LC CDR3 comprise the amino acid sequences of SEQ ID NOs: 44, 45, 84, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 46, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 68, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 76, 54, 55, and 56, respectively.

In some embodiments, the HC CDR1, HC CDR2, HC CDR3, LC CDR1, LC CDR2, and LC CDR3 comprise the amino acid sequences of SEQ ID NOs: 47, 48, 84, 57, 58, and 59, respectively. In some embodiments, the HC CDR1, HC CDR2, HC CDR3, LC CDR1, LC CDR2, and LC CDR3 comprise the amino acid sequences of SEQ ID NOs: 47, 48, 46, 57, 58, and 59, respectively. In some embodiments, the HC CDR1, HC CDR2, HC CDR3, LC CDR1, LC CDR2, and LC CDR3 comprise the amino acid sequences of SEQ ID NOs: 47, 48, 68, 57, 58, and 59, respectively. In some embodiments, the HC CDR1, HC CDR2, HC CDR3, LC CDR1, LC CDR2, and LC CDR3 comprise the amino acid sequences of SEQ ID NOs: 47, 48, 76, 57, 58, and 59, respectively.

In some embodiments, the HC CDR1, HC CDR2, HC CDR3, LC CDR1, LC CDR2, and LC CDR3 comprise the amino acid sequences of SEQ ID NOs: 49, 50, 85, 60, 58, and 56, respectively. In some embodiments, the HC CDR1, HC CDR2, HC CDR3, LC CDR1, LC CDR2, and LC CDR3 comprise the amino acid sequences of SEQ ID NOs: 49, 50, 51, 60, 58, and 56, respectively. In some embodiments, the HC CDR1, HC CDR2, HC CDR3, LC CDR1, LC CDR2, and LC CDR3 comprise the amino acid sequences of SEQ ID NOs: 49, 50, 69, 60, 58, and 56, respectively. In some embodiments, the HC CDR1, HC CDR2, HC CDR3, LC CDR1, LC CDR2, and LC CDR3 comprise the amino acid sequences of SEQ ID NOs: 49, 50, 77, 60, 58, and 56, respectively.

In some embodiments, a BCMA CAR comprises a sequence, for example, a CDR, VH, VL, scFv, or full-CAR sequence, disclosed in Tables 2-14, or a sequence having at least 80%, 85%, 90%, 95%, or 99% identity thereto.

TABLE 2
Amino acid and nucleic acid sequences of exemplary
PALLAS-derived anti-BCMA molecules

| SEQ ID NO | Name/Description | Sequence |
|---|---|---|
| R1B6 | | |
| SEQ ID | HCDR1 | SYAMS |
| NO: 44 | (Kabat) | |
| SEQ ID | HCDR2 | AISGSGGSTYYADSVKG |
| NO: 45 | (Kabat) | |
| SEQ ID | HCDR3 | REWVPYDVSWYFDY |
| NO: 46 | (Kabat) | |
| SEQ ID | HCDR1 | GFTFSSY |
| NO: 47 | (Chothia) | |
| SEQ ID | HCDR2 | SGSGGS |
| NO: 48 | (Chothia) | |
| SEQ ID | HCDR3 | REWVPYDVSWYFDY |
| NO: 46 | (Chothia) | |
| SEQ ID | HCDR1 | GFTFSSYA |
| NO: 49 | (IMGT) | |
| SEQ ID | HCDR2 | ISGSGGST |
| NO: 50 | (IMGT) | |
| SEQ ID | HCDR3 | ARREWVPYDVSWYFDY |
| NO: 51 | (IMGT) | |
| SEQ ID | VH | EVQLLESGGGLVQPGGSLRLSCAASGFTFSSYAMSWVRQAPG |
| NO: 52 | | KGLEWVSAISGSGGSTYYADSVKGRFTISRDNSKNTLYLQMNS |
| | | LRAEDTAVYYCARREWVPYDVSWYFDYWGQGTLVTVSS |
| SEQ ID | DNA VH | GAAGTGCAGTTGCTGGAGTCAGGCGGAGGACTGGTGCAGCC |
| NO: 53 | | CGGAGGATCGCTTCGCTTGAGCTGCGCAGCCTCAGGCTTTA |
| | | CCTTCTCCTCCTACGCCATGTCCTGGGTCAGACAGGCTCCCG |
| | | GGAAGGGACTGGAATGGGTGTCCGCCATTAGCGGTTCCGGC |
| | | GGAAGCACTTACTATGCCGACTCTGTGAAGGGCCGCTTCAC |
| | | TATCTCCCGGGACAACTCCAAGAACACCCTGTATCTCCAAA |
| | | TGAATTCCCTGAGGGCCGAAGATACCGCGGTGTACTACTGC |
| | | GCTAGACGGGAGTGGGTGCCCTACGATGTCAGCTGGTACTT |
| | | CGACTACTGGGGACAGGGCACTCTCGTGACTGTGTCCTCC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPLT |
| NO: 56 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPL |
| NO: 59 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPLT |
| NO: 56 | (IMGT) | |
| SEQ ID | VL | DIQMTQSPSSLSASVGDRVTITCRASQSISSYLNWYQQKPGKAP |
| NO: 61 | | KLLIYAASSLQSGVPSRFSGSGSGTDFTLTISSLQPEDFATYYCQ |
| | | QSYSTPLTFGQGTKVEIK |
| SEQ ID | DNA VL | GACATTCAAATGACTCAGTCCCCGTCCTCCCTCTCCGCCTCC |
| NO: 62 | | GTGGGAGATCGCGTCACGATCACGTGCAGGGCCAGCCAGAG |
| | | CATCTCCAGCTACCTGAACTGGTACCAGCAGAAGCCAGGGA |
| | | AGGCACCGAAGCTCCTGATCTACGCCGCTAGCTCGCTGCAG |
| | | TCCGGCGTCCCTTCACGGTTCTCGGGATCGGGCTCAGGCACC |
| | | GACTTCACCCTGACCATTAGCAGCCTGCAGCCGGAGGACTT |
| | | CGCGACATACTACTGTCAGCAGTCATACTCCACCCCTCTGAC |
| | | CTTCGGCCAAGGGACCAAAGTGGAGATCAAG |
| SEQ ID | Linker | GGGGSGGGGSGGGGSGGGGS |
| NO: 63 | | |
| SEQ ID | scFv (VH- | EVQLLESGGGLVQPGGSLRLSCAASGFTFSSYAMSWVRQAPG |
| NO: 64 | linker-VL) | KGLEWVSAISGSGGSTYYADSVKGRFTISRDNSKNTLYLQMNS |
| | | LRAEDTAVYYCARREWVPYDVSWYFDYWGQGTLVTVSSGGG |
| | | GSGGGGSGGGGSGGGGSDIQMTQSPSSLSASVGDRVTITCRAS |
| | | QSISSYLNWYQQKPGKAPKLLIYAASSLQSGVPSRFSGSGSGTD |
| | | FTLTISSLQPEDFATYYCQQSYSTPLTFGQGTKVEIK |
| SEQ ID | DNA scFv | GAAGTGCAGTTGCTGGAGTCAGGCGGAGGACTGGTGCAGCC |
| NO: 65 | | CGGAGGATCGCTTCGCTTGAGCTGCGCAGCCTCAGGCTTTA |
| | | CCTTCTCCTCCTACGCCATGTCCTGGGTCAGACAGGCTCCCG |
| | | GGAAGGGACTGGAATGGGTGTCCGCCATTAGCGGTTCCGGC |
| | | GGAAGCACTTACTATGCCGACTCTGTGAAGGGCCGCTTCAC |
| | | TATCTCCCGGGACAACTCCAAGAACACCCTGTATCTCCAAA |
| | | TGAATTCCCTGAGGGCCGAAGATACCGCGGTGTACTACTGC |
| | | GCTAGACGGGAGTGGGTGCCCTACGATGTCAGCTGGTACTT |
| | | CGACTACTGGGGACAGGGCACTCTCGTGACTGTGTCCTCCG |
| | | GTGGTGGTGGATCGGGGGGTGGTGGTTCGGGCGGAGGAGG |
| | | ATCTGGAGGAGGAGGGTCGGACATTCAAATGACTCAGTCCC |
| | | CGTCCTCCCTCTCCGCCTCCGTGGGAGATCGCGTCACGATCA |
| | | CGTGCAGGGCCAGCCAGAGCATCTCCAGCTACCTGAACTGG |
| | | TACCAGCAGAAGCCAGGGAAGGCACCGAAGCTCCTGATCTA |
| | | CGCCGCTAGCTCGCTGCAGTCCGGCGTCCCTTCACGGTTCTC |
| | | GGGATCGGGCTCAGGCACCGACTTCACCCTGACCATTAGCA |
| | | GCCTGCAGCCGGAGGACTTCGCGACATACTACTGTCAGCAG |
| | | TCATACTCCACCCCTCTGACCTTCGGCCAAGGGACCAAAGT |
| | | GGAGATCAAG |
| SEQ ID | Full CAR | EVQLLESGGGLVQPGGSLRLSCAASGFTFSSYAMSWVRQAPG |
| NO: 66 | amino acid | KGLEWVSAISGSGGSTYYADSVKGRFTISRDNSKNTLYLQMNS |
| | sequence | LRAEDTAVYYCARREWVPYDVSWYFDYWGQGTLVTVSSGGG |
| | | GSGGGGSGGGGSGGGGSDIQMTQSPSSLSASVGDRVTITCRAS |
| | | QSISSYLNWYQQKPGKAPKLLIYAASSLQSGVPSRFSGSGSGTD |
| | | FTLTISSLQPEDFATYYCQQSYSTPLTFGQGTKVEIKTTTPAPRP |
| | | PTPAPTIASQPLSLRPEACRPAAGGAVHTRGLDFACDIYIWAPL |
| | | AGTCGVLLLSLVITLYCKRGRKKLLYIFKQPFMRPVQTTQEED |
| | | GCSCRFPEEEEGGCELRVKFSRSADAPAYQQGQNQLYNELNLG |
| | | RREEYDVLDKRRGRDPEMGGKPRRKNPQEGLYNELQKDKMA |
| | | EAYSEIGMKGERRRGKGHDGLYQGLSTATKDTYDALHMQAL |
| | | PPR |
| SEQ ID | Full CAR | GAAGTGCAGTTGCTGGAGTCAGGCGGAGGACTGGTGCAGCC |
| NO: 67 | DNA | CGGAGGATCGCTTCGCTTGAGCTGCGCAGCCTCAGGCTTTA |
| | sequence | CCTTCTCCTCCTACGCCATGTCCTGGGTCAGACAGGCTCCCG |
| | | GGAAGGGACTGGAATGGGTGTCCGCCATTAGCGGTTCCGGC |
| | | GGAAGCACTTACTATGCCGACTCTGTGAAGGGCCGCTTCAC |
| | | TATCTCCCGGGACAACTCCAAGAACACCCTGTATCTCCAAA |
| | | TGAATTCCCTGAGGGCCGAAGATACCGCGGTGTACTACTGC |
| | | GCTAGACGGGAGTGGGTGCCCTACGATGTCAGCTGGTACTT |
| | | CGACTACTGGGGACAGGGCACTCTCGTGACTGTGTCCTCCG |
| | | GTGGTGGTGGATCGGGGGGTGGTGGTTCGGGCGGAGGAGG |
| | | ATCTGGAGGAGGAGGGTCGGACATTCAAATGACTCAGTCCC |
| | | CGTCCTCCCTCTCCGCCTCCGTGGGAGATCGCGTCACGATCA |
| | | CGTGCAGGGCCAGCCAGAGCATCTCCAGCTACCTGAACTGG |
| | | TACCAGCAGAAGCCAGGGAAGGCACCGAAGCTCCTGATCTA |
| | | CGCCGCTAGCTCGCTGCAGTCCGGCGTCCCTTCACGGTTCTC |
| | | GGGATCGGGCTCAGGCACCGACTTCACCCTGACCATTAGCA |
| | | GCCTGCAGCCGGAGGACTTCGCGACATACTACTGTCAGCAG |
| | | TCATACTCCACCCCTCTGACCTTCGGCCAAGGGACCAAAGT |
| | | GGAGATCAAGACCACTACCCCAGCACCGAGGCCACCCACCC |
| | | CGGCTCCTACCATCGCCTCCCAGCCTCTGTCCCTGCGTCCGG |
| | | AGGCATGTAGACCCGCAGCTGGTGGGGCCGTGCATACCCGG |
| | | GGTCTTGACTTCGCCTGCGATATCTACATTTGGGCCCCTCTG |
| | | GCTGGTACTTGCGGGGTCCTGCTGCTTTCACTCGTGATCACT |
| | | CTTTACTGTAAGCGCGGTCGGAAGAAGCTGCTGTACATCTTT |
| | | AAGCAACCCTTCATGAGGCCTGTGCAGACTACTCAAGAGGA |
| | | GGACGGCTGTTCATGCCGGTTCCCAGAGGAGGAGGAAGGCG |
| | | GCTGCGAACTGCGCGTGAAATTCAGCCGCAGCGCAGATGCT |
| | | CCAGCCTACCAGCAGGGGCAGAACCAGCTCTACAACGAACT |
| | | CAATCTTGGTCGGAGAGAGGAGTACGACGTGCTGGACAAGC |
| | | GGAGAGGACGGGACCCAGAAATGGGCGGGAAGCCGCGCAG |
| | | AAAGAATCCCCAAGAGGGCCTGTACAACGAGCTCCAAAAG |
| | | GATAAGATGGCAGAAGCCTATAGCGAGATTGGTATGAAAG |
| | | GGGAACGCAGAAGAGGCAAAGGCCACGACGGACTGTACCA |
| | | GGGACTCAGCACCGCCACCAAGGACACCTATGACGCTCTTC |
| | | ACATGCAGGCCCTGCCGCCTCGG |
| R1F2 | | |
| SEQ ID | HCDR1 | SYAMS |
| NO: 44 | (Kabat) | |
| SEQ ID | HCDR2 | AISGSGGSTYYADSVKG |
| NO: 45 | (Kabat) | |
| SEQ ID | HCDR3 | REWWYDDWYLDY |
| NO: 68 | (Kabat) | |
| SEQ ID | HCDR1 | GFTFSSY |
| NO: 47 | (Chothia) | |
| SEQ ID | HCDR2 | SGSGGS |
| NO: 48 | (Chothia) | |
| SEQ ID | HCDR3 | REWWYDDWYLDY |
| NO: 68 | (Chothia) | |
| SEQ ID | HCDR1 | GFTFSSYA |
| NO: 49 | (IMGT) | |
| SEQ ID | HCDR2 | ISGSGGST |
| NO: 50 | (IMGT) | |
| SEQ ID | HCDR3 | ARREWWYDDWYLDY |
| NO: 69 | (IMGT) | |
| SEQ ID | VH | EVQLLESGGGLVQPGGSLRLSCAASGFTFSSYAMSWVRQAPG |
| NO: 70 | | KGLEWVSAISGSGGSTYYADSVKGRFTISRDNSKNTLYLQMNS |
| | | LRAEDTAVYYCARREWWYDDWYLDYWGQGTLVTVSS |
| SEQ ID | DNA VH | GAAGTGCAGTTGCTGGAGTCAGGCGGAGGACTGGTGCAGCC |
| NO: 71 | | CGGAGGATCGCTTCGCTTGAGCTGCGCAGCCTCAGGCTTTA |
| | | CCTTCTCCTCCTACGCCATGTCCTGGGTCAGACAGGCTCCCG |
| | | GGAAGGGACTGGAATGGGTGTCCGCCATTAGCGGTTCCGGC |
| | | GGAAGCACTTACTATGCCGACTCTGTGAAGGGCCGCTTCAC |
| | | TATCTCCCGGGACAACTCCAAGAACACCCTGTATCTCCAAA |
| | | TGAATTCCCTGAGGGCCGAAGATACCGCGGTGTACTACTGC |
| | | GCTAGACGGGAGTGGTGGTACGACGATTGGTACCTGGACTA |
| | | CTGGGGACAGGGCACTCTCGTGACTGTGTCCTCC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPLT |
| NO: 56 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPL |
| NO: 59 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPLT |
| NO: 56 | (IMGT) | |
| SEQ ID | VL | DIQMTQSPSSLSASVGDRVTITCRASQSISSYLNWYQQKPGKAP |
| NO: 61 | | KLLIYAASSLQSGVPSRFSGSGSGTDFTLTISSLQPEDFATYYCQ |
| | | QSYSTPLTFGQGTKVEIK |
| SEQ ID | DNA VL | GACATTCAAATGACTCAGTCCCCGTCCTCCCTCTCCGCCTCC |
| NO: 62 | | GTGGGAGATCGCGTCACGATCACGTGCAGGGCCAGCCAGAG |
| | | CATCTCCAGCTACCTGAACTGGTACCAGCAGAAGCCAGGGA |
| | | AGGCACCGAAGCTCCTGATCTACGCCGCTAGCTCGCTGCAG |
| | | TCCGGCGTCCCTTCACGGTTCTCGGGATCGGGCTCAGGCACC |
| | | GACTTCACCCTGACCATTAGCAGCCTGCAGCCGGAGGACTT |
| | | CGCGACATACTACTGTCAGCAGTCATACTCCACCCCTCTGAC |
| | | CTTCGGCCAAGGGACCAAAGTGGAGATCAAG |
| SEQ ID | Linker | GGGGSGGGGSGGGGSGGGGS |
| NO: 63 | | |
| SEQ ID | scFv (VH- | EVQLLESGGGLVQPGGSLRLSCAASGFTFSSYAMSWVRQAPG |
| NO: 72 | linker-VL) | KGLEWVSAISGSGGSTYYADSVKGRFTISRDNSKNTLYLQMNS |
| | | LRAEDTAVYYCARREWWYDDWYLDYWGQGTLVTVSSGGGG |
| | | SGGGGSGGGGSGGGGSDIQMTQSPSSLSASVGDRVTITCRASQ |
| | | SISSYLNWYQQKPGKAPKLLIYAASSLQSGVPSRFSGSGSGTDF |
| | | TLTISSLQPEDFATYYCQQSYSTPLTFGQGTKVEIK |
| SEQ ID | DNA scFv | GAAGTGCAGTTGCTGGAGTCAGGCGGAGGACTGGTGCAGCC |
| NO: 73 | | CGGAGGATCGCTTCGCTTGAGCTGCGCAGCCTCAGGCTTTA |
| | | CCTTCTCCTCCTACGCCATGTCCTGGGTCAGACAGGCTCCCG |
| | | GGAAGGGACTGGAATGGGTGTCCGCCATTAGCGGTTCCGGC |
| | | GGAAGCACTTACTATGCCGACTCTGTGAAGGGCCGCTTCAC |
| | | TATCTCCCGGGACAACTCCAAGAACACCCTGTATCTCCAAA |
| | | TGAATTCCCTGAGGGCCGAAGATACCGCGGTGTACTACTGC |
| | | GCTAGACGGGAGTGGTGGTACGACGATTGGTACCTGGACTA |
| | | CTGGGGACAGGGCACTCTCGTGACTGTGTCCTCCGGTGGTG |
| | | GTGGATCGGGGGGTGGTGGTTCGGGCGGAGGAGGATCTGG |
| | | AGGAGGAGGGTCGGACATTCAAATGACTCAGTCCCCGTCCT |
| | | CCCTCTCCGCCTCCGTGGGAGATCGCGTCACGATCACGTGC |
| | | AGGGCCAGCCAGAGCATCTCCAGCTACCTGAACTGGTACCA |
| | | GCAGAAGCCAGGGAAGGCACCGAAGCTCCTGATCTACGCCG |
| | | CTAGCTCGCTGCAGTCCGGCGTCCCTTCACGGTTCTCGGGAT |
| | | CGGGCTCAGGCACCGACTTCACCCTGACCATTAGCAGCCTG |
| | | CAGCCGGAGGACTTCGCGACATACTACTGTCAGCAGTCATA |
| | | CTCCACCCCTCTGACCTTCGGCCAAGGGACCAAAGTGGAGA |
| | | TCAAG |
| SEQ ID | Full CAR | EVQLLESGGGLVQPGGSLRLSCAASGFTFSSYAMSWVRQAPG |
| NO: 74 | amino acid | KGLEWVSAISGSGGSTYYADSVKGRFTISRDNSKNTLYLQMNS |
| | sequence | LRAEDTAVYYCARREWWYDDWYLDYWGQGTLVTVSSGGGG |
| | | SGGGGSGGGGSGGGGSDIQMTQSPSSLSASVGDRVTITCRASQ |
| | Full CAR | SISSYLNWYQQKPGKAPKLLIYAASSLQSGVPSRFSGSGSGTDF |
| | DNA | TLTISSLOPEDFATYYCQQSYSTPLTFGQGTKVEIKTTTPAPRPP |
| | | TPAPTIASQPLSLRPEACRPAAGGAVHTRGLDFACDIYIWAPLA |
| | | GTCGVLLLSLVITLYCKRGRKKLLYIFKQPFMRPVQTTQEEDG |
| | | CSCRFPEEEEGGCELRVKFSRSADAPAYQQGQNQLYNELNLGR |
| | | REEYDVLDKRRGRDPEMGGKPRRKNPQEGLYNELQKDKMAE |
| | | AYSEIGMKGERRRGKGHDGLYQGLSTATKDTYDALHMQALPP |
| | | R |
| | | GAAGTGCAGTTGCTGGAGTCAGGCGGAGGACTGGTGCAGCC |
| | | CGGAGGATCGCTTCGCTTGAGCTGCGCAGCCTCAGGCTTTA |
| | | CCTTCTCCTCCTACGCCATGTCCTGGGTCAGACAGGCTCCCG |
| | | GGAAGGGACTGGAATGGGTGTCCGCCATTAGCGGTTCCGGC |
| | | GGAAGCACTTACTATGCCGACTCTGTGAAGGGCCGCTTCAC |
| | | TATCTCCCGGGACAACTCCAAGAACACCCTGTATCTCCAAA |
| | | TGAATTCCCTGAGGGCCGAAGATACCGCGGTGTACTACTGC |
| | | GCTAGACGGGAGTGGTGGTACGACGATTGGTACCTGGACTA |
| | | CTGGGGACAGGGCACTCTCGTGACTGTGTCCTCCGGTGGTG |
| | | GTGGATCGGGGGGTGGTGGTTCGGGCGGAGGAGGATCTGG |
| SEQ ID | sequence | AGGAGGAGGGTCGGACATTCAAATGACTCAGTCCCCGTCCT |
| NO: 75 | | CCCTCTCCGCCTCCGTGGGAGATCGCGTCACGATCACGTGC |
| | | AGGGCCAGCCAGAGCATCTCCAGCTACCTGAACTGGTACCA |
| | | GCAGAAGCCAGGGAAGGCACCGAAGCTCCTGATCTACGCCG |
| | | CTAGCTCGCTGCAGTCCGGCGTCCCTTCACGGTTCTCGGGAT |
| | | CGGGCTCAGGCACCGACTTCACCCTGACCATTAGCAGCCTG |
| | | CAGCCGGAGGACTTCGCGACATACTACTGTCAGCAGTCATA |
| | | CTCCACCCCTCTGACCTTCGGCCAAGGGACCAAAGTGGAGA |
| | | TCAAGACCACTACCCCAGCACCGAGGCCACCCACCCCGGCT |
| | | CCTACCATCGCCTCCCAGCCTCTGTCCCTGCGTCCGGAGGCA |
| | | TGTAGACCCGCAGCTGGTGGGGCCGTGCATACCCGGGGTCT |
| | | TGACTTCGCCTGCGATATCTACATTTGGGCCCCTCTGGCTGG |
| | | TACTTGCGGGGTCCTGCTGCTTTCACTCGTGATCACTCTTTA |
| | | CTGTAAGCGCGGTCGGAAGAAGCTGCTGTACATCTTTAAGC |
| | | AACCCTTCATGAGGCCTGTGCAGACTACTCAAGAGGAGGAC |
| | | GGCTGTTCATGCCGGTTCCCAGAGGAGGAGGAAGGCGGCTG |
| | | CGAACTGCGCGTGAAATTCAGCCGCAGCGCAGATGCTCCAG |
| | | CCTACCAGCAGGGGCAGAACCAGCTCTACAACGAACTCAAT |
| | | CTTGGTCGGAGAGAGGAGTACGACGTGCTGGACAAGCGGA |
| | | GAGGACGGGACCCAGAAATGGGCGGGAAGCCGCGCAGAAA |
| | | GAATCCCCAAGAGGGCCTGTACAACGAGCTCCAAAAGGATA |
| | | AGATGGCAGAAGCCTATAGCGAGATTGGTATGAAAGGGGA |
| | | ACGCAGAAGAGGCAAAGGCCACGACGGACTGTACCAGGGA |
| | | CTCAGCACCGCCACCAAGGACACCTATGACGCTCTTCACAT |
| | | GCAGGCCCTGCCGCCTCGG |
| R1G5 | | |
| SEQ ID | HCDR1 | SYAMS |
| NO: 44 | (Kabat) | |
| SEQ ID | HCDR2 | AISGSGGSTYYADSVKG |
| NO: 45 | (Kabat) | |
| SEQ ID | HCDR3 | REWWGESWLFDY |
| NO: 76 | (Kabat) | |
| SEQ ID | HCDR1 | GFTFSSY |
| NO: 47 | (Chothia) | |
| SEQ ID | HCDR2 | SGSGGS |
| NO: 48 | (Chothia) | |
| SEQ ID | HCDR3 | REWWGESWLFDY |
| NO: 76 | (Chothia) | |
| SEQ ID | HCDR1 | GFTFSSYA |
| NO: 49 | (IMGT) | |
| SEQ ID | HCDR2 | ISGSGGST |
| NO: 50 | (IMGT) | |
| SEQ ID | HCDR3 | ARREWWGESWLFDY |
| NO: 77 | (IMGT) | |
| SEQ ID | VH | EVQLLESGGGLVQPGGSLRLSCAASGFTFSSYAMSWVRQAPG |
| NO: 78 | | KGLEWVSAISGSGGSTYYADSVKGRFTISRDNSKNTLYLQMNS |
| | | LRAEDTAVYYCARREWWGESWLFDYWGQGTLVTVSS |
| SEQ ID | DNA VH | GAAGTGCAGTTGCTGGAGTCAGGCGGAGGACTGGTGCAGCC |
| NO: 79 | | CGGAGGATCGCTTCGCTTGAGCTGCGCAGCCTCAGGCTTTA |
| | | CCTTCTCCTCCTACGCCATGTCCTGGGTCAGACAGGCTCCCG |
| | | GGAAGGGACTGGAATGGGTGTCCGCCATTAGCGGTTCCGGC |
| | | GGAAGCACTTACTATGCCGACTCTGTGAAGGGCCGCTTCAC |
| | | TATCTCCCGGGACAACTCCAAGAACACCCTGTATCTCCAAA |
| | | TGAATTCCCTGAGGGCCGAAGATACCGCGGTGTACTACTGC |
| | | GCTAGACGGGAGTGGTGGGGAGAAAGCTGGCTGTTCGACTA |
| | | CTGGGGACAGGGCACTCTCGTGACTGTGTCCTCC |
| SEQ ID | LCDR1 | RASQSISSYLN |
| NO: 54 | (Kabat) | |
| SEQ ID | LCDR2 | AASSLOS |
| NO: 55 | (Kabat) | |
| SEQ ID | LCDR3 | QQSYSTPLT |
| NO: 56 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPL |
| NO: 59 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPLT |
| NO: 56 | (IMGT) | |
| SEQ ID | VL | DIQMTQSPSSLSASVGDRVTITCRASQSISSYLNWYQQKPGKAP |
| NO: 61 | | KLLIYAASSLQSGVPSRFSGSGSGTDFTLTISSLQPEDFATYYCQ |
| | | QSYSTPLTFGQGTKVEIK |
| SEQ ID | DNA VL | GACATTCAAATGACTCAGTCCCCGTCCTCCCTCTCCGCCTCC |
| NO: 62 | | GTGGGAGATCGCGTCACGATCACGTGCAGGGCCAGCCAGAG |
| | | CATCTCCAGCTACCTGAACTGGTACCAGCAGAAGCCAGGGA |
| | | AGGCACCGAAGCTCCTGATCTACGCCGCTAGCTCGCTGCAG |
| | | TCCGGCGTCCCTTCACGGTTCTCGGGATCGGGCTCAGGCACC |
| | | GACTTCACCCTGACCATTAGCAGCCTGCAGCCGGAGGACTT |
| | | CGCGACATACTACTGTCAGCAGTCATACTCCACCCCTCTGAC |
| | | CTTCGGCCAAGGGACCAAAGTGGAGATCAAG |
| SEQ ID | Linker | GGGGSGGGGSGGGGSGGGGS |
| NO: 63 | | |
| SEQ ID | scFv (VH- | EVQLLESGGGLVQPGGSLRLSCAASGFTFSSYAMSWVRQAPG |
| NO: 80 | linker-VL) | KGLEWVSAISGSGGSTYYADSVKGRFTISRDNSKNTLYLQMNS |
| | | LRAEDTAVYYCARREWWGESWLFDYWGQGTLVTVSSGGGGS |
| | DNA scFv | GGGGSGGGGSGGGGSDIQMTQSPSSLSASVGDRVTITCRASQSI |
| | | SSYLNWYQQKPGKAPKLLIYAASSLQSGVPSRFSGSGSGTDFTL |
| | | TISSLOPEDFATYYCQQSYSTPLTFGQGTKVEIK |
| | | GAAGTGCAGTTGCTGGAGTCAGGCGGAGGACTGGTGCAGCC |
| | | CGGAGGATCGCTTCGCTTGAGCTGCGCAGCCTCAGGCTTTA |
| | | CCTTCTCCTCCTACGCCATGTCCTGGGTCAGACAGGCTCCCG |
| | | GGAAGGGACTGGAATGGGTGTCCGCCATTAGCGGTTCCGGC |
| | | GGAAGCACTTACTATGCCGACTCTGTGAAGGGCCGCTTCAC |
| | | TATCTCCCGGGACAACTCCAAGAACACCCTGTATCTCCAAA |
| | | TGAATTCCCTGAGGGCCGAAGATACCGCGGTGTACTACTGC |
| | | GCTAGACGGGAGTGGTGGGGAGAAAGCTGGCTGTTCGACTA |
| SEQ ID | | CTGGGGACAGGGCACTCTCGTGACTGTGTCCTCCGGTGGTG |
| NO: 81 | | GTGGATCGGGGGGTGGTGGTTCGGGCGGAGGAGGATCTGG |
| | | AGGAGGAGGGTCGGACATTCAAATGACTCAGTCCCCGTCCT |
| | | CCCTCTCCGCCTCCGTGGGAGATCGCGTCACGATCACGTGC |
| | | AGGGCCAGCCAGAGCATCTCCAGCTACCTGAACTGGTACCA |
| | | GCAGAAGCCAGGGAAGGCACCGAAGCTCCTGATCTACGCCG |
| | | CTAGCTCGCTGCAGTCCGGCGTCCCTTCACGGTTCTCGGGAT |
| | | CGGGCTCAGGCACCGACTTCACCCTGACCATTAGCAGCCTG |
| | | CAGCCGGAGGACTTCGCGACATACTACTGTCAGCAGTCATA |
| | | CTCCACCCCTCTGACCTTCGGCCAAGGGACCAAAGTGGAGA |
| | | TCAAG |
| SEQ ID | Full CAR | EVQLLESGGGLVQPGGSLRLSCAASGFTFSSYAMSWVRQAPG |
| NO: 82 | amino acid | KGLEWVSAISGSGGSTYYADSVKGRFTISRDNSKNTLYLQMNS |
| | sequence | LRAEDTAVYYCARREWWGESWLFDYWGQGTLVTVSSGGGGS |
| | | GGGGSGGGGSGGGGSDIQMTQSPSSLSASVGDRVTITCRASQSI |
| | | SSYLNWYQQKPGKAPKLLIYAASSLQSGVPSRFSGSGSGTDFTL |
| | | TISSLOPEDFATYYCQQSYSTPLTFGQGTKVEIKTTTPAPRPPTP |
| | | APTIASQPLSLRPEACRPAAGGAVHTRGLDFACDIYIWAPLAGT |
| | | CGVLLLSLVITLYCKRGRKKLLYIFKQPFMRPVQTTQEEDGCSC |
| | | RFPEEEEGGCELRVKFSRSADAPAYQQGQNQLYNELNLGRREE |
| | | YDVLDKRRGRDPEMGGKPRRKNPQEGLYNELQKDKMAEAYS |
| | | EIGMKGERRRGKGHDGLYQGLSTATKDTYDALHMQALPPR |
| SEQ ID | Full CAR | GAAGTGCAGTTGCTGGAGTCAGGCGGAGGACTGGTGCAGCC |
| NO: 83 | DNA | CGGAGGATCGCTTCGCTTGAGCTGCGCAGCCTCAGGCTTTA |
| | sequence | CCTTCTCCTCCTACGCCATGTCCTGGGTCAGACAGGCTCCCG |
| | | GGAAGGGACTGGAATGGGTGTCCGCCATTAGCGGTTCCGGC |
| | | GGAAGCACTTACTATGCCGACTCTGTGAAGGGCCGCTTCAC |
| | | TATCTCCCGGGACAACTCCAAGAACACCCTGTATCTCCAAA |
| | | TGAATTCCCTGAGGGCCGAAGATACCGCGGTGTACTACTGC |
| | | GCTAGACGGGAGTGGTGGGGAGAAAGCTGGCTGTTCGACTA |
| | | CTGGGGACAGGGCACTCTCGTGACTGTGTCCTCCGGTGGTG |
| | | GTGGATCGGGGGGTGGTGGTTCGGGCGGAGGAGGATCTGG |
| | | AGGAGGAGGGTCGGACATTCAAATGACTCAGTCCCCGTCCT |
| | | CCCTCTCCGCCTCCGTGGGAGATCGCGTCACGATCACGTGC |
| | | AGGGCCAGCCAGAGCATCTCCAGCTACCTGAACTGGTACCA |
| | | GCAGAAGCCAGGGAAGGCACCGAAGCTCCTGATCTACGCCG |
| | | CTAGCTCGCTGCAGTCCGGCGTCCCTTCACGGTTCTCGGGAT |
| | | CGGGCTCAGGCACCGACTTCACCCTGACCATTAGCAGCCTG |
| | | CAGCCGGAGGACTTCGCGACATACTACTGTCAGCAGTCATA |
| | | CTCCACCCCTCTGACCTTCGGCCAAGGGACCAAAGTGGAGA |
| | | TCAAGACCACTACCCCAGCACCGAGGCCACCCACCCCGGCT |
| | | CCTACCATCGCCTCCCAGCCTCTGTCCCTGCGTCCGGAGGCA |
| | | TGTAGACCCGCAGCTGGTGGGGCCGTGCATACCCGGGGTCT |
| | | TGACTTCGCCTGCGATATCTACATTTGGGCCCCTCTGGCTGG |
| | | TACTTGCGGGGTCCTGCTGCTTTCACTCGTGATCACTCTTTA |
| | | CTGTAAGCGCGGTCGGAAGAAGCTGCTGTACATCTTTAAGC |
| | | AACCCTTCATGAGGCCTGTGCAGACTACTCAAGAGGAGGAC |
| | | GGCTGTTCATGCCGGTTCCCAGAGGAGGAGGAAGGCGGCTG |
| | | CGAACTGCGCGTGAAATTCAGCCGCAGCGCAGATGCTCCAG |
| | | CCTACCAGCAGGGGCAGAACCAGCTCTACAACGAACTCAAT |
| | | CTTGGTCGGAGAGAGGAGTACGACGTGCTGGACAAGCGGA |
| | | GAGGACGGGACCCAGAAATGGGCGGGAAGCCGCGCAGAAA |
| | | GAATCCCCAAGAGGGCCTGTACAACGAGCTCCAAAAGGATA |
| | | AGATGGCAGAAGCCTATAGCGAGATTGGTATGAAAGGGGA |
| | | ACGCAGAAGAGGCAAAGGCCACGACGGACTGTACCAGGGA |
| | | CTCAGCACCGCCACCAAGGACACCTATGACGCTCTTCACAT |
| | | GCAGGCCCTGCCGCCTCGG |

TABLE 3
Kabat CDRs of exemplary PALLAS-derived anti-BCMA molecules

| Kabat | HCDR1 | HCDR2 | HCDR3 | LCDR1 | LCDR2 | LCDR3 |
|---|---|---|---|---|---|---|
| R1B6 | SYAMS | AISGSGGSTY | REWVPYDVS | RASQSISSY | AASSL | QQSYST |
| | (SEQ ID | YADSVKG | WYFDY (SEQ | LN (SEQ ID | QS | PLT |
| | NO: 44) | (SEQ ID NO: | ID NO: 46) | NO: 54) | (SEQ | (SEQ ID |
| | | 45) | | | ID NO: | NO: 56) |
| | | | | | 55) | |
| R1F2 | SYAMS | AISGSGGSTY | REWWYDDW | RASQSISSY | AASSL | QQSYST |
| | (SEQ ID | YADSVKG | YLDY (SEQ | LN (SEQ ID | QS | PLT |
| | NO: 44) | (SEQ ID NO: | ID NO: 68) | NO: 54) | (SEQ | (SEQ ID |
| | | 45) | | | ID NO: | NO: 56) |
| | | | | | 55) | |
| R1G5 | SYAMS | AISGSGGSTY | REWWGESW | RASQSISSY | AASSL | QQSYST |
| | (SEQ ID | YADSVKG | LFDY (SEQ | LN (SEQ ID | QS | PLT |
| | NO: 44) | (SEQ ID NO: | ID NO: 76) | NO: 54) | (SEQ | (SEQ ID |
| | | 45) | | | ID NO: | NO: 56) |
| | | | | | 55) | |
| Consensus | SYAMS | AISGSGGSTY | REWX₁X₂X₃X₄ | RASQSISSY | AASSL | QQSYST |
| | (SEQ ID | YADSVKG | X₅X₆WX₇X₈DY, | LN (SEQ ID | QS | PLT |
| | NO: 44) | (SEQ ID NO: | wherein X₁ | NO: 54) | (SEQ | (SEQ ID |
| | | 45) | is absent or V; | | ID NO: | NO: 56) |
| | | | X₂ is absent or | | 55) | |
| | | | P; X₃ is W or | | | |
| | | | Y; X₄ is G, Y, | | | |
| | | | or D; X₅ is E, | | | |
| | | | D, or V; X₆ is S | | | |
| | | | or D; X₇ is L or | | | |
| | | | Y; and X₈ is F | | | |
| | | | or L (SEQ ID | | | |
| | | | NO: 84) | | | |

TABLE 4
Chothia CDRs of exemplary PALLAS-derived anti-BCMA molecules

| Chothia | HCDR1 | HCDR2 | HCDR3 | LCDR1 | LCDR2 | LCDR3 |
|---|---|---|---|---|---|---|
| R1B6 | GFTFSSY | SGSGGS (SEQ | REWVPYDVS | SQSISSY | AAS | SYSTPL |
| | (SEQ ID | ID NO: 48) | WYFDY (SEQ | (SEQ ID | (SEQ | (SEQ ID |
| | NO: 47) | | ID NO: 46) | NO: 57) | ID NO: | NO: 59) |
| | | | | | 58) | |
| R1F2 | GFTFSSY | SGSGGS (SEQ | REWWYDDW | SQSISSY | AAS | SYSTPL |
| | (SEQ ID | ID NO: 48) | YLDY (SEQ ID | (SEQ ID | (SEQ | (SEQ ID |
| | NO: 47) | | NO: 68) | NO: 57) | ID NO: | NO: 59) |
| | | | | | 58) | |
| R1G5 | GFTFSSY | SGSGGS (SEQ | REWWGESWL | SQSISSY | AAS | SYSTPL |
| | (SEQ ID | ID NO: 48) | FDY (SEQ ID | (SEQ ID | (SEQ | (SEQ ID |
| | NO: 47) | | NO: 76) | NO: 57) | ID NO: | NO: 59) |
| | | | | | 58) | |
| Consensus | GFTFSSY | SGSGGS (SEQ | REWX₁X₂X₃X₄ | SQSISSY | AAS | SYSTPL |
| | (SEQ ID | ID NO: 48) | X₅X₆WX₇X₈DY, | (SEQ ID | (SEQ | (SEQ ID |
| | NO: 47) | | wherein X₁ is | NO: 57) | ID NO: | NO: 59) |
| | | | absent or V; X₂ | | 58) | |
| | | | is absent or P; | | | |
| | | | X₃ is W or Y; X₄ | | | |
| | | | is G, Y, or D; X₅ | | | |
| | | | is E, D, or V; X₆ | | | |
| | | | is S or D; X₇ is | | | |
| | | | L or Y; and X₈ | | | |
| | | | is F or L (SEQ | | | |
| | | | ID NO: 84) | | | |

TABLE 5
IMGT CDRs of exemplary PALLAS-derived anti-BCMA molecules

| IMGT | HCDR1 | HCDR2 | HCDR3 | LCDR1 | LCDR2 | LCDR3 |
|---|---|---|---|---|---|---|
| R1B6 | GFTFSSYA | ISGSGGST | ARREWVPYD | QSISSY | AAS (SEQ | QQSYST |
| | (SEQ ID | (SEQ ID NO: | VSWYFDY | (SEQ ID | ID NO: 58) | PLT (SEQ |
| | NO: 49) | 50) | (SEQ ID NO: | NO: 60) | | ID NO: |
| | | | 51) | | | 56) |
| R1F2 | GFTFSSYA | ISGSGGST | ARREWWYDD | QSISSY | AAS (SEQ | QQSYST |
| | (SEQ ID | (SEQ ID NO: | WYLDY (SEQ | (SEQ ID | ID NO: 58) | PLT (SEQ |
| | NO: 49) | 50) | ID NO: 69) | NO: 60) | | ID NO: |
| | | | | | | 56) |
| R1G5 | GFTFSSYA | ISGSGGST | ARREWWGES | QSISSY | AAS (SEQ | QQSYST |
| | (SEQ ID | (SEQ ID NO: | WLFDY (SEQ | (SEQ ID | ID NO: 58) | PLT (SEQ |
| | NO: 49) | 50) | ID NO: 77) | NO: 60) | | ID NO: |
| | | | | | | 56) |
| Consensus | GFTFSSYA | ISGSGGST | ARREWX₁X₂X₃ | QSISSY | AAS (SEQ | QQSYST |
| | (SEQ ID | (SEQ ID NO: | X₄X₅X₆WX₇X₈ | (SEQ ID | ID NO: 58) | PLT (SEQ |
| | NO: 49) | 50) | DY, wherein X₁ | NO: 60) | | ID NO: |
| | | | is absent or V; | | | 56) |
| | | | X₂ is absent or | | | |
| | | | P; X₃ is W or Y; | | | |
| | | | X₄ is G, Y, or D; | | | |
| | | | X₅ is E, D, or V; | | | |
| | | | X₆ is S or D; X₇ | | | |
| | | | is L or Y; and | | | |
| | | | X₈ is F or L | | | |
| | | | (SEQ ID NO: | | | |
| | | | 85) | | | |

TABLE 6
Amino acid and nucleic acid sequences
of exemplary B cell-derived anti-BCMA molecules

| SEQ ID | Name/ | |
|---|---|---|
| NO | Description | Sequence |
| PI61 | | |
| SEQ ID | HCDR1 | SYGMH |
| NO: 86 | (Kabat) | |
| SEQ ID | HCDR2 | VISYDGSNKYYADSVKG |
| NO: 87 | (Kabat) | |
| SEQ ID | HCDR3 | SGYALHDDYYGLDV |
| NO: 88 | (Kabat) | |
| SEQ ID | HCDR1 | GFTFSSY |
| NO: 47 | (Chothia) | |
| SEQ ID | HCDR2 | SYDGSN |
| NO: 89 | (Chothia) | |
| SEQ ID | HCDR3 | SGYALHDDYYGLDV |
| NO: 88 | (Chothia) | |
| SEQ ID | HCDR1 | GFTFSSYG |
| NO: 90 | (IMGT) | |
| SEQ ID | HCDR2 | ISYDGSNK |
| NO: 91 | (IMGT) | |
| SEQ ID | HCDR3 | GGSGYALHDDYYGLDV |
| NO: 92 | (IMGT) | |
| SEQ ID | VH | QVQLQESGGGVVQPGRSLRLSCAASGFTFSSYGMHWVRQAPG |
| NO: 93 | | KGLEWVAVISYDGSNKYYADSVKGRFTISRDNSKNTLYLQMNS |
| | | LRAEDTAVYYCGGSGYALHDDYYGLDVWGQGTLVTVSS |
| SEQ ID | DNA VH | CAAGTGCAGCTGCAGGAATCCGGTGGCGGAGTCGTGCAGCC |
| NO: 94 | | TGGAAGGAGCCTGAGACTCTCATGCGCCGCGTCAGGGTTCAC |
| | | CTTTTCCTCCTACGGGATGCATTGGGTCAGACAGGCCCCCGG |
| | | AAAGGGACTCGAATGGGTGGCTGTGATCAGCTACGACGGCT |
| | | CCAACAAGTACTACGCCGACTCCGTGAAAGGCCGGTTCACTA |
| | | TCTCCCGGGACAACTCCAAGAACACGCTGTATCTGCAAATGA |
| | | ATTCACTGCGCGCGGAGGATACCGCTGTGTACTACTGCGGTG |
| | | GCTCCGGTTACGCCCTGCACGATGACTATTACGGCCTTGACG |
| | | TCTGGGGCCAGGGAACCCTCGTGACTGTGTCCAGC |
| SEQ ID | LCDR1 | TGTSSDVGGYNYVS |
| NO: 95 | (Kabat) | |
| SEQ ID | LCDR2 | DVSNRPS |
| NO: 96 | (Kabat) | |
| SEQ ID | LCDR3 | SSYTSSSTLYV |
| NO: 97 | (Kabat) | |
| SEQ ID | LCDR1 | TSSDVGGYNY |
| NO: 98 | (Chothia) | |
| SEQ ID | LCDR2 | DVS |
| NO: 99 | (Chothia) | |
| SEQ ID | LCDR3 | YTSSSTLY |
| NO: 100 | (Chothia) | |
| SEQ ID | LCDR1 | SSDVGGYNY |
| NO: 101 | (IMGT) | |
| SEQ ID | LCDR2 | DVS |
| NO: 99 | (IMGT) | |
| SEQ ID | LCDR3 | SSYTSSSTLYV |
| NO: 97 | (IMGT) | |
| SEQ ID | VL | QSALTQPASVSGSPGQSITISCTGTSSDVGGYNYVSWYQQHPGK |
| NO: 102 | | APKLMIYDVSNRPSGVSNRFSGSKSGNTASLTISGLQAEDEADY |
| | | YCSSYTSSSTLYVFGSGTKVTVL |
| SEQ ID | DNA VL | CAGAGCGCACTGACTCAGCCGGCATCCGTGTCCGGTAGCCCC |
| NO: 103 | | GGACAGTCGATTACCATCTCCTGTACCGGCACCTCCTCCGAC |
| | | GTGGGAGGGTACAACTACGTGTCGTGGTACCAGCAGCACCC |
| | | AGGAAAGGCCCCTAAGTTGATGATCTACGATGTGTCAAACCG |
| | | CCCGTCTGGAGTCTCCAACCGGTTCTCCGGCTCCAAGTCCGG |
| | | CAACACCGCCAGCCTGACCATTAGCGGGCTGCAAGCCGAGG |
| | | ATGAGGCCGACTACTACTGCTCGAGCTACACATCCTCGAGCA |
| | | CCCTCTACGTGTTCGGCTCGGGGACTAAGGTCACCGTGCTG |
| SEQ ID | Linker | GGGGSGGGGSGGGGS |
| NO: 104 | | |
| SEQ ID | scFv (VH- | QVQLQESGGGVVQPGRSLRLSCAASGFTFSSYGMHWVRQAPG |
| NO: 105 | linker-VL) | KGLEWVAVISYDGSNKYYADSVKGRFTISRDNSKNTLYLQMNS |
| | | LRAEDTAVYYCGGSGYALHDDYYGLDVWGQGTLVTVSSGGG |
| | | GSGGGGSGGGGSQSALTQPASVSGSPGQSITISCTGTSSDVGGY |
| | | NYVSWYQQHPGKAPKLMIYDVSNRPSGVSNRFSGSKSGNTASL |
| | | TISGLQAEDEADYYCSSYTSSSTLYVFGSGTKVTVL |
| SEQ ID | DNA scFv | CAAGTGCAGCTGCAGGAATCCGGTGGCGGAGTCGTGCAGCC |
| NO: 106 | | TGGAAGGAGCCTGAGACTCTCATGCGCCGCGTCAGGGTTCAC |
| | | CTTTTCCTCCTACGGGATGCATTGGGTCAGACAGGCCCCCGG |
| | | AAAGGGACTCGAATGGGTGGCTGTGATCAGCTACGACGGCT |
| | | CCAACAAGTACTACGCCGACTCCGTGAAAGGCCGGTTCACTA |
| | | TCTCCCGGGACAACTCCAAGAACACGCTGTATCTGCAAATGA |
| | | ATTCACTGCGCGCGGAGGATACCGCTGTGTACTACTGCGGTG |
| | | GCTCCGGTTACGCCCTGCACGATGACTATTACGGCCTTGACG |
| | | TCTGGGGCCAGGGAACCCTCGTGACTGTGTCCAGCGGTGGAG |
| | | GAGGTTCGGGCGGAGGAGGATCAGGAGGGGGTGGATCGCAG |
| | | AGCGCACTGACTCAGCCGGCATCCGTGTCCGGTAGCCCCGGA |
| | | CAGTCGATTACCATCTCCTGTACCGGCACCTCCTCCGACGTG |
| | | GGAGGGTACAACTACGTGTCGTGGTACCAGCAGCACCCAGG |
| | | AAAGGCCCCTAAGTTGATGATCTACGATGTGTCAAACCGCCC |
| | | GTCTGGAGTCTCCAACCGGTTCTCCGGCTCCAAGTCCGGCAA |
| | | CACCGCCAGCCTGACCATTAGCGGGCTGCAAGCCGAGGATG |
| | | AGGCCGACTACTACTGCTCGAGCTACACATCCTCGAGCACCC |
| | | TCTACGTGTTCGGCTCGGGGACTAAGGTCACCGTGCTG |
| SEQ ID | Full CAR | QVQLQESGGGVVQPGRSLRLSCAASGFTFSSYGMHWVRQAPG |
| NO: 107 | amino acid | KGLEWVAVISYDGSNKYYADSVKGRFTISRDNSKNTLYLQMNS |
| | sequence | LRAEDTAVYYCGGSGYALHDDYYGLDVWGQGTLVTVSSGGG |
| | | GSGGGGSGGGGSQSALTQPASVSGSPGQSITISCTGTSSDVGGY |
| | | NYVSWYQQHPGKAPKLMIYDVSNRPSGVSNRFSGSKSGNTASL |
| | | TISGLQAEDEADYYCSSYTSSSTLYVFGSGTKVTVLTTTPAPRPP |
| | | TPAPTIASQPLSLRPEACRPAAGGAVHTRGLDFACDIYIWAPLA |
| | | GTCGVLLLSLVITLYCKRGRKKLLYIFKQPFMRPVQTTQEEDGC |
| | | SCRFPEEEEGGCELRVKFSRSADAPAYQQGQNQLYNELNLGRR |
| | | EEYDVLDKRRGRDPEMGGKPRRKNPQEGLYNELQKDKMAEA |
| | | YSEIGMKGERRRGKGHDGLYQGLSTATKDTYDALHMQALPPR |
| SEQ ID | Full CAR | CAAGTGCAGCTGCAGGAATCCGGTGGCGGAGTCGTGCAGCC |
| NO: 108 | DNA | TGGAAGGAGCCTGAGACTCTCATGCGCCGCGTCAGGGTTCAC |
| | sequence | CTTTTCCTCCTACGGGATGCATTGGGTCAGACAGGCCCCCGG |
| | | AAAGGGACTCGAATGGGTGGCTGTGATCAGCTACGACGGCT |
| | | CCAACAAGTACTACGCCGACTCCGTGAAAGGCCGGTTCACTA |
| | | TCTCCCGGGACAACTCCAAGAACACGCTGTATCTGCAAATGA |
| | | ATTCACTGCGCGCGGAGGATACCGCTGTGTACTACTGCGGTG |
| | | GCTCCGGTTACGCCCTGCACGATGACTATTACGGCCTTGACG |
| | | TCTGGGGCCAGGGAACCCTCGTGACTGTGTCCAGCGGTGGAG |
| | | GAGGTTCGGGCGGAGGAGGATCAGGAGGGGGTGGATCGCAG |
| | | AGCGCACTGACTCAGCCGGCATCCGTGTCCGGTAGCCCCGGA |
| | | CAGTCGATTACCATCTCCTGTACCGGCACCTCCTCCGACGTG |
| | | GGAGGGTACAACTACGTGTCGTGGTACCAGCAGCACCCAGG |
| | | AAAGGCCCCTAAGTTGATGATCTACGATGTGTCAAACCGCCC |
| | | GTCTGGAGTCTCCAACCGGTTCTCCGGCTCCAAGTCCGGCAA |
| | | CACCGCCAGCCTGACCATTAGCGGGCTGCAAGCCGAGGATG |
| | | AGGCCGACTACTACTGCTCGAGCTACACATCCTCGAGCACCC |
| | | TCTACGTGTTCGGCTCGGGGACTAAGGTCACCGTGCTGACCA |
| | | CTACCCCAGCACCGAGGCCACCCACCCCGGCTCCTACCATCG |
| | | CCTCCCAGCCTCTGTCCCTGCGTCCGGAGGCATGTAGACCCG |
| | | CAGCTGGTGGGGCCGTGCATACCCGGGGTCTTGACTTCGCCT |
| | | GCGATATCTACATTTGGGCCCCTCTGGCTGGTACTTGCGGGG |
| | | TCCTGCTGCTTTCACTCGTGATCACTCTTTACTGTAAGCGCGG |
| | | TCGGAAGAAGCTGCTGTACATCTTTAAGCAACCCTTCATGAG |
| | | GCCTGTGCAGACTACTCAAGAGGAGGACGGCTGTTCATGCCG |
| | | GTTCCCAGAGGAGGAGGAAGGCGGCTGCGAACTGCGCGTGA |
| | | AATTCAGCCGCAGCGCAGATGCTCCAGCCTACCAGCAGGGG |
| | | CAGAACCAGCTCTACAACGAACTCAATCTTGGTCGGAGAGA |
| | | GGAGTACGACGTGCTGGACAAGCGGAGAGGACGGGACCCAG |
| | | AAATGGGCGGGAAGCCGCGCAGAAAGAATCCCCAAGAGGGC |
| | | CTGTACAACGAGCTCCAAAAGGATAAGATGGCAGAAGCCTA |
| | | TAGCGAGATTGGTATGAAAGGGGAACGCAGAAGAGGCAAAG |
| | | GCCACGACGGACTGTACCAGGGACTCAGCACCGCCACcaaggac |
| | | acctatgacgctcttcacatgcaggccctgccgcctcgg |
| B61-02 | | |
| SEQ ID | HCDR1 | SYGMH |
| NO: 86 | (Kabat) | |
| SEQ ID | HCDR2 | VISYKGSNKYYADSVKG |
| NO: 109 | (Kabat) | |
| SEQ ID | HCDR3 | SGYALHDDYYGLDV |
| NO: 88 | (Kabat) | |
| SEQ ID | HCDR1 | GFTFSSY |
| NO: 47 | (Chothia) | |
| SEQ ID | HCDR2 | SYKGSN |
| NO: 110 | (Chothia) | |
| SEQ ID | HCDR3 | SGYALHDDYYGLDV |
| NO: 88 | (Chothia) | |
| SEQ ID | HCDR1 | GFTFSSYG |
| NO: 90 | (IMGT) | |
| SEQ ID | HCDR2 | ISYKGSNK |
| NO: 111 | (IMGT) | |
| SEQ ID | HCDR3 | GGSGYALHDDYYGLDV |
| NO: 92 | (IMGT) | |
| SEQ ID | VH | QVQLVESGGGVVQPGRSLRLSCAASGFTFSSYGMHWVRQAPG |
| NO: 112 | | KGLEWVAVISYKGSNKYYADSVKGRFTISRDNSKNTLYLQMNS |
| | | LRAEDTAVYYCGGSGYALHDDYYGLDVWGQGTLVTVSS |
| SEQ ID | DNA VH | CAAGTGCAGCTTGTCGAATCGGGAGGCGGAGTGGTGCAGCC |
| NO: 113 | | TGGACGATCGCTCCGGCTCTCATGTGCCGCGAGCGGATTCAC |
| | | CTTCTCGAGCTACGGCATGCACTGGGTCAGACAAGCCCCAGG |
| | | AAAGGGCCTGGAATGGGTGGCTGTCATCTCGTACAAGGGCTC |
| | | AAACAAGTACTACGCCGACTCCGTGAAGGGCCGGTTCACCAT |
| | | CTCCCGCGATAACTCCAAGAATACCCTCTATCTGCAAATGAA |
| | | CAGCCTGAGGGCCGAGGATACTGCAGTGTACTACTGCGGGG |
| | | GTTCAGGCTACGCGCTGCACGACGACTACTACGGATTGGACG |
| | | TCTGGGGCCAAGGAACTCTTGTGACCGTGTCCTCT |
| SEQ ID | LCDR1 | TGTSSDVGGYNYVS |
| NO: 95 | (Kabat) | |
| SEQ ID | LCDR2 | EVSNRLR |
| NO: 114 | (Kabat) | |
| SEQ ID | LCDR3 | SSYTSSSALYV |
| NO: 115 | (Kabat) | |
| SEQ ID | LCDR1 | TSSDVGGYNY |
| NO: 98 | (Chothia) | |
| SEQ ID | LCDR2 | EVS |
| NO: 116 | (Chothia) | |
| SEQ ID | LCDR3 | YTSSSALY |
| NO: 117 | (Chothia) | |
| SEQ ID | LCDR1 | SSDVGGYNY |
| NO: 101 | (IMGT) | |
| SEQ ID | LCDR2 | EVS |
| NO: 116 | (IMGT) | |
| SEQ ID | LCDR3 | SSYTSSSALYV |
| NO: 115 | (IMGT) | |
| SEQ ID | VL | QSALTQPASVSGSPGQSITISCTGTSSDVGGYNYVSWYQQHPGK |
| NO: 118 | | APKLMIYEVSNRLRGVSNRFSGSKSGNTASLTISGLQAEDEADY |
| | | YCSSYTSSSALYVFGSGTKVTVL |
| SEQ ID | DNA VL | CAGAGCGCGCTGACTCAGCCTGCCTCCGTGAGCGGTTCGCCG |
| NO: 119 | | GGACAGTCCATTACCATTTCGTGCACCGGGACCTCCTCCGAC |
| | | GTGGGAGGCTACAACTACGTGTCCTGGTACCAGCAGCATCCC |
| | | GGAAAGGCCCCGAAGCTGATGATCTACGAAGTGTCGAACAG |
| | | ACTGCGGGGAGTCTCCAACCGCTTTTCCGGGTCCAAGTCCGG |
| | | CAACACCGCCAGCCTGACCATCAGCGGGCTCCAGGCAGAAG |
| | | ATGAGGCTGACTATTACTGCTCCTCCTACACGTCAAGCTCCG |
| | | CCCTCTACGTGTTCGGGTCCGGGACCAAAGTCACTGTGCTG |
| SEQ ID | Linker | GGGGSGGGGSGGGGSGGGGS |
| NO: 63 | | |
| SEQ ID | scFv (VH- | QVQLVESGGGVVQPGRSLRLSCAASGFTFSSYGMHWVRQAPG |
| NO: 120 | linker-VL) | KGLEWVAVISYKGSNKYYADSVKGRFTISRDNSKNTLYLQMNS |
| | | LRAEDTAVYYCGGSGYALHDDYYGLDVWGQGTLVTVSSGGG |
| | | GSGGGGGGGGSGGGGSQSALTQPASVSGSPGQSITISCTGTSSD |
| | | VGGYNYVSWYQQHPGKAPKLMIYEVSNRLRGVSNRFSGSKSG |
| | | NTASLTISGLQAEDEADYYCSSYTSSSALYVFGSGTKVTVL |
| SEQ ID | DNA scFv | CAAGTGCAGCTTGTCGAATCGGGAGGCGGAGTGGTGCAGCC |
| NO: 121 | | TGGACGATCGCTCCGGCTCTCATGTGCCGCGAGCGGATTCAC |
| | | CTTCTCGAGCTACGGCATGCACTGGGTCAGACAAGCCCCAGG |
| | | AAAGGGCCTGGAATGGGTGGCTGTCATCTCGTACAAGGGCTC |
| | | AAACAAGTACTACGCCGACTCCGTGAAGGGCCGGTTCACCAT |
| | | CTCCCGCGATAACTCCAAGAATACCCTCTATCTGCAAATGAA |
| | | CAGCCTGAGGGCCGAGGATACTGCAGTGTACTACTGCGGGG |
| | | GTTCAGGCTACGCGCTGCACGACGACTACTACGGATTGGACG |
| | | TCTGGGGCCAAGGAACTCTTGTGACCGTGTCCTCTGGTGGAG |
| | | GCGGATCAGGGGGTGGCGGATCTGGGGGTGGTGGTTCCGGG |
| | | GGAGGAGGATCGCAGAGCGCGCTGACTCAGCCTGCCTCCGT |
| | | GAGCGGTTCGCCGGGACAGTCCATTACCATTTCGTGCACCGG |
| | | GACCTCCTCCGACGTGGGAGGCTACAACTACGTGTCCTGGTA |
| | | CCAGCAGCATCCCGGAAAGGCCCCGAAGCTGATGATCTACG |
| | | AAGTGTCGAACAGACTGCGGGGAGTCTCCAACCGCTTTTCCG |
| | | GGTCCAAGTCCGGCAACACCGCCAGCCTGACCATCAGCGGG |
| | | CTCCAGGCAGAAGATGAGGCTGACTATTACTGCTCCTCCTAC |
| | | ACGTCAAGCTCCGCCCTCTACGTGTTCGGGTCCGGGACCAAA |
| | | GTCACTGTGCTG |
| SEQ ID | Full CAR | QVQLVESGGGVVQPGRSLRLSCAASGFTFSSYGMHWVRQAPG |
| NO: 122 | amino acid | KGLEWVAVISYKGSNKYYADSVKGRFTISRDNSKNTLYLQMNS |
| | sequence | LRAEDTAVYYCGGSGYALHDDYYGLDVWGQGTLVTVSSGGG |
| | | GSGGGGSGGGGSGGGGSQSALTQPASVSGSPGQSITISCTGTSSD |
| | | VGGYNYVSWYQQHPGKAPKLMIYEVSNRLRGVSNRFSGSKSG |
| | | NTASLTISGLQAEDEADYYCSSYTSSSALYVFGSGTKVTVLTTTP |
| | | APRPPTPAPTIASQPLSLRPEACRPAAGGAVHTRGLDFACDIYIW |
| | | APLAGTCGVLLLSLVITLYCKRGRKKLLYIFKQPFMRPVQTTQE |
| | | EDGCSCRFPEEEEGGCELRVKFSRSADAPAYQQGQNQLYNELN |
| | | LGRREEYDVLDKRRGRDPEMGGKPRRKNPQEGLYNELQKDKM |
| | | AEAYSEIGMKGERRRGKGHDGLYQGLSTATKDTYDALHMQAL |
| | | PPR |
| SEQ ID | Full CAR | CAAGTGCAGCTTGTCGAATCGGGAGGCGGAGTGGTGCAGCC |
| NO: 123 | DNA | TGGACGATCGCTCCGGCTCTCATGTGCCGCGAGCGGATTCAC |
| | sequence | CTTCTCGAGCTACGGCATGCACTGGGTCAGACAAGCCCCAGG |
| | | AAAGGGCCTGGAATGGGTGGCTGTCATCTCGTACAAGGGCTC |
| | | AAACAAGTACTACGCCGACTCCGTGAAGGGCCGGTTCACCAT |
| | | CTCCCGCGATAACTCCAAGAATACCCTCTATCTGCAAATGAA |
| | | CAGCCTGAGGGCCGAGGATACTGCAGTGTACTACTGCGGGG |
| | | GTTCAGGCTACGCGCTGCACGACGACTACTACGGATTGGACG |
| | | TCTGGGGCCAAGGAACTCTTGTGACCGTGTCCTCTGGTGGAG |
| | | GCGGATCAGGGGGTGGCGGATCTGGGGGTGGTGGTTCCGGG |
| | | GGAGGAGGATCGCAGAGCGCGCTGACTCAGCCTGCCTCCGT |
| | | GAGCGGTTCGCCGGGACAGTCCATTACCATTTCGTGCACCGG |
| | | GACCTCCTCCGACGTGGGAGGCTACAACTACGTGTCCTGGTA |
| | | CCAGCAGCATCCCGGAAAGGCCCCGAAGCTGATGATCTACG |
| | | AAGTGTCGAACAGACTGCGGGGAGTCTCCAACCGCTTTTCCG |
| | | GGTCCAAGTCCGGCAACACCGCCAGCCTGACCATCAGCGGG |
| | | CTCCAGGCAGAAGATGAGGCTGACTATTACTGCTCCTCCTAC |
| | | ACGTCAAGCTCCGCCCTCTACGTGTTCGGGTCCGGGACCAAA |
| | | GTCACTGTGCTGACCACTACCCCAGCACCGAGGCCACCCACC |
| | | CCGGCTCCTACCATCGCCTCCCAGCCTCTGTCCCTGCGTCCGG |
| | | AGGCATGTAGACCCGCAGCTGGTGGGGCCGTGCATACCCGG |
| | | GGTCTTGACTTCGCCTGCGATATCTACATTTGGGCCCCTCTGG |
| | | CTGGTACTTGCGGGGTCCTGCTGCTTTCACTCGTGATCACTCT |
| | | TTACTGTAAGCGCGGTCGGAAGAAGCTGCTGTACATCTTTAA |
| | | GCAACCCTTCATGAGGCCTGTGCAGACTACTCAAGAGGAGG |
| | | ACGGCTGTTCATGCCGGTTCCCAGAGGAGGAGGAAGGCGGC |
| | | TGCGAACTGCGCGTGAAATTCAGCCGCAGCGCAGATGCTCCA |
| | | GCCTACCAGCAGGGGCAGAACCAGCTCTACAACGAACTCAA |
| | | TCTTGGTCGGAGAGAGGAGTACGACGTGCTGGACAAGCGGA |
| | | GAGGACGGGACCCAGAAATGGGCGGGAAGCCGCGCAGAAA |
| | | GAATCCCCAAGAGGGCCTGTACAACGAGCTCCAAAAGGATA |
| | | AGATGGCAGAAGCCTATAGCGAGATTGGTATGAAAGGGGAA |
| | | CGCAGAAGAGGCAAAGGCCACGACGGACTGTACCAGGGACT |
| | | CAGCACCGCCACCAAGGACACCTATGACGCTCTTCACATGCA |
| | | GGCCCTGCCGCCTCGG |
| B61-10 | | |
| SEQ ID | HCDR1 | SYGMH |
| NO: 86 | (Kabat) | |
| SEQ ID | HCDR2 | VISYKGSNKYYADSVKG |
| NO: 109 | (Kabat) | |
| SEQ ID | HCDR3 | SGYALHDDYYGLDV |
| NO: 88 | (Kabat) | |
| SEQ ID | HCDR1 | GFTFSSY |
| NO: 47 | (Chothia) | |
| SEQ ID | HCDR2 | SYKGSN |
| NO: 110 | (Chothia) | |
| SEQ ID | HCDR3 | SGYALHDDYYGLDV |
| NO: 88 | (Chothia) | |
| SEQ ID | HCDR1 | GFTFSSYG |
| NO: 90 | (IMGT) | |
| SEQ ID | HCDR2 | ISYKGSNK |
| NO: 111 | (IMGT) | |
| SEQ ID | HCDR3 | GGSGYALHDDYYGLDV |
| NO: 92 | (IMGT) | |
| SEQ ID | VH | QVQLVESGGGVVQPGRSLRLSCAASGFTFSSYGMHWVRQAPG |
| NO: 112 | | KGLEWVAVISYKGSNKYYADSVKGRFTISRDNSKNTLYLQMNS |
| | | LRAEDTAVYYCGGSGYALHDDYYGLDVWGQGTLVTVSS |
| SEQ ID | DNA VH | CAAGTGCAGCTTGTCGAATCGGGAGGCGGAGTGGTGCAGCC |
| NO: 113 | | TGGACGATCGCTCCGGCTCTCATGTGCCGCGAGCGGATTCAC |
| | | CTTCTCGAGCTACGGCATGCACTGGGTCAGACAAGCCCCAGG |
| | | AAAGGGCCTGGAATGGGTGGCTGTCATCTCGTACAAGGGCTC |
| | | AAACAAGTACTACGCCGACTCCGTGAAGGGCCGGTTCACCAT |
| | | CTCCCGCGATAACTCCAAGAATACCCTCTATCTGCAAATGAA |
| | | CAGCCTGAGGGCCGAGGATACTGCAGTGTACTACTGCGGGG |
| | | GTTCAGGCTACGCGCTGCACGACGACTACTACGGATTGGACG |
| | | TCTGGGGCCAAGGAACTCTTGTGACCGTGTCCTCT |
| SEQ ID | LCDR1 | TGTSSDVGGYNYVS |
| NO: 95 | (Kabat) | |
| SEQ ID | LCDR2 | EVSNRLR |
| NO: 114 | (Kabat) | |
| SEQ ID | LCDR3 | SSYTSSSTLYV |
| NO: 97 | (Kabat) | |
| SEQ ID | LCDR1 | TSSDVGGYNY |
| NO: 98 | (Chothia) | |
| SEQ ID | LCDR2 | EVS |
| NO: 116 | (Chothia) | |
| SEQ ID | LCDR3 | YTSSSTLY |
| NO: 100 | (Chothia) | |
| SEQ ID | LCDR1 | SSDVGGYNY |
| NO: 101 | (IMGT) | |
| SEQ ID | LCDR2 | EVS |
| NO: 116 | (IMGT) | |
| SEQ ID | LCDR3 | SSYTSSSTLYV |
| NO: 97 | (IMGT) | |
| SEQ ID | VL | QSALTQPASVSGSPGQSITISCTGTSSDVGGYNYVSWYQQHPGK |
| NO: 124 | | APKLMIYEVSNRLRGVSNRFSGSKSGNTASLTISGLQAEDEADY |
| | | YCSSYTSSSTLYVFGSGTKVTVL |
| SEQ ID | DNA VL | CAGAGCGCGCTGACTCAGCCTGCCTCCGTGAGCGGTTCGCCG |
| NO: 125 | | GGACAGTCCATTACCATTTCGTGCACCGGGACCTCCTCCGAC |
| | | GTGGGAGGCTACAACTACGTGTCCTGGTACCAGCAGCATCCC |
| | | GGAAAGGCCCCGAAGCTGATGATCTACGAAGTGTCGAACAG |
| | | ACTGCGGGGAGTCTCCAACCGCTTTTCCGGGTCCAAGTCCGG |
| | | CAACACCGCCAGCCTGACCATCAGCGGGCTCCAGGCAGAAG |
| | | ATGAGGCTGACTATTACTGCTCCTCCTACACGTCAAGCTCCA |
| | | CCCTCTACGTGTTCGGGTCCGGGACCAAAGTCACTGTGCTG |
| SEQ ID | Linker | GGGGSGGGGSGGGGSGGGGS |
| NO: 63 | | |
| SEQ ID | scFv (VH- | QVQLVESGGGVVQPGRSLRLSCAASGFTFSSYGMHWVRQAPG |
| NO: 126 | linker-VL) | KGLEWVAVISYKGSNKYYADSVKGRFTISRDNSKNTLYLQMNS |
| | | LRAEDTAVYYCGGSGYALHDDYYGLDVWGQGTLVTVSSGGG |
| | | GSGGGGSGGGGSGGGGSQSALTQPASVSGSPGQSITISCTGTSSD |
| | | VGGYNYVSWYQQHPGKAPKLMIYEVSNRLRGVSNRFSGSKSG |
| | | NTASLTISGLQAEDEADYYCSSYTSSSTLYVFGSGTKVTVL |
| SEQ ID | DNA scFv | CAAGTGCAGCTTGTCGAATCGGGAGGCGGAGTGGTGCAGCC |
| NO: 127 | | TGGACGATCGCTCCGGCTCTCATGTGCCGCGAGCGGATTCAC |
| | | CTTCTCGAGCTACGGCATGCACTGGGTCAGACAAGCCCCAGG |
| | | AAAGGGCCTGGAATGGGTGGCTGTCATCTCGTACAAGGGCTC |
| | | AAACAAGTACTACGCCGACTCCGTGAAGGGCCGGTTCACCAT |
| | | CTCCCGCGATAACTCCAAGAATACCCTCTATCTGCAAATGAA |
| | | CAGCCTGAGGGCCGAGGATACTGCAGTGTACTACTGCGGGG |
| | | GTTCAGGCTACGCGCTGCACGACGACTACTACGGATTGGACG |
| | | TCTGGGGCCAAGGAACTCTTGTGACCGTGTCCTCTGGTGGAG |
| | | GCGGATCAGGGGGTGGCGGATCTGGGGGTGGTGGTTCCGGG |
| | | GGAGGAGGATCGCAGAGCGCGCTGACTCAGCCTGCCTCCGT |
| | | GAGCGGTTCGCCGGGACAGTCCATTACCATTTCGTGCACCGG |
| | | GACCTCCTCCGACGTGGGAGGCTACAACTACGTGTCCTGGTA |
| | | CCAGCAGCATCCCGGAAAGGCCCCGAAGCTGATGATCTACG |
| | | AAGTGTCGAACAGACTGCGGGGAGTCTCCAACCGCTTTTCCG |
| | | GGTCCAAGTCCGGCAACACCGCCAGCCTGACCATCAGCGGG |
| | | CTCCAGGCAGAAGATGAGGCTGACTATTACTGCTCCTCCTAC |
| | | ACGTCAAGCTCCACCCTCTACGTGTTCGGGTCCGGGACCAAA |
| | | GTCACTGTGCTG |
| SEQ ID | Full CAR | QVQLVESGGGVVQPGRSLRLSCAASGFTFSSYGMHWVRQAPG |
| NO: 128 | amino acid | KGLEWVAVISYKGSNKYYADSVKGRFTISRDNSKNTLYLQMNS |
| | sequence | LRAEDTAVYYCGGSGYALHDDYYGLDVWGQGTLVTVSSGGG |
| | | GSGGGGSGGGGSGGGGSQSALTQPASVSGSPGQSITISCTGTSSD |
| | | VGGYNYVSWYQQHPGKAPKLMIYEVSNRLRGVSNRFSGSKSG |
| | | NTASLTISGLQAEDEADYYCSSYTSSSTLYVFGSGTKVTVLTTTP |
| | | APRPPTPAPTIASQPLSLRPEACRPAAGGAVHTRGLDFACDIYIW |
| | | APLAGTCGVLLLSLVITLYCKRGRKKLLYIFKQPFMRPVQTTQE |
| | | EDGCSCRFPEEEEGGCELRVKFSRSADAPAYQQGQNQLYNELN |
| | | LGRREEYDVLDKRRGRDPEMGGKPRRKNPQEGLYNELQKDKM |
| | | AEAYSEIGMKGERRRGKGHDGLYQGLSTATKDTYDALHMQAL |
| | | PPR |
| SEQ ID | Full CAR | CAAGTGCAGCTTGTCGAATCGGGAGGCGGAGTGGTGCAGCC |
| NO: 129 | DNA | TGGACGATCGCTCCGGCTCTCATGTGCCGCGAGCGGATTCAC |
| | sequence | CTTCTCGAGCTACGGCATGCACTGGGTCAGACAAGCCCCAGG |
| | | AAAGGGCCTGGAATGGGTGGCTGTCATCTCGTACAAGGGCTC |
| | | AAACAAGTACTACGCCGACTCCGTGAAGGGCCGGTTCACCAT |
| | | CTCCCGCGATAACTCCAAGAATACCCTCTATCTGCAAATGAA |
| | | CAGCCTGAGGGCCGAGGATACTGCAGTGTACTACTGCGGGG |
| | | GTTCAGGCTACGCGCTGCACGACGACTACTACGGATTGGACG |
| | | TCTGGGGCCAAGGAACTCTTGTGACCGTGTCCTCTGGTGGAG |
| | | GCGGATCAGGGGGTGGCGGATCTGGGGGTGGTGGTTCCGGG |
| | | GGAGGAGGATCGCAGAGCGCGCTGACTCAGCCTGCCTCCGT |
| | | GAGCGGTTCGCCGGGACAGTCCATTACCATTTCGTGCACCGG |
| | | GACCTCCTCCGACGTGGGAGGCTACAACTACGTGTCCTGGTA |
| | | CCAGCAGCATCCCGGAAAGGCCCCGAAGCTGATGATCTACG |
| | | AAGTGTCGAACAGACTGCGGGGAGTCTCCAACCGCTTTTCCG |
| | | GGTCCAAGTCCGGCAACACCGCCAGCCTGACCATCAGCGGG |
| | | CTCCAGGCAGAAGATGAGGCTGACTATTACTGCTCCTCCTAC |
| | | ACGTCAAGCTCCACCCTCTACGTGTTCGGGTCCGGGACCAAA |
| | | GTCACTGTGCTGACCACTACCCCAGCACCGAGGCCACCCACC |
| | | CCGGCTCCTACCATCGCCTCCCAGCCTCTGTCCCTGCGTCCGG |
| | | AGGCATGTAGACCCGCAGCTGGTGGGGCCGTGCATACCCGG |
| | | GGTCTTGACTTCGCCTGCGATATCTACATTTGGGCCCCTCTGG |
| | | CTGGTACTTGCGGGGTCCTGCTGCTTTCACTCGTGATCACTCT |
| | | TTACTGTAAGCGCGGTCGGAAGAAGCTGCTGTACATCTTTAA |
| | | GCAACCCTTCATGAGGCCTGTGCAGACTACTCAAGAGGAGG |
| | | ACGGCTGTTCATGCCGGTTCCCAGAGGAGGAGGAAGGCGGC |
| | | TGCGAACTGCGCGTGAAATTCAGCCGCAGCGCAGATGCTCCA |
| | | GCCTACCAGCAGGGGCAGAACCAGCTCTACAACGAACTCAA |
| | | TCTTGGTCGGAGAGAGGAGTACGACGTGCTGGACAAGCGGA |
| | | GAGGACGGGACCCAGAAATGGGCGGGAAGCCGCGCAGAAA |
| | | GAATCCCCAAGAGGGCCTGTACAACGAGCTCCAAAAGGATA |
| | | AGATGGCAGAAGCCTATAGCGAGATTGGTATGAAAGGGGAA |
| | | CGCAGAAGAGGCAAAGGCCACGACGGACTGTACCAGGGACT |
| | | CAGCACCGCCACCAAGGACACCTATGACGCTCTTCACATGCA |
| | | GGCCCTGCCGCCTCGG |

TABLE 7
Kabat CDRs of exemplary B cell-derived anti-BCMA molecules

| Kabat | HCDR1 | HCDR2 | HCDR3 | LCDR1 | LCDR2 | LCDR3 |
|---|---|---|---|---|---|---|
| PI61 | SYGMH | VISYDGSN | SGYALHDD | TGTSSDV | DVSNRPS | SSYTSSS |
| | (SEQ ID | KYYADSV | YYGLDV | GGYNYVS | (SEQ ID NO: | TLYV |
| | NO: 86) | KG (SEQ | (SEQ ID NO: | (SEQ ID | 96) | (SEQ ID |
| | | ID NO: 87) | 88) | NO: 95) | | NO: 97) |
| B61- | SYGMH | VISYKGSN | SGYALHDD | TGTSSDV | EVSNRLR | SSYTSSS |
| 02 | (SEQ ID | KYYADSV | YYGLDV | GGYNYVS | (SEQ ID NO: | ALYV |
| | NO: 86) | KG (SEQ | (SEQ ID NO: | (SEQ ID | 114) | (SEQ ID |
| | | ID NO: 109) | 88) | NO: 95) | | NO: 115) |
| B61- | SYGMH | VISYKGSN | SGYALHDD | TGTSSDV | EVSNRLR | SSYTSSS |
| 10 | (SEQ ID | KYYADSV | YYGLDV | GGYNYVS | (SEQ ID NO: | TLYV |
| | NO: 86) | KG (SEQ | (SEQ ID NO: | (SEQ ID | 114) | (SEQ ID |
| | | ID NO: 109) | 88) | NO: 95) | | NO: 97) |
| Consensus | SYGMH | VISYXGSN | SGYALHDD | TGTSSDV | X₁VSNRX₂X₃, | SSYTSSS |
| | (SEQ ID | KYYADSV | YYGLDV | GGYNYVS | wherein X₁ is | XLYV, |
| | NO: 86) | KG, | (SEQ ID NO: | (SEQ ID | D or E; X₂ is P | wherein X |
| | | wherein X is | 88) | NO: 95) | or L; and X₃ is | is T or A |
| | | D or K | | | S or R (SEQ | (SEQ ID |
| | | (SEQ ID | | | ID NO: 131) | NO: 132) |
| | | NO: 130) | | | | |

TABLE 8
Chothia CDRs of exemplary B cell-derived anti-BCMA molecules

| Chothia | HCDR1 | HCDR2 | HCDR3 | LCDR1 | LCDR2 | LCDR3 |
|---|---|---|---|---|---|---|
| PI61 | GFTFS | SYDGSN | SGYALHDDY | TSSDVGG | DVS (SEQ | YTSSSTLY |
| | SY | (SEQ ID NO: | YGLDV (SEQ | YNY (SEQ | ID NO: 99) | (SEQ ID |
| | (SEQ | 89) | ID NO: 88) | ID NO: 98) | | NO: 100) |
| | ID NO: | | | | | |
| | 47 | | | | | |
| B61-02 | GFTFS | SYKGSN | SGYALHDDY | TSSDVGG | EVS (SEQ | YTSSSALY |
| | SY | (SEQ ID NO: | YGLDV (SEQ | YNY (SEQ | ID NO: | (SEQ ID |
| | (SEQ | 110) | ID NO: 88) | ID NO: 98) | 116) | NO: 117) |
| | ID NO: | | | | | |
| | 47) | | | | | |
| B61-10 | GFTFS | SYKGSN | SGYALHDDY | TSSDVGG | EVS (SEQ | YTSSSTLY |
| | SY | (SEQ ID NO: | YGLDV (SEQ | YNY (SEQ | ID NO: | (SEQ ID |
| | (SEQ | 110) | ID NO: 88) | ID NO: 98) | 116) | NO: 100) |
| | ID NO: | | | | | |
| | 47) | | | | | |
| Con- | GFTFS | SYXGSN, | SGYALHDDY | TSSDVGG | XVS, | YTSSSXLY, |
| sensus | SY | wherein X | YGLDV (SEQ | YNY (SEQ | wherein X | wherein X |
| | (SEQ | is D or K | ID NO: 88) | ID NO: 98) | is D or E | is T or A |
| | ID NO: | (SEQ | | | (SEQ ID | (SEQ ID |
| | 47) | ID NO: 133) | | | NO: 134) | NO: 135) |

TABLE 9
IMGT CDRs of exemplary B cell-derived anti-BCMA molecules

| IMGT | HCDR1 | HCDR2 | HCDR3 | LCDR1 | LCDR2 | LCDR3 |
|---|---|---|---|---|---|---|
| PI61 | GFTFSSY | ISYDGSNK | GGSGYALHD | SSDVGGYN | DVS | SSYTSSST |
| | G (SEQ ID | (SEQ ID NO: | DYYGLDV | Y (SEQ ID | (SEQ ID | LYV (SEQ |
| | NO: 90) | 91) | (SEQ ID NO: | NO: 101) | NO: 99) | ID NO: 97) |
| | | | 92) | | | |
| B61-02 | GFTFSSY | ISYKGSNK | GGSGYALHD | SSDVGGYN | EVS | SSYTSSS |
| | G (SEQ ID | (SEQ ID NO: | DYYGLDV | Y (SEQ ID | (SEQ ID | ALYV |
| | NO: 90) | 111) | (SEQ ID NO: | NO: 101) | NO: 116) | (SEQ ID |
| | | | 92) | | | NO: 115) |
| B61-10 | GFTFSSY | ISYKGSNK | GGSGYALHD | SSDVGGYN | EVS | SSYTSSST |
| | G (SEQ ID | (SEQ ID NO: | DYYGLDV | Y (SEQ ID | (SEQ ID | LYV (SEQ |
| | NO: 90) | 111) | (SEQ ID NO: | NO: 101) | NO: 116) | ID NO: 97) |
| | | | 92) | | | |
| Con- | GFTFSSY | ISYXGSNK, | GGSGYALHD | SSDVGGYN | XVS, | SSYTSSS |
| sensus | G (SEQ ID | wherein X | DYYGLDV | Y (SEQ ID | wherein X | XLYV, |
| | NO: 90) | is D or K | (SEQ ID NO: | NO: 101) | is D or E | wherein X |
| | | (SEQ | 92) | | (SEQ ID | is T or A |
| | | ID NO: 136) | | | NO: 134) | (SEQ ID |
| | | | | | | NO: 132) |

TABLE 14
Amino acid and nucleic acid sequences of exemplary anti-BCMA molecules based on PI61

| Identification | Protein sequence | DNA sequence (5′-3′) |
|---|---|---|
| Signal peptide | MALPVTALLLPLALLLHAA | Atggccctccctgtcaccgctctgttgctgccgcttgctctgctg |
| | RP (SEQ ID NO: 2) | ctccacgcagegegaccg (SEQ ID NO: 252) |
| ScFv PI61 | QVQLQESGGGVVQPGRSLR | CaggtacaattgcaggagtctggaggcggtgtgGtgcaacc |
| | LSCAASGFTFSSYGMHWVR | cggtcgcagcttgcgcctgagttgtGctgcgtctggatttacatt |
| | QAPGKGLEWVAVISYDGSN | ttcatcttacggaAtgcattgggtacgccaggcaccggggaa |
| | KYYADSVKGRFTISRDNSK | aggcCttgaatgggtggctgtaatttcatacgatggtTccaac |
| | NTLYLQMNSLRAEDTAVYY | aaatactatgctgactcagtcaagggtCgatttacaattagtcg |
| | CGGSGYALHDDYYGLDVW | ggacaactccaagaacAccctttatcttcaaatgaattcccttag |
| | GQGTLVTVSSGGGGSGGGG | agcaGaggatacggcggtctattactgtggtggcagtGgttat |
| | SGGGGSQSALTQPASVSGSP | gcacttcatgatgattactatggcttgGatgtctgggggcaagg |
| | GQSITISCTGTSSDVGGYNY | gacgcttgtaactgtaTcctctggtggtggtggtagtggtggg |
| | VSWYQQHPGKAPKLMIYD | ggaggcTccggcggtggcggctctcaatctgctctgactCaa |
| | VSNRPSGVSNRFSGSKSGNT | ccagcaagcgtatcagggtcaccgggacagAgtattaccata |
| | ASLTISGLQAEDEADYYCSS | agttgcacggggacctctagcGatgtaggggggtataattatg |
| | YTSSSTLYVFGSGTKVTVL | tatcttggtatCaacaacaccccgggaaagcccctaaattgatg |
| | (SEQ ID NO: 105) | AtctacgacgtgagcaatcgacctagtggcgtaTcaaatcgc |
| | | ttctctggtagcaagagtgggaatAcggcgtcccttactattag |
| | | cggattgcaagcaGaagatgaggccgattactactgcagctc |
| | | ctatActagctcttctacattgtacgtctttgggagcggaacaaa |
| | | agtaacagtactc (SEQ ID NO: 253) |
| Transmembrane | TTTPAPRPPTPAPTIASQPLS | AcaacaacacctgccccgagaccgcctacaccaGccccga |
| domain and hinge | LRPEACRPAAGGAVHTRGL | ctattgccagccagcctctgagcctcAggcctgaggcctgtag |
| | DFACDIYIWAPLAGTCGVLL | gcccgcagcgggcggcGcagttcatacacggggcttggattt |
| | LSLVITLYC (SEQ ID NO: | cgcttgtGatatttatatttgggctcctttggcggggacaTgtgg |
| | 202) | cgtgctgcttctgtcacttgttattacactgtactgt (SEQ ID |
| | | NO: 254) |
| 4-1BB | KRGRKKLLYIFKQPFMRPV | AaacgcgggcgaaaaaaattgctgtatatttttAagcagccat |
| | QTTQEEDGCSCRFPEEEEGG | ttatgaggcccgttcagacgacgCaggaggaggacggttgct |
| | CEL (SEQ ID NO: 14) | cttgcaggttcccagaagaggaagaagggggctgtgaattg |
| | | (SEQ ID NO: 255) |
| CD3zeta | RVKFSRSADAPAYQQGQNQ | CgggttaaattttcaagatccgcagacgctccaGcataccaac |
| | LYNELNLGRREEYDVLDKR | agggacaaaaccaactctataacGagctgaatcttggaagaa |
| | RGRDPEMGGKPRRKNPQEG | gggaggaatatgatGtgctggataaacggcgcggtagagatc |
| | LYNELQKDKMAEAYSEIGM | cggagAtgggcggaaaaccaaggcgaaaaaaccctcagG |
| | KGERRRGKGHDGLYQGLST | agggactctacaacgaactgcagaaagacaaaAtggcggag |
| | ATKDTYDALHMQALPPR | gcttattccgaaataggcatgaagGgcgagcggaggcgagg |
| | (SEQ ID NO: 20) | gaaagggcacgacggaCtgtatcaaggcctctcaaccgcga |
| | | ctaaggatAcgtacgacgccctgcacatgcaggccctgcctc |
| | | cgaga (SEQ ID NO: 256) |
| PI61 full CAR | MALPVTALLLPLALLLHAA | ATGGCCCTCCCTGTCACCGCTCTGTTG |
| construct | RPQVQLQESGGGVVQPGRS | CTGCCGCTTGCTCTGCTGCTCCACGCA |
| | LRLSCAASGFTFSSYGMHW | GCGCGACCGCAGGTACAATTGCAGGA |
| | VRQAPGKGLEWVAVISYDG | GTCTGGAGGCGGTGTGGTGCAACCCG |
| | SNKYYADSVKGRFTISRDNS | GTCGCAGCTTGCGCCTGAGTTGTGCTG |
| | KNTLYLQMNSLRAEDTAVY | CGTCTGGATTTACATTTTCATCTTACGG |
| | YCGGSGYALHDDYYGLDV | AATGCATTGGGTACGCCAGGCACCGG |
| | WGQGTLVTVSSGGGGSGG | GGAAAGGCCTTGAATGGGTGGCTGTA |
| | GGSGGGGSQSALTQPASVS | ATTTCATACGATGGTTCCAACAAATAC |
| | GSPGQSITISCTGTSSDVGGY | TATGCTGACTCAGTCAAGGGTCGATTT |
| | NYVSWYQQHPGKAPKLMI | ACAATTAGTCGGGACAACTCCAAGAA |
| | YDVSNRPSGVSNRFSGSKSG | CACCCTTTATCTTCAAATGAATTCCCTT |
| | NTASLTISGLQAEDEADYYC | AGAGCAGAGGATACGGCGGTCTATTA |
| | SSYTSSSTLYVFGSGTKVTV | CTGTGGTGGCAGTGGTTATGCACTTCA |
| | LTTTPAPRPPTPAPTIASQPL | TGATGATTACTATGGCTTGGATGTCTG |
| | SLRPEACRPAAGGAVHTRG | GGGGCAAGGGACGCTTGTAACTGTATC |
| | LDFACDIYIWAPLAGTCGVL | CTCTGGTGGTGGTGGTAGTGGTGGGGG |
| | LLSLVITLYCKRGRKKLLYI | AGGCTCCGGCGGTGGCGGCTCTCAATC |
| | FKQPFMRPVQTTQEEDGCS | TGCTCTGACTCAACCAGCAAGCGTATC |
| | CRFPEEEEGGCELRVKFSRS | AGGGTCACCGGGACAGAGTATTACCA |
| | ADAPAYQQGQNQLYNELN | TAAGTTGCACGGGGACCTCTAGCGATG |
| | LGRREEYDVLDKRRGRDPE | TAGGGGGGTATAATTATGTATCTTGGT |
| | MGGKPRRKNPQEGLYNELQ | ATCAACAACACCCCGGGAAAGCCCCT |
| | KDKMAEAYSEIGMKGERRR | AAATTGATGATCTACGACGTGAGCAAT |
| | GKGHDGLYQGLSTATKDTY | CGACCTAGTGGCGTATCAAATCGCTTC |
| | DALHMQALPPR (SEQ ID | TCTGGTAGCAAGAGTGGGAATACGGC |
| | NO: 257) | GTCCCTTACTATTAGCGGATTGCAAGC |
| | | AGAAGATGAGGCCGATTACTACTGCA |
| | | GCTCCTATACTAGCTCTTCTACATTGTA |
| | | CGTCTTTGGGAGCGGAACAAAAGTAA |
| | | CAGTACTCACAACAACACCTGCCCCGA |
| | | GACCGCCTACACCAGCCCCGACTATTG |
| | | CCAGCCAGCCTCTGAGCCTCAGGCCTG |
| | | AGGCCTGTAGGCCCGCAGCGGGCGGC |
| | | GCAGTTCATACACGGGGCTTGGATTTC |
| | | GCTTGTGATATTTATATTTGGGCTCCTT |
| | | TGGCGGGGACATGTGGCGTGCTGCTTC |
| | | TGTCACTTGTTATTACACTGTACTGTA |
| | | AACGCGGGCGAAAAAAATTGCTGTAT |
| | | ATTTTTAAGCAGCCATTTATGAGGCCC |
| | | GTTCAGACGACGCAGGAGGAGGACGG |
| | | TTGCTCTTGCAGGTTCCCAGAAGAGGA |
| | | AGAAGGGGGCTGTGAATTGCGGGTTA |
| | | AATTTTCAAGATCCGCAGACGCTCCAG |
| | | CATACCAACAGGGACAAAACCAACTC |
| | | TATAACGAGCTGAATCTTGGAAGAAG |
| | | GGAGGAATATGATGTGCTGGATAAAC |
| | | GGCGCGGTAGAGATCCGGAGATGGGC |
| | | GGAAAACCAAGGCGAAAAAACCCTCA |
| | | GGAGGGACTCTACAACGAACTGCAGA |
| | | AAGACAAAATGGCGGAGGCTTATTCC |
| | | GAAATAGGCATGAAGGGCGAGCGGAG |
| | | GCGAGGGAAAGGGCACGACGGACTGT |
| | | ATCAAGGCCTCTCAACCGCGACTAAGG |
| | | ATACGTACGACGCCCTGCACATGCAGG |
| | | CCCTGCCTCCGAGA (SEQ ID NO: 258) |
| PI61 mature | QVQLQESGGGVVQPGRSLR | |
| CAR protein | LSCAASGFTFSSYGMHWVR | |
| | QAPGKGLEWVAVISYDGSN | |
| | KYYADSVKGRFTISRDNSK | |
| | NTLYLQMNSLRAEDTAVYY | |
| | CGGSGYALHDDYYGLDVW | |
| | GQGTLVTVSSGGGGSGGGG | |
| | SGGGGSQSALTQPASVSGSP | |
| | GQSITISCTGTSSDVGGYNY | |
| | VSWYQQHPGKAPKLMIYD | |
| | VSNRPSGVSNRFSGSKSGNT | |
| | ASLTISGLQAEDEADYYCSS | |
| | YTSSSTLYVFGSGTKVTVLT | |
| | TTPAPRPPTPAPTIASQPLSL | |
| | RPEACRPAAGGAVHTRGLD | |
| | FACDIYIWAPLAGTCGVLLL | |
| | SLVITLYCKRGRKKLLYIFK | |
| | QPFMRPVQTTQEEDGCSCR | |
| | FPEEEEGGCELRVKFSRSAD | |
| | APAYQQGQNQLYNELNLG | |
| | RREEYDVLDKRRGRDPEMG | |
| | GKPRRKNPQEGLYNELQKD | |
| | KMAEAYSEIGMKGERRRGK | |
| | GHDGLYQGLSTATKDTYDA | |
| | LHMQALPPR (SEQ ID NO: | |
| | 107) | |

TABLE 10
Amino acid and nucleic acid sequences of exemplary hybridoma-derived anti-BCMA
molecules

| SEQ ID | Name/ | |
|---|---|---|
| NO | Description | Sequence |
| Hy03 | | |
| SEQ ID | HCDR1 | GFWMS |
| NO: 137 | (Kabat) | |
| SEQ ID | HCDR2 | NIKQDGSEKYYVDSVRG |
| NO: 138 | (Kabat) | |
| SEQ ID | HCDR3 | ALDYYGMDV |
| NO: 139 | (Kabat) | |
| SEQ ID | HCDR1 | GFTFSGF |
| NO: 140 | (Chothia) | |
| SEQ ID | HCDR2 | KQDGSE |
| NO: 141 | (Chothia) | |
| SEQ ID | HCDR3 | ALDYYGMDV |
| NO: 139 | (Chothia) | |
| SEQ ID | HCDR1 | GFTFSGFW |
| NO: 142 | (IMGT) | |
| SEQ ID | HCDR2 | IKQDGSEK |
| NO: 143 | (IMGT) | |
| SEQ ID | HCDR3 | ARALDYYGMDV |
| NO: 144 | (IMGT) | |
| SEQ ID | VH | EVQLVESGGGLVQPGGSLRLSCAASGFTFSGFWMSWVRQAPGKG |
| NO: 145 | | LEWVANIKQDGSEKYYVDSVRGRFTISRDNAKNSLYLQMNSLRA |
| | | EDTAVYYCARALDYYGMDVWGQGTTVTVSS |
| SEQ ID | DNA VH | GAAGTGCAACTGGTGGAGAGCGGTGGAGGGCTTGTCCAGCCC |
| NO: 146 | | GGAGGATCGCTGCGGCTGTCCTGTGCTGCGTCCGGGTTCACCT |
| | | TCTCCGGCTTCTGGATGTCCTGGGTCAGACAGGCACCGGGAAA |
| | | GGGCCTCGAATGGGTGGCCAACATCAAGCAGGATGGCTCCGA |
| | | GAAGTACTACGTCGACTCCGTGAGAGGCCGCTTCACCATCTCC |
| | | CGGGACAACGCCAAGAACTCGCTGTACCTCCAAATGAATAGCC |
| | | TCAGGGCGGAAGATACTGCTGTGTATTACTGCGCACGCGCCCT |
| | | TGACTACTACGGCATGGACGTCTGGGGCCAAGGGACCACTGTG |
| | | ACCGTGTCTAGC |
| SEQ ID | LCDR1 | RSSQSLLDSDDGNTYLD |
| NO: 147 | (Kabat) | |
| SEQ ID | LCDR2 | TLSYRAS |
| NO: 148 | (Kabat) | |
| SEQ ID | LCDR3 | TQRLEFPSIT |
| NO: 149 | (Kabat) | |
| SEQ ID | LCDR1 | SQSLLDSDDGNTY |
| NO: 150 | (Chothia) | |
| SEQ ID | LCDR2 | TLS |
| NO: 151 | (Chothia) | |
| SEQ ID | LCDR3 | RLEFPSI |
| NO: 152 | (Chothia) | |
| SEQ ID | LCDR1 | QSLLDSDDGNTY |
| NO: 153 | (IMGT) | |
| SEQ ID | LCDR2 | TLS |
| NO: 151 | (IMGT) | |
| SEQ ID | LCDR3 | TQRLEFPSIT |
| NO: 149 | (IMGT) | |
| SEQ ID | VL | DIVMTQTPLSLPVTPGEPASISCRSSQSLLDSDDGNTYLDWYLQKP |
| NO: 154 | | GQSPRLLIYTLSYRASGVPDRFSGSGSGTDFTLKISRVEAEDVGLY |
| | | YCTQRLEFPSITFGQGTRLEIK |
| SEQ ID | DNA VL | GATATCGTGATGACCCAGACTCCCCTGTCCCTGCCTGTGACTCC |
| NO: 155 | | CGGAGAACCAGCCTCCATTTCCTGCCGGTCCTCCCAGTCCCTGC |
| | | TGGACAGCGACGACGGCAACACTTACCTGGACTGGTACTTGCA |
| | | GAAGCCGGGCCAATCGCCTCGCCTGCTGATCTATACCCTGTCA |
| | | TACCGGGCCTCAGGAGTGCCTGACCGCTTCTCGGGATCAGGGA |
| | | GCGGGACCGATTTCACCCTGAAAATTTCCCGAGTGGAAGCCGA |
| | | GGACGTCGGACTGTACTACTGCACCCAGCGCCTCGAATTCCCG |
| | | TCGATTACGTTTGGACAGGGTACCCGGCTTGAGATCAAG |
| SEQ ID | Linker | GGGGSGGGGSGGGGSGGGGS |
| NO: 63 | | |
| SEQ ID | scFv (VH- | EVQLVESGGGLVQPGGSLRLSCAASGFTFSGFWMSWVRQAPGKG |
| NO: 156 | linker-VL) | LEWVANIKQDGSEKYYVDSVRGRFTISRDNAKNSLYLQMNSLRA |
| | | EDTAVYYCARALDYYGMDVWGQGTTVTVSSGGGGSGGGGSGG |
| | | GGSGGGGSDIVMTQTPLSLPVTPGEPASISCRSSQSLLDSDDGNTY |
| | | LDWYLQKPGQSPRLLIYTLSYRASGVPDRFSGSGSGTDFTLKISRV |
| | | EAEDVGLYYCTQRLEFPSITFGQGTRLEIK |
| SEQ ID | DNA scFv | GAAGTGCAACTGGTGGAGAGCGGTGGAGGGCTTGTCCAGCCC |
| NO: 157 | | GGAGGATCGCTGCGGCTGTCCTGTGCTGCGTCCGGGTTCACCT |
| | | TCTCCGGCTTCTGGATGTCCTGGGTCAGACAGGCACCGGGAAA |
| | | GGGCCTCGAATGGGTGGCCAACATCAAGCAGGATGGCTCCGA |
| | | GAAGTACTACGTCGACTCCGTGAGAGGCCGCTTCACCATCTCC |
| | | CGGGACAACGCCAAGAACTCGCTGTACCTCCAAATGAATAGCC |
| | | TCAGGGCGGAAGATACTGCTGTGTATTACTGCGCACGCGCCCT |
| | | TGACTACTACGGCATGGACGTCTGGGGCCAAGGGACCACTGTG |
| | | ACCGTGTCTAGCGGAGGCGGAGGTTCAGGGGGCGGTGGATCA |
| | | GGCGGAGGAGGATCGGGGGGTGGTGGATCGGATATCGTGATG |
| | | ACCCAGACTCCCCTGTCCCTGCCTGTGACTCCCGGAGAACCAG |
| | | CCTCCATTTCCTGCCGGTCCTCCCAGTCCCTGCTGGACAGCGAC |
| | | GACGGCAACACTTACCTGGACTGGTACTTGCAGAAGCCGGGCC |
| | | AATCGCCTCGCCTGCTGATCTATACCCTGTCATACCGGGCCTCA |
| | | GGAGTGCCTGACCGCTTCTCGGGATCAGGGAGCGGGACCGATT |
| | | TCACCCTGAAAATTTCCCGAGTGGAAGCCGAGGACGTCGGACT |
| | | GTACTACTGCACCCAGCGCCTCGAATTCCCGTCGATTACGTTTG |
| | | GACAGGGTACCCGGCTTGAGATCAAG |
| SEQ ID | Full CAR | EVQLVESGGGLVQPGGSLRLSCAASGFTFSGFWMSWVRQAPGKG |
| NO: 158 | amino acid | LEWVANIKQDGSEKYYVDSVRGRFTISRDNAKNSLYLQMNSLRA |
| | sequence | EDTAVYYCARALDYYGMDVWGQGTTVTVSSGGGGSGGGGSGG |
| | | GGSGGGGSDIVMTQTPLSLPVTPGEPASISCRSSQSLLDSDDGNTY |
| | | LDWYLQKPGQSPRLLIYTLSYRASGVPDRFSGSGSGTDFTLKISRV |
| | | EAEDVGLYYCTQRLEFPSITFGQGTRLEIKTTTPAPRPPTPAPTIAS |
| | | QPLSLRPEACRPAAGGAVHTRGLDFACDIYIWAPLAGTCGVLLLS |
| | | LVITLYCKRGRKKLLYIFKQPFMRPVQTTQEEDGCSCRFPEEEEGG |
| | | CELRVKFSRSADAPAYQQGQNQLYNELNLGRREEYDVLDKRRGR |
| | | DPEMGGKPRRKNPQEGLYNELQKDKMAEAYSEIGMKGERRRGK |
| | | GHDGLYQGLSTATKDTYDALHMQALPPR |
| SEQ ID | Full CAR | GAAGTGCAACTGGTGGAGAGCGGTGGAGGGCTTGTCCAGCCC |
| NO: 159 | DNA | GGAGGATCGCTGCGGCTGTCCTGTGCTGCGTCCGGGTTCACCT |
| | sequence | TCTCCGGCTTCTGGATGTCCTGGGTCAGACAGGCACCGGGAAA |
| | | GGGCCTCGAATGGGTGGCCAACATCAAGCAGGATGGCTCCGA |
| | | GAAGTACTACGTCGACTCCGTGAGAGGCCGCTTCACCATCTCC |
| | | CGGGACAACGCCAAGAACTCGCTGTACCTCCAAATGAATAGCC |
| | | TCAGGGCGGAAGATACTGCTGTGTATTACTGCGCACGCGCCCT |
| | | TGACTACTACGGCATGGACGTCTGGGGCCAAGGGACCACTGTG |
| | | ACCGTGTCTAGCGGAGGCGGAGGTTCAGGGGGCGGTGGATCA |
| | | GGCGGAGGAGGATCGGGGGGTGGTGGATCGGATATCGTGATG |
| | | ACCCAGACTCCCCTGTCCCTGCCTGTGACTCCCGGAGAACCAG |
| | | CCTCCATTTCCTGCCGGTCCTCCCAGTCCCTGCTGGACAGCGAC |
| | | GACGGCAACACTTACCTGGACTGGTACTTGCAGAAGCCGGGCC |
| | | AATCGCCTCGCCTGCTGATCTATACCCTGTCATACCGGGCCTCA |
| | | GGAGTGCCTGACCGCTTCTCGGGATCAGGGAGCGGGACCGATT |
| | | TCACCCTGAAAATTTCCCGAGTGGAAGCCGAGGACGTCGGACT |
| | | GTACTACTGCACCCAGCGCCTCGAATTCCCGTCGATTACGTTTG |
| | | GACAGGGTACCCGGCTTGAGATCAAGACCACTACCCCAGCACC |
| | | GAGGCCACCCACCCCGGCTCCTACCATCGCCTCCCAGCCTCTG |
| | | TCCCTGCGTCCGGAGGCATGTAGACCCGCAGCTGGTGGGGCCG |
| | | TGCATACCCGGGGTCTTGACTTCGCCTGCGATATCTACATTTGG |
| | | GCCCCTCTGGCTGGTACTTGCGGGGTCCTGCTGCTTTCACTCGT |
| | | GATCACTCTTTACTGTAAGCGCGGTCGGAAGAAGCTGCTGTAC |
| | | ATCTTTAAGCAACCCTTCATGAGGCCTGTGCAGACTACTCAAG |
| | | AGGAGGACGGCTGTTCATGCCGGTTCCCAGAGGAGGAGGAAG |
| | | GCGGCTGCGAACTGCGCGTGAAATTCAGCCGCAGCGCAGATGC |
| | | TCCAGCCTACCAGCAGGGGCAGAACCAGCTCTACAACGAACTC |
| | | AATCTTGGTCGGAGAGAGGAGTACGACGTGCTGGACAAGCGG |
| | | AGAGGACGGGACCCAGAAATGGGCGGGAAGCCGCGCAGAAA |
| | | GAATCCCCAAGAGGGCCTGTACAACGAGCTCCAAAAGGATAA |
| | | GATGGCAGAAGCCTATAGCGAGATTGGTATGAAAGGGGAACG |
| | | CAGAAGAGGCAAAGGCCACGACGGACTGTACCAGGGACTCAG |
| | | CACCGCCACCAAGGACACCTATGACGCTCTTCACATGCAGGCC |
| | | CTGCCGCCTCGG |
| Hy52 | | |
| SEQ ID | HCDR1 | SFRMN |
| NO: 160 | (Kabat) | |
| SEQ ID | HCDR2 | SISSSSSYIYYADSVKG |
| NO: 161 | (Kabat) | |
| SEQ ID | HCDR3 | WLSYYGMDV |
| NO: 162 | (Kabat) | |
| SEQ ID | HCDR1 | GFTFSSF |
| NO: 163 | (Chothia) | |
| SEQ ID | HCDR2 | SSSSSY |
| NO: 164 | (Chothia) | |
| SEQ ID | HCDR3 | WLSYYGMDV |
| NO: 162 | (Chothia) | |
| SEQ ID | HCDR1 | GFTFSSFR |
| NO: 165 | (IMGT) | |
| SEQ ID | HCDR2 | ISSSSSYI |
| NO: 166 | (IMGT) | |
| SEQ ID | HCDR3 | ARWLSYYGMDV |
| NO: 167 | (IMGT) | |
| SEQ ID | VH | EVQLVESGGGLVKPGGSLRLSCAASGFTFSSFRMNWVRQAPGKG |
| NO: 168 | | LEWVSSISSSSSYIYYADSVKGRFTISRDNAKNSLYLQMNSLRAED |
| | | TAVYYCARWLSYYGMDVWGQGTTVTVSS |
| SEQ ID | DNA VH | GAAGTGCAACTGGTGGAGAGCGGTGGAGGGCTTGTCAAGCCC |
| NO: 169 | | GGAGGATCGCTGCGGCTGTCCTGTGCTGCGTCCGGGTTCACCT |
| | | TCTCCTCGTTCCGCATGAACTGGGTCAGACAGGCACCGGGAAA |
| | | GGGCCTCGAATGGGTGTCCTCAATCTCATCGTCCTCGTCCTACA |
| | | TCTACTACGCCGACTCCGTGAAAGGCCGCTTCACCATCTCCCG |
| | | GGACAACGCCAAGAACTCGCTGTACCTCCAAATGAATAGCCTC |
| | | AGGGCGGAAGATACTGCTGTGTATTACTGCGCACGCTGGCTTT |
| | | CCTACTACGGCATGGACGTCTGGGGCCAAGGGACCACTGTGAC |
| | | CGTGTCTAGC |
| SEQ ID | LCDR1 | RSSQSLLDSDDGNTYLD |
| NO: 147 | (Kabat) | |
| SEQ ID | LCDR2 | TLSFRAS |
| NO: 170 | (Kabat) | |
| SEQ ID | LCDR3 | MQRIGFPIT |
| NO: 171 | (Kabat) | |
| SEQ ID | LCDR1 | SQSLLDSDDGNTY |
| NO: 150 | (Chothia) | |
| SEQ ID | LCDR2 | TLS |
| NO: 151 | (Chothia) | |
| SEQ ID | LCDR3 | RIGFPI |
| NO: 172 | (Chothia) | |
| SEQ ID | LCDR1 | QSLLDSDDGNTY |
| NO: 153 | (IMGT) | |
| SEQ ID | LCDR2 | TLS |
| NO: 151 | (IMGT) | |
| SEQ ID | LCDR3 | MQRIGFPIT |
| NO: 171 | (IMGT) | |
| SEQ ID | VL | DIVMTQTPLSLPVTPGEPASISCRSSQSLLDSDDGNTYLDWYLQKP |
| NO: 173 | | GQSPQLLIYTLSFRASGVPDRFSGSGSGTDFTLKIRRVEAEDVGVY |
| | | YCMQRIGFPITFGQGTRLEIK |
| SEQ ID | DNA VL | GATATCGTGATGACCCAGACTCCCCTGTCCCTGCCTGTGACTCC |
| NO: 174 | | CGGAGAACCAGCCTCCATTTCCTGCCGGTCCTCCCAGTCCCTGC |
| | | TGGACAGCGACGACGGCAACACTTACCTGGACTGGTACTTGCA |
| | | GAAGCCGGGCCAATCGCCTCAGCTGCTGATCTATACCCTGTCA |
| | | TTCCGGGCCTCAGGAGTGCCTGACCGCTTCTCGGGATCAGGGA |
| | | GCGGGACCGATTTCACCCTGAAAATTAGGCGAGTGGAAGCCG |
| | | AGGACGTCGGAGTGTACTACTGCATGCAGCGCATCGGCTTCCC |
| | | GATTACGTTTGGACAGGGTACCCGGCTTGAGATCAAG |
| SEQ ID | Linker | GGGGSGGGGSGGGGSGGGGS |
| NO: 63 | | |
| SEQ ID | scFv (VH- | EVQLVESGGGLVKPGGSLRLSCAASGFTFSSFRMNWVRQAPGKG |
| NO: 175 | linker-VL) | LEWVSSISSSSSYIYYADSVKGRFTISRDNAKNSLYLQMNSLRAED |
| | | TAVYYCARWLSYYGMDVWGQGTTVTVSSGGGGSGGGGSGGGG |
| | | SGGGGSDIVMTQTPLSLPVTPGEPASISCRSSQSLLDSDDGNTYLD |
| | | WYLQKPGQSPQLLIYTLSFRASGVPDRFSGSGSGTDFTLKIRRVEA |
| | | EDVGVYYCMQRIGFPITFGQGTRLEIK |
| SEQ ID | DNA scFv | GAAGTGCAACTGGTGGAGAGCGGTGGAGGGCTTGTCAAGCCC |
| NO: 176 | | GGAGGATCGCTGCGGCTGTCCTGTGCTGCGTCCGGGTTCACCT |
| | | TCTCCTCGTTCCGCATGAACTGGGTCAGACAGGCACCGGGAAA |
| | | GGGCCTCGAATGGGTGTCCTCAATCTCATCGTCCTCGTCCTACA |
| | | TCTACTACGCCGACTCCGTGAAAGGCCGCTTCACCATCTCCCG |
| | | GGACAACGCCAAGAACTCGCTGTACCTCCAAATGAATAGCCTC |
| | | AGGGCGGAAGATACTGCTGTGTATTACTGCGCACGCTGGCTTT |
| | | CCTACTACGGCATGGACGTCTGGGGCCAAGGGACCACTGTGAC |
| | | CGTGTCTAGCGGAGGCGGAGGTTCAGGGGGCGGTGGATCAGG |
| | | CGGAGGAGGATCGGGGGGTGGTGGATCGGATATCGTGATGAC |
| | | CCAGACTCCCCTGTCCCTGCCTGTGACTCCCGGAGAACCAGCC |
| | | TCCATTTCCTGCCGGTCCTCCCAGTCCCTGCTGGACAGCGACGA |
| | | CGGCAACACTTACCTGGACTGGTACTTGCAGAAGCCGGGCCAA |
| | | TCGCCTCAGCTGCTGATCTATACCCTGTCATTCCGGGCCTCAGG |
| | | AGTGCCTGACCGCTTCTCGGGATCAGGGAGCGGGACCGATTTC |
| | | ACCCTGAAAATTAGGCGAGTGGAAGCCGAGGACGTCGGAGTG |
| | | TACTACTGCATGCAGCGCATCGGCTTCCCGATTACGTTTGGAC |
| | | AGGGTACCCGGCTTGAGATCAAG |
| SEQ ID | Full CAR | EVQLVESGGGLVKPGGSLRLSCAASGFTFSSFRMNWVRQAPGKG |
| NO: 177 | amino acid | LEWVSSISSSSSYIYYADSVKGRFTISRDNAKNSLYLQMNSLRAED |
| | sequence | TAVYYCARWLSYYGMDVWGQGTTVTVSSGGGGSGGGGSGGGG |
| | | SGGGGSDIVMTQTPLSLPVTPGEPASISCRSSQSLLDSDDGNTYLD |
| | | WYLQKPGQSPQLLIYTLSFRASGVPDRFSGSGSGTDFTLKIRRVEA |
| | | EDVGVYYCMQRIGFPITFGQGTRLEIKTTTPAPRPPTPAPTIASQPL |
| | | SLRPEACRPAAGGAVHTRGLDFACDIYIWAPLAGTCGVLLLSLVI |
| | | TLYCKRGRKKLLYIFKQPFMRPVQTTQEEDGCSCRFPEEEEGGCE |
| | | LRVKFSRSADAPAYQQGQNQLYNELNLGRREEYDVLDKRRGRDP |
| | | EMGGKPRRKNPQEGLYNELQKDKMAEAYSEIGMKGERRRGKGH |
| | | DGLYQGLSTATKDTYDALHMQALPPR |
| SEQ ID | Full CAR | GAAGTGCAACTGGTGGAGAGCGGTGGAGGGCTTGTCAAGCCC |
| NO: 178 | DNA | GGAGGATCGCTGCGGCTGTCCTGTGCTGCGTCCGGGTTCACCT |
| | sequence | TCTCCTCGTTCCGCATGAACTGGGTCAGACAGGCACCGGGAAA |
| | | GGGCCTCGAATGGGTGTCCTCAATCTCATCGTCCTCGTCCTACA |
| | | TCTACTACGCCGACTCCGTGAAAGGCCGCTTCACCATCTCCCG |
| | | GGACAACGCCAAGAACTCGCTGTACCTCCAAATGAATAGCCTC |
| | | AGGGCGGAAGATACTGCTGTGTATTACTGCGCACGCTGGCTTT |
| | | CCTACTACGGCATGGACGTCTGGGGCCAAGGGACCACTGTGAC |
| | | CGTGTCTAGCGGAGGCGGAGGTTCAGGGGGCGGTGGATCAGG |
| | | CGGAGGAGGATCGGGGGGTGGTGGATCGGATATCGTGATGAC |
| | | CCAGACTCCCCTGTCCCTGCCTGTGACTCCCGGAGAACCAGCC |
| | | TCCATTTCCTGCCGGTCCTCCCAGTCCCTGCTGGACAGCGACGA |
| | | CGGCAACACTTACCTGGACTGGTACTTGCAGAAGCCGGGCCAA |
| | | TCGCCTCAGCTGCTGATCTATACCCTGTCATTCCGGGCCTCAGG |
| | | AGTGCCTGACCGCTTCTCGGGATCAGGGAGCGGGACCGATTTC |
| | | ACCCTGAAAATTAGGCGAGTGGAAGCCGAGGACGTCGGAGTG |
| | | TACTACTGCATGCAGCGCATCGGCTTCCCGATTACGTTTGGAC |
| | | AGGGTACCCGGCTTGAGATCAAGACCACTACCCCAGCACCGAG |
| | | GCCACCCACCCCGGCTCCTACCATCGCCTCCCAGCCTCTGTCCC |
| | | TGCGTCCGGAGGCATGTAGACCCGCAGCTGGTGGGGCCGTGCA |
| | | TACCCGGGGTCTTGACTTCGCCTGCGATATCTACATTTGGGCCC |
| | | CTCTGGCTGGTACTTGCGGGGTCCTGCTGCTTTCACTCGTGATC |
| | | ACTCTTTACTGTAAGCGCGGTCGGAAGAAGCTGCTGTACATCT |
| | | TTAAGCAACCCTTCATGAGGCCTGTGCAGACTACTCAAGAGGA |
| | | GGACGGCTGTTCATGCCGGTTCCCAGAGGAGGAGGAAGGCGG |
| | | CTGCGAACTGCGCGTGAAATTCAGCCGCAGCGCAGATGCTCCA |
| | | GCCTACCAGCAGGGGCAGAACCAGCTCTACAACGAACTCAATC |
| | | TTGGTCGGAGAGAGGAGTACGACGTGCTGGACAAGCGGAGAG |
| | | GACGGGACCCAGAAATGGGCGGGAAGCCGCGCAGAAAGAATC |
| | | CCCAAGAGGGCCTGTACAACGAGCTCCAAAAGGATAAGATGG |
| | | CAGAAGCCTATAGCGAGATTGGTATGAAAGGGGAACGCAGAA |
| | | GAGGCAAAGGCCACGACGGACTGTACCAGGGACTCAGCACCG |
| | | CCACCAAGGACACCTATGACGCTCTTCACATGCAGGCCCTGCC |
| | | GCCTCGG |

TABLE 11
Kabat CDRs of exemplary hybridoma-derived anti-BCMA molecules

| Kabat | HCDR1 | HCDR2 | HCDR3 | LCDR1 | LCDR2 | LCDR3 |
|---|---|---|---|---|---|---|
| Hy03 | GFWMS | NIKQDGSE | ALDYYGMD | RSSQSLLDS | TLSYRA | TQRLEFPS |
| | (SEQ ID | KYYVDSVR | V (SEQ ID | DDGNTYLD | S (SEQ ID | IT (SEQ ID |
| | NO: 137) | G (SEQ ID | NO: 139) | (SEQ ID NO: | NO: 148) | NO: 149) |
| | | NO: 138) | | 147) | | |
| Hy52 | SFRMN | SISSSSSYIY | WLSYYGMD | RSSQSLLDS | TLSFRAS | MQRIGFPI |
| | (SEQ ID | YADSVKG | V (SEQ ID | DDGNTYLD | (SEQ ID | T (SEQ ID |
| | NO: 160) | (SEQ ID NO: | NO: 162) | (SEQ ID NO: | NO: 170) | NO: 171) |
| | | 161) | | 147) | | |
| Con- | X₁FX₂MX₃, | X₁IX₂X₃X₄X₅ | X₁LX₂YYGM | RSSQSLLDS | TLSXRAS, | X₁QRX₂X₃F |
| sensus | wherein | SX₆X₇YYX₈ | DV, wherein | DDGNTYLD | wherein | PX₄IT, |
| | X₁ is G or | DSVX₉G, | X₁ is A or W; | (SEQ ID NO: | X is Y or | wherein X₁ |
| | S; X₂ is W | wherein X₁ is | and X₂ is D | 147) | F (SEQ ID | is T or M; |
| | or R; and | Nor S; X₂ is | or S (SEQ ID | | NO: 182) | X₂ is L or I; |
| | X₃ is S or | K or S; X₃ is | NO: 181) | | | X₃ is E or G; |
| | N (SEQ | Q or S; X₄ is | | | | and X₄ is S |
| | ID NO: | D or S; X₅ is | | | | or absent |
| | 179) | G or S; X₆ is | | | | (SEQ ID |
| | | E or Y; X₇ is | | | | NO: 183) |
| | | K or I; X₈ is | | | | |
| | | V or A; and | | | | |
| | | X₉ is R or K | | | | |
| | | (SEQ ID NO: | | | | |
| | | 180) | | | | |

TABLE 12
Chothia CDRs of exemplary hybridoma-derived anti-BCMA molecules

| Chothia | HCDR1 | HCDR2 | HCDR3 | LCDR1 | LCDR2 | LCDR3 |
|---|---|---|---|---|---|---|
| Hy03 | GFTFSGF | KQDGSE | ALDYYGMD | SQSLLDSDD | TLS | RLEFPSI |
| | (SEQ ID | (SEQ ID NO: | V (SEQ ID | GNTY (SEQ | (SEQ ID | (SEQ ID |
| | NO: 140) | 141) | NO: 139) | ID NO: 150) | NO: 151) | NO: 152) |
| Hy52 | GFTFSSF | SSSSSY | WLSYYGMD | SQSLLDSDD | TLS | RIGFPI |
| | (SEQ ID | (SEQ ID NO: | V (SEQ ID | GNTY (SEQ | (SEQ ID | (SEQ ID |
| | NO: 163) | 164) | NO: 162) | ID NO: 150) | NO: 151) | NO: 172) |
| Con- | GFTFSXF, | X₁X₂X₃X₄SX₅, | X₁LX₂YYGM | SQSLLDSDD | TLS | RX₁X₂FPX₃I, |
| sensus | wherein X is | wherein X₁ | DV, wherein | GNTY (SEQ | (SEQ ID | wherein |
| | G or S (SEQ | is K or S; X₂ | X₁ is A or W; | ID NO: 150) | NO: 151) | X₁ is L or |
| | ID NO: 184) | is Q or S; X₃ | and X₂ is D | | | I; X₂ is E |
| | | is D or S; X₄ | or S (SEQ ID | | | or G; and |
| | | is G or S; and | NO: 181) | | | X₃ is S or |
| | | X₅ is E or Y | | | | absent |
| | | (SEQ ID NO: | | | | (SEQ ID |
| | | 185) | | | | NO: 186) |

TABLE 13
IMGT CDRs of exemplary hybridoma-derived anti-BCMA molecules

| IMGT | HCDR1 | HCDR2 | HCDR3 | LCDR1 | LCDR2 | LCDR3 |
|---|---|---|---|---|---|---|
| Hy03 | GFTFSGF | IKQDGSEK | ARALDYYG | QSLLDSDD | TLS (SEQ | TQRLEF |
| | W (SEQ ID | (SEQ ID NO: | MDV (SEQ ID | GNTY (SEQ | ID NO: | PSIT |
| | NO: 142) | 143) | NO: 144) | ID NO: 153) | 151) | (SEQ ID |
| | | | | | | NO: 149) |
| Hy52 | GFTFSSFR | ISSSSSYI | ARWLSYYG | QSLLDSDD | TLS (SEQ | MQRIGF |
| | (SEQ ID | (SEQ ID NO: | MDV (SEQ ID | GNTY (SEQ | ID NO: | PIT |
| | NO: 165) | 166) | NO: 167) | ID NO: 153) | 151) | (SEQ ID |
| | | | | | | NO: 171) |
| Con- | GFTFSX₁F | IX₁X₂X₃X₄S | ARX₁LX₂YYG | QSLLDSDD | TLS (SEQ | X₁QRX₂ |
| sensus | X₂, wherein | X₅X₆, | MDV, wherein | GNTY (SEQ | ID NO: | X₃FPX₄IT, |
| | X₁ is G or S; | wherein X₁ is | X₁ is A or W; | ID NO: 153) | 151) | wherein |
| | and X₂ is W | K or S; X₂ is | and X₂ is D or | | | X₁ is Tor |
| | or R (SEQ | Q or S; X₃ is | S (SEQ ID NO: | | | M; X₂ is |
| | ID NO: 187) | D or S; X₄ is | 189) | | | L or I; X₃ |
| | | G or S; X₅ is | | | | is E or G; |
| | | E or Y; and | | | | and X₄ is |
| | | X₆ is K or I | | | | S or |
| | | (SEQ ID NO: | | | | absent |
| | | 188) | | | | (SEQ ID |
| | | | | | | NO: 183) |

In some embodiments, BCMA CARs may be generated using the VH and VL sequences from WO2012/0163805 (the contents of which are hereby incorporated by reference in its entirety). In some embodiments, BCMA CARs may be generated using the CDRs, VHs, VLs, scFvs, or full-CAR sequences from WO2019/241426 (the contents of which are hereby incorporated by reference in its entirety).

### Exemplary BCMA CAR D

In some embodiments, the BCMA CAR comprises a murine extracellular single-chain variable fragment (scFv) specific for recognizing B cell maturation antigen (BCMA) followed by a human CD8α hinge and transmembrane domain fused to the T cell cytoplasmic signaling domains of CD137 (4-1BB) and CD3ζ chain, in tandem. Binding of BCMA CARD to BCMA-expressing target cells leads to signaling initiated by CD3ζ and 4-1BB domains, and subsequent CAR-positive T cell activation. Antigen-specific activation of BCMA CAR D results in CAR-positive T cell proliferation, cytokine secretion, and subsequent cytolytic killing of BCMA-expressing cells. In some embodiments, the BCMA CAR is encoded by a nucleotide sequence of Table 28, or a nucleotide sequence having at least 80%, 85%, 90%, 95%, 97%, or 100% or no more than 20, 15, 10, 8, 5, 4, 3, 2, or 1 variations thereto. In some embodiments, the BCMA CAR comprises a polypeptide encoded by a nucleotide sequence of Table 28, or a nucleotide sequence having at least 80%, 85%, 90%, 95%, 97%, or 100% or no more than 20, 15, 10, 8, 5, 4, 3, 2, or 1 variations thereto. In some embodiments, the BCMA CAR comprises a polypeptide sequence of Table 28, or a polypeptide sequence having at least 80%, 85%, 90%, 95%, 97%, or 100% or no more than 20, 15, 10, 8, 5, 4, 3, 2, or 1 variations thereto. In some embodiments, the BCMA CAR comprises a heavy chain CDR1-3 and a light chain CDR1-3, of a sequence of Table 28. In some embodiments, the BCMA CAR comprises a heavy chain CDR1-3 and a light chain CDR1-3, of a sequence of Table 28 according to Kabat. In some embodiments, the BCMA CAR comprises a heavy chain CDR1-3 and a light chain CDR1-3, of a sequence of Table 28 according to Chothia. In some embodiments, the BCMA CAR comprises a heavy chain CDR1-3 of a sequence of Table 28. In some embodiments, the BCMA CAR comprises a heavy chain CDR1-3 of a sequence of Table 28 according to Kabat. In some embodiments, the BCMA CAR comprises a heavy chain CDR1-3, of a sequence of Table 28 according to Chothia. In some embodiments, the BCMA CAR comprises a light chain CDR1-3 of a sequence of Table 28. In some embodiments, the BCMA CAR comprises a light chain CDR1-3 of a sequence of Table 28 according to Kabat. In some embodiments, the BCMA CAR comprises a light chain CDR1-3 of a sequence of Table 28 according to Chothia.

TABLE 28
Amino acid sequence of an exemplary BCMA CAR

| SEQ ID | | |
|---|---|---|
| NO: | Description | Sequence |
| 375 | Exemplary | MALPVTALLLPLALLLHAARPDIVLTQSPPSLAMSLGKRATISCRA |
| | BCMA | SESVTILGSHLIHWYQQKPGQPPTLLIQLASNVQTGVPARFSGSGS |
| | CAR D | RTDFTLTIDPVEEDDVAVYYCLQSRTIPRTFGGGTKLEIKGSTSGS |
| | Protein | GKPGSGEGSTKGQIQLVQSGPELKKPGETVKISCKASGYTFTDYSI |
| | Sequence | NWVKRAPGKGLKWMGWINTETREPAYAYDFRGRFAFSLETSASTAY |
| | | LQINNLKYEDTATYFCALDYSYAMDYWGQGTSVTVSSAAATTTPAP |
| | | RPPTPAPTIASQPLSLRPEACRPAAGGAVHTRGLDFACDIYIWAPL |
| | | AGTCGVLLLSLVITLYCKRGRKKLLYIFKQPFMRPVQTTQEEDGCS |
| | | CRFPEEEEGGCELRVKFSRSADAPAYQQGQNQLYNELNLGRREEYD |
| | | VLDKRRGRDPEMGGKPRRKNPQEGLYNELQKDKMAEAYSEIGMKGE |
| | | RRRGKGHDGLYQGLSTATKDTYDALHMQALPPR |
| 376 | Exemplary | atggcactcc ccgtcaccgc ccttctcttg |
| | BCMA | cccctcgccc tgctgctgca tgctgccaggcccgacattg |
| | CAR D | tgctcactca gtcacctccc agcctggcca |
| | nucleotide | tgagcctggg aaaaagggccaccatctcct gtagagccag |
| | Sequence | tgagtccgtc acaatcttgg ggagccatct |
| | | tattcactggtatcagcaga agcccgggca gcctccaacc |
| | | cttcttattc agctcgcgtc aaacgtccagacgggtgtac |
| | | ctgccagatt ttctggtagc gggtcccgca |
| | | ctgattttac actgaccatagatccagtgg aagaagacga |
| | | tgtggccgtg tattattgtc tgcagagcag |
| | | aacgattcctcgcacatttg gtgggggtac taagctggag |
| | | attaagggaa gcacgtccgg ctcagggaagccgggctccg |
| | | gcgagggaag cacgaagggg caaattcagc |
| | | tggtccagag cggacctgagctgaaaaaac ccggcgagac |
| | | tgttaagatc agttgtaaag catctggcta |
| | | taccttcaccgactacagca taaattgggt gaaacgggcc |
| | | cctggaaagg gcctcaaatg gatgggttggatcaataccg |
| | | aaactaggga gcctgcttat gcatatgact |
| | | tccgcgggag attcgccttttcactcgaga catctgcctc |
| | | tactgcttac ctccaaataa acaacctcaa |
| | | gtatgaagatacagccactt acttttgcgc cctcgactat |
| | | agttacgcca tggactactg gggacagggaacctccgtta |
| | | ccgtcagttc cgcggccgca accacaacac |
| | | ctgctccaag gccccccacacccgctccaa ctatagccag |
| | | ccaaccattg agcctcagac ctgaagcttg |
| | | caggcccgcagcaggaggcg ccgtccatac gcgaggcctg |
| | | gacttcgcgt gtgatattta tatttgggcccctttggccg |
| | | gaacatgtgg ggtgttgctt ctctcccttg |
| | | tgatcactct gtattgtaagcgcgggagaa agaagctcct |
| | | gtacatcttc aagcagcctt ttatgcgacc |
| | | tgtgcaaaccactcaggaag aagatgggtg ttcatgccgc |
| | | ttccccgagg aggaagaagg agggtgtgaactgagggtga |
| | | aattttctag aagcgccgat gctcccgcat |
| | | atcagcaggg tcagaatcagctctacaatg aattgaatct |
| | | cggcaggcga gaagagtacg atgttctgga |
| | | caagagacggggcagggatc ccgagatggg gggaaagccc |
| | | cggagaaaaa atcctcagga ggggttgtacaatgagctgc |
| | | agaaggacaa gatggctgaa gcctatagcg |
| | | agatcggaat gaaaggcgaaagacgcagag gcaaggggca |
| | | tgacggtctg taccagggtc tctctacagc |
| | | caccaaggacacttatgatg cgttgcatat gcaagccttg |
| | | ccaccccgct aatga |

### Exemplary BCMA CAR E

In some embodiments, the BCMA CAR comprises two single-domain antibodies linked to a 4-1BB costimulatory domain and a CD3-zeta signaling domain. In some embodiments, the chimeric antigen receptor described herein comprises a polypeptide comprising, (a) an extracellular antigen binding domain comprising a first anti-BCMA single domain antibody (sdAb), and a second anti-BCMA sdAb. In some embodiments, each of the first and second anti-BCMA antibody are independently a VhH domain. In certain embodiments, the first anti-BCMA sdAb comprises a CDR1, a CDR2, and a CDR3 as set forth in the VhH domain comprising the amino acid sequence of SEQ ID NO: 377, or a peptide sequence having at least 80%, 85%, 90%, 95%, 97%, or 100% or no more than 20, 15, 10, 8, 5, 4, 3, 2, or 1 variations thereto. In certain embodiments, the second anti-BCMA sdAb comprises a CDR1, a CDR2, and a CDR3 as set forth in the VhH domain comprising the amino acid sequence of SEQ ID NO: 381, or a peptide sequence having at least 80%, 85%, 90%, 95%, 97%, or 100% or no more than 20, 15, 10, 8, 5, 4, 3, 2, or 1 variations thereto. In certain embodiments, the BCMA CAR is any BCMA CAR described in U.S. Pat. No. 11,186,647, the entire contents of which are incorporated herein by reference. In some embodiments, the CD19 CAR is encoded by a nucleotide sequence of Table 29, or a nucleotide sequence having at least 80%, 85%, 90%, 95%, 97%, or 100% or no more than 20, 15, 10, 8, 5, 4, 3, 2, or 1 variations thereto. In some embodiments, the CD19 CAR comprises a polypeptide encoded by a nucleotide sequence of Table 29, or a nucleotide sequence having at least 80%, 85%, 90%, 95%, 97%, or 100% or no more than 20, 15, 10, 8, 5, 4, 3, 2, or 1 variations thereto. In some embodiments, the CD19 CAR comprises a polypeptide sequence of Table 29, or a polypeptide sequence having at least 80%, 85%, 90%, 95%, 97%, or 100% or no more than 20, 15, 10, 8, 5, 4, 3, 2, or 1 variations thereto. In some embodiments, the BCMA CAR comprises a heavy chain CDR1-3 and a light chain CDR1-3, of a sequence of Table 29. In some embodiments, the BCMA CAR comprises a heavy chain CDR1-3 and a light chain CDR1-3, of a sequence of Table 29 according to Kabat. In some embodiments, the BCMA CAR comprises a heavy chain CDR1-3 and a light chain CDR1-3, of a sequence of Table 29 according to Chothia. In some embodiments, the BCMA CAR comprises a heavy chain CDR1-3 of a sequence of Table 29. In some embodiments, the BCMA CAR comprises a heavy chain CDR1-3 of a sequence of Table 29 according to Kabat. In some embodiments, the BCMA CAR comprises a heavy chain CDR1-3 of a sequence of Table 29 according to Chothia. In some embodiments, the BCMA CAR comprises a light chain CDR1-3 of a sequence of Table 29. In some embodiments, the BCMA CAR comprises a light chain CDR1-3 of a sequence of Table 29 according to Kabat. In some embodiments, the BCMA CAR comprises a light chain CDR1-3 of a sequence of Table 29 according to Chothia.

TABLE 29
Amino acid and nucleic acid sequences of an exemplary BCMA CAR D

| SEQ ID | | |
|---|---|---|
| NO: | Description | Sequence |
| 377 | Antigen | QVKLEESGGGLVQAGRSLRLSCAASEHTFSSHVMGWFRQAPGKERE |
| | Binding | SVAVIGWRDISTSYADSVKGRFTISRDNAKKTLYLQMNSLKPEDTA |
| | Domain | VYYCAARRIDAADFDSWGQGTQVTVSS |
| | VhH One | |
| 378 | Antigen | SHVMG |
| | Binding | |
| | Domain | |
| | VhH One | |
| | CDR1 | |
| 379 | Antigen | VIGWRDISTSYADSVK |
| | Binding | |
| | Domain | |
| | VhH One | |
| | CDR2 | |
| 380 | Antigen | ARRIDAADFDS |
| | Binding | |
| | Domain | |
| | VhH One | |
| | CDR3 | |
| 381 | Antigen | EVQLVESGGGLVQAGGSLRLSCAASGRTFTMGWFRQAPGKEREFVA |
| | Binding | AISLSPTLAYYAESVKGRFTISRDNAKNTVVLQMNSLKPEDTALYY |
| | Domain | CAADRKSVMSIRPDYWGQGTQVTVSS |
| | VhH Two | |
| 382 | Antigen | TFTMG |
| | Binding | |
| | Domain | |
| | VhH Two | |
| | CDR1 | |
| 383 | Antigen | AISLSPTLAYYAESVK |
| | Binding | |
| | Domain | |
| | VhH Two | |
| | CDR2 | |
| 384 | Antigen | ADRKSVMSIRPDY |
| | Binding | |
| | Domain | |
| | VhH Two | |
| | CDR3 | |
| 385 | Exemplary | CAGGTCAAACTGGAAGAATCTGGCGGAGGCCTGGTGCAGGCAGGAC |
| | BCMA | GGAGCCTGCGCCTGAGCTGCGCAGCATCCGAGCACACCTTCAGCTC |
| | CARD | CCACGTGATGGGCTGGTTTCGGCAGGCCCCAGGCAAGGAGAGAGAG |
| | Nucleic | AGCGTGGCCGTGATCGGCTGGAGGGACATCTCCACATCTTACGCCG |
| | Acid | ATTCCGTGAAGGGCCGGTTCACCATCAGCCGGGACAACGCCAAGAA |
| | | GACACTGTATCTGCAGATGAACAGCCTGAAGCCCGAGGACACCGCC |
| | | GTGTACTATTGCGCAGCAAGGAGAATCGACGCAGCAGACTTTGATT |
| | | CCTGGGGCCAGGGCACCCAGGTGACAGTGTCTAGCGGAGGAGGAGG |
| | | ATCTGAGGTGCAGCTGGTGGAGAGCGGAGGCGGCCTGGTGCAGGCC |
| | | GGAGGCTCTCTGAGGCTGAGCTGTGCAGCATCCGGAAGAACCTTCA |
| | | CAATGGGCTGGTTTAGGCAGGCACCAGGAAAGGAGAGGGAGTTCGT |
| | | GGCAGCAATCAGCCTGTCCCCTACCCTGGCCTACTATGCCGAGAGC |
| | | GTGAAGGGCAGGTTTACCATCTCCCGCGATAACGCCAAGAATACAG |
| | | TGGTGCTGCAGATGAACTCCCTGAAACCTGAGGACACAGCCCTGTA |
| | | CTATTGTGCCGCCGATCGGAAGAGCGTGATGAGCATTAGACCAGAC |
| | | TATTGGGGGCAGGGAACACAGGTGACCGTGAGCAGCACTAGTACCA |
| | | CGACGCCAGCGCCGCGACCACCAACACCGGCGCCCACCATCGCGTC |
| | | GCAGCCCCTGTCCCTGCGCCCAGAGGCGTGCCGGCCAGCGGCGGGG |
| | | GGCGCAGTGCACACGAGGGGGCTGGACTTCGCCTGTGATATCTACA |
| | | TCTGGGCGCCCTTGGCCGGGACTTGTGGGGTCCTTCTCCTGTCACT |
| | | GGTTATCACCCTTTACTGCAAACGGGGCAGAAAGAAACTCCTGTAT |
| | | ATATTCAAACAACCATTTATGAGACCAGTACAAACTACTCAAGAGG |
| | | AAGATGGCTGTAGCTGCCGATTTCCAGAAGAAGAAGAAGGAGGATG |
| | | TGAACTGAGAGTGAAGTTCAGCAGGAGCGCAGACGCCCCCGCGTAC |
| | | CAGCAGGGCCAGAACCAGCTCTATAACGAGCTCAATCTAGGACGAA |
| | | GAGAGGAGTACGATGTTTTGGACAAGAGACGTGGCCGGGACCCTGA |
| | | GATGGGGGGAAAGCCGAGAAGGAAGAACCCTCAGGAAGGCCTGTAC |
| | | AATGAACTGCAGAAAGATAAGATGGCGGAGGCCTACAGTGAGATTG |
| | | GGATGAAAGGCGAGCGCCGGAGGGGCAAGGGGCACGATGGCCTTTA |
| | | CCAGGGTCTCAGTACAGCCACCAAGGACACCTACGACGCCCTTCAC |
| | | ATGCAGGCCCTGCCCCCTCGCTAA |
| 386 | Exemplary | QVKLEESGGGLVQAGRSLRLSCAASEHTFSSHVMGWFRQAPGKERE |
| | BCMA | SVAVIGWRDISTSYADSVKGRFTISRDNAKKTLYLQMNSLKPEDTA |
| | CAR D | VYYCAARRIDAADFDSWGQGTQVTVSSGGGGSEVQLVESGGGLVQA |
| | Peptide | GGSLRLSCAASGRTFTMGWFRQAPGKEREFVAAISLSPTLAYYAES |
| | sequence | VKGRFTISRDNAKNTVVLQMNSLKPEDTALYYCAADRKSVMSIRPD |
| | | YWGQGTQVTVSSTSTTTPAPRPPTPAPTIASQPLSLRPEACRPAAG |
| | | GAVHTRGLDFACDIYIWAPLAGTCGVLLLSLVITLYCKRGRKKLLY |
| | | IFKQPFMRPVQTTQEEDGCSCRFPEEEEGGCELRVKFSRSADAPAY |
| | | QQGQNQLYNELNLGRREEYDVLDKRRGRDPEMGGKPRRKNPQEGLY |
| | | NELQKDKMAEAYSEIGMKGERRRGKGHDGLYQGLSTATKDTYDALH |
| | | MQALPPR |

### Other Exemplary Targets

Further non-limiting exemplary tumor antigens include CD20, CD22, EGFR, CD123, and CLL-1.

CARs that bind to CD20 are known in the art. For example, those disclosed in WO2018/067992 or WO2016/164731, incorporated by reference herein, can be used in accordance with the present disclosure. Any known CD20 CAR, for example, the CD20 antigen binding domain of any known CD20 CAR, in the art can be used in accordance with the present disclosure. Exemplary CD20-binding sequences or CD20 CAR sequences are disclosed in, for example, Tables 1-5 of WO2018/067992, incorporated by reference. In some embodiments, the CD20 CAR comprises a CDR, variable region, scFv, or full-length sequence of a CD20 CAR disclosed in WO2018/067992 or WO2016/164731, both incorporated by reference herein. In some embodiments, CD20 CARs comprise a sequence, for example, a CDR, VH, VL, scFv, or full-CAR sequence, disclosed in Table 23 below, or a sequence having at least 70%, 75%, 80%, 85%, 90%, 95%, 96%, 97%, 98%, or 99% identity thereto.

Exemplary antigen binding domains that bind CD20 are described in WO2016/164731 and WO2018/067992, incorporated herein by reference. In some embodiments, the antigen binding domain of one or more of the CD20 antigen binding domains disclosed therein.

Exemplary antigen binding domains that bind CD22 are described in WO2016/164731 and WO2018/067992, incorporated herein by reference.

In some embodiments, the antigen binding domain comprises a HC CDR1, a HC CDR2, and a HC CDR3 of any heavy chain binding domain amino acid sequences listed in Table 15. In embodiments, the antigen binding domain further comprises a LC CDR1, a LC CDR2, and a LC CDR3. In embodiments, the antigen binding domain comprises a LC CDR1, a LC CDR2, and a LC CDR3 amino acid sequences listed in Table 16.

In some embodiments, the antigen binding domain comprises one, two or all of LC CDR1, LC CDR2, and LC CDR3 of any light chain binding domain amino acid sequences listed in Table 16, and one, two or all of HC CDR1, HC CDR2, and HC CDR3 of any heavy chain binding domain amino acid sequences listed in Table 15.

Exemplary antigen binding domains that bind EGFRvIII are described in in WO2014/130657.

Exemplary antigen binding domains that bind CD123 are described in WO 2014/130635 and WO2016/028896, incorporated herein by reference.

In some embodiments, the antigen binding domain comprises a sequence from Tables 1-2 of WO2014/130635, incorporated herein by reference.

In some embodiments, the antigen binding domain comprises a sequence from Tables 2, 6, and 9 of WO2016/028896, incorporated herein by reference.

Exemplary antigen binding domains that bind CLL-1 are disclosed in WO2016/014535, incorporated herein by reference.

In some embodiments, the antigen binding domain comprises one, two three (for example, all three) heavy chain CDRs, HC CDR1, HC CDR2 and HC CDR3, from an antibody described herein (for example, an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference), and/or one, two, three (for example, all three) light chain CDRs, LC CDR1, LC CDR2 and LC CDR3, from an antibody described herein (for example, an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference). In some embodiments, the antigen binding domain comprises a heavy chain variable region and/or a variable light chain region of an antibody listed above.

In embodiments, the antigen binding domain is an antigen binding domain described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference.

Exemplary target antigens that can be targeted using the CAR-expressing cells, include, but are not limited to, CD19, CD123, EGFRvIII, CD33, mesothelin, BCMA, and GFR ALPHA-4, among others, as described in, for example, WO2014/153270, WO 2014/130635, WO2016/028896, WO 2014/130657, WO2016/014576, WO 2015/090230, WO2016/014565, WO2016/014535, and WO2016/025880, each of which is herein incorporated by reference in its entirety.

In some embodiments, the antigen binding domain of any of the CARs described herein (for example, any of CD19, CD123, EGFRvIII, CD33, mesothelin, BCMA, and GFR ALPHA-4) comprises one, two three (for example, all three) heavy chain CDRs, HC CDR1, HC CDR2 and HC CDR3, from an antibody listed above, and/or one, two, three (for example, all three) light chain CDRs, LC CDR1, LC CDR2 and LC CDR3, from an antigen binding domain listed above. In some embodiments, the antigen binding domain comprises a heavy chain variable region and/or a variable light chain region of an antibody listed or described above.

In some embodiments, the antigen binding domain comprises one, two three (for example, all three) heavy chain CDRs, HC CDR1, HC CDR2 and HC CDR3, from an antibody listed above, and/or one, two, three (for example, all three) light chain CDRs, LC CDR1, LC CDR2 and LC CDR3, from an antibody listed above. In some embodiments, the antigen binding domain comprises a heavy chain variable region and/or a variable light chain region of an antibody listed or described above.

TABLE 23
Amino acid sequences of exemplary anti-CD20 molecules

| SEQ ID NO: | Region | Sequence |
|---|---|---|
| CD20-C3H2 | | |
| 769 | HCDR1 | NYNLH |
| | (Kabat) | |
| 770 | HCDR2 | AIYPGNYDTSYNQKFKG |
| | (Kabat) | |
| 771 | HCDR3 | VDFGHSRYWYFDV |
| | (Kabat) | |
| 772 | HCDR1 | GYTFTNY |
| | (Chothia) | |
| 773 | HCDR2 | YPGNYD |
| | (Chothia) | |
| 771 | HCDR3 | VDFGHSRYWYFDV |
| | (Chothia) | |
| 774 | HCDR1 | GYTFTNYN |
| | (IMGT) | |
| 775 | HCDR2 | IYPGNYDT |
| | (IMGT) | |
| 776 | HCDR3 | ARVDFGHSRYWYFDV |
| | (IMGT) | |
| 777 | HCDR1 | GYTFTNYNLH |
| | (Combined) | |
| 770 | HCDR2 | AIYPGNYDTSYNQKFKG |
| | (Combined) | |
| 771 | HCDR3 | VDFGHSRYWYFDV |
| | (Combined) | |
| 778 | VH | QVQLVQSGAEVKKPGASVKVSCKASGYTFTNYNLHWVRQ |
| | | APGQGLEWMGAIYPGNYDTSYNQKFKGRVTMTADKSTSTA |
| | | YMELSSLRSEDTAVYYCARVDFGHSRYWYFDVWGQGTTV |
| | | TVSS |
| 779 | DNA VH | CAAGTCCAACTCGTCCAGTCCGGTGCAGAAGTCAAGAAA |
| | | CCTGGAGCATCCGTGAAAGTGTCTTGCAAAGCCTCCGGCT |
| | | ACACCTTCACCAACTACAACCTCCATTGGGTCAGACAGG |
| | | CCCCCGGACAAGGACTCGAATGGATGGGAGCGATCTACC |
| | | CGGGAAACTACGACACCAGCTACAACCAGAAGTTCAAGG |
| | | GCCGCGTGACTATGACCGCCGATAAGAGCACCTCCACCG |
| | | CCTACATGGAACTGTCCTCGCTGAGGTCCGAGGACACTG |
| | | CGGTGTACTACTGCGCCCGCGTGGACTTCGGACACTCACG |
| | | GTATTGGTACTTCGACGTCTGGGGACAGGGCACTACCGT |
| | | GACCGTGTCGAGC |
| 780 | LCDR1 | RATSSVSSMN |
| | (Kabat) | |
| 781 | LCDR2 | ATSNLAS |
| | (Kabat) | |
| 782 | LCDR3 | QQWTFNPPT |
| | (Kabat) | |
| 783 | LCDR1 | TSSVSS |
| | (Chothia) | |
| 784 | LCDR2 | ATS |
| | (Chothia) | |
| 785 | LCDR3 | WTFNPP |
| | (Chothia) | |
| 786 | LCDR1 | SSVSS |
| | (IMGT) | |
| 784 | LCDR2 | ATS |
| | (IMGT) | |
| 782 | LCDR3 | QQWTFNPPT |
| | (IMGT) | |
| 780 | LCDR1 | RATSSVSSMN |
| | (Combined) | |
| 781 | LCDR2 | ATSNLAS |
| | (Combined) | |
| 782 | LCDR3 | QQWTFNPPT |
| | (Combined) | |
| 787 | VL | DIQLTQSPSFLSASVGDRVTITCRATSSVSSMNWYQQKPGK |
| | | APKPLIHATSNLASGVPSRFSGSGSGTEYTLTISSLQPEDFAT |
| | | YYCQQWTFNPPTFGQGTKLEIK |
| 788 | DNA VL | GATATCCAGCTGACTCAGTCCCCGTCATTCCTGTCCGCCT |
| | | CCGTGGGAGACAGAGTGACCATCACCTGTCGGGCCACTT |
| | | CCTCCGTGTCAAGCATGAACTGGTATCAGCAGAAGCCCG |
| | | GGAAGGCCCCAAAGCCGCTGATTCACGCGACGTCCAACC |
| | | TGGCTTCCGGCGTGCCGAGCCGGTTCTCCGGCTCGGGGA |
| | | GCGGGACTGAGTACACCCTGACTATTTCCTCGCTTCAACC |
| | | CGAGGACTTTGCTACCTACTACTGCCAACAGTGGACCTTC |
| | | AATCCTCCGACATTCGGACAGGGTACCAAGTTGGAAATC |
| | | AAG |
| 789 | scFv (VH- | QVQLVQSGAEVKKPGASVKVSCKASGYTFTNYNLHWVRQ |
| | linker-VL) | APGQGLEWMGAIYPGNYDTSYNQKFKGRVTMTADKSTSTA |
| | | YMELSSLRSEDTAVYYCARVDFGHSRYWYFDVWGQGTTV |
| | | TVSSGGGGSGGGGSGGGGSGGGGSDIQLTQSPSFLSASVGD |
| | | RVTITCRATSSVSSMNWYQQKPGKAPKPLIHATSNLASGVP |
| | | SRFSGSGSGTEYTLTISSLQPEDFATYYCQQWTFNPPTFGQG |
| | | TKLEIK |
| 790 | DNA scFv | caagtccaactcgtccagtccggtgcagaagtcaagaaacctggagcatccgtgaaagtgtctt |
| | (VH-linker- | gcaaagcctccggctacaccttcaccaactacaacctccattgggtcagacaggcccccggac |
| | VL | aaggactcgaatggatgggagcgatctacccgggaaactacgacaccagctacaaccagaa |
| | | gttcaagggccgcgtgactatgaccgccgataagagcacctccaccgcctacatggaactgtc |
| | | ctcgctgaggtccgaggacactgcggtgtactactgcgcccgcgtggacttcggacactcacg |
| | | gtattggtacttcgacgtctggggacagggcactaccgtgaccgtgtcgagcggcggaggag |
| | | gttcggggggggcggatcagggggcggcggcagcggtggagggggctcggatatccag |
| | | ctgactcagtccccgtcattcctgtccgcctccgtgggagacagagtgaccatcacctgtcggg |
| | | ccacttcctccgtgtcaagcatgaactggtatcagcagaagcccgggaaggccccaaagccg |
| | | ctgattcacgcgacgtccaacctggcttccggcgtgccgagccggttctccggctcggggagc |
| | | gggactgagtacaccctgactatttcctcgcttcaacccgaggactttgctacctactactgccaa |
| | | cagtggaccttcaatcctccgacattcggacagggtaccaagttggaaatcaag |
| 791 | Full CAR | MALPVTALLLPLALLLHAARPQVQLVQSGAEVKKPGASVK |
| | amino acid | VSCKASGYTFTNYNLHWVRQAPGQGLEWMGAIYPGNYDT |
| | sequence | SYNQKFKGRVTMTADKSTSTAYMELSSLRSEDTAVYYCAR |
| | | VDFGHSRYWYFDVWGQGTTVTVSSGGGGSGGGGSGGGGS |
| | | GGGGSDIQLTQSPSFLSASVGDRVTITCRATSSVSSMNWYQ |
| | | QKPGKAPKPLIHATSNLASGVPSRFSGSGSGTEYTLTISSLQP |
| | | EDFATYYCQQWTFNPPTFGQGTKLEIKTTTPAPRPPTPAPTIA |
| | | SQPLSLRPEACRPAAGGAVHTRGLDFACDIYIWAPLAGTCG |
| | | VLLLSLVITLYCKRGRKKLLYIFKQPFMRPVQTTQEEDGCSC |
| | | RFPEEEEGGCELRVKFSRSADAPAYQQGQNQLYNELNLGRR |
| | | EEYDVLDKRRGRDPEMGGKPRRKNPQEGLYNELQKDKMA |
| | | EAYSEIGMKGERRRGKGHDGLYQGLSTATKDTYDALHMQ |
| | | ALPPR |
| 792 | Full CAR | ATGGCCCTCCCTGTCACCGCCCTGCTGCTTCCGCTGGCTC |
| | nucleotide | TTCTGCTCCACGCCGCTCGGCCCCAAGTCCAACTCGTCCA |
| | sequence | GTCCGGTGCAGAAGTCAAGAAACCTGGAGCATCCGTGAA |
| | | AGTGTCTTGCAAAGCCTCCGGCTACACCTTCACCAACTAC |
| | | AACCTCCATTGGGTCAGACAGGCCCCCGGACAAGGACTC |
| | | GAATGGATGGGAGCGATCTACCCGGGAAACTACGACACC |
| | | AGCTACAACCAGAAGTTCAAGGGCCGCGTGACTATGACC |
| | | GCCGATAAGAGCACCTCCACCGCCTACATGGAACTGTCC |
| | | TCGCTGAGGTCCGAGGACACTGCGGTGTACTACTGCGCC |
| | | CGCGTGGACTTCGGACACTCACGGTATTGGTACTTCGACG |
| | | TCTGGGGACAGGGCACTACCGTGACCGTGTCGAGCGGCG |
| | | GAGGAGGTTCGGGAGGGGGCGGATCAGGGGGCGGCGGC |
| | | AGCGGTGGAGGGGGCTCGGATATCCAGCTGACTCAGTCC |
| | | CCGTCATTCCTGTCCGCCTCCGTGGGAGACAGAGTGACCA |
| | | TCACCTGTCGGGCCACTTCCTCCGTGTCAAGCATGAACTG |
| | | GTATCAGCAGAAGCCCGGGAAGGCCCCAAAGCCGCTGAT |
| | | TCACGCGACGTCCAACCTGGCTTCCGGCGTGCCGAGCCG |
| | | GTTCTCCGGCTCGGGGAGCGGGACTGAGTACACCCTGAC |
| | | TATTTCCTCGCTTCAACCCGAGGACTTTGCTACCTACTAC |
| | | TGCCAACAGTGGACCTTCAATCCTCCGACATTCGGACAG |
| | | GGTACCAAGTTGGAAATCAAGACCACTACCCCAGCACCG |
| | | AGGCCACCCACCCCGGCTCCTACCATCGCCTCCCAGCCTC |
| | | TGTCCCTGCGTCCGGAGGCATGTAGACCCGCAGCTGGTG |
| | | GGGCCGTGCATACCCGGGGTCTTGACTTCGCCTGCGATAT |
| | | CTACATTTGGGCCCCTCTGGCTGGTACTTGCGGGGTCCTG |
| | | CTGCTTTCACTCGTGATCACTCTTTACTGTAAGCGCGGTC |
| | | GGAAGAAGCTGCTGTACATCTTTAAGCAACCCTTCATGA |
| | | GGCCTGTGCAGACTACTCAAGAGGAGGACGGCTGTTCAT |
| | | GCCGGTTCCCAGAGGAGGAGGAAGGCGGCTGCGAACTGC |
| | | GCGTGAAATTCAGCCGCAGCGCAGATGCTCCAGCCTACC |
| | | AGCAGGGGCAGAACCAGCTCTACAACGAACTCAATCTTG |
| | | GTCGGAGAGAGGAGTACGACGTGCTGGACAAGCGGAGA |
| | | GGACGGGACCCAGAAATGGGCGGGAAGCCGCGCAGAAA |
| | | GAATCCCCAAGAGGGCCTGTACAACGAGCTCCAAAAGGA |
| | | TAAGATGGCAGAAGCCTATAGCGAGATTGGTATGAAAGG |
| | | GGAACGCAGAAGAGGCAAAGGCCACGACGGACTGTACC |
| | | AGGGACTCAGCACCGCCACCAAGGACACCTATGACGCTC |
| | | TTCACATGCAGGCCCTGCCGCCTCGG |
| CD20-C5H1 | | |
| 793 | HCDR1 | SYNMH |
| | (Kabat) | |
| 794 | HCDR2 | AIYPGNGDTSYNPKFKG |
| | (Kabat) | |
| 795 | HCDR3 | SYFYGSSSWYFDV |
| | (Kabat) | |
| 523 | HCDR1 | GYTFTSY |
| | (Chothia) | |
| 796 | HCDR2 | YPGNGD |
| | (Chothia) | |
| 795 | HCDR3 | SYFYGSSSWYFDV |
| | (Chothia) | |
| 797 | HCDR1 | GYTFTSYN |
| | (IMGT) | |
| 798 | HCDR2 | IYPGNGDT |
| | (IMGT) | |
| 799 | HCDR3 | ARSYFYGSSSWYFDV |
| | (IMGT) | |
| 800 | HCDR1 | GYTFTSYNMH |
| | (Combined) | |
| 794 | HCDR2 | AIYPGNGDTSYNPKFKG |
| | (Combined) | |
| 795 | HCDR3 | SYFYGSSSWYFDV |
| | (Combined) | |
| 801 | VH | QVQLVQSGAEVKKPGASVKVSCKASGYTFTSYNMHWVRQ |
| | | APGQGLEWMGAIYPGNGDTSYNPKFKGRVTMTADKSTRTA |
| | | YMELSSLRSEDTAVYYCARSYFYGSSSWYFDVWGQGTTVT |
| | | VSS |
| 802 | DNA VH | CAAGTGCAGCTCGTCCAGTCCGGTGCAGAAGTCAAGAAA |
| | | CCCGGTGCTTCAGTGAAAGTGTCCTGCAAGGCCTCCGGTT |
| | | ACACCTTCACCTCCTACAACATGCACTGGGTCCGCCAAGC |
| | | CCCGGGCCAGGGACTCGAATGGATGGGAGCCATCTACCC |
| | | TGGCAACGGGGACACCTCATACAACCCTAAGTTCAAGGG |
| | | CAGAGTGACCATGACTGCGGACAAGTCCACTAGAACAGC |
| | | GTACATGGAGCTGAGCAGCCTGCGGTCCGAGGATACTGC |
| | | CGTGTACTACTGCGCCCGCTCCTACTTCTACGGAAGCTCG |
| | | TCGTGGTACTTCGATGTCTGGGGACAGGGCACCACTGTG |
| | | ACTGTGTCCTCC |
| 803 | LCDR1 | RASSSVSSMH |
| | (Kabat) | |
| 781 | LCDR2 | ATSNLAS |
| | (Kabat) | |
| 804 | LCDR3 | QQWIFNPPT |
| | (Kabat) | |
| 805 | LCDR1 | SSSVSS |
| | (Chothia) | |
| 784 | LCDR2 | ATS |
| | (Chothia) | |
| 806 | LCDR3 | WIFNPP |
| | (Chothia) | |
| 786 | LCDR1 | SSVSS |
| | (IMGT) | |
| 784 | LCDR2 | ATS |
| | (IMGT) | |
| 804 | LCDR3 | QQWIFNPPT |
| | (IMGT) | |
| 803 | LCDR1 | RASSSVSSMH |
| | (Combined) | |
| 781 | LCDR2 | ATSNLAS |
| | (Combined) | |
| 804 | LCDR3 | QQWIFNPPT |
| | (Combined) | |
| 807 | VL | EIVLTQSPATLSLSPGERATLSCRASSSVSSMHWYQQKPGQA |
| | | PRPLIFATSNLASGIPARFSGSGSGTDYTLTISSLEPEDAAVYY |
| | | CQQWIFNPPTFGGGTKVEIK |
| 808 | DNA VL | GAAATTGTGCTGACTCAGAGCCCCGCCACCCTGAGCTTGT |
| | | CCCCCGGGGAAAGGGCAACGCTGTCATGCCGCGCCTCGT |
| | | CATCCGTGTCCTCCATGCATTGGTACCAGCAGAAGCCGG |
| | | GACAGGCCCCTCGGCCGCTGATCTTCGCCACCTCCAATCT |
| | | CGCTTCCGGCATTCCGGCCCGGTTCTCGGGAAGCGGGTCG |
| | | GGGACCGACTATACCCTGACCATCTCTAGCCTTGAACCTG |
| | | AGGACGCCGCGGTGTACTATTGTCAACAGTGGATCTTTAA |
| | | CCCCCCAACCTTCGGTGGAGGCACCAAAGTGGAGATTAA |
| | | G |
| 809 | scFv (VH- | QVQLVQSGAEVKKPGASVKVSCKASGYTFTSYNMHWVRQ |
| | linker-VL) | APGQGLEWMGAIYPGNGDTSYNPKFKGRVTMTADKSTRTA |
| | | YMELSSLRSEDTAVYYCARSYFYGSSSWYFDVWGQGTTVT |
| | | VSSGGGGSGGGGSGGGGSGGGGSEIVLTQSPATLSLSPGER |
| | | ATLSCRASSSVSSMHWYQQKPGQAPRPLIFATSNLASGIPAR |
| | | FSGSGSGTDYTLTISSLEPEDAAVYYCQQWIFNPPTFGGGTK |
| | | VEIK |
| 810 | DNA scFv | Caagtgcagctcgtccagtccggtgcagaagtcaagaaacccggtgcttcagtgaaagtgtcc |
| | (VH-linker- | tgcaaggcctccggttacaccttcacctcctacaacatgcactgggtccgccaagccccgggc |
| | VL) | cagggactcgaatggatgggagccatctaccctggcaacggggacacctcatacaaccctaa |
| | | gttcaagggcagagtgaccatgactgcggacaagtccactagaacagcgtacatggagctga |
| | | gcagcctgcggtccgaggatactgccgtgtactactgcgcccgctcctacttctacggaagctc |
| | | gtcgtggtacttcgatgtctggggacagggcaccactgtgactgtgtcctccggtggcggagg |
| | | ctcgggggaggcggaagcggcggcgggggatcgggaggaggagggtccgaaattgtgc |
| | | tgactcagagccccgccaccctgagcttgtcccccggggaaagggcaacgctgtcatgccgc |
| | | gcctcgtcatccgtgtcctccatgcattggtaccagcagaagccgggacaggcccctcggccg |
| | | ctgatcttcgccacctccaatctcgcttccggcattccggcccggttctcgggaagcgggtcgg |
| | | ggaccgactataccctgaccatctctagccttgaacctgaggacgccgcggtgtactattgtcaa |
| | | cagtggatctttaaccccccaaccttcggtggaggcaccaaagtggagattaag |
| 811 | Full CAR | MALPVTALLLPLALLLHAARPQVQLVQSGAEVKKPGASVK |
| | amino acid | VSCKASGYTFTSYNMHWVRQAPGQGLEWMGAIYPGNGDT |
| | sequence | SYNPKFKGRVTMTADKSTRTAYMELSSLRSEDTAVYYCAR |
| | | SYFYGSSSWYFDVWGQGTTVTVSSGGGGSGGGGSGGGGSG |
| | | GGGSEIVLTQSPATLSLSPGERATLSCRASSSVSSMHWYQQK |
| | | PGQAPRPLIFATSNLASGIPARFSGSGSGTDYTLTISSLEPEDA |
| | | AVYYCQQWIFNPPTFGGGTKVEIKTTTPAPRPPTPAPTIASQP |
| | | LSLRPEACRPAAGGAVHTRGLDFACDIYIWAPLAGTCGVLL |
| | | LSLVITLYCKRGRKKLLYIFKQPFMRPVQTTQEEDGCSCRFP |
| | | EEEEGGCELRVKFSRSADAPAYQQGQNQLYNELNLGRREE |
| | | YDVLDKRRGRDPEMGGKPRRKNPQEGLYNELQKDKMAEA |
| | | YSEIGMKGERRRGKGHDGLYQGLSTATKDTYDALHMQALP |
| | | PR |
| 812 | Full CAR | ATGGCCCTCCCTGTCACCGCCCTGCTGCTTCCGCTGGCTC |
| | nucleotide | TTCTGCTCCACGCCGCTCGGCCCCAAGTGCAGCTCGTCCA |
| | sequence | GTCCGGTGCAGAAGTCAAGAAACCCGGTGCTTCAGTGAA |
| | | AGTGTCCTGCAAGGCCTCCGGTTACACCTTCACCTCCTAC |
| | | AACATGCACTGGGTCCGCCAAGCCCCGGGCCAGGGACTC |
| | | GAATGGATGGGAGCCATCTACCCTGGCAACGGGGACACC |
| | | TCATACAACCCTAAGTTCAAGGGCAGAGTGACCATGACT |
| | | GCGGACAAGTCCACTAGAACAGCGTACATGGAGCTGAGC |
| | | AGCCTGCGGTCCGAGGATACTGCCGTGTACTACTGCGCCC |
| | | GCTCCTACTTCTACGGAAGCTCGTCGTGGTACTTCGATGT |
| | | CTGGGGACAGGGCACCACTGTGACTGTGTCCTCCGGTGG |
| | | CGGAGGCTCGGGCGGAGGCGGAAGCGGCGGCGGGGGAT |
| | | CGGGAGGAGGAGGGTCCGAAATTGTGCTGACTCAGAGCC |
| | | CCGCCACCCTGAGCTTGTCCCCCGGGGAAAGGGCAACGC |
| | | TGTCATGCCGCGCCTCGTCATCCGTGTCCTCCATGCATTG |
| | | GTACCAGCAGAAGCCGGGACAGGCCCCTCGGCCGCTGAT |
| | | CTTCGCCACCTCCAATCTCGCTTCCGGCATTCCGGCCCGG |
| | | TTCTCGGGAAGCGGGTCGGGGACCGACTATACCCTGACC |
| | | ATCTCTAGCCTTGAACCTGAGGACGCCGCGGTGTACTATT |
| | | GTCAACAGTGGATCTTTAACCCCCCAACCTTCGGTGGAGG |
| | | CACCAAAGTGGAGATTAAGACCACTACCCCAGCACCGAG |
| | | GCCACCCACCCCGGCTCCTACCATCGCCTCCCAGCCTCTG |
| | | TCCCTGCGTCCGGAGGCATGTAGACCCGCAGCTGGTGGG |
| | | GCCGTGCATACCCGGGGTCTTGACTTCGCCTGCGATATCT |
| | | ACATTTGGGCCCCTCTGGCTGGTACTTGCGGGGTCCTGCT |
| | | GCTTTCACTCGTGATCACTCTTTACTGTAAGCGCGGTCGG |
| | | AAGAAGCTGCTGTACATCTTTAAGCAACCCTTCATGAGGC |
| | | CTGTGCAGACTACTCAAGAGGAGGACGGCTGTTCATGCC |
| | | GGTTCCCAGAGGAGGAGGAAGGCGGCTGCGAACTGCGCG |
| | | TGAAATTCAGCCGCAGCGCAGATGCTCCAGCCTACCAGC |
| | | AGGGGCAGAACCAGCTCTACAACGAACTCAATCTTGGTC |
| | | GGAGAGAGGAGTACGACGTGCTGGACAAGCGGAGAGGA |
| | | CGGGACCCAGAAATGGGCGGGAAGCCGCGCAGAAAGAA |
| | | TCCCCAAGAGGGCCTGTACAACGAGCTCCAAAAGGATAA |
| | | GATGGCAGAAGCCTATAGCGAGATTGGTATGAAAGGGGA |
| | | ACGCAGAAGAGGCAAAGGCCACGACGGACTGTACCAGG |
| | | GACTCAGCACCGCCACCAAGGACACCTATGACGCTCTTC |
| | | ACATGCAGGCCCTGCCGCCTCGG |
| CD20-C3H1 | | |
| 896 | HCDR1 | NYNLH |
| | (Kabat) | |
| 897 | HCDR2 | AIYPGNYDTSYNQKFKG |
| | (Kabat) | |
| 898 | HCDR3 | VDFGHSRYWYFDV |
| | (Kabat) | |
| 899 | HCDR1 | GYTFTNY |
| | (Chothia) | |
| 900 | HCDR2 | YPGNYD |
| | (Chothia) | |
| 901 | HCDR3 | VDFGHSRYWYFDV |
| | (Chothia) | |
| 903 | HCDR1 | GYTFTNYN |
| | (IMGT) | |
| 904 | HCDR2 | IYPGNYDT |
| | (IMGT) | |
| 905 | HCDR3 | ARVDFGHSRYWYFDV |
| | (IMGT) | |
| 906 | HCDR1 | GYTFTNYNLH |
| | (Combined) | |
| 907 | HCDR2 | AIYPGNYDTSYNQKFKG |
| | (Combined) | |
| 908 | HCDR3 | VDFGHSRYWYFDV |
| | (Combined) | |
| 909 | VH | QVQLVQSGAEVKKPGASVKVSCKASGYTFTNYNLHWVRQ |
| | | APGQGLEWMGAIYPGNYDTSYNQKFKGRVTMTADKSTSTA |
| | | YMELSSLRSEDTAVYYCARVDFGHSRYWYFDVWGQGTTV |
| | | TVSS |
| 910 | LCDR1 | RATSSVSSMN |
| | (Kabat) | |
| 911 | LCDR2 | ATSNLAS |
| | (Kabat) | |
| 912 | LCDR3 | QQWTFNPPT |
| | (Kabat) | |
| 913 | LCDR1 | TSSVSS |
| | (Chothia) | |
| 914 | LCDR2 | ATS |
| | (Chothia) | |
| 915 | LCDR3 | WTFNPP |
| | (Chothia) | |
| 916 | LCDR1 | SSVSS |
| | (IMGT) | |
| 917 | LCDR2 | ATS |
| | (IMGT) | |
| 918 | LCDR3 | QQWTFNPPT |
| | (IMGT) | |
| 919 | LCDR1 | RATSSVSSMN |
| | (Combined) | |
| 920 | LCDR2 | ATSNLAS |
| | (Combined) | |
| 921 | LCDR3 | QQWTFNPPT |
| | (Combined) | |
| 922 | VL | EIVLTQSPATLSLSPGERATLSCRATSSVSSMNWYQQKPGQA |
| | | PRPLIHATSNLASGIPARFSGSGSGTDYTLTISSLEPEDAAVY |
| | | YCQQWTFNPPTFGQGTKLEIK |
| 923 | scFv (VH- | QVQLVQSGAEVKKPGASVKVSCKASGYTFTNYNLHWVRQ |
| | linker-VL) | APGQGLEWMGAIYPGNYDTSYNQKFKGRVTMTADKSTSTA |
| | | YMELSSLRSEDTAVYYCARVDFGHSRYWYFDVWGQGTTV |
| | | TVSSGGGGSGGGGSGGGGSGGGGSEIVLTQSPATLSLSPGER |
| | | ATLSCRATSSVSSMNWYQQKPGQAPRPLIHATSNLASGIPAR |
| | | FSGSGSGTDYTLTISSLEPEDAAVYYCQQWTFNPPTFGQGTK |
| | | LEIK |
| 924 | Full CAR | MALPVTALLLPLALLLHAARPQVQLVQSGAEVKKPGASVK |
| | | VSCKASGYTFTNYNLHWVRQAPGQGLEWMGAIYPGNYDT |
| | | SYNQKFKGRVTMTADKSTSTAYMELSSLRSEDTAVYYCAR |
| | | VDFGHSRYWYFDVWGQGTTVTVSSGGGGSGGGGSGGGGS |
| | | GGGGSEIVLTQSPATLSLSPGERATLSCRATSSVSSMNWYQQ |
| | | KPGQAPRPLIHATSNLASGIPARFSGSGSGTDYTLTISSLEPED |
| | | AAVYYCQQWTFNPPTFGQGTKLEIKTTTPAPRPPTPAPTIAS |
| | | QPLSLRPEACRPAAGGAVHTRGLDFACDIYIWAPLAGTCGV |
| | | LLLSLVITLYCKRGRKKLLYIFKQPFMRPVQTTQEEDGCSCR |
| | | FPEEEEGGCELRVKFSRSADAPAYQQGQNQLYNELNLGRRE |
| | | EYDVLDKRRGRDPEMGGKPRRKNPQEGLYNELQKDKMAE |
| | | AYSEIGMKGERRRGKGHDGLYQGLSTATKDTYDALHMQA |
| | | LPPR |
| CD20-3H3 | | |
| 925 | HCDR1 | NYNLH |
| | (Kabat) | |
| 926 | HCDR2 | AIYPGNYDTSYNQKFKG |
| | (Kabat) | |
| 927 | HCDR3 | VDFGHSRYWYFDV |
| | (Kabat) | |
| 928 | HCDR1 | GYTFTNY |
| | (Chothia) | |
| 929 | HCDR2 | YPGNYD |
| | (Chothia) | |
| 930 | HCDR3 | VDFGHSRYWYFDV |
| | (Chothia) | |
| 931 | HCDR1 | GYTFTNYN |
| | (IMGT) | |
| 932 | HCDR2 | IYPGNYDT |
| | (IMGT) | |
| 933 | HCDR3 | ARVDFGHSRYWYFDV |
| | (IMGT) | |
| 934 | VH | QVQLVQSGAEVKKPGSSVKVSCKASGYTFTNYNLHWVRQ |
| | | APGQGLEWMGAIYPGNYDTSYNQKFKGRVTITADKSTSTA |
| | | YMELSSLRSEDTAVYYCARVDFGHSRYWYFDVWGQGTTV |
| | | TVSS |
| 935 | LCDR1 | RATSSVSSMN |
| | (Kabat) | |
| 936 | LCDR2 | ATSNLAS |
| | (Kabat) | |
| 937 | LCDR3 | QQWTFNPPT |
| | (Kabat) | |
| 938 | LCDR1 | TSSVSS |
| | (Chothia) | |
| 939 | LCDR2 | ATS |
| | (Chothia) | |
| 940 | LCDR3 | WTFNPP |
| | (Chothia) | |
| 941 | LCDR1 | SSVSS |
| | (IMGT) | |
| 942 | LCDR2 | ATS |
| | (IMGT) | |
| 943 | LCDR3 | QQWTFNPPT |
| | (IMGT) | |
| 944 | LCDR1 | RATSSVSSMN |
| | (Combined) | |
| 945 | LCDR2 | ATSNLAS |
| | (Combined) | |
| 946 | LCDR3 | QQWTFNPPT |
| | (Combined) | |
| 947 | VL | EIVLTQSPATLSLSPGERATLSCRATSSVSSMNWYQQKPGQA |
| | | PRPLIHATSNLASGIPARFSGSGSGTDYTLTISSLEPEDAAVY |
| | | YCQQWTFNPPTFGQGTKLEIK |
| 948 | scFv (VH- | QVQLVQSGAEVKKPGSSVKVSCKASGYTFTNYNLHWVRQ |
| | linker-VL) | APGQGLEWMGAIYPGNYDTSYNQKFKGRVTITADKSTSTA |
| | | YMELSSLRSEDTAVYYCARVDFGHSRYWYFDVWGQGTTV |
| | | TVSSGGGGSGGGGSGGGGSGGGGSEIVLTQSPATLSLSPGER |
| | | ATLSCRATSSVSSMNWYQQKPGQAPRPLIHATSNLASGIPAR |
| | | FSGSGSGTDYTLTISSLEPEDAAVYYCQQWTFNPPTFGQGTK |
| | | LEIK |
| 949 | Full CAR | MALPVTALLLPLALLLHAARPQVQLVQSGAEVKKPGSSVK |
| | | VSCKASGYTFTNYNLHWVRQAPGQGLEWMGAIYPGNYDT |
| | | SYNQKFKGRVTITADKSTSTAYMELSSLRSEDTAVYYCARV |
| | | DFGHSRYWYFDVWGQGTTVTVSSGGGGSGGGGSGGGGSG |
| | | GGGSEIVLTQSPATLSLSPGERATLSCRATSSVSSMNWYQQK |
| | | PGQAPRPLIHATSNLASGIPARFSGSGSGTDYTLTISSLEPEDA |
| | | AVYYCQQWTFNPPTFGQGTKLEIKTTTPAPRPPTPAPTIASQ |
| | | PLSLRPEACRPAAGGAVHTRGLDFACDIYIWAPLAGTCGVL |
| | | LLSLVITLYCKRGRKKLLYIFKQPFMRPVQTTQEEDGCSCRF |
| | | PEEEEGGCELRVKFSRSADAPAYQQGQNQLYNELNLGRREE |
| | | YDVLDKRRGRDPEMGGKPRRKNPQEGLYNELQKDKMAEA |
| | | YSEIGMKGERRRGKGHDGLYQGLSTATKDTYDALHMQALP |
| | | PR |
| CD20-C3H4 | | |
| 950 | HCDR1 | NYNLH |
| | (Kabat) | |
| 951 | HCDR2 | AIYPGNYDTSYNQKFKG |
| | (Kabat) | |
| 952 | HCDR3 | VDFGHSRYWYFDV |
| | (Kabat) | |
| 953 | HCDR1 | GYTFTNY |
| | (Chothia) | |
| 954 | HCDR2 | YPGNYD |
| | (Chothia) | |
| 955 | HCDR3 | VDFGHSRYWYFDV |
| | (Chothia) | |
| 956 | HCDR1 | GYTFTNYN |
| | (IMGT) | |
| 957 | HCDR2 | IYPGNYDT |
| | (IMGT) | |
| 958 | HCDR3 | ARVDFGHSRYWYFDV |
| | (IMGT) | |
| 959 | HCDR1 | GYTFTNYNLH |
| | (Combined) | |
| 960 | HCDR2 | AIYPGNYDTSYNQKFKG |
| | (Combined) | |
| 961 | HCDR3 | VDFGHSRYWYFDV |
| | (Combined) | |
| 962 | VH | QVQLVQSGAEVKKPGSSVKVSCKASGYTFTNYNLHWVRQ |
| | | APGQGLEWMGAIYPGNYDTSYNQKFKGRVTITADKSTSTA |
| | | YMELSSLRSEDTAVYYCARVDFGHSRYWYFDVWGQGTTV |
| | | TVSS |
| 963 | LCDR1 | RATSSVSSMN |
| | (Kabat) | |
| 964 | LCDR2 | ATSNLAS |
| | (Kabat) | |
| 965 | LCDR3 | QQWTFNPPT |
| | (Kabat) | |
| 966 | LCDR1 | TSSVSS |
| | (Chothia) | |
| 967 | LCDR2 | ATS |
| | (Chothia) | |
| 968 | LCDR3 | WTFNPP |
| | (Chothia) | |
| 969 | LCDR1 | SSVSS |
| | (IMGT) | |
| 970 | LCDR2 | ATS |
| | (IMGT) | |
| 971 | LCDR3 | QQWTFNPPT |
| | (IMGT) | |
| 972 | LCDR1 | RATSSVSSMN |
| | (Combined) | |
| 973 | LCDR2 | ATSNLAS |
| | (Combined) | |
| 974 | LCDR3 | QQWTFNPPT |
| | (Combined) | |
| 975 | VL | DIQLTQSPSFLSASVGDRVTITCRATSSVSSMNWYQQKPGK |
| | | APKPLIHATSNLASGVPSRFSGSGSGTEYTLTISSLQPEDFAT |
| | | YYCQQWTFNPPTFGQGTKLEIK |
| 976 | scFv (VH- | QVQLVQSGAEVKKPGSSVKVSCKASGYTFTNYNLHWVRQ |
| | linker-VL) | APGQGLEWMGAIYPGNYDTSYNQKFKGRVTITADKSTSTA |
| | | YMELSSLRSEDTAVYYCARVDFGHSRYWYFDVWGQGTTV |
| | | TVSSGGGGSGGGGSGGGGSGGGGSDIQLTQSPSFLSASVGD |
| | | RVTITCRATSSVSSMNWYQQKPGKAPKPLIHATSNLASGVP |
| | | SRFSGSGSGTEYTLTISSLQPEDFATYYCQQWTFNPPTFGQG |
| | | TKLEIK |
| 977 | Full CAR | MALPVTALLLPLALLLHAARPQVQLVQSGAEVKKPGSSVK |
| | | VSCKASGYTFTNYNLHWVRQAPGQGLEWMGAIYPGNYDT |
| | | SYNQKFKGRVTITADKSTSTAYMELSSLRSEDTAVYYCARV |
| | | DFGHSRYWYFDVWGQGTTVTVSSGGGGSGGGGSGGGGSG |
| | | GGGSDIQLTQSPSFLSASVGDRVTITCRATSSVSSMNWYQQ |
| | | KPGKAPKPLIHATSNLASGVPSRFSGSGSGTEYTLTISSLQPE |
| | | DFATYYCQQWTFNPPTFGQGTKLEIKTTTPAPRPPTPAPTIAS |
| | | QPLSLRPEACRPAAGGAVHTRGLDFACDIYIWAPLAGTCGV |
| | | LLLSLVITLYCKRGRKKLLYIFKQPFMRPVQTTQEEDGCSCR |
| | | FPEEEEGGCELRVKFSRSADAPAYQQGQNQLYNELNLGRRE |
| | | EYDVLDKRRGRDPEMGGKPRRKNPQEGLYNELQKDKMAE |
| | | AYSEIGMKGERRRGKGHDGLYQGLSTATKDTYDALHMQA |
| | | LPPR |
| CD20-C2H1 | | |
| 978 | HCDR1 | NYWMH |
| | (Kabat) | |
| 979 | HCDR2 | FITPTTGYPEYNQKFKD |
| | (Kabat) | |
| 980 | HCDR3 | RKVGKGVYYALDY |
| | (Kabat) | |
| 981 | HCDR1 | GYTFTNY |
| | (Chothia) | |
| 982 | HCDR2 | TPTTGY |
| | (Chothia) | |
| 983 | HCDR3 | RKVGKGVYYALDY |
| | (Chothia) | |
| 984 | HCDR1 | GYTFTNYW |
| | (IMGT) | |
| 985 | HCDR2 | ITPTTGYP |
| | (IMGT) | |
| 986 | HCDR3 | ARRKVGKGVYYALDY |
| | (IMGT) | |
| 987 | HCDR1 | GYTFTNYWMH |
| | (Combined) | |
| 988 | HCDR2 | FITPTTGYPEYNQKFKD |
| | (Combined) | |
| 989 | HCDR3 | RKVGKGVYYALDY |
| | (Combined) | |
| 990 | VH | QVQLVQSGAEVKKPGASVKVSCKASGYTFTNYWMHWVRQ |
| | | APGQGLEWMGFITPTTGYPEYNQKFKDRVTMTADKSTSTA |
| | | YMELSSLRSEDTAVYYCARRKVGKGVYYALDYWGQGTTV |
| | | TVSS |
| 991 | LCDR1 | RASGNIHNYLA |
| | (Kabat) | |
| 992 | LCDR2 | NTKTLAD |
| | (Kabat) | |
| 993 | LCDR3 | QHFWSSPWT |
| | (Kabat) | |
| 994 | LCDR1 | SGNIHNY |
| | (Chothia) | |
| 995 | LCDR2 | NTK |
| | (Chothia) | |
| 996 | LCDR3 | FWSSPW |
| | (Chothia) | |
| 997 | LCDR1 | GNIHNY |
| | (IMGT) | |
| 998 | LCDR2 | NTK |
| | (IMGT) | |
| 999 | LCDR3 | QHFWSSPWT |
| | (IMGT) | |
| 1000 | LCDR1 | RASGNIHNYLA |
| | (Combined) | |
| 1001 | LCDR2 | NTKTLAD |
| | (Combined) | |
| 1002 | LCDR3 | QHFWSSPWT |
| | (Combined) | |
| 1003 | VL | DIQMTQSPSSLSASVGDRVTITCRASGNIHNYLAWYQQKPG |
| | | KVPKLLIYNTKTLADGVPSRFSGSGSGTDYTLTISSLQPEDV |
| | | ATYYCQHFWSSPWTFGGGTKVEIK |
| 1004 | scFv (VH- | QVQLVQSGAEVKKPGASVKVSCKASGYTFTNYWMHWVRQ |
| | linker-VL) | APGQGLEWMGFITPTTGYPEYNQKFKDRVTMTADKSTSTA |
| | | YMELSSLRSEDTAVYYCARRKVGKGVYYALDYWGQGTTV |
| | | TVSSGGGGSGGGGSGGGGSGGGGSDIQMTQSPSSLSASVGD |
| | | RVTITCRASGNIHNYLAWYQQKPGKVPKLLIYNTKTLADGV |
| | | PSRFSGSGSGTDYTLTISSLQPEDVATYYCQHFWSSPWTFGG |
| | | GTKVEIK |
| 1005 | Full CAR | MALPVTALLLPLALLLHAARPQVQLVQSGAEVKKPGASVK |
| | | VSCKASGYTFTNYWMHWVRQAPGQGLEWMGFITPTTGYP |
| | | EYNQKFKDRVTMTADKSTSTAYMELSSLRSEDTAVYYCAR |
| | | RKVGKGVYYALDYWGQGTTVTVSSGGGGSGGGGSGGGGS |
| | | GGGGSDIQMTQSPSSLSASVGDRVTITCRASGNIHNYLAWY |
| | | QQKPGKVPKLLIYNTKTLADGVPSRFSGSGSGTDYTLTISSL |
| | | QPEDVATYYCQHFWSSPWTFGGGTKVEIKTTTPAPRPPTPA |
| | | PTIASQPLSLRPEACRPAAGGAVHTRGLDFACDIYIWAPLAG |
| | | TCGVLLLSLVITLYCKRGRKKLLYIFKQPFMRPVQTTQEEDG |
| | | CSCRFPEEEEGGCELRVKFSRSADAPAYQQGQNQLYNELNL |
| | | GRREEYDVLDKRRGRDPEMGGKPRRKNPQEGLYNELQKD |
| | | KMAEAYSEIGMKGERRRGKGHDGLYQGLSTATKDTYDAL |
| | | HMQALPPR |
| CD20-C2H2 | | |
| 1006 | HCDR1 | NYWMH |
| | (Kabat) | |
| 1007 | HCDR2 | FITPTTGYPEYNQKFKD |
| | (Kabat) | |
| 1008 | HCDR3 | RKVGKGVYYALDY |
| | (Kabat) | |
| 1009 | HCDR1 | GYTFTNY |
| | (Chothia) | |
| 1010 | HCDR2 | TPTTGY |
| | (Chothia) | |
| 1011 | HCDR3 | RKVGKGVYYALDY |
| | (Chothia) | |
| 1012 | HCDR1 | GYTFTNYW |
| | (IMGT) | |
| 1013 | HCDR2 | ITPTTGYP |
| | (IMGT) | |
| 1014 | HCDR3 | ARRKVGKGVYYALDY |
| | (IMGT) | |
| 1015 | HCDR1 | GYTFTNYWMH |
| | (Combined) | |
| 1016 | HCDR2 | FITPTTGYPEYNQKFKD |
| | (Combined) | |
| 1017 | HCDR3 | RKVGKGVYYALDY |
| | (Combined) | |
| 1018 | VH | QVQLVQSGAEVKKPGSSVKVSCKASGYTFTNYWMHWVRQ |
| | | APGQGLEWMGFITPTTGYPEYNQKFKDRVTITADKSTSTAY |
| | | MELSSLRSEDTAVYYCARRKVGKGVYYALDYWGQGTTVT |
| | | VSS |
| 1019 | LCDR1 | RASGNIHNYLA |
| | (Kabat) | |
| 1020 | LCDR2 | NTKTLAD |
| | (Kabat) | |
| 1021 | LCDR3 | QHFWSSPWT |
| | (Kabat) | |
| 1022 | LCDR1 | SGNIHNY |
| | (Chothia) | |
| 1023 | LCDR2 | NTK |
| | (Chothia) | |
| 1024 | LCDR3 | FWSSPW |
| | (Chothia) | |
| 1025 | LCDR1 | GNIHNY |
| | (IMGT) | |
| 1026 | LCDR2 | NTK |
| | (IMGT) | |
| 1027 | LCDR3 | QHFWSSPWT |
| | (IMGT) | |
| 1028 | LCDR1 | RASGNIHNYLA |
| | (Combined) | |
| 1029 | LCDR2 | NTKTLAD |
| | (Combined) | |
| 1030 | LCDR3 | QHFWSSPWT |
| | (Combined) | |
| 1031 | VL | DIQMTQSPSSLSASVGDRVTITCRASGNIHNYLAWYQQKPG |
| | | KVPKLLIYNTKTLADGVPSRFSGSGSGTDYTLTISSLQPEDV |
| | | ATYYCQHFWSSPWTFGGGTKVEIK |
| 1032 | scFv (VH- | QVQLVQSGAEVKKPGSSVKVSCKASGYTFTNYWMHWVRQ |
| | linker-VL) | APGQGLEWMGFITPTTGYPEYNQKFKDRVTITADKSTSTAY |
| | | MELSSLRSEDTAVYYCARRKVGKGVYYALDYWGQGTTVT |
| | | VSSGGGGSGGGGSGGGGSGGGGSDIQMTQSPSSLSASVGDR |
| | | VTITCRASGNIHNYLAWYQQKPGKVPKLLIYNTKTLADGVP |
| | | SRFSGSGSGTDYTLTISSLQPEDVATYYCQHFWSSPWTFGGG |
| | | TKVEIK |
| 1033 | Full CAR | MALPVTALLLPLALLLHAARPQVQLVQSGAEVKKPGSSVK |
| | | VSCKASGYTFTNYWMHWVRQAPGQGLEWMGFITPTTGYP |
| | | EYNQKFKDRVTITADKSTSTAYMELSSLRSEDTAVYYCARR |
| | | KVGKGVYYALDYWGQGTTVTVSSGGGGSGGGGSGGGGSG |
| | | GGGSDIQMTQSPSSLSASVGDRVTITCRASGNIHNYLAWYQ |
| | | QKPGKVPKLLIYNTKTLADGVPSRFSGSGSGTDYTLTISSLQ |
| | | PEDVATYYCQHFWSSPWTFGGGTKVEIKTTTPAPRPPTPAPT |
| | | IASQPLSLRPEACRPAAGGAVHTRGLDFACDIYIWAPLAGTC |
| | | GVLLLSLVITLYCKRGRKKLLYIFKQPFMRPVQTTQEEDGCS |
| | | CRFPEEEEGGCELRVKFSRSADAPAYQQGQNQLYNELNLGR |
| | | REEYDVLDKRRGRDPEMGGKPRRKNPQEGLYNELQKDKM |
| | | AEAYSEIGMKGERRRGKGHDGLYQGLSTATKDTYDALHM |
| | | QALPPR |
| CD20-C2H3 | | |
| 1034 | HCDR1 | NYWMH |
| | (Kabat) | |
| 1035 | HCDR2 | FITPTTGYPEYNQKFKD |
| | (Kabat) | |
| 1036 | HCDR3 | RKVGKGVYYALDY |
| | (Kabat) | |
| 1037 | HCDR1 | GYTFTNY |
| | (Chothia) | |
| 1038 | HCDR2 | TPTTGY |
| | (Chothia) | |
| 1039 | HCDR3 | RKVGKGVYYALDY |
| | (Chothia) | |
| 1040 | HCDR1 | GYTFTNYW |
| | (IMGT) | |
| 1041 | HCDR2 | ITPTTGYP |
| | (IMGT) | |
| 1042 | HCDR3 | ARRKVGKGVYYALDY |
| | (IMGT) | |
| 1043 | HCDR1 | GYTFTNYWMH |
| | (Combined) | |
| 1044 | HCDR2 | FITPTTGYPEYNQKFKD |
| | (Combined) | |
| 1045 | HCDR3 | RKVGKGVYYALDY |
| | (Combined) | |
| 1046 | VH | QVQLVQSGAEVKKPGASVKVSCKASGYTFTNYWMHWVRQ |
| | | APGQGLEWMGFITPTTGYPEYNQKFKDRVTMTADKSTSTA |
| | | YMELSSLRSEDTAVYYCARRKVGKGVYYALDYWGQGTTV |
| | | TVSS |
| 1047 | LCDR1 | RASGNIHNYLA |
| | (Kabat) | |
| 1048 | LCDR2 | NTKTLAD |
| | (Kabat) | |
| 1049 | LCDR3 | QHFWSSPWT |
| | (Kabat) | |
| 1050 | LCDR1 | SGNIHNY |
| | (Chothia) | |
| 1051 | LCDR2 | NTK |
| | (Chothia) | |
| 1052 | LCDR3 | FWSSPW |
| | (Chothia) | |
| 1053 | LCDR1 | GNIHNY |
| | (IMGT) | |
| 1054 | LCDR2 | NTK |
| | (IMGT) | |
| 1055 | LCDR3 | QHFWSSPWT |
| | (IMGT) | |
| 1056 | LCDR1 | RASGNIHNYLA |
| | (Combined) | |
| 1057 | LCDR2 | NTKTLAD |
| | (Combined) | |
| 1058 | LCDR3 | QHFWSSPWT |
| | (Combined) | |
| 1059 | VL | AIRMTQSPFSLSASVGDRVTITCRASGNIHNYLAWYQQKPA |
| | | KAPKLFIYNTKTLADGVPSRFSGSGSGTDYTLTISSLQPEDFA |
| | | TYYCQHFWSSPWTFGGGTKVEIK |
| 1060 | scFv (VH- | QVQLVQSGAEVKKPGASVKVSCKASGYTFTNYWMHWVRQ |
| | linker-VL) | APGQGLEWMGFITPTTGYPEYNQKFKDRVTMTADKSTSTA |
| | | YMELSSLRSEDTAVYYCARRKVGKGVYYALDYWGQGTTV |
| | | TVSSGGGGSGGGGSGGGGSGGGGSAIRMTQSPFSLSASVGD |
| | | RVTITCRASGNIHNYLAWYQQKPAKAPKLFIYNTKTLADGV |
| | | PSRFSGSGSGTDYTLTISSLQPEDFATYYCQHFWSSPWTFGG |
| | | GTKVEIK |
| 1061 | Full CAR | MALPVTALLLPLALLLHAARPQVQLVQSGAEVKKPGASVK |
| | | VSCKASGYTFTNYWMHWVRQAPGQGLEWMGFITPTTGYP |
| | | EYNQKFKDRVTMTADKSTSTAYMELSSLRSEDTAVYYCAR |
| | | RKVGKGVYYALDYWGQGTTVTVSSGGGGSGGGGSGGGGS |
| | | GGGGSAIRMTQSPFSLSASVGDRVTITCRASGNIHNYLAWY |
| | | QQKPAKAPKLFIYNTKTLADGVPSRFSGSGSGTDYTLTISSL |
| | | QPEDFATYYCQHFWSSPWTFGGGTKVEIKTTTPAPRPPTPAP |
| | | TIASQPLSLRPEACRPAAGGAVHTRGLDFACDIYIWAPLAGT |
| | | CGVLLLSLVITLYCKRGRKKLLYIFKQPFMRPVQTTQEEDGC |
| | | SCRFPEEEEGGCELRVKFSRSADAPAYQQGQNQLYNELNLG |
| | | RREEYDVLDKRRGRDPEMGGKPRRKNPQEGLYNELQKDK |
| | | MAEAYSEIGMKGERRRGKGHDGLYQGLSTATKDTYDALH |
| | | MQALPPR |
| CD20-C2H4 | | |
| 1062 | HCDR1 | NYWMH |
| | (Kabat) | |
| 1063 | HCDR2 | FITPTTGYPEYNQKFKD |
| | (Kabat) | |
| 1064 | HCDR3 | RKVGKGVYYALDY |
| | (Kabat) | |
| 1065 | HCDR1 | GYTFTNY |
| | (Chothia) | |
| 1066 | HCDR2 | TPTTGY |
| | (Chothia) | |
| 1067 | HCDR3 | RKVGKGVYYALDY |
| | (Chothia) | |
| 1068 | HCDR1 | GYTFTNYW |
| | (IMGT) | |
| 1069 | HCDR2 | ITPTTGYP |
| | (IMGT) | |
| 1070 | HCDR3 | ARRKVGKGVYYALDY |
| | (IMGT) | |
| 1071 | HCDR1 | GYTFTNYWMH |
| | (Combined) | |
| 1072 | HCDR2 | FITPTTGYPEYNQKFKD |
| | (Combined) | |
| 1073 | HCDR3 | RKVGKGVYYALDY |
| | (Combined) | |
| 1074 | VH | QVQLVQSGAEVKKPGSSVKVSCKASGYTFTNYWMHWVRQ |
| | | APGQGLEWMGFITPTTGYPEYNQKFKDRVTITADKSTSTAY |
| | | MELSSLRSEDTAVYYCARRKVGKGVYYALDYWGQGTTVT |
| | | VSS |
| 1075 | LCDR1 | RASGNIHNYLA |
| | (Kabat) | |
| 1076 | LCDR2 | NTKTLAD |
| | (Kabat) | |
| 1077 | LCDR3 | QHFWSSPWT |
| | (Kabat) | |
| 1078 | LCDR1 | SGNIHNY |
| | (Chothia) | |
| 1079 | LCDR2 | NTK |
| | (Chothia) | |
| 1080 | LCDR3 | FWSSPW |
| | (Chothia) | |
| 1081 | LCDR1 | GNIHNY |
| | (IMGT) | |
| 1082 | LCDR2 | NTK |
| | (IMGT) | |
| 1083 | LCDR3 | QHFWSSPWT |
| | (IMGT) | |
| 1084 | LCDR1 | RASGNIHNYLA |
| | (Combined) | |
| 1085 | LCDR2 | NTKTLAD |
| | (Combined) | |
| 1086 | LCDR3 | QHFWSSPWT |
| | (Combined) | |
| 1087 | VL | AIRMTQSPFSLSASVGDRVTITCRASGNIHNYLAWYQQKPA |
| | | KAPKLFIYNTKTLADGVPSRFSGSGSGTDYTLTISSLQPEDFA |
| | | TYYCQHFWSSPWTFGGGTKVEIK |
| 1088 | scFv (VH- | QVQLVQSGAEVKKPGSSVKVSCKASGYTFTNYWMHWVRQ |
| | linker-VL) | APGQGLEWMGFITPTTGYPEYNQKFKDRVTITADKSTSTAY |
| | | MELSSLRSEDTAVYYCARRKVGKGVYYALDYWGQGTTVT |
| | | VSSGGGGSGGGGSGGGGSGGGGSAIRMTQSPFSLSASVGDR |
| | | VTITCRASGNIHNYLAWYQQKPAKAPKLFIYNTKTLADGVP |
| | | SRFSGSGSGTDYTLTISSLQPEDFATYYCQHFWSSPWTFGGG |
| | | TKVEIK |
| 1089 | Full CAR | MALPVTALLLPLALLLHAARPQVQLVQSGAEVKKPGSSVK |
| | | VSCKASGYTFTNYWMHWVRQAPGQGLEWMGFITPTTGYP |
| | | EYNQKFKDRVTITADKSTSTAYMELSSLRSEDTAVYYCARR |
| | | KVGKGVYYALDYWGQGTTVTVSSGGGGSGGGGSGGGGSG |
| | | GGGSAIRMTQSPFSLSASVGDRVTITCRASGNIHNYLAWYQ |
| | | QKPAKAPKLFIYNTKTLADGVPSRFSGSGSGTDYTLTISSLQP |
| | | EDFATYYCQHFWSSPWTFGGGTKVEIKTTTPAPRPPTPAPTI |
| | | ASQPLSLRPEACRPAAGGAVHTRGLDFACDIYIWAPLAGTC |
| | | GVLLLSLVITLYCKRGRKKLLYIFKQPFMRPVQTTQEEDGCS |
| | | CRFPEEEEGGCELRVKFSRSADAPAYQQGQNQLYNELNLGR |
| | | REEYDVLDKRRGRDPEMGGKPRRKNPQEGLYNELQKDKM |
| | | AEAYSEIGMKGERRRGKGHDGLYQGLSTATKDTYDALHM |
| | | QALPPR |
| CD20-C5H2 | | |
| 1090 | HCDR1 | SYNMH |
| | (Kabat) | |
| 1091 | HCDR2 | AIYPGNGDTSYNPKFKG |
| | (Kabat) | |
| 1092 | HCDR3 | SYFYGSSSWYFDV |
| | (Kabat) | |
| 1093 | HCDR1 | GYTFTSY |
| | (Chothia) | |
| 1094 | HCDR2 | YPGNGD |
| | (Chothia) | |
| 1095 | HCDR3 | SYFYGSSSWYFDV |
| | (Chothia) | |
| 1096 | HCDR1 | GYTFTSYN |
| | (IMGT) | |
| 1097 | HCDR2 | IYPGNGDT |
| | (IMGT) | |
| 1098 | HCDR3 | ARSYFYGSSSWYFDV |
| | (IMGT) | |
| 1099 | HCDR1 | GYTFTSYNMH |
| | (Combined) | |
| 1100 | HCDR2 | AIYPGNGDTSYNPKFKG |
| | (Combined) | |
| 1101 | HCDR3 | SYFYGSSSWYFDV |
| | (Combined) | |
| 1102 | VH | QVQLVQSGAEVKKPGASVKVSCKASGYTFTSYNMHWVRQ |
| | | APGQGLEWMGAIYPGNGDTSYNPKFKGRVTMTADKSTRTA |
| | | YMELSSLRSEDTAVYYCARSYFYGSSSWYFDVWGQGTTVT |
| | | VSS |
| 1103 | LCDR1 | RASSSVSSMH |
| | (Kabat) | |
| 1104 | LCDR2 | ATSNLAS |
| | (Kabat) | |
| 1105 | LCDR3 | QQWIFNPPT |
| | (Kabat) | |
| 1106 | LCDR1 | SSSVSS |
| | (Chothia) | |
| 1107 | LCDR2 | ATS |
| | (Chothia) | |
| 1108 | LCDR3 | WIFNPP |
| | (Chothia) | |
| 1109 | LCDR1 | SSVSS |
| | (IMGT) | |
| 1110 | LCDR2 | ATS |
| | (IMGT) | |
| 1111 | LCDR3 | QQWIFNPPT |
| | (IMGT) | |
| 1112 | LCDR1 | RASSSVSSMH |
| | (Combined) | |
| 1113 | LCDR2 | ATSNLAS |
| | (Combined) | |
| 1114 | LCDR3 | QQWIFNPPT |
| | (Combined) | |
| 1115 | VL | DIQLTQSPSFLSASVGDRVTITCRASSSVSSMHWYQQKPGKA |
| | | PKPLIFATSNLASGVPSRFSGSGSGTEYTLTISSLQPEDFATYY |
| | | CQQWIFNPPTFGGGTKVEIK |
| 1116 | scFv (VH- | QVQLVQSGAEVKKPGASVKVSCKASGYTFTSYNMHWVRQ |
| | linker-VL) | APGQGLEWMGAIYPGNGDTSYNPKFKGRVTMTADKSTRTA |
| | | YMELSSLRSEDTAVYYCARSYFYGSSSWYFDVWGQGTTVT |
| | | VSSGGGGGGGGSGGGGSGGGGSDIQLTQSPSFLSASVGDR |
| | | VTITCRASSSVSSMHWYQQKPGKAPKPLIFATSNLASGVPSR |
| | | FSGSGSGTEYTLTISSLQPEDFATYYCQQWIFNPPTFGGGTK |
| | | VEIK |
| 1117 | Full CAR | MALPVTALLLPLALLLHAARPQVQLVQSGAEVKKPGASVK |
| | | VSCKASGYTFTSYNMHWVRQAPGQGLEWMGAIYPGNGDT |
| | | SYNPKFKGRVTMTADKSTRTAYMELSSLRSEDTAVYYCAR |
| | | SYFYGSSSWYFDVWGQGTTVTVSSGGGGSGGGGSGGGGSG |
| | | GGGSDIQLTQSPSFLSASVGDRVTITCRASSSVSSMHWYQQK |
| | | PGKAPKPLIFATSNLASGVPSRFSGSGSGTEYTLTISSLQPEDF |
| | | ATYYCQQWIFNPPTFGGGTKVEIKTTTPAPRPPTPAPTIASQP |
| | | LSLRPEACRPAAGGAVHTRGLDFACDIYIWAPLAGTCGVLL |
| | | LSLVITLYCKRGRKKLLYIFKQPFMRPVQTTQEEDGCSCRFP |
| | | EEEEGGCELRVKFSRSADAPAYQQGQNQLYNELNLGRREE |
| | | YDVLDKRRGRDPEMGGKPRRKNPQEGLYNELQKDKMAEA |
| | | YSEIGMKGERRRGKGHDGLYQGLSTATKDTYDALHMQALP |
| | | PR |
| CD20-C5H3 | | |
| 1118 | HCDR1 | SYNMH |
| | (Kabat) | |
| 1119 | HCDR2 | AIYPGNGDTSYNPKFKG |
| | (Kabat) | |
| 1120 | HCDR3 | SYFYGSSSWYFDV |
| | (Kabat) | |
| 1121 | HCDR1 | GYTFTSY |
| | (Chothia) | |
| 1122 | HCDR2 | YPGNGD |
| | (Chothia) | |
| 1123 | HCDR3 | SYFYGSSSWYFDV |
| | (Chothia) | |
| 1124 | HCDR1 | GYTFTSYN |
| | (IMGT) | |
| 1125 | HCDR2 | IYPGNGDT |
| | (IMGT) | |
| 1126 | HCDR3 | ARSYFYGSSSWYFDV |
| | (IMGT) | |
| 1127 | HCDR1 | GYTFTSYNMH |
| | (Combined) | |
| 1128 | HCDR2 | AIYPGNGDTSYNPKFKG |
| | (Combined) | |
| 1129 | HCDR3 | SYFYGSSSWYFDV |
| | (Combined) | |
| 1130 | VH | QVQLVQSGAEVKKPGSSVKVSCKASGYTFTSYNMHWVRQ |
| | | APGQGLEWMGAIYPGNGDTSYNPKFKGRVTITADKSTRTA |
| | | YMELSSLRSEDTAVYYCARSYFYGSSSWYFDVWGQGTTVT |
| | | VSS |
| 1131 | LCDR1 | RASSSVSSMH |
| | (Kabat) | |
| 1132 | LCDR2 | ATSNLAS |
| | (Kabat) | |
| 1133 | LCDR3 | QQWIFNPPT |
| | (Kabat) | |
| 1134 | LCDR1 | SSSVSS |
| | (Chothia) | |
| 1135 | LCDR2 | ATS |
| | (Chothia) | |
| 1136 | LCDR3 | WIFNPP |
| | (Chothia) | |
| 1137 | LCDR1 | SSVSS |
| | (IMGT) | |
| 1138 | LCDR2 | ATS |
| | (IMGT) | |
| 1139 | LCDR3 | QQWIFNPPT |
| | (IMGT) | |
| 1140 | LCDR1 | RASSSVSSMH |
| | (Combined) | |
| 1141 | LCDR2 | ATSNLAS |
| | (Combined) | |
| 1142 | LCDR3 | QQWIFNPPT |
| | (Combined) | |
| 1143 | VL | EIVLTQSPATLSLSPGERATLSCRASSSVSSMHWYQQKPGQA |
| | | PRPLIFATSNLASGIPARFSGSGSGTDYTLTISSLEPEDAAVYY |
| | | CQQWIFNPPTFGGGTKVEIK |
| 1144 | scFv (VH- | QVQLVQSGAEVKKPGSSVKVSCKASGYTFTSYNMHWVRQ |
| | linker-VL) | APGQGLEWMGAIYPGNGDTSYNPKFKGRVTITADKSTRTA |
| | | YMELSSLRSEDTAVYYCARSYFYGSSSWYFDVWGQGTTVT |
| | | VSSGGGGSGGGGSGGGGSGGGGSEIVLTQSPATLSLSPGER |
| | | ATLSCRASSSVSSMHWYQQKPGQAPRPLIFATSNLASGIPAR |
| | | FSGSGSGTDYTLTISSLEPEDAAVYYCQQWIFNPPTFGGGTK |
| | | VEIK |
| 1145 | Full CAR | MALPVTALLLPLALLLHAARPQVQLVQSGAEVKKPGSSVK |
| | | VSCKASGYTFTSYNMHWVRQAPGQGLEWMGAIYPGNGDT |
| | | SYNPKFKGRVTITADKSTRTAYMELSSLRSEDTAVYYCARS |
| | | YFYGSSSWYFDVWGQGTTVTVSSGGGGSGGGGSGGGGSG |
| | | GGGSEIVLTQSPATLSLSPGERATLSCRASSSVSSMHWYQQK |
| | | PGQAPRPLIFATSNLASGIPARFSGSGSGTDYTLTISSLEPEDA |
| | | AVYYCQQWIFNPPTFGGGTKVEIKTTTPAPRPPTPAPTIASQP |
| | | LSLRPEACRPAAGGAVHTRGLDFACDIYIWAPLAGTCGVLL |
| | | LSLVITLYCKRGRKKLLYIFKQPFMRPVQTTQEEDGCSCRFP |
| | | EEEEGGCELRVKFSRSADAPAYQQGQNQLYNELNLGRREE |
| | | YDVLDKRRGRDPEMGGKPRRKNPQEGLYNELQKDKMAEA |
| | | YSEIGMKGERRRGKGHDGLYQGLSTATKDTYDALHMQALP |
| | | PR |
| CD20-C5H4 | | |
| 1146 | HCDR1 | SYNMH |
| | (Kabat) | |
| 1147 | HCDR2 | AIYPGNGDTSYNPKFKG |
| | (Kabat) | |
| 1148 | HCDR3 | SYFYGSSSWYFDV |
| | (Kabat) | |
| 1149 | HCDR1 | GYTFTSY |
| | (Chothia) | |
| 1150 | HCDR2 | YPGNGD |
| | (Chothia) | |
| 1151 | HCDR3 | SYFYGSSSWYFDV |
| | (Chothia) | |
| 1152 | HCDR1 | GYTFTSYN |
| | (IMGT) | |
| 1153 | HCDR2 | IYPGNGDT |
| | (IMGT) | |
| 1154 | HCDR3 | ARSYFYGSSSWYFDV |
| | (IMGT) | |
| 1155 | HCDR1 | GYTFTSYNMH |
| | (Combined) | |
| 1156 | HCDR2 | AIYPGNGDTSYNPKFKG |
| | (Combined) | |
| 1157 | HCDR3 | SYFYGSSSWYFDV |
| | (Combined) | |
| 1158 | VH | QVQLVQSGAEVKKPGSSVKVSCKASGYTFTSYNMHWVRQ |
| | | APGQGLEWMGAIYPGNGDTSYNPKFKGRVTITADKSTRTA |
| | | YMELSSLRSEDTAVYYCARSYFYGSSSWYFDVWGQGTTVT |
| | | VSS |
| 1159 | LCDR1 | RASSSVSSMH |
| | (Kabat) | |
| 1160 | LCDR2 | ATSNLAS |
| | (Kabat) | |
| 1161 | LCDR3 | QQWIFNPPT |
| | (Kabat) | |
| 1162 | LCDR1 | SSSVSS |
| | (Chothia) | |
| 1163 | LCDR2 | ATS |
| | (Chothia) | |
| 1164 | LCDR3 | WIFNPP |
| | (Chothia) | |
| 1165 | LCDR1 | SSVSS |
| | (IMGT) | |
| 1166 | LCDR2 | ATS |
| | (IMGT) | |
| 1167 | LCDR3 | QQWIFNPPT |
| | (IMGT) | |
| 1168 | LCDR1 | RASSSVSSMH |
| | (Combined) | |
| 1169 | LCDR2 | ATSNLAS |
| | (Combined) | |
| 1170 | LCDR3 | QQWIFNPPT |
| | (Combined) | |
| 1171 | VL | DIQLTQSPSFLSASVGDRVTITCRASSSVSSMHWYQQKPGKA |
| | | PKPLIFATSNLASGVPSRFSGSGSGTEYTLTISSLQPEDFATYY |
| | | CQQWIFNPPTFGGGTKVEIK |
| 1172 | scFv (VH- | QVQLVQSGAEVKKPGSSVKVSCKASGYTFTSYNMHWVRQ |
| | linker-VL) | APGQGLEWMGAIYPGNGDTSYNPKFKGRVTITADKSTRTA |
| | | YMELSSLRSEDTAVYYCARSYFYGSSSWYFDVWGQGTTVT |
| | | VSSGGGGSGGGGSGGGGSGGGGSDIQLTQSPSFLSASVGDR |
| | | VTITCRASSSVSSMHWYQQKPGKAPKPLIFATSNLASGVPSR |
| | | FSGSGSGTEYTLTISSLQPEDFATYYCQQWIFNPPTFGGGTK |
| | | VEIK |
| 1173 | Full CAR | MALPVTALLLPLALLLHAARPQVQLVQSGAEVKKPGSSVK |
| | | VSCKASGYTFTSYNMHWVRQAPGQGLEWMGAIYPGNGDT |
| | | SYNPKFKGRVTITADKSTRTAYMELSSLRSEDTAVYYCARS |
| | | YFYGSSSWYFDVWGQGTTVTVSSGGGGSGGGGSGGGGSG |
| | | GGGSDIQLTQSPSFLSASVGDRVTITCRASSSVSSMHWYQQK |
| | | PGKAPKPLIFATSNLASGVPSRFSGSGSGTEYTLTISSLQPEDF |
| | | ATYYCQQWIFNPPTFGGGTKVEIKTTTPAPRPPTPAPTIASQP |
| | | LSLRPEACRPAAGGAVHTRGLDFACDIYIWAPLAGTCGVLL |
| | | LSLVITLYCKRGRKKLLYIFKQPFMRPVQTTQEEDGCSCRFP |
| | | EEEEGGCELRVKFSRSADAPAYQQGQNQLYNELNLGRREE |
| | | YDVLDKRRGRDPEMGGKPRRKNPQEGLYNELQKDKMAEA |
| | | YSEIGMKGERRRGKGHDGLYQGLSTATKDTYDALHMQALP |
| | | PR |
| CD20-C3 | | |
| 1174 | VH | QVQLQQPGAELVKPGASVKMSCKASGYTFTNYNLHWVKQ |
| | | TPGQGLEWIGAIYPGNYDTSYNQKFKGKATLTADKSSSTAY |
| | | MLLSSLTSEDSAVYFCARVDFGHSRYWYFDVWGAGTTVTV |
| | | SS |
| 1175 | VL | QIVLSQSPAILSASPGEKVTMTCRATSSVSSMNWYQQKPGSF |
| | | PRPWIHATSNLASGVPARFSGSGSGTSYSLTISRVEAEDAAT |
| | | YYCQQWTFNPPTFGAGAKLELK |
| CD20-C5 | | |
| 1176 | VH | QVQLQQPGAELVKPGASVKMSCKASGYTFTSYNMHWVKQ |
| | | TPGQGLEWIGAIYPGNGDTSYNPKFKGKATLTADKSSRTAY |
| | | IHLSSLTSEDSVVYYCARSYFYGSSSWYFDVWGAGTTVTVS |
| | | S |
| 1177 | VL | QIILSQSPAILSASPGEKVTLTCRASSSVSSMHWYQQKPGSSP |
| | | KPWIFATSNLASGVPARFTGSGSGTSYSLTISRVEAEDAATY |
| | | YCQQWIFNPPTFGGGTSLEIK |
| CD20-C2 | | |
| 1178 | VH | QVHLQQSGAELAKPGASVKMSCKASGYTFTNYWMHWVK |
| | | QRPGQGLEWIGFITPTTGYPEYNQKFKDKATLTADKSSSTA |
| | | YMQLSSLTSEDSAVYYCARRKVGKGVYYALDYWGQGTSV |
| | | TVSS |
| 1179 | VL | DILMTQSPASLSASVGETVTITCRASGNIHNYLAWYQQKQG |
| | | NSPQLLVYNTKTLADGVPSRFSGSGSGTQYSLKINSLQTEDF |
| | | GTYYCQHFWSSPWTFGGGTKLEIK |

CARs that bind to CD22 are known in the art. For example, those disclosed in WO2018/067992 or WO2016/164731 can be used in accordance with the present disclosure. Any known CD22 CAR, for example, the CD22 antigen binding domain of any known CD22 CAR, in the art can be used in accordance with the present disclosure.

Exemplary CD22-binding sequences or CD22 CAR sequences are disclosed in, for example, Tables 6A, 6B, 7A, 7B, 7C, 8A, 8B, 9A, 9B, 10A, and 10B of WO2016164731 and Tables 6-10 of WO2018067992. In some embodiments, the CD22 CAR sequences comprise a CDR, variable region, scFv or full-length sequence of a CD22 CAR disclosed in WO2018067992 or WO2016164731.

In embodiments, the CAR comprises an antigen binding domain that binds to CD22 (CD22 CAR). In some embodiments, the antigen binding domain targets human CD22. In some embodiments, the antigen binding domain includes a single chain Fv sequence as described herein.

The sequences of human CD22 CAR are provided below. In some embodiments, a human CD22 CAR is CAR22-65.

Human CD22 CAR scFv sequence
(SEQ ID NO: 753)
EVQLQQSGPGLVKPSQTLSLTCAISGDSMLSNSDTWNWIRQSPSR
GLEWLGRTYHRSTWYDDYASSVRGRVSINVDTSKNQYSLQLNAVT
PEDTGVYYCARVRLQDGNSWSDAFDVWGQGTMVTVSSGGGGSGGG
GGGGGSQSALTQPASASGSPGQSVTISCTGTSSDVGGYNYVSWYQ
QHPGKAPKLMIYDVSNRPSGVSNRFSGSKSGNTASLTISGLQAED
EADYYCSSYTSSSTLYVFGTGTQLTVL
Human CD22 CAR heavy chain variable region
(SEQ ID NO: 754)
EVQLQQSGPGLVKPSQTLSLTCAISGDSMLSNSDTWNWIRQSPSR
GLEWLGRTYHRSTWYDDYASSVRGRVSINVDTSKNQYSLQLNAVT
PEDTGVYYCARVRLQDGNSWSDAFDVWGQGTMVTVSS
Human CD22 CAR light chain variable region
(SEQ ID NO 755)
QSALTQPASASGSPGQSVTISCTGTSSDVGGYNYVSWYQQHPGKA
PKLMIYDVSNRPSGVSNRFSGSKSGNTASLTISGLQAEDEADYYC
SSYTSSSTLYVFGTGTQLTVL

In some embodiments, CD22 CARs comprise a sequence, for example, a CDR, VH, VL, scFv, or full-CAR sequence, disclosed in Tables 15-16 and Table 24 below, or a sequence having at least 70%, 75%, 80%, 85%, 90%, 95%, 96%, 97%, 98%, or 99% identity thereto.

TABLE 15
Heavy Chain Variable Domain CDRs
of CD22 CAR (CAR22-65)

| | | SEQ | | SEQ | | SEQ |
|---|---|---|---|---|---|---|
| | | ID | | ID | | ID |
| Candidate | HCDR1 | NO: | HCDR2 | NO: | HCDR3 | NO: |
| CAR22-65 | GDSML | 744 | RTYHR | 746 | VRLQD | 748 |
| Combined | SNSDT | | STWYD | | GNSWS | |
| | WN | | DYASS | | DAFDV | |
| | | | VRG | | | |
| CAR22-65 | SNSDT | 745 | RTYHR | 747 | VRLQD | 749 |
| Kabat | WN | | STWYD | | GNSWS | |
| | | | DYASS | | DAFDV | |
| | | | VRG | | | |
| CD22-65 | GDSML | 1180 | YHRST | 1181 | VRLQD | 1182 |
| Chothia | SNSD | | WY | | GNSWS | |
| | | | | | DAFDV | |
| CD22-65 | GDSML | 1183 | TYHRS | 1184 | ARVRL | 1185 |
| IMGT | SNSDT | | TWYD | | QDGNS | |
| | | | | | WSDAF | |
| | | | | | DV | |

TABLE 16
Light Chain Variable Domain CDRs
of CD22 CAR (CAR22-65).
The LC CDR sequences in this
table have the same sequence
under the Kabat or combined
definitions.

| | | SEQ | | SEQ | | SEQ |
|---|---|---|---|---|---|---|
| | | ID | | ID | | ID |
| Candidate | LCDR1 | NO: | LCDR2 | NO: | LCDR3 | NO: |
| CAR22-65 | TGTSS | 750 | DVSNR | 751 | SSYTS | 752 |
| Combined | DVGGY | | PS | | SSTLY | |
| | NYVS | | | | V | |
| CD22-65 | TGTSS | 1186 | DVSNR | 1189 | SSYTS | 1192 |
| Kabat | DVGGY | | PS | | SSTLY | |
| | NYVS | | | | V | |
| CD22-65 | TSSDV | 1187 | DVS | 1190 | YTSSS | 1193 |
| Chothia | GGYNY | | | | TLY | |
| CD22-65 | SSDVG | 1188 | DVS | 1191 | SSYTS | 1194 |
| IMGT | GYNY | | | | SSTLY | |
| | | | | | V | |

Table 24
Amino acid sequences of exemplary
anti-CD22 molecules

| SEQ ID | | |
|---|---|---|
| NO: | Region | Sequence |
| CD22-65s | | |
| 1195 | scFv (VH- | EVQLQQSGPGLVKPSQTLSLTCAISGDSML |
| | linker-VL) | SNSDTWNWIRQSPSRGLEWLGRTYHRSTWY |
| | (linker | DDYASSVRGRVSINVDTSKNQYSLQLNAVT |
| | shown by | PEDTGVYYCARVRLQDGNSWSDAFDVWGQG |
| | italics and | TMVTVSSGGGGSQSALTQPASASGSPGQSV |
| | underline) | TISCTGTSSDVGGYNYVSWYQQHPGKAPKL |
| | | MIYDVSNRPSGVSNRFSGSKSGNTASLTIS |
| | | GLQAEDEADYYCSSYTSSSTLYVFGTGTQL |
| | | TVL |
| CD22-65ss | | |
| 1196 | scFv (VH- | EVQLQQSGPGLVKPSQTLSLTCAISGDSML |
| | VL) | SNSDTWNWIRQSPSRGLEWLGRTYHRSTWY |
| | (no linker | DDYASSVRGRVSINVDTSKNQYSLQLNAVT |
| | between | PEDTGVYYCARVRLQDGNSWSDAFDVWGQG |
| | VH-VL) | TMVTVSSQSALTQPASASGSPGQSVTISCT |
| | | GTSSDVGGYNYVSWYQQHPGKAPKLMIYDV |
| | | SNRPSGVSNRFSGSKSGNTASLTISGLQAE |
| | | DEADYYCSSYTSSSTLYVFGTGTQLTVL |
| CD22-65sKD | | |
| 1197 | VH | EVQLQQSGPGLVKPSQTLSLTCAISGDSML |
| | | SNSDTWNWIRKSPSRGLEWLGRTYHRSTWY |
| | | DDYASSVRGRVSINVDTSKNQYSLQLNAVT |
| | | PEDTGVYYCARVRLQDGNSWSDAFDVWGQG |
| | | TMVTVSS |
| 1198 | VL | QSALTQPASASGSPGQSVTISCTGTSSDVG |
| | | GYNYVSWYQDHPGKAPKLMIYDVSNRPSGV |
| | | SNRFSGSKSGNTASLTISGLQAEDEADYYC |
| | | SSYTSSSTLYVFGTGTQLTVL |
| 1199 | scFv (VH- | EVQLQQSGPGLVKPSQTLSLTCAISGDSML |
| | linker-VL) | SNSDTWNWIRKSPSRGLEWLGRTYHRSTWY |
| | | DDYASSVRGRVSINVDTSKNQYSLQLNAVT |
| | | PEDTGVYYCARVRLQDGNSWSDAFDVWGQG |
| | | TMVTVSSGGGGSQSALTQPASASGSPGQSV |
| | | TISCTGTSSDVGGYNYVSWYQDHPGKAPKL |
| | | MIYDVSNRPSGVSNRFSGSKSGNTASLTIS |
| | | GLQAEDEADYYCSSYTSSSTLYVFGTGTQL |
| | | TVL |
| CD22-64 | | |
| 1200 | HC CDR1 | SNSDTWN |
| | (Kabat) | |
| 1201 | HC CDR2 | RTYHRSTWYDDYASSVRG |
| | (Kabat) | |
| 1202 | HC CDR3 | VRLQDGNSWSDAFDV |
| | (Kabat) | |
| 1203 | HC CDR1 | GDSVLSNSD |
| | (Chothia) | |
| 1204 | HC CDR2 | YHRSTWY |
| | (Chothia) | |
| 1205 | HC CDR3 | VRLQDGNSWSDAFDV |
| | (Chothia) | |
| 1206 | HC CDR1 | GDSVLSNSDT |
| | (IMGT) | |
| 1207 | HC CDR2 | TYHRSTWYD |
| | (IMGT) | |
| 1208 | HC CDR3 | ARVRLQDGNSWSDAFDV |
| | (IMGT) | |
| 1209 | HC CDR1 | GDSVLSNSDTWN |
| | (Combined) | |
| 1210 | HC CDR2 | RTYHRSTWYDDYASSVRG |
| | (Combined) | |
| 1211 | HC CDR3 | VRLQDGNSWSDAFDV |
| | (Combined) | |
| 1212 | VH | EVQLQQSGPGLVKPSQTLPLTCAISGDSVL |
| | | SNSDTWNWIRQSPSRGLEWLGRTYHRSTWY |
| | | DDYASSVRGRVSINVDTSKNQYSLQLNAVT |
| | | PEDTGVYYCARVRLQDGNSWSDAFDVWGQG |
| | | TMVTVSS |
| 1213 | LC CDR1 | TGTSSDVGGYNYVS |
| | (Kabat) | |
| 1214 | LC CDR2 | DVSNRPS |
| | (Kabat) | |
| 1215 | LC CDR3 | SSYTSSSTLYV |
| | (Kabat) | |
| 1216 | LC CDR1 | TSSDVGGYNY |
| | (Chothia) | |
| 1217 | LC CDR2 | DVS |
| | (Chothia) | |
| 1218 | LC CDR3 | YTSSSTLY |
| | (Chothia) | |
| 1219 | LC CDR1 | SSDVGGYNY |
| | (IMGT) | |
| 1220 | LC CDR2 | DVS |
| | (IMGT) | |
| 1221 | LC CDR3 | SSYTSSSTLYV |
| | (IMGT) | |
| 1222 | LC CDR1 | TGTSSDVGGYNYVS |
| | (Combined) | |
| 1223 | LC CDR2 | DVSNRPS |
| | (Combined) | |
| 1224 | LC CDR3 | SSYTSSSTLYV |
| | (Combined) | |
| 1225 | VL | QSALTQPASASGSPGQSVTISCTGTSSDVG |
| | | GYNYVSWYQQHPGKAPKLMIYDVSNRPSGV |
| | | SNRFSGSKSGNTASLTISGLQAEDEADYYC |
| | | SSYTSSSTLYVFGTGTQLTVL |
| 1226 | scFv (VH- | EVQLQQSGPGLVKPSQTLPLTCAISGDSVL |
| | linker-VL) | SNSDTWNWIRQSPSRGLEWLGRTYHRSTWY |
| | | DDYASSVRGRVSINVDTSKNQYSLQLNAVT |
| | | PEDTGVYYCARVRLQDGNSWSDAFDVWGQG |
| | | TMVTVSSGGGGSGGGGSGGGGPQSALTQPA |
| | | SASGSPGQSVTISCTGTSSDVGGYNYVSWY |
| | | QQHPGKAPKLMIYDVSNRPSGVSNRFSGSK |
| | | SGNTASLTISGLQAEDEADYYCSSYTSSST |
| | | LYVFGTGTQLTVL |
| CD22-57 | | |
| 1227 | HC CDR1 | NNNAAWN |
| | (Kabat) | |
| 1228 | HC CDR2 | RTYHRSTWYNDYVGSVKS |
| | (Kabat) | |
| 1229 | HC CDR3 | ETDYGDYGAFDI |
| | (Kabat) | |
| 1230 | HC CDR1 | GDSVSNNNA |
| | (Chothia) | |
| 1231 | HC CDR2 | YHRSTWY |
| | (Chothia) | |
| 1232 | HC CDR3 | ETDYGDYGAFDI |
| | (Chothia) | |
| 1233 | HC CDR1 | GDSVSNNNAA |
| | (IMGT) | |
| 1234 | HC CDR2 | TYHRSTWYN |
| | (IMGT) | |
| 1235 | HC CDR3 | ARETDYGDYGAFDI |
| | (IMGT) | |
| 1236 | HC CDR1 | GDSVSNNNAAWN |
| | (Combined) | |
| 1237 | HC CDR2 | RTYHRSTWYNDYVGSVKS |
| | (Combined) | |
| 1238 | HC CDR3 | ETDYGDYGAFDI |
| | (Combined) | |
| 1239 | VH | EVQLQQSGPGLVKPSQTLSLTCAISGDSVS |
| | | NNNAAWNWIRQSPSRGLEWLGRTYHRSTWY |
| | | NDYVGSVKSRITINPDTSKNQFSLQLNSVT |
| | | PEDTAVYYCARETDYGDYGAFDIWGQGTTV |
| | | TVSS |
| 1240 | LC CDR1 | TGSRNDIGAYESVS |
| | (Kabat) | |
| 1241 | LC CDR2 | GVNNRPS |
| | (Kabat) | |
| 1242 | LC CDR3 | SSHTTTSTLYV |
| | (Kabat) | |
| 1243 | LC CDR1 | SRNDIGAYES |
| | (Chothia) | |
| 1244 | LC CDR2 | GVN |
| | (Chothia) | |
| 1245 | LC CDR3 | HTTTSTLY |
| | (Chothia) | |
| 1246 | LC CDR1 | RNDIGAYES |
| | (IMGT) | |
| 1247 | LC CDR2 | GVN |
| | (IMGT) | |
| 1248 | LC CDR3 | SSHTTTSTLYV |
| | (IMGT) | |
| 1249 | LC CDR1 | TGSRNDIGAYESVS |
| | (Combined) | |
| 1250 | LC CDR2 | GVNNRPS |
| | (Combined) | |
| 1251 | LC CDR3 | SSHTTTSTLYV |
| | (Combined) | |
| 1252 | VL | QSALTQPASVSGSPGQSITISCTGSRNDIG |
| | | AYESVSWYQQHPGNAPKLIIHGVNNRPSGV |
| | | FDRFSVSQSGNTASLTISGLQAEDEADYYC |
| | | SSHTTTSTLYVFGTGTKVTVL |
| 1253 | scFv (VH- | EVQLQQSGPGLVKPSQTLSLTCAISGDSVS |
| | linker-VL) | NNNAAWNWIRQSPSRGLEWLGRTYHRSTWY |
| | | NDYVGSVKSRITINPDTSKNQFSLQLNSVT |
| | | PEDTAVYYCARETDYGDYGAFDIWGQGTTV |
| | | TVSSGGGGSGGGGSGGGGSQSALTQPASVS |
| | | GSPGQSITISCTGSRNDIGAYESVSWYQQH |
| | | PGNAPKLIIHGVNNRPSGVFDRFSVSQSGN |
| | | TASLTISGLQAEDEADYYCSSHTTTSTLYV |
| | | FGTGTKVTVL |
| CD22-58 | | |
| 1254 | HC CDR1 | SNSAAWN |
| | (Kabat) | |
| 1255 | HC CDR2 | RTFYRSKWYNDYAVSVKG |
| | (Kabat) | |
| 1256 | HC CDR3 | GDYYYGLDV |
| | (Kabat) | |
| 1257 | HC CDR1 | GDSVSSNSA |
| | (Chothia) | |
| 1258 | HC CDR2 | FYRSKWY |
| | (Chothia) | |
| 1259 | HC CDR3 | GDYYYGLDV |
| | (Chothia) | |
| 1260 | HC CDR1 | GDSVSSNSAA |
| | (IMGT) | |
| 1261 | HC CDR2 | TFYRSKWYN |
| | (IMGT) | |
| 1262 | HC CDR3 | AGGDYYYGLDV |
| | (IMGT) | |
| 1263 | HC CDR1 | GDSVSSNSAAWN |
| | (Combined) | |
| 1264 | HC CDR2 | RTFYRSKWYNDYAVSVKG |
| | (Combined) | |
| 1265 | HC CDR3 | GDYYYGLDV |
| | (Combined) | |
| 1266 | VH | EVQLQQSGPGLVNPSQTLSITCAISGDSVS |
| | | SNSAAWNWIRQSPSRGLEWLGRTFYRSKWY |
| | | NDYAVSVKGRITISPDTSKNQFSLQLNSVT |
| | | PEDTAVYYCAGGDYYYGLDVWGQGTTVTVS |
| | | S |
| 1267 | LC CDR1 | TGSSSDVGGYNSVS |
| | (Kabat) | |
| 1268 | LC CDR2 | EVINRPS |
| | (Kabat) | |
| 1269 | LC CDR3 | SSYTSSSTYV |
| | (Kabat) | |
| 1270 | LC CDR1 | SSSDVGGYNS |
| | (Chothia) | |
| 1271 | LC CDR2 | EVI |
| | (Chothia) | |
| 1272 | LC CDR3 | YTSSSTY |
| | (Chothia) | |
| 1273 | LC CDR1 | SSDVGGYNS |
| | (IMGT) | |
| 1274 | LC CDR2 | EVI |
| | (IMGT) | |
| 1275 | LC CDR3 | SSYTSSSTYV |
| | (IMGT) | |
| 1276 | LC CDR1 | TGSSSDVGGYNSVS |
| | (Combined) | |
| 1277 | LC CDR2 | EVINRPS |
| | (Combined) | |
| 1278 | LC CDR3 | SSYTSSSTYV |
| | (Combined) | |
| 1279 | VL | QSALTQPASVSGSPGQSITISCTGSSSDVG |
| | | GYNSVSWYQQHPGKAPKLMIYEVINRPSGV |
| | | SHRFSGSKSGNTASLTISGLQAEDEADYYC |
| | | SSYTSSSTYVFGTGTKVTVL |
| 1280 | scFv (VH- | EVQLQQSGPGLVNPSQTLSITCAISGDSVS |
| | linker-VL) | SNSAAWNWIRQSPSRGLEWLGRTFYRSKWY |
| | | NDYAVSVKGRITISPDTSKNQFSLQLNSVT |
| | | PEDTAVYYCAGGDYYYGLDVWGQGTTVTVS |
| | | SGGGGSGGGGSGGGGSQSALTQPASVSGSP |
| | | GQSITISCTGSSSDVGGYNSVSWYQQHPGK |
| | | APKLMIYEVINRPSGVSHRFSGSKSGNTAS |
| | | LTISGLQAEDEADYYCSSYTSSSTYVFGTG |
| | | TKVTVL |
| CD22-59 | | |
| 1281 | HC CDR1 | SNSDTWN |
| | (Kabat) | |
| 1282 | HC CDR2 | RTYHRSTWYDDYASSVRG |
| | (Kabat) | |
| 1283 | HC CDR3 | DRLQDGNSWSDAFDV |
| | (Kabat) | |
| 1284 | HC CDR1 | GDSVLSNSD |
| | (Chothia) | |
| 1285 | HC CDR2 | YHRSTWY |
| | (Chothia) | |
| 1286 | HC CDR3 | DRLQDGNSWSDAFDV |
| | (Chothia) | |
| 1287 | HC CDR1 | GDSVLSNSDT |
| | (IMGT) | |
| 1288 | HC CDR2 | TYHRSTWYD |
| | (IMGT) | |
| 1289 | HC CDR3 | ARDRLQDGNSWSDAFDV |
| | (IMGT) | |
| 1290 | HC CDR1 | GDSVLSNSDTWN |
| | (Combined) | |
| 1291 | HC CDR2 | RTYHRSTWYDDYASSVRG |
| | (Combined) | |
| 1292 | HC CDR3 | DRLQDGNSWSDAFDV |
| | (Combined) | |
| 1293 | VH | EVQLQQSGPGLVKPSQTLSLTCAISGDSVL |
| | | SNSDTWNWIRQSPSRGLEWLGRTYHRSTWY |
| | | DDYASSVRGRVSINVDTSKNQYSLQLNAVT |
| | | PEDTGVYYCARDRLQDGNSWSDAFDVWGQG |
| | | TMVTVSS |
| 1294 | LC CDR1 | TGSSSDIGGFNYVS |
| | (Kabat) | |
| 1295 | LC CDR2 | EVTNRPS |
| | (Kabat) | |
| 1296 | LC CDR3 | SSYASGSPLYV |
| | (Kabat) | |
| 1297 | LC CDR1 | SSSDIGGFNY |
| | (Chothia) | |
| 1298 | LC CDR2 | EVT |
| | (Chothia) | |
| 1299 | LC CDR3 | YASGSPLY |
| | (Chothia) | |
| 1300 | LC CDR1 | SSDIGGFNY |
| | (IMGT) | |
| 1301 | LC CDR2 | EVT |
| | (IMGT) | |
| 1302 | LC CDR3 | SSYASGSPLYV |
| | (IMGT) | |
| 1303 | LC CDR1 | TGSSSDIGGFNYVS |
| | (Combined) | |
| 1304 | LC CDR2 | EVTNRPS |
| | (Combined) | |
| 1305 | LC CDR3 | SSYASGSPLYV |
| | (Combined) | |
| 1306 | VL | QSALTQPASVSGSPGQSITISCTGSSSDIG |
| | | GFNYVSWYQQHAGEAPKLMIYEVTNRPSGV |
| | | SDRFSGSKSDNTASLTISGLQAEDEADYYC |
| | | SSYASGSPLYVFGTGTKVTVL |
| 1307 | scFv (VH- | EVQLQQSGPGLVKPSQTLSLTCAISGDSVL |
| | linker-VL) | SNSDTWNWIRQSPSRGLEWLGRTYHRSTWY |
| | | DDYASSVRGRVSINVDTSKNQ |
| | | YSLQLNAVTPEDTGVYYCARDRLQDGNSWS |
| | | DAFDVWGQGTMVTVSSGGGGSGGGGSGGGG |
| | | SQSALTQPASVSGSPGQSITISCTGSSSDI |
| | | GGFNYVSWYQQHAGEAPKLMIYEVTNRPSG |
| | | VSDRFSGSKSDNTASLTISGLQAEDEADYY |
| | | CSSYASGSPLYVFGTGTKVTVL |
| CD22-60 | | |
| 1308 | HC CDR1 | SNSDTWN |
| | (Kabat) | |
| 1309 | HC CDR2 | RTYHRSTWYDDYASSVRG |
| | (Kabat) | |
| 1310 | HC CDR3 | DRLQDGNSWSDAFDV |
| | (Kabat) | |
| 1311 | HC CDR1 | GDSVLSNSD |
| | (Chothia) | |
| 1312 | HC CDR2 | YHRSTWY |
| | (Chothia) | |
| 1313 | HC CDR3 | DRLQDGNSWSDAFDV |
| | (Chothia) | |
| 1314 | HC CDR1 | GDSVLSNSDT |
| | (IMGT) | |
| 1315 | HC CDR2 | TYHRSTWYD |
| | (IMGT) | |
| 1316 | HC CDR3 | ARDRLQDGNSWSDAFDV |
| | (IMGT) | |
| 1317 | HC CDR1 | GDSVLSNSDTWN |
| | (Combined) | |
| 1318 | HC CDR2 | RTYHRSTWYDDYASSVRG |
| | (Combined) | |
| 1319 | HC CDR3 | DRLQDGNSWSDAFDV |
| | (Combined) | |
| 1320 | VH | EVQLQQSGPGLVKPSQTLSLTCAISGDSVL |
| | | SNSDTWNWIRQSPSRGLEWLGRTYHRSTWY |
| | | DDYASSVRGRVSINVDTSKNQYSLQLNAVT |
| | | PEDTGVYYCARDRLQDGNSWSDAFDVWGQG |
| | | TMVTVSS |
| 1321 | LC CDR1 | TGTSSDIGGYNYVS |
| | (Kabat) | |
| 1322 | LC CDR2 | EVSNRPS |
| | (Kabat) | |
| 1323 | LC CDR3 | SSYTSSSTLYV |
| | (Kabat) | |
| 1324 | LC CDR1 | TSSDIGGYNY |
| | (Chothia) | |
| 1325 | LC CDR2 | EVS |
| | (Chothia) | |
| 1326 | LC CDR3 | YTSSSTLY |
| | (Chothia) | |
| 1327 | LC CDR1 | SSDIGGYNY |
| | (IMGT) | |
| 1328 | LC CDR2 | EVS |
| | (IMGT) | |
| 1329 | LC CDR3 | SSYTSSSTLYV |
| | (IMGT) | |
| 1330 | LC CDR1 | TGTSSDIGGYNYVS |
| | (Combined) | |
| 1331 | LC CDR2 | EVSNRPS |
| | (Combined) | |
| 1332 | LC CDR3 | SSYTSSSTLYV |
| | (Combined) | |
| 1333 | VL | QSALTQPASVSGSPGQSITFSCTGTSSDIG |
| | | GYNYVSWYQQHPGKAPKLMIYEVSNRPSGV |
| | | SNRFSGTKSGNTASLTISGLQAEDEADYYC |
| | | SSYTSSSTLYVFGTGTKLTVL |
| 1334 | scFv (VH- | EVQLQQSGPGLVKPSQTLSLTCAISGDSVL |
| | linker-VL) | SNSDTWNWIRQSPSRGLEWLGRTYHRSTWY |
| | | DDYASSVRGRVSINVDTSKNQYSLQLNAVT |
| | | PEDTGVYYCARDRLQDGNSWSDAFDVWGQG |
| | | TMVTVSSGGGGSGGGGSGSGGSQSALTQPA |
| | | SVSGSPGQSITFSCTGTSSDIGGYNYVSWY |
| | | QQHPGKAPKLMIYEVSNRPSGVSNRFSGTK |
| | | SGNTASLTISGLQAEDEADYYCSSYTSSST |
| | | LYVFGTGTKLTVL |
| CD22-61 | | |
| 1335 | HC CDR1 | SNSDTWN |
| | (Kabat) | |
| 1336 | HC CDR2 | RTYHRSTWYDDYASSVRG |
| | (Kabat) | |
| 1337 | HC CDR3 | DRLQDGNSWSDAFDV |
| | (Kabat) | |
| 1338 | HC CDR1 | GDSVLSNSD |
| | (Chothia) | |
| 1339 | HC CDR2 | YHRSTWY |
| | (Chothia) | |
| 1340 | HC CDR3 | DRLQDGNSWSDAFDV |
| | (Chothia) | |
| 1341 | HC CDR1 | GDSVLSNSDT |
| | (IMGT) | |
| 1342 | HC CDR2 | TYHRSTWYD |
| | (IMGT) | |
| 1343 | HC CDR3 | ARDRLQDGNSWSDAFDV |
| | (IMGT) | |
| 1344 | HC CDR1 | GDSVLSNSDTWN |
| | (Combined) | |
| 1345 | HC CDR2 | RTYHRSTWYDDYASSVRG |
| | (Combined) | |
| 1346 | HC CDR3 | DRLQDGNSWSDAFDV |
| | (Combined) | |
| 1347 | VH | QVQLQESGPGLVKPSQTLSLTCAISGDSVL |
| | | SNSDTWNWIRQSPSRGLEWLGRTYHRSTWY |
| | | DDYASSVRGRVSINVDTSKNQYSLQLNAVT |
| | | PEDTGVYYCARDRLQDGNSWSDAFDVWGQG |
| | | TMVTVSS |
| 1348 | LC CDR1 | TGTSSDVGGYNYVS |
| | (Kabat) | |
| 1349 | LC CDR2 | EVSNRPS |
| | (Kabat) | |
| 1350 | LC CDR3 | SSYTSSSTLYV |
| | (Kabat) | |
| 1351 | LC CDR1 | TSSDVGGYNY |
| | (Chothia) | |
| 1352 | LC CDR2 | EVS |
| | (Chothia) | |
| 1353 | LC CDR3 | YTSSSTLY |
| | (Chothia) | |
| 1354 | LC CDR1 | SSDVGGYNY |
| | (IMGT) | |
| 1355 | LC CDR2 | EVS |
| | (IMGT) | |
| 1356 | LC CDR3 | SSYTSSSTLYV |
| | (IMGT) | |
| 1357 | LC CDR1 | TGTSSDVGGYNYVS |
| | (Combined) | |
| 1358 | LC CDR2 | EVSNRPS |
| | (Combined) | |
| 1359 | LC CDR3 | SSYTSSSTLYV |
| | (Combined) | |
| 1360 | VL | QSALTQPASVSGSPGQSITISCTGTSSDVG |
| | | GYNYVSWYQQHPGKAPKLMIYEVSNRPSGV |
| | | SNRFSGSKSGNTASLTISGLQAEDEADYYC |
| | | SSYTSSSTLYVFGTGTKVTVL |
| 1361 | scFv (VH- | QVQLQESGPGLVKPSQTLSLTCAISGDSVL |
| | linker-VL) | SNSDTWNWIRQSPSRGLEWLGRTYHRSTWY |
| | | DDYASSVRGRVSINVDTSKNQYSLQLNAVT |
| | | PEDTGVYYCARDRLQDGNSWSDAFDVWGQG |
| | | TMVTVSSGGGGSGGGGSGSGGSQSALTQPA |
| | | SVSGSPGQSITISCTGTSSDVGGYNYVSWY |
| | | QQHPGKAPKLMIYEVSNRPSGVSNRFSGSK |
| | | SGNTASLTISGLQAEDEADYYCSSYTSSST |
| | | LYVFGTGTKVTVL |
| CD22-62 | | |
| 1362 | HC CDR1 | SNSDTWN |
| | (Kabat) | |
| 1363 | HC CDR2 | RTYHRSTWYDDYASSVRG |
| | (Kabat) | |
| 1364 | HC CDR3 | DRLQDGNSWSDAFDV |
| | (Kabat) | |
| 1365 | HC CDR1 | GDSVLSNSD |
| | (Chothia) | |
| 1366 | HC CDR2 | YHRSTWY |
| | (Chothia) | |
| 1367 | HC CDR3 | DRLQDGNSWSDAFDV |
| | (Chothia) | |
| 1368 | HC CDR1 | GDSVLSNSDT |
| | (IMGT) | |
| 1369 | HC CDR2 | TYHRSTWYD |
| | (IMGT) | |
| 1370 | HC CDR3 | ARDRLQDGNSWSDAFDV |
| | (IMGT) | |
| 1371 | HC CDR1 | GDSVLSNSDTWN |
| | (Combined) | |
| 1372 | HC CDR2 | RTYHRSTWYDDYASSVRG |
| | (Combined) | |
| 1373 | HC CDR3 | DRLQDGNSWSDAFDV |
| | (Combined) | |
| 1374 | VH | EVQLQQSGPGLVKPSQTLSLTCAISGDSVL |
| | | SNSDTWNWIRQSPSRGLEWLGRTYHRSTWY |
| | | DDYASSVRGRVSINVDTSKNQYSLQLNAVT |
| | | PEDTGVYYCARDRLQDGNSWSDAFDVWGQG |
| | | TMVTVSS |
| 1375 | LC CDR1 | TGTSSDVGGYNYVS |
| | (Kabat) | |
| 1376 | LC CDR2 | DVSNRPS |
| | (Kabat) | |
| 1377 | LC CDR3 | SSYTSSSTLYV |
| | (Kabat) | |
| 1378 | LC CDR1 | TSSDVGGYNY |
| | (Chothia) | |
| 1379 | LC CDR2 | DVS |
| | (Chothia) | |
| 1380 | LC CDR3 | YTSSSTLY |
| | (Chothia) | |
| 1381 | LC CDR1 | SSDVGGYNY |
| | (IMGT) | |
| 1382 | LC CDR2 | DVS |
| | (IMGT) | |
| 1383 | LC CDR3 | SSYTSSSTLYV |
| | (IMGT) | |
| 1384 | LC CDR1 | TGTSSDVGGYNYVS |
| | (Combined) | |
| 1385 | LC CDR2 | DVSNRPS |
| | (Combined) | |
| 1386 | LC CDR3 | SSYTSSSTLYV |
| | (Combined) | |
| 1387 | VL | QSALTQPASVSGSPGQSITISCTGTSSDVG |
| | | GYNYVSWYQQHPGKAPKLMIYDVSNRPSGV |
| | | SNRFSGSKSGNTASLTISGLQAEDEADYYC |
| | | SSYTSSSTLYVFGTGTKVTVL |
| 1388 | scFv (VH- | EVQLQQSGPGLVKPSQTLSLTCAISGDSVL |
| | linker-VL) | SNSDTWNWIRQSPSRGLEWLGRTYHRSTWY |
| | | DDYASSVRGRVSINVDTSKNQYSLQLNAVT |
| | | PEDTGVYYCARDRLQDGNSWSDAFDVWGQG |
| | | TMVTVSSGGGGSGGGGSGGGGSQSALTQPA |
| | | SVSGSPGQSITISCTGTSSDVGGYNYVSWY |
| | | QQHPGKAPKLMIYDVSNRPSGVSNRFSGSK |
| | | SGNTASLTISGLQAEDEADYYCSSYTSSST |
| | | LYVFGTGTKVTVL |
| CD22-63 | | |
| 1389 | HC CDR1 | SNSDTWN |
| | (Kabat) | |
| 1390 | HC CDR2 | RTYHRSTWYDDYASSVRG |
| | (Kabat) | |
| 1391 | HC CDR3 | DRLQDGNSWSDAFDV |
| | (Kabat) | |
| 1392 | HC CDR1 | GDSVLSNSD |
| | (Chothia) | |
| 1393 | HC CDR2 | YHRSTWY |
| | (Chothia) | |
| 1394 | HC CDR3 | DRLQDGNSWSDAFDV |
| | (Chothia) | |
| 1395 | HC CDR1 | GDSVLSNSDT |
| | (IMGT) | |
| 1396 | HC CDR2 | TYHRSTWYD |
| | (IMGT) | |
| 1397 | HC CDR3 | ARDRLQDGNSWSDAFDV |
| | (IMGT) | |
| 1398 | HC CDR1 | GDSVLSNSDTWN |
| | (Combined) | |
| 1399 | HC CDR2 | RTYHRSTWYDDYASSVRG |
| | (Combined) | |
| 1400 | HC CDR3 | DRLQDGNSWSDAFDV |
| | (Combined) | |
| 1401 | VH | EVQLQQSGPGLVKPSQTLSLTCAISGDSVL |
| | | SNSDTWNWIRQSPSRGLEWLGRTYHRSTWY |
| | | DDYASSVRGRVSINVDTSKNQYSLQLNAVT |
| | | PEDTGVYYCARDRLQDGNSWSDAFDVWGQG |
| | | TMVTVSS |
| 1402 | LC CDR1 | TGTSSDVGGYNYVS |
| | (Kabat) | |
| 1403 | LC CDR2 | EVSNRPS |
| | (Kabat) | |
| 1404 | LC CDR3 | SSYTSSSTLYI |
| | (Kabat) | |
| 1405 | LC CDR1 | TSSDVGGYNY |
| | (Chothia) | |
| 1406 | LC CDR2 | EVS |
| | (Chothia) | |
| 1407 | LC CDR3 | YTSSSTLY |
| | (Chothia) | |
| 1408 | LC CDR1 | SSDVGGYNY |
| | (IMGT) | |
| 1409 | LC CDR2 | EVS |
| | (IMGT) | |
| 1410 | LC CDR3 | SSYTSSSTLYI |
| | (IMGT) | |
| 1411 | LC CDR1 | TGTSSDVGGYNYVS |
| | (Combined) | |
| 1412 | LC CDR2 | EVSNRPS |
| | (Combined) | |
| 1413 | LC CDR3 | SSYTSSSTLYI |
| | (Combined) | |
| 1414 | VL | QSALTQPASVSGSPGQSITISCTGTSSDVG |
| | | GYNYVSWYQQHPGKAPKLMIYEVSNRPSGV |
| | | SNRFSGSKSGNTASLTISGLQAEDEADYYC |
| | | SSYTSSSTLYIFGTGTKVTVL |
| 1415 | scFv (VH- | EVQLQQSGPGLVKPSQTLSLTCAISGDSVL |
| | linker-VL) | SNSDTWNWIRQSPSRGLEWLGRTYHRSTWY |
| | | DDYASSVRGRVSINVDTSKNQYSLQLNAVT |
| | | PEDTGVYYCARDRLQDGNSWSDAFDVWGQG |
| | | TMVTVSSGGGGGGGGSGGGGSQSALTQPAS |
| | | VSGSPGQSITISCTGTSSDVGGYNYVSWYQ |
| | | QHPGKAPKLMIYEVSNRPSGVSNRFSGSKS |
| | | GNTASLTISGLQAEDEADYYCSSYTSSSTL |
| | | YIFGTGTKVTVL |

CARs that bind to EGFR are known in the art. For example, those disclosed in WO2014/130657, incorporated by reference herein, can be used in accordance with the present disclosure. Any known EGFR CAR, for example, the EGFR antigen binding domain of any known EGFR CAR, in the art can be used in accordance with the present disclosure. Exemplary EGFRvIII CARs can include a CDR, a variable region, an scFv, or a full-length CAR sequence disclosed in WO2014/130657, for example, Table 2 of WO2014/130657, incorporated herein by reference.

CARs that bind to CD123 are known in the art. For example, those disclosed in WO2014/130635 or WO2016/028896 can be used in accordance with the present disclosure. Any known CD123 CAR, for example, the CD123 antigen binding domain of any known CD123 CAR, in the art can be used in accordance with the present disclosure. For example, CAR1 to CAR8 disclosed in WO2014/130635; or CAR123-1 to CAR123-4 and hzCAR123-1 to hzCAR123-32, disclosed in WO2016/028896. The amino acid and nucleotide sequences encoding the CD123 CAR molecules and antigen binding domains (for example, including one, two, three VH CDRs; and one, two, three VL CDRs according to Kabat or Chothia), are specified in WO 2014/130635 and WO2016/028896. CARs that bind to CLL-1 are known in the art. For example, those disclosed in US2016/0051651A1, incorporated herein by reference. Any known CLL-1 CAR, for example, the CLL-1 antigen binding domain of any known CLL-1 CAR, in the art can be used in accordance with the present disclosure.

In some embodiments, the CAR comprises a CLL-1 CAR or antigen binding domain according to Table 2 of WO2016/014535, incorporated herein by reference. The amino acid and nucleotide sequences encoding the CLL-1 CAR molecules and antigen binding domains (for example, including one, two, three VH CDRs; and one, two, three VL CDRs according to Kabat or Chothia), are specified in WO2016/014535.

CARs that bind to CD33 are known in the art. For example, those disclosed in US2016/0096892A1 and WO2016/014576, incorporated by reference herein, can be used in accordance with the present disclosure. Any known CD33 CAR, for example, the CD33 antigen binding domain of any known CD33 CAR, in the art can be used in accordance with the present disclosure. For example, CAR33-1 to CAR33-9 disclosed in WO2016/014576 can be used in accordance with the present disclosure.

In some embodiments, the CAR comprises a CD33 CAR or antigen binding domain according to Table 2 or 9 of WO2016/014576, incorporated herein by reference. The amino acid and nucleotide sequences encoding the CD33 CAR molecules and antigen binding domains (for example, including one, two, three VH CDRs; and one, two, three VL CDRs according to Kabat or Chothia), are specified in WO2016/014576.

In one embodiment, an antigen binding domain against CD33 is an antigen binding portion, e.g., CDRs, of an antibody described in, e.g., Bross et al., Clin Cancer Res 7(6):1490-1496 (2001) (Gemtuzumab Ozogamicin, hP67.6), Caron et al., Cancer Res 52(24):6761-6767 (1992) (Lintuzumab, HuM195), Lapusan et al., Invest New Drugs 30(3):1121-1131 (2012) (AVE9633), Aigner et al., Leukemia 27(5): 1107-1115 (2013) (AMG330, CD33 BiTE), Dutour et al., Adv hematol 2012:683065 (2012), and Pizzitola et al., Leukemia doi:10.1038/Lue.2014.62 (2014). In one embodiment, an antigen binding domain against CD33 is an antigen binding portion, e.g., CDRs, of an antibody, antigen-binding fragment, or CAR described in WO/2016/014576.

In one embodiment, an antigen binding domain against GD2 is an antigen binding portion, e.g., CDRs, of an antibody described in, e.g., Mujoo et al., Cancer Res. 47(4):1098-1104 (1987); Cheung et al., Cancer Res 45(6):2642-2649 (1985), Cheung et al., J Clin Oncol 5(9):1430-1440 (1987), Cheung et al., J Clin Oncol 16(9):3053-3060 (1998), Handgretinger et al., Cancer Immunol Immunother 35(3):199-204 (1992). In some embodiments, an antigen binding domain against GD2 is an antigen binding portion of an antibody selected from mAb 14.18, 14G2a, ch14.18, hu14.18, 3F8, hu3F8, 3G6, 8B6, 60C3, 10B8, ME36.1, and 8H9, see e.g., WO2012033885, WO2013040371, WO2013192294, WO2013061273, WO2013123061, WO2013074916, and WO201385552. In some embodiments, an antigen binding domain against GD2 is an antigen binding portion of an antibody described in US Publication No.: 20100150910 or PCT Publication No.: WO 2011160119.

In one embodiment, an antigen binding domain against Tn antigen is an antigen binding portion, e.g., CDRs, of an antibody described in, e.g., U.S. Pat. No. 8,440,798, Brooks et al., PNAS 107(22):10056-10061 (2010), and Stone et al., OncoImmunology 1(6):863-873(2012).

In one embodiment, an antigen binding domain against PSMA is an antigen binding portion, e.g., CDRs, of an antibody described in, e.g., Parker et al., Protein Expr Purif 89(2):136-145 (2013), US 20110268656 (J591 ScFv); Frigerio et al, European J Cancer 49(9):2223-2232 (2013) (scFvD2B); WO 2006125481 (mAbs 3/A12, 3/E7 and 3/F11) and single chain antibody fragments (scFv A5 and D7).

In one embodiment, an antigen binding domain against ROR1 is an antigen binding portion, e.g., CDRs, of an antibody described in, e.g., Hudecek et al., Clin Cancer Res 19(12):3153-3164 (2013); WO 2011159847; and US20130101607.

In one embodiment, an antigen binding domain against FLT3 is an antigen binding portion, e.g., CDRs, of an antibody described in, e.g., WO2011076922, U.S. Pat. No. 5,777,084, EP0754230, US20090297529, and several commercial catalog antibodies (R&D, ebiosciences, Abcam).

In one embodiment, an antigen binding domain against TAG72 is an antigen binding portion, e.g., CDRs, of an antibody described in, e.g., Hombach et al., Gastroenterology 113(4):1163-1170 (1997); and Abcam ab691.

In one embodiment, an antigen binding domain against FAP is an antigen binding portion, e.g., CDRs, of an antibody described in, e.g., Ostermann et al., Clinical Cancer Research 14:4584-4592 (2008) (FAP5), US Pat. Publication No. 2009/0304718; sibrotuzumab (see e.g., Hofheinz et al., Oncology Research and Treatment 26(1), 2003); and Tran et al., J Exp Med 210(6):1125-1135 (2013).

In one embodiment, an antigen binding domain against CD38 is an antigen binding portion, e.g., CDRs, of daratumumab (see, e.g., Groen et al., Blood 116(21):1261-1262 (2010); MOR202 (see, e.g., U.S. Pat. No. 8,263,746); or antibodies described in U.S. Pat. No. 8,362,211.

In one embodiment, an antigen binding domain against CD44v6 is an antigen binding portion, e.g., CDRs, of an antibody described in, e.g., Casucci et al., Blood 122(20):3461-3472 (2013).

In one embodiment, an antigen binding domain against CEA is an antigen binding portion, e.g., CDRs, of an antibody described in, e.g., Chmielewski et al., Gastoenterology 143(4):1095-1107 (2012).

In one embodiment, an antigen binding domain against EPCAM is an antigen binding portion, e.g., CDRs, of an antibody selected from MT110, EpCAM-CD3 bispecific Ab (see, e.g., clinicaltrials.gov/ct2/show/NCT00635596); Edrecolomab; 3622W94; ING-1; and adecatumumab (MT201).

In one embodiment, an antigen binding domain against PRSS21 is an antigen binding portion, e.g., CDRs, of an antibody described in U.S. Pat. No. 8,080,650.

In one embodiment, an antigen binding domain against B7H3 is an antigen binding portion, e.g., CDRs, of an antibody MGA271 (Macrogenics).

In one embodiment, an antigen binding domain against KIT is an antigen binding portion, e.g., CDRs, of an antibody described in, e.g., U.S. Pat. No. 7,915,391, US20120288506, and several commercial catalog antibodies.

In one embodiment, an antigen binding domain against IL-13Ra2 is an antigen binding portion, e.g., CDRs, of an antibody described in, e.g., WO2008/146911, WO2004087758, several commercial catalog antibodies, and WO2004087758.

In one embodiment, an antigen binding domain against CD30 is an antigen binding portion, e.g., CDRs, of an antibody described in, e.g., U.S. Pat. No. 7,090,843 B1, and EP0805871.

In one embodiment, an antigen binding domain against GD3 is an antigen binding portion, e.g., CDRs, of an antibody described in, e.g., U.S. Pat. Nos. 7,253,263; 8,207,308; US 20120276046; EP1013761; WO2005035577; and U.S. Pat. No. 6,437,098.

In one embodiment, an antigen binding domain against CD171 is an antigen binding portion, e.g., CDRs, of an antibody described in, e.g., Hong et al., J Immunother 37(2):93-104 (2014).

In one embodiment, an antigen binding domain against IL-11Ra is an antigen binding portion, e.g., CDRs, of an antibody available from Abcam (cat #ab55262) or Novus Biologicals (cat #EPR5446). In another embodiment, an antigen binding domain again IL-11Ra is a peptide, see, e.g., Huang et al., Cancer Res 72(1):271-281 (2012).

In one embodiment, an antigen binding domain against PSCA is an antigen binding portion, e.g., CDRs, of an antibody described in, e.g., Morgenroth et al., Prostate 67(10):1121-1131 (2007) (scFv 7F5); Nejatollahi et al., J of Oncology 2013(2013), article ID 839831 (scFv C5-II); and US Pat Publication No. 20090311181.

In one embodiment, an antigen binding domain against VEGFR2 is an antigen binding portion, e.g., CDRs, of an antibody described in, e.g., Chinnasamy et al., J Clin Invest 120(11):3953-3968 (2010).

In one embodiment, an antigen binding domain against LewisY is an antigen binding portion, e.g., CDRs, of an antibody described in, e.g., Kelly et al., Cancer Biother Radiopharm 23(4):411-423 (2008) (hu3S193 Ab (scFvs)); Dolezal et al., Protein Engineering 16(1):47-56 (2003) (NC10 scFv).

In one embodiment, an antigen binding domain against CD24 is an antigen binding portion, e.g., CDRs, of an antibody described in, e.g., Maliar et al., Gastroenterology 143(5):1375-1384 (2012).

In one embodiment, an antigen binding domain against PDGFR-beta is an antigen binding portion, e.g., CDRs, of an antibody Abcam ab32570.

In one embodiment, an antigen binding domain against SSEA-4 is an antigen binding portion, e.g., CDRs, of antibody MC813 (Cell Signaling), or other commercially available antibodies.

In one embodiment, an antigen binding domain against CD20 is an antigen binding portion, e.g., CDRs, of the antibody Rituximab, Ofatumumab, Ocrelizumab, Veltuzumab, or GA101; or antibodies described in WO2016/164731.

In one embodiment, an antigen binding domain against Folate receptor alpha is an antigen binding portion, e.g., CDRs, of the antibody IMGN853, or an antibody described in US20120009181; U.S. Pat. No. 4,851,332, LK26: U.S. Pat. No. 5,952,484.

In one embodiment, an antigen binding domain against ERBB2 (Her2/neu) is an antigen binding portion, e.g., CDRs, of the antibody trastuzumab, or pertuzumab.

In one embodiment, an antigen binding domain against MUC1 is an antigen binding portion, e.g., CDRs, of the antibody SAR566658.

In one embodiment, the antigen binding domain against EGFR is antigen binding portion, e.g., CDRs, of the antibody cetuximab, panitumumab, zalutumumab, nimotuzumab, or matuzumab.

In one embodiment, an antigen binding domain against NCAM is an antigen binding portion, e.g., CDRs, of the antibody clone 2-2B: MAB5324 (EMD Millipore).

In one embodiment, an antigen binding domain against Ephrin B2 is an antigen binding portion, e.g., CDRs, of an antibody described in, e.g., Abengozar et al., Blood 119(19):4565-4576 (2012).

In one embodiment, an antigen binding domain against IGF-I receptor is an antigen binding portion, e.g., CDRs, of an antibody described in, e.g., U.S. Pat. No. 8,344,112 B2; EP2322550 A1; WO 2006/138315, or PCT/US2006/022995.

In one embodiment, an antigen binding domain against CAIX is an antigen binding portion, e.g., CDRs, of the antibody clone 303123 (R&D Systems).

In one embodiment, an antigen binding domain against LMP2 is an antigen binding portion, e.g., CDRs, of an antibody described in, e.g., U.S. Pat. No. 7,410,640, or US20050129701.

In one embodiment, an antigen binding domain against gp100 is an antigen binding portion, e.g., CDRs, of the antibody HMB45, NKIbetaB, or an antibody described in WO2013165940, or US20130295007

In one embodiment, an antigen binding domain against tyrosinase is an antigen binding portion, e.g., CDRs, of an antibody described in, e.g., U.S. Pat. No. 5,843,674; or US19950504048.

In one embodiment, an antigen binding domain against EphA2 is an antigen binding portion, e.g., CDRs, of an antibody described in, e.g., Yu et al., Mol Ther 22(1):102-111 (2014).

In one embodiment, an antigen binding domain against GD3 is an antigen binding portion, e.g., CDRs, of an antibody described in, e.g., U.S. Pat. Nos. 7,253,263; 8,207,308; US 20120276046; EP1013761 A3; 20120276046; WO2005035577; or U.S. Pat. No. 6,437,098.

In one embodiment, an antigen binding domain against fucosyl GM1 is an antigen binding portion, e.g., CDRs, of an antibody described in, e.g., US20100297138; or WO2007/067992.

In one embodiment, an antigen binding domain against sLe is an antigen binding portion, e.g., CDRs, of the antibody G193 (for lewis Y), see Scott A M et al, Cancer Res 60: 3254-61 (2000), also as described in Neeson et al, J Immunol May 2013 190 (Meeting Abstract Supplement) 177.10.

In one embodiment, an antigen binding domain against GM3 is an antigen binding portion, e.g., CDRs, of the antibody CA 2523449 (mAb 14F7).

In one embodiment, an antigen binding domain against HMWMAA is an antigen binding portion, e.g., CDRs, of an antibody described in, e.g., Kmiecik et al., Oncoimmunology 3(1):e27185 (2014) (PMID: 24575382) (mAb9.2.27); U.S. Pat. No. 6,528,481; WO2010033866; or US 20140004124.

In one embodiment, an antigen binding domain against o-acetyl-GD2 is an antigen binding portion, e.g., CDRs, of the antibody 8B6.

In one embodiment, an antigen binding domain against TEM1/CD248 is an antigen binding portion, e.g., CDRs, of an antibody described in, e.g., Marty et al., Cancer Lett 235(2):298-308 (2006); Zhao et al., J Immunol Methods 363(2):221-232 (2011).

In one embodiment, an antigen binding domain against CLDN6 is an antigen binding portion, e.g., CDRs, of the antibody IMAB027 (Ganymed Pharmaceuticals), see e.g., clinicaltrial.gov/show/NCT02054351.

In one embodiment, an antigen binding domain against TSHR is an antigen binding portion, e.g., CDRs, of an antibody described in, e.g., U.S. Pat. Nos. 8,603,466; 8,501,415; or U.S. Pat. No. 8,309,693.

In one embodiment, an antigen binding domain against GPRC5D is an antigen binding portion, e.g., CDRs, of the antibody FAB6300A (R&D Systems); or LS-A4180 (Lifespan Biosciences).

In one embodiment, an antigen binding domain against CD97 is an antigen binding portion, e.g., CDRs, of an antibody described in, e.g., U.S. Pat. No. 6,846,911; de Groot et al., J Immunol 183(6):4127-4134 (2009); or an antibody from R&D:MAB3734.

In one embodiment, an antigen binding domain against ALK is an antigen binding portion, e.g., CDRs, of an antibody described in, e.g., Mino-Kenudson et al., Clin Cancer Res 16(5):1561-1571 (2010).

In one embodiment, an antigen binding domain against polysialic acid is an antigen binding portion, e.g., CDRs, of an antibody described in, e.g., Nagae et al., J Biol Chem 288(47):33784-33796 (2013).

In one embodiment, an antigen binding domain against PLAC1 is an antigen binding portion, e.g., CDRs, of an antibody described in, e.g., Ghods et al., Biotechnol Appl Biochem 2013 doi:10.1002/bab.1177.

In one embodiment, an antigen binding domain against GloboH is an antigen binding portion of the antibody VK9; or an antibody described in, e.g., Kudryashov V et al, Glycoconj J. 15(3):243-9 (1998), Lou et al., Proc Natl Acad Sci USA 111(7):2482-2487 (2014); MBr1: Bremer E-G et al. J Biol Chem 259:14773-14777 (1984).

In one embodiment, an antigen binding domain against NY-BR-1 is an antigen binding portion, e.g., CDRs of an antibody described in, e.g., Jager et al., Appl Immunohistochem Mol Morphol 15(1):77-83 (2007).

In one embodiment, an antigen binding domain against WT-1 is an antigen binding portion, e.g., CDRs, of an antibody described in, e.g., Dao et al., Sci Transl Med 5(176):176ra33 (2013); or WO2012/135854.

In one embodiment, an antigen binding domain against MAGE-A1 is an antigen binding portion, e.g., CDRs, of an antibody described in, e.g., Willemsen et al., J Immunol 174(12):7853-7858 (2005) (TCR-like scFv).

In one embodiment, an antigen binding domain against sperm protein 17 is an antigen binding portion, e.g., CDRs, of an antibody described in, e.g., Song et al., Target Oncol 2013 Aug. 14 (PMID: 23943313); Song et al., Med Oncol 29(4):2923-2931 (2012).

In one embodiment, an antigen binding domain against Tie 2 is an antigen binding portion, e.g., CDRs, of the antibody AB33 (Cell Signaling Technology).

In one embodiment, an antigen binding domain against MAD-CT-2 is an antigen binding portion, e.g., CDRs, of an antibody described in, e.g., PMID: 2450952; U.S. Pat. No. 7,635,753.

In one embodiment, an antigen binding domain against Fos-related antigen 1 is an antigen binding portion, e.g., CDRs, of the antibody 12F9 (Novus Biologicals).

In one embodiment, an antigen binding domain against MelanA/MART1 is an antigen binding portion, e.g., CDRs, of an antibody described in, EP2514766 A2; or U.S. Pat. No. 7,749,719.

In one embodiment, an antigen binding domain against sarcoma translocation breakpoints is an antigen binding portion, e.g., CDRs, of an antibody described in, e.g., Luo et al, EMBO Mol. Med. 4(6):453-461 (2012).

In one embodiment, an antigen binding domain against TRP-2 is an antigen binding portion, e.g., CDRs, of an antibody described in, e.g., Wang et al, J Exp Med. 184(6):2207-16 (1996).

In one embodiment, an antigen binding domain against CYP1B1 is an antigen binding portion, e.g., CDRs, of an antibody described in, e.g., Maecker et al, Blood 102 (9): 3287-3294 (2003).

In one embodiment, an antigen binding domain against RAGE-1 is an antigen binding portion, e.g., CDRs, of the antibody MAB5328 (EMD Millipore).

In one embodiment, an antigen binding domain against human telomerase reverse transcriptase is an antigen binding portion, e.g., CDRs, of the antibody cat no: LS-B95-100 (Lifespan Biosciences)

In one embodiment, an antigen binding domain against intestinal carboxyl esterase is an antigen binding portion, e.g., CDRs, of the antibody 4F12: cat no: LS-B6190-50 (Lifespan Biosciences).

In one embodiment, an antigen binding domain against mut hsp70-2 is an antigen binding portion, e.g., CDRs, of the antibody LifespanBiosciences: monoclonal: cat no: LS-C133261-100 (Lifespan Biosciences).

In one embodiment, an antigen binding domain against CD79a is an antigen binding portion, e.g., CDRs, of the antibody Anti-CD79a antibody [HM47/A9] (ab3121), available from Abcam; antibody CD79A Antibody #3351 available from Cell Signalling Technology; or antibody HPA017748—Anti-CD79A antibody produced in rabbit, available from Sigma Aldrich.

In one embodiment, an antigen binding domain against CD79b is an antigen binding portion, e.g., CDRs, of the antibody polatuzumab vedotin, anti-CD79b described in Dornan et al., “Therapeutic potential of an anti-CD79b antibody-drug conjugate, anti-CD79b-vc-MMAE, for the treatment of non-Hodgkin lymphoma” Blood. 2009 Sep. 24; 114(13):2721-9. doi: 10.1182/blood-2009-02-205500. Epub 2009 Jul. 24, or the bispecific antibody Anti-CD79b/CD3 described in “4507 Pre-Clinical Characterization of T Cell-Dependent Bispecific Antibody Anti-CD79b/CD3 As a Potential Therapy for B Cell Malignancies” Abstracts of 56^{th} ASH Annual Meeting and Exposition, San Francisco, CA Dec. 6-9, 2014.

In one embodiment, an antigen binding domain against CD72 is an antigen binding portion, e.g., CDRs, of the antibody J3-109 described in Myers, and Uckun, “An anti-CD72 immunotoxin against therapy-refractory B-lineage acute lymphoblastic leukemia.” Leuk Lymphoma. 1995 June; 18(1-2):119-22, or anti-CD72 (10D6.8.1, mIgGI) described in Polson et al., “Antibody-Drug Conjugates for the Treatment of Non-Hodgkin's Lymphoma: Target and Linker-Drug Selection” Cancer Res Mar. 15, 2009 69; 2358.

In one embodiment, an antigen binding domain against LAIR1 is an antigen binding portion, e.g., CDRs, of the antibody ANT-301 LAIR1 antibody, available from ProSpec; or anti-human CD305 (LAIR1) Antibody, available from BioLegend.

In one embodiment, an antigen binding domain against FCAR is an antigen binding portion, e.g., CDRs, of the antibody CD89/FCARAntibody (Catalog #10414-H08H), available from Sino Biological Inc.

In one embodiment, an antigen binding domain against LILRA2 is an antigen binding portion, e.g., CDRs, of the antibody LILRA2 monoclonal antibody (M17), clone 3C7, available from Abnova, or Mouse Anti-LILRA2 antibody, Monoclonal (2D7), available from Lifespan Biosciences.

In one embodiment, an antigen binding domain against CD300LF is an antigen binding portion, e.g., CDRs, of the antibody Mouse Anti-CMRF35-like molecule 1 antibody, Monoclonal[UP-D2], available from BioLegend, or Rat Anti-CMRF35-like molecule 1 antibody, Monoclonal[234903], available from R&D Systems.

In one embodiment, an antigen binding domain against CLEC12A is an antigen binding portion, e.g., CDRs, of the antibody Bispecific T cell Engager (BiTE) scFv-antibody and ADC described in Noordhuis et al., “Targeting of CLEC12A In Acute Myeloid Leukemia by Antibody-Drug-Conjugates and Bispecific CLL-1×CD3 BiTE Antibody” 53^{rd} ASH Annual Meeting and Exposition, Dec. 10-13, 2011, and MCLA-117 (Merus).

In one embodiment, an antigen binding domain against BST2 (also called CD317) is an antigen binding portion, e.g., CDRs, of the antibody Mouse Anti-CD317 antibody, Monoclonal[3H4], available from Antibodies-Online or Mouse Anti-CD317 antibody, Monoclonal[696739], available from R&D Systems.

In one embodiment, an antigen binding domain against EMR2 (also called CD312) is an antigen binding portion, e.g., CDRs, of the antibody Mouse Anti-CD312 antibody, Monoclonal[LS-B8033] available from Lifespan Biosciences, or Mouse Anti-CD312 antibody, Monoclonal[494025] available from R&D Systems.

In one embodiment, an antigen binding domain against LY75 is an antigen binding portion, e.g., CDRs, of the antibody Mouse Anti-Lymphocyte antigen 75 antibody, Monoclonal[HD30] available from EMD Millipore or Mouse Anti-Lymphocyte antigen 75 antibody, Monoclonal[A15797] available from Life Technologies.

In one embodiment, an antigen binding domain against GPC3 is an antigen binding portion, e.g., CDRs, of the antibody hGC33 described in Nakano K, Ishiguro T, Konishi H, et al. Generation of a humanized anti-glypican 3 antibody by CDR grafting and stability optimization. Anticancer Drugs. 2010 November; 21(10):907-916, or MDX-1414, HN3, or YP7, all three of which are described in Feng et al., “Glypican-3 antibodies: a new therapeutic target for liver cancer.” FEBS Lett. 2014 Jan. 21; 588(2):377-82.

In one embodiment, an antigen binding domain against FCRL5 is an antigen binding portion, e.g., CDRs, of the anti-FcRL5 antibody described in Elkins et al., “FcRL5 as a target of antibody-drug conjugates for the treatment of multiple myeloma” Mol Cancer Ther. 2012 October; 11(10):2222-32. In one embodiment, an antigen binding domain against FCRL5 is an antigen binding portion, e.g., CDRs, of the anti-FcRL5 antibody described in, for example, WO2001/038490, WO/2005/117986, WO2006/039238, WO2006/076691, WO2010/114940, WO2010/120561, or WO2014/210064.

In one embodiment, an antigen binding domain against IGLL1 is an antigen binding portion, e.g., CDRs, of the antibody Mouse Anti-Immunoglobulin lambda-like polypeptide 1 antibody, Monoclonal[AT1G4] available from Lifespan Biosciences, Mouse Anti-Immunoglobulin lambda-like polypeptide 1 antibody, Monoclonal[HSL11] available from BioLegend.

In one embodiment, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In one embodiment, the antigen binding domain comprises a heavy chain variable region and/or a variable light chain region of an antibody listed above.

In another aspect, the antigen binding domain comprises a humanized antibody or an antibody fragment. In some aspects, a non-human antibody is humanized, where specific sequences or regions of the antibody are modified to increase similarity to an antibody naturally produced in a human or fragment thereof. In one aspect, the antigen binding domain is humanized.

CARs that bind to mesothelin are known in the art. For example, those disclosed in WO2015090230 and WO2017112741, for example, Tables 2, 3, 4, and 5 of WO2017112741, incorporated herein by reference, that bind human mesothelin. Any known mesothelin CAR, for example, the mesothelin antigen binding domain of any known mesothelin CAR, in the art can be used in accordance with the present disclosure.

CARs that bind to GFR ALPHA-4 are known in the art. For example, those disclosed in WO2016/025880 can be used in accordance with the present disclosure. Any known GFR ALPHA-4 CAR, for example, the GFR ALPHA-4 antigen binding domain of any known GFR ALPHA-4 CAR, in the art can be used in accordance with the present disclosure. The amino acid and nucleotide sequences encoding the GFR ALPHA-4 CAR molecules and antigen binding domains (for example, including one, two, three VH CDRs; and one, two, three VL CDRs according to Kabat or Chothia), are specified in WO2016/025880.

### Antigen Binding Domain Structures

In some embodiments, the antigen binding domain of the encoded CAR molecule comprises an antibody, an antibody fragment, an scFv, a Fv, a Fab, a (Fab′)2, a single domain antibody (SDAB), a VH or VL domain, a camelid VHH domain or a bi-functional (e.g. bi-specific) hybrid antibody (e.g., Lanzavecchia et al., Eur. J. Immunol. 17, 105 (1987)).

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)ₙ, where n is a positive integer equal to or greater than 1 (SEQ ID NO:22). In some embodiments, the linker can be (Gly₄Ser)₄ (SEQ ID NO:29) or (Gly₄Ser)₃ (SEQ ID NO:30). Variation in the linker length may retain or enhance activity, giving rise to superior efficacy in activity studies.

In another aspect, the antigen binding domain is a T cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Va and VP genes from a T cell clone linked by a linker (e.g., a flexible peptide). This approach is very useful to cancer associated target that itself is intracellular, however, a fragment of such antigen (peptide) is presented on the surface of the cancer cells by MHC.

In certain embodiments, the encoded antigen binding domain has a binding affinity KD of 10⁻⁴ M to 10⁻⁸ M.

In some embodiments, the encoded CAR molecule comprises an antigen binding domain that has a binding affinity KD of 10⁻⁴ M to 10⁻⁸ M, e.g., 10⁻⁵ M to 10⁻⁷ M, e.g., 10⁻⁶ M or 10⁻⁷ M, for the target antigen. In some embodiments, the antigen binding domain has a binding affinity that is at least five-fold, 10-fold, 20-fold, 30-fold, 50-fold, 100-fold or 1,000-fold less than a reference antibody, e.g., an antibody described herein. In some embodiments, the encoded antigen binding domain has a binding affinity at least 5-fold less than a reference antibody (e.g., an antibody from which the antigen binding domain is derived). In some aspects such antibody fragments are functional in that they provide a biological response that can include, but is not limited to, activation of an immune response, inhibition of signal-transduction origination from its target antigen, inhibition of kinase activity, and the like, as will be understood by a skilled artisan.

In some aspects, the antigen binding domain of the CAR is a scFv antibody fragment that is humanized compared to the murine sequence of the scFv from which it is derived.

In some aspects, the antigen binding domain of a CAR described herein (e.g., a scFv) is encoded by a nucleic acid molecule whose sequence has been codon optimized for expression in a mammalian cell. In some aspects, entire CAR construct is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

Specific antigen antibody pairs are known in the art. Non-limiting exemplary embodiments of antigen antibody pairs and components thereof are provided herein above in the section titled Targets and below.

### Bispecific CARs

In certain embodiments, the antigen binding domain is a bi- or multi-specific molecule (e.g., a multispecific antibody molecule). In some embodiments a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some embodiments the first and second epitopes are on the same antigen, e.g., the same protein (or subunit of a multimeric protein). In some embodiments the first and second epitopes overlap. In some embodiments the first and second epitopes do not overlap. In some embodiments the first and second epitopes are on different antigens, e.g., different proteins (or different subunits of a multimeric protein). In some embodiments a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In some embodiments a bispecific antibody molecule comprises a half antibody having binding specificity for a first epitope and a half antibody having binding specificity for a second epitope. In some embodiments a bispecific antibody molecule comprises a half antibody, or fragment thereof, having binding specificity for a first epitope and a half antibody, or fragment thereof, having binding specificity for a second epitope. In some embodiments a bispecific antibody molecule comprises a scFv, or fragment thereof, have binding specificity for a first epitope and a scFv, or fragment thereof, have binding specificity for a second epitope.

In some embodiments, the antibody molecule is a multi-specific (e.g., a bispecific or a trispecific) antibody molecule. Such molecules include bispecific fusion proteins, e.g., an expression construct containing two scFvs with a hydrophilic helical peptide linker between them and a full constant region, as described in, e.g., U.S. Pat. No. 5,637,481; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. The contents of the above-referenced applications are incorporated herein by reference in their entireties.

Within each antibody or antibody fragment (e.g., scFv) of a bispecific antibody molecule, the VH can be upstream or downstream of the VL. In some embodiments, the upstream antibody or antibody fragment (e.g., scFv) is arranged with its VH (VH1) upstream of its VL (VL1) and the downstream antibody or antibody fragment (e.g., scFv) is arranged with its VL (VL2) upstream of its VH (VH2), such that the overall bispecific antibody molecule has the arrangement VH1-VL1-VL2-VH2. In other embodiments, the upstream antibody or antibody fragment (e.g., scFv) is arranged with its VL (VL1) upstream of its VH (VH1) and the downstream antibody or antibody fragment (e.g., scFv) is arranged with its VH (VH2) upstream of its VL (VL2), such that the overall bispecific antibody molecule has the arrangement VL1-VH1-VH2-VL2. Optionally, a linker is disposed between the two antibodies or antibody fragments (e.g., scFvs), e.g., between VL1 and VL2 if the construct is arranged as VH1-VL1-VL2-VH2, or between VH1 and VH2 if the construct is arranged as VL1-VH1-VH2-VL2. The linker may be a linker as described herein, e.g., a (Gly₄-Ser)ₙ linker, wherein n is 1, 2, 3, 4, 5, or 6, preferably 4 (SEQ ID NO: 691). In general, the linker between the two scFvs should be long enough to avoid mispairing between the domains of the two scFvs. Optionally, a linker is disposed between the VL and VH of the first scFv. Optionally, a linker is disposed between the VL and VH of the second scFv. In constructs that have multiple linkers, any two or more of the linkers can be the same or different. Accordingly, in some embodiments, a bispecific CAR comprises VLs, VHs, and optionally one or more linkers in an arrangement as described herein.

### Transmembrane Domains

With respect to the transmembrane domain, in various embodiments, a chimeric molecule as described herein (e.g., a CAR) can be designed to comprise a transmembrane domain that is attached to the extracellular domain of the chimeric molecule. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In some aspects, the transmembrane domain is one that is associated with one of the other domains of the chimeric protein (e.g., CAR) e.g., in some embodiments, the transmembrane domain may be from the same protein that the signaling domain, costimulatory domain or the hinge domain is derived from. In another aspect, the transmembrane domain is not derived from the same protein that any other domain of the chimeric protein (e.g., CAR) is derived from. In some instances, the transmembrane domain can be selected or modified by amino acid substitution to avoid binding of such domains to the transmembrane domains of the same or different surface membrane proteins, e.g., to minimize interactions with other members of the receptor complex. In some aspects, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CAR-expressing cell.

The transmembrane domain may be derived either from a natural or from a recombinant source. Where the source is natural, the domain may be derived from any membrane-bound or transmembrane protein. In some aspects the transmembrane domain is capable of signaling to the intracellular domain(s) whenever the CAR has bound to a target. A transmembrane domain of particular use in this invention may include at least the transmembrane region(s) of e.g., the alpha, beta or zeta chain of the T-cell receptor, CD28, CD27, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL,7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, or NKG2C.

In some instances, the transmembrane domain can be attached to the extracellular region of the CAR, e.g., the antigen binding domain of the CAR, via a hinge, e.g., a hinge from a human protein. For example, in some embodiments, the hinge can be a human Ig (immunoglobulin) hinge (e.g., an IgG4 hinge, an IgD hinge), a GS linker (e.g., a GS linker described herein), a KIR2DS2 hinge or a CD8a hinge. In some embodiments, the hinge or spacer comprises (e.g., consists of) the amino acid sequence of SEQ ID NO:4. In some aspects, the transmembrane domain comprises (e.g., consists of) a transmembrane domain of SEQ ID NO: 12.

In some embodiments, the encoded transmembrane domain comprises an amino acid sequence of a CD8 transmembrane domain having at least one, two or three modifications but not more than 20, 10 or 5 modifications of an amino acid sequence of SEQ ID NO: 12, or a sequence with 95-99% identity to an amino acid sequence of SEQ ID NO: 12. In some embodiments, the encoded transmembrane domain comprises the sequence of SEQ ID NO: 12.

In other embodiments, the nucleic acid molecule encoding the CAR comprises a nucleotide sequence of a CD8 transmembrane domain, e.g., comprising the sequence of SEQ ID NO: 13, or a sequence with 95-99% identity thereof.

In some embodiments, the encoded antigen binding domain is connected to the transmembrane domain by a hinge region. In some embodiments, the encoded hinge region comprises the amino acid sequence of a CD8 hinge, e.g., SEQ ID NO: 4; or the amino acid sequence of an IgG4 hinge, e.g., SEQ ID NO: 6, or a sequence with 95-99% identity to SEQ ID NO:4 or 6. In other embodiments, the nucleic acid sequence encoding the hinge region comprises a sequence of SEQ ID NO: 5 or SEQ ID NO: 7, corresponding to a CD8 hinge or an IgG4 hinge, respectively, or a sequence with 95-99% identity to SEQ ID NO:5 or 7.

In some aspects, the hinge or spacer comprises an IgG4 hinge. For example, in some embodiments, the hinge or spacer comprises a hinge of the amino acid sequence ESKYGPPCPPCPAPEFLGGPSVFLFPPKPKDTLMISRTPEVTCVVVDVSQEDPEVQFNWYVDG VEVHNAKTKPREEQFNSTYRVVSVLTVLHQDWLNGKEYKCKVSNKGLPSSIEKTISKAKGQP REPQVYTLPPSQEEMTKNQVSLTCLVKGFYPSDIAVEWESNGQPENNYKTTPPVLDSDGSFFL YSRLTVDKSRWQEGNVFSCSVMHEALHNHYTQKSLSLSLGKM (SEQ ID NO:6). In some embodiments, the hinge or spacer comprises a hinge encoded by a nucleotide sequence of

(SEQ ID NO: 7)
GAGAGCAAGTACGGCCCTCCCTGCCCCCCTTGCCCTGCCCCCGAG
TTCCTGGGCGGACCCAGCGTGTTCCTGTTCCCCCCCAAGCCCAAG
GACACCCTGATGATCAGCCGGACCCCCGAGGTGACCTGTGTGGTG
GTGGACGTGTCCCAGGAGGACCCCGAGGTCCAGTTCAACTGGTAC
GTGGACGGCGTGGAGGTGCACAACGCCAAGACCAAGCCCCGGGAG
GAGCAGTTCAATAGCACCTACCGGGTGGTGTCCGTGCTGACCGTG
CTGCACCAGGACTGGCTGAACGGCAAGGAATACAAGTGTAAGGTG
TCCAACAAGGGCCTGCCCAGCAGCATCGAGAAAACCATCAGCAAG
GCCAAGGGCCAGCCTCGGGAGCCCCAGGTGTACACCCTGCCCCCT
AGCCAAGAGGAGATGACCAAGAACCAGGTGTCCCTGACCTGCCTG
GTGAAGGGCTTCTACCCCAGCGACATCGCCGTGGAGTGGGAGAGC
AACGGCCAGCCCGAGAACAACTACAAGACCACCCCCCCTGTGCTG
GACAGCGACGGCAGCTTCTTCCTGTACAGCCGGCTGACCGTGGAC
AAGAGCCGGTGGCAGGAGGGCAACGTCTTTAGCTGCTCCGTGATG
CACGAGGCCCTGCACAACCACTACACCCAGAAGAGCCTGAGCCTG
TCCCTGGGCAAGATG.

In some aspects, the hinge or spacer comprises an IgD hinge. For example, in some embodiments, the hinge or spacer comprises a hinge of the amino acid sequence RWPESPKAQASSVPTAQPQAEGSLAKATTAPATTRNTGRGGEEKKKEKEKEEQEERETKTPE CPSHTQPLGVYLLTPAVQDLWLRDKATFTCFVVGSDLKDAHLTWEVAGKVPTGGVEEGLLE RHSNGSQSQHSRLTLPRSLWNAGTSVTCTLNHPSLPPQRLMALREPAAQAPVKLSLNLLASS DPPEAASWLLCEVSGFSPPNILLMWLEDQREVNTSGFAPARPPPQPGSTTFWAWSVLRVPAPP SPQPATYTCVVSHEDSRTLLNASRSLEVSYVTDH (SEQ ID NO:8). In some embodiments, the hinge or spacer comprises a hinge encoded by a nucleotide sequence of

(SEQ ID NO: 9)
AGGTGGCCCGAAAGTCCCAAGGCCCAGGCATCTAGTGTTCCTACT
GCACAGCCCCAGGCAGAAGGCAGCCTAGCCAAAGCTACTACTGCA
CCTGCCACTACGCGCAATACTGGCCGTGGCGGGGAGGAGAAGAAA
AAGGAGAAAGAGAAAGAAGAACAGGAAGAGAGGGAGACCAAGACC
CCTGAATGTCCATCCCATACCCAGCCGCTGGGCGTCTATCTCTTG
ACTCCCGCAGTACAGGACTTGTGGCTTAGAGATAAGGCCACCTTT
ACATGTTTCGTCGTGGGCTCTGACCTGAAGGATGCCCATTTGACT
TGGGAGGTTGCCGGAAAGGTACCCACAGGGGGGGTTGAGGAAGGG
TTGCTGGAGCGCCATTCCAATGGCTCTCAGAGCCAGCACTCAAGA
CTCACCCTTCCGAGATCCCTGTGGAACGCCGGGACCTCTGTCACA
TGTACTCTAAATCATCCTAGCCTGCCCCCACAGCGTCTGATGGCC
CTTAGAGAGCCAGCCGCCCAGGCACCAGTTAAGCTTAGCCTGAAT
CTGCTCGCCAGTAGTGATCCCCCAGAGGCCGCCAGCTGGCTCTTA
TGCGAAGTGTCCGGCTTTAGCCCGCCCAACATCTTGCTCATGTGG
CTGGAGGACCAGCGAGAAGTGAACACCAGCGGCTTCGCTCCAGCC
CGGCCCCCACCCCAGCCGGGTTCTACCACATTCTGGGCCTGGAGT
GTCTTAAGGGTCCCAGCACCACCTAGCCCCCAGCCAGCCACATAC
ACCTGTGTTGTGTCCCATGAAGATAGCAGGACCCTGCTAAATGCT
TCTAGGAGTCTGGAGGTTTCCTACGTGACTGACCATT.

In some aspects, the transmembrane domain may be recombinant, in which case it will comprise predominantly hydrophobic residues such as leucine and valine. In some aspects a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in some aspects, the linker comprises the amino acid sequence of GGGGSGGGGS (SEQ ID NO:10). In some embodiments, the linker is encoded by a nucleotide sequence of GGTGGCGGAGGTTCTGGAGGTGGAGGTTCC (SEQ ID NO:11). In some embodiments, the linker comprises the amino acid sequence of GGGGS (SEQ ID NO: 877). In some embodiments the linker is encoded by a nucleotide sequence of SEQ ID NO: 876).

In some aspects, the hinge or spacer comprises a KIR2DS2 hinge.

### Signaling Domains

In embodiments of the invention having an intracellular signaling domain, such a domain can contain, e.g., one or more of a primary signaling domain and/or a costimulatory signaling domain. In some embodiments, the intracellular signaling domain comprises a sequence encoding a primary signaling domain. In some embodiments, the intracellular signaling domain comprises a costimulatory signaling domain. In some embodiments, the intracellular signaling domain comprises a primary signaling domain and a costimulatory signaling domain.

The intracellular signaling sequences within the cytoplasmic portion of the CAR of the invention may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences. In some embodiments, a glycine-serine doublet can be used as a suitable linker. In some embodiments, a single amino acid, e.g., an alanine, a glycine, can be used as a suitable linker.

In some aspects, the intracellular signaling domain is designed to comprise two or more, e.g., 2, 3, 4, 5, or more, costimulatory signaling domains. In some embodiments, the two or more, e.g., 2, 3, 4, 5, or more, costimulatory signaling domains, are separated by a linker molecule, e.g., a linker molecule described herein. In some embodiments, the intracellular signaling domain comprises two costimulatory signaling domains. In some embodiments, the linker molecule is a glycine residue. In some embodiments, the linker is an alanine residue.

### Primary Signaling Domains

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs. In CARs such domains are used for the same purpose. Examples of ITAM containing primary intracellular signaling domains that are of particular use in the invention include those of CD3 zeta, common FcR gamma (FCER1G). Fc gamma RIIa. FcR beta(Fc Epsilon R1b), CD3 gamma, CD3 delta, CD3 epsilon CD79a, CD79b, DAP10, and DAP12. In some embodiments, a CAR of the invention comprises an intracellular signaling domain, e.g., a primary signaling domain of CD3-zeta.

In some embodiments, the encoded primary signaling domain comprises a functional signaling domain of CD3 zeta. The encoded CD3 zeta primary signaling domain can comprise an amino acid sequence having at least one, two or three modifications but not more than 20, 10 or 5 modifications of an amino acid sequence of SEQ ID NO: 18 or SEQ ID NO: 20, or a sequence with 95-99% identity to an amino acid sequence of SEQ ID NO:18 or SEQ ID NO: 20. In some embodiments, the encoded primary signaling domain comprises a sequence of SEQ ID NO:18 or SEQ ID NO: 20. In other embodiments, the nucleic acid sequence encoding the primary signaling domain comprises a sequence of SEQ ID NO:19 or SEQ ID NO: 21, or a sequence with 95-99% identity thereof.

### Costimulatory Signaling Domains

In some embodiments, the encoded intracellular signaling domain comprises a costimulatory signaling domain. For example, the intracellular signaling domain can comprise a primary signaling domain and a costimulatory signaling domain. In some embodiments, the encoded costimulatory signaling domain comprises a functional signaling domain of a protein chosen from one or more of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CDIIc, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.

In some embodiments, the encoded costimulatory signaling domain comprises an amino acid sequence having at least one, two or three modifications but not more than 20, 10 or 5 modifications of an amino acid sequence of SEQ ID NO:14 or SEQ ID NO: 16, or a sequence with 95-99% identity to an amino acid sequence of SEQ ID NO:14 or SEQ ID NO: 16. In some embodiments, the encoded costimulatory signaling domain comprises a sequence of SEQ ID NO: 14 or SEQ ID NO: 16. In other embodiments, the nucleic acid sequence encoding the costimulatory signaling domain comprises a sequence of SEQ ID NO:15 or SEQ ID NO: 17, or a sequence with 95-99% identity thereof.

In other embodiments, the encoded intracellular domain comprises the sequence of SEQ ID NO: 14 or SEQ ID NO: 16, and the sequence of SEQ ID NO: 18 or SEQ ID NO: 20, wherein the sequences comprising the intracellular signaling domain are expressed in the same frame and as a single polypeptide chain.

In some embodiments, the nucleic acid sequence encoding the intracellular signaling domain comprises a sequence of SEQ ID NO:15 or SEQ ID NO: 17, or a sequence with 95-99% identity thereof, and a sequence of SEQ ID NO:19 or SEQ ID NO:21, or a sequence with 95-99% identity thereof.

In some embodiments, the nucleic acid molecule further encodes a leader sequence. In some embodiments, the leader sequence comprises the sequence of SEQ ID NO: 2.

In some aspects, the intracellular signaling domain is designed to comprise the signaling domain of CD3-zeta and the signaling domain of CD28. In some aspects, the intracellular signaling domain is designed to comprise the signaling domain of CD3-zeta and the signaling domain of 4-1BB. In some aspects, the signaling domain of 4-1BB is a signaling domain of SEQ ID NO: 14. In some aspects, the signaling domain of CD3-zeta is a signaling domain of SEQ ID NO: 18.

In some aspects, the intracellular signaling domain is designed to comprise the signaling domain of CD3-zeta and the signaling domain of CD27. In some aspects, the signaling domain of CD27 comprises an amino acid sequence of QRRKYRSNKGESPVEPAEPCRYSCPREEEGSTIPIQEDYRKPEPACSP (SEQ ID NO:16). In some aspects, the signaling domain of CD27 is encoded by a nucleic acid sequence of

(SEQ ID NO: 17)
AGGAGTAAGAGGAGCAGGCTCCTGCACAGTGACTACATGAACATG
ACTCCCCGCCGCCCCGGGCCCACCCGCAAGCATTACCAGCCCTAT
GCCCCACCACGCGACTTCGCAGCCTATCGCTCC

### Inhibitory Domains

In some embodiments, the vector comprises a nucleic acid sequence that encodes a CAR, e.g., a CAR described herein, and a nucleic acid sequence that encodes an inhibitory molecule comprising: an inhKIR cytoplasmic domain; a transmembrane domain, e.g., a KIR transmembrane domain; and an inhibitor cytoplasmic domain, e.g., an ITIM domain, e.g., an inhKIR ITIM domain. In some embodiments the inhibitory molecule is a naturally occurring inhKIR, or a sequence sharing at least 50, 60, 70, 80, 85, 90, 95, or 99% homology with, or that differs by no more than 1, 2, 3, 4, 5, 6, 7, 8, 9, 10, 15, or 20 residues from, a naturally occurring inhKIR.

In some embodiments, the nucleic acid sequence that encodes an inhibitory molecule comprises: a SLAM family cytoplasmic domain; a transmembrane domain, e.g., a SLAM family transmembrane domain; and an inhibitor cytoplasmic domain, e.g., a SLAM family domain, e.g., an SLAM family ITIM domain. In some embodiments the inhibitory molecule is a naturally occurring SLAM family member, or a sequence sharing at least 50, 60, 70, 80, 85, 90, 95, or 99% homology with, or that differs by no more than 1, 2, 3, 4, 5, 6, 7, 8, 9, 10, 15, or 20 residues from, a naturally occurring SLAM family member.

In some embodiments, the vector is an in vitro transcribed vector, e.g., a vector that transcribes RNA of a nucleic acid molecule described herein. In some embodiments, the nucleic acid sequence in the vector further comprises a poly(A) tail, e.g., a poly A tail. In some embodiments, the nucleic acid sequence in the vector further comprises a 3′UTR, e.g., a 3′ UTR described herein, e.g., comprising at least one repeat of a 3′UTR derived from human beta-globulin. In some embodiments, the nucleic acid sequence in the vector further comprises promoter, e.g., a T2A promoter.

### Promoters

In some embodiments, the vector further comprises a promoter. In some embodiments, the promoter is chosen from an EF-1 promoter, a CMV IE gene promoter, an EF-1α promoter, an ubiquitin C promoter, or a phosphoglycerate kinase (PGK) promoter. In some embodiments, the promoter is an EF-1 promoter. In some embodiments, the EF-1 promoter comprises a sequence of SEQ ID NO: 1.

In some aspects of the present invention, immune effector cells, e.g., T cells, can be obtained from a unit of blood collected from a subject using any number of techniques known to the skilled artisan, such as Ficoll™ separation. In some aspects, cells from the circulating blood of an individual are obtained by apheresis. The apheresis product typically contains lymphocytes, including T cells, monocytes, granulocytes, B cells, other nucleated white blood cells, red blood cells, and platelets. In some aspects, the cells collected by apheresis may be washed to remove the plasma fraction and, optionally, to suspend the cells in a buffer or medium for subsequent processing steps. In some embodiments, the cells are washed with phosphate buffered saline (PBS). In an alternative embodiment, the wash solution lacks calcium and may lack magnesium or may lack many if not all divalent cations.

TABLE 17
Sequences of various components of CAR
(aa-amino acids, na-nucleic acids that
encodes the corresponding protein)

| SEQ | | |
|---|---|---|
| ID | | |
| NO | description | Sequence |
| 1 | EF-1 promoter | AGCGCACATCGCCCACAGTCCCCGAGAAG |
| | | TTGGGGGGAGGGGTCGGCAATTGAACCGGT |
| | | GCCTAGAGAAGGTGGCGCGGGGTAAACTGG |
| | | GAAAGTGATGTCGTGTACTGGCTCCGCCTT |
| | | TTTCCCGAGGGTGGGGGAGAACCGTATATA |
| | | AGTGCAGTAGTCGCCGTGAACGTTCTTTTT |
| | | CGCAACGGGTTTGCCGCCAGAACACAGGTA |
| | | AGTGCCGTGTGTGGTTCCCGCGGGCCTGGC |
| | | CTCTTTACGGGTTATGGCCCTTGCGTGCCT |
| | | TGAATTACTTCCACCTGGCTGCAGTACGTG |
| | | ATTCTTGATCCCGAGCTTCGGGTTGGAAGT |
| | | GGGTGGGAGAGTTCGAGGCCTTGCGCTTAA |
| | | GGAGCCCCTTCGCCTCGTGCTTGAGTTGAG |
| | | GCCTGGCCTGGGCGCTGGGGCCGCCGCGTG |
| | | CGAATCTGGTGGCACCTTCGCGCCTGTCTC |
| | | GCTGCTTTCGATAAGTCTCTAGCCATTTAA |
| | | AATTTTTGATGACCTGCTGCGACGCTTTTT |
| | | TTCTGGCAAGATAGTCTTGTAAATGCGGGC |
| | | CAAGATCTGCACACTGGTATTTCGGTTTTT |
| | | GGGGCCGCGGGCGGCGACGGGGCCCGTGCG |
| | | TCCCAGCGCACATGTTCGGCGAGGCGGGGC |
| | | CTGCGAGCGCGGCCACCGAGAATCGGACGG |
| | | GGGTAGTCTCAAGCTGGCCGGCCTGCTCTG |
| | | GTGCCTGGCCTCGCGCCGCCGTGTATCGCC |
| | | CCGCCCTGGGCGGCAAGGCTGGCCCGGTCG |
| | | GCACCAGTTGCGTGAGCGGAAAGATGGCCG |
| | | CTTCCCGGCCCTGCTGCAGGGAGCTCAAAA |
| | | TGGAGGACGCGGCGCTCGGGAGAGCGGGCG |
| | | GGTGAGTCACCCACACAAAGGAAAAGGGCC |
| | | TTTCCGTCCTCAGCCGTCGCTTCATGTGAC |
| | | TCCACGGAGTACCGGGCGCCGTCCAGGCAC |
| | | CTCGATTAGTTCTCGAGCTTTTGGAGTACG |
| | | TCGTCTTTAGGTTGGGGGGAGGGGTTTTAT |
| | | GCGATGGAGTTTCCCCACACTGAGTGGGTG |
| | | GAGACTGAAGTTAGGCCAGCTTGGCACTTG |
| | | ATGTAATTCTCCTTGGAATTTGCCCTTTTT |
| | | GAGTTTGGATCTTGGTTCATTCTCAAGCCT |
| | | CAGACAGTGGTTCAAAGTTTTTTTCTTCCA |
| | | TTTCAGGTGTCGTGA |
| 2 | Leader (aa) | MALPVTALLLPLALLLHAARP |
| 3 | Leader (na) | ATGGCCCTGCCTGTGACAGCCCTGCTGCTG |
| | | CCTCTGGCTCTGCTGCTGCATGCCGCTAGA |
| | | CCC |
| 4 | CD 8 hinge | TTTPAPRPPTPAPTIASQPLSLRPEACRPA |
| | (aa) | AGGAVHTRGLDFACD |
| 5 | CD8 hinge (na) | ACCACGACGCCAGCGCCGCGACCACCAACA |
| | | CCGGCGCCCACCATCGCGTCGCAGCCCCTG |
| | | TCCCTGCGCCCAGAGGCGTGCCGGCCAGCG |
| | | GCGGGGGGCGCAGTGCACACGAGGGGGCTG |
| | | GACTTCGCCTGTGAT |
| 6 | Ig4 hinge (aa) | ESKYGPPCPPCPAPEFLGGPSVFLFPPKPK |
| | | DTLMISRTPEVTCVVVDVSQEDPEVQFNWY |
| | | VDGVEVHNAKTKPREEQFNSTYRVVSVLTV |
| | | LHQDWLNGKEYKCKVSNKGLPSSIEKTISK |
| | | AKGQPREPQVYTLPPSQEEMTKNQVSLTCL |
| | | VKGFYPSDIAVEWESNGQPENNYKTTPPV |
| | | LDSDGSFFLYSRLTVDKSRWQEGNVFSCSV |
| | | MHEALHNHYTQKSLSLSLGKM |
| 7 | Ig4 hinge (na) | GAGAGCAAGTACGGCCCTCCCTGCCCCCCT |
| | | TGCCCTGCCCCCGAGTTCCTGGGCGGACCC |
| | | AGCGTGTTCCTGTTCCCCCCCAAGCCCAAG |
| | | GACACCCTGATGATCAGCCGGACCCCCGAG |
| | | GTGACCTGTGTGGTGGTGGACGTGTCCCAG |
| | | GAGGACCCCGAGGTCCAGTTCAACTGGTAC |
| | | GTGGACGGCGTGGAGGTGCACAACGCCAAG |
| | | ACCAAGCCCCGGGAGGAGCAGTTCAATAGC |
| | | ACCTACCGGGTGGTGTCCGTGCTGACCGTG |
| | | CTGCACCAGGACTGGCTGAACGGCAAGGAA |
| | | TACAAGTGTAAGGTGTCCAACAAGGGCCTG |
| | | CCCAGCAGCATCGAGAAAACCATCAGCAAG |
| | | GCCAAGGGCCAGCCTCGGGAGCCCCAGGTG |
| | | TACACCCTGCCCCCTAGCCAAGAGGAGATG |
| | | ACCAAGAACCAGGTGTCCCTGACCTGCCTG |
| | | GTGAAGGGCTTCTACCCCAGCGACATCGCC |
| | | GTGGAGTGGGAGAGCAACGGCCAGCCCGAG |
| | | AACAACTACAAGACCACCCCCCCTGTGCTG |
| | | GACAGCGACGGCAGCTTCTTCCTGTACAGC |
| | | CGGCTGACCGTGGACAAGAGCCGGTGGCAG |
| | | GAGGGCAACGTCTTTAGCTGCTCCGTGATG |
| | | CACGAGGCCCTGCACAACCACTACACCCAG |
| | | AAGAGCCTGAGCCTGTCCCTGGGCAAGATG |
| 8 | IgD hinge (aa) | RWPESPKAQASSVPTAQPQAEGSLAKATTA |
| | | PATTRNTGRGGEEKKKEKEKEEQEERETKT |
| | | PECPSHTQPLGVYLLTPAVQDLWLRDKATF |
| | | TCFVVGSDLKDAHLTWEVAGKVPTGGVEEG |
| | | LLERHSNGSQSQHSRLTLPRSLWNAGTSVT |
| | | CTLNHPSLPPQRLMALREPAAQAPVKLSLN |
| | | LLASSDPPEAASWLLCEVSGFSPPNILLMW |
| | | LEDQREVNTSGFAPARPPPQPGSTTFWAWS |
| | | VLRVPAPPSPQPATYTCVVSHEDSRTLLNA |
| | | SRSLEVSYVTDH |
| 9 | IgD hinge (na) | AGGTGGCCCGAAAGTCCCAAGGCCCAGGCA |
| | | TCTAGTGTTCCTACTGCACAGCCCCAGGCA |
| | | GAAGGCAGCCTAGCCAAAGCTACTACTGCA |
| | | CCTGCCACTACGCGCAATACTGGCCGTGGC |
| | | GGGGAGGAGAAGAAAAAGGAGAAAGAGAAA |
| | | GAAGAACAGGAAGAGAGGGAGACCAAGACC |
| | | CCTGAATGTCCATCCCATACCCAGCCGCTG |
| | | GGCGTCTATCTCTTGACTCCCGCAGTACAG |
| | | GACTTGTGGCTTAGAGATAAGGCCACCTTT |
| | | ACATGTTTCGTCGTGGGCTCTGACCTGAAG |
| | | GATGCCCATTTGACTTGGGAGGTTGCCGGA |
| | | AAGGTACCCACAGGGGGGGTTGAGGAAGGG |
| | | TTGCTGGAGCGCCATTCCAATGGCTCTCAG |
| | | AGCCAGCACTCAAGACTCACCCTTCCGAGA |
| | | TCCCTGTGGAACGCCGGGACCTCTGTCACA |
| | | TGTACTCTAAATCATCCTAGCCTGCCCCCA |
| | | CAGCGTCTGATGGCCCTTAGAGAGCCAGCC |
| | | GCCCAGGCACCAGTTAAGCTTAGCCTGAAT |
| | | CTGCTCGCCAGTAGTGATCCCCCAGAGGCC |
| | | GCCAGCTGGCTCTTATGCGAAGTGTCCGGC |
| | | TTTAGCCCGCCCAACATCTTGCTCATGTGG |
| | | CTGGAGGACCAGCGAGAAGTGAACACCAGC |
| | | GGCTTCGCTCCAGCCCGGCCCCCACCCCAG |
| | | CCGGGTTCTACCACATTCTGGGCCTGGAGT |
| | | GTCTTAAGGGTCCCAGCACCACCTAGCCCC |
| | | CAGCCAGCCACATACACCTGTGTTGTGTCC |
| | | CATGAAGATAGCAGGACCCTGCTAAATGCT |
| | | TCTAGGAGTCTGGAGGTTTCCTACGTGACT |
| | | GACCATT |
| 10 | GS hinge/ | GGGGSGGGGS |
| | linker | |
| | (aa) | |
| 11 | GS hinge/ | GGTGGCGGAGGTTCTGGAGGTGGAGGTTCC |
| | linker | |
| | (na) | |
| 12 | CD8 TM (aa) | IYIWAPLAGTCGVLLLSLVITLYC |
| 13 | CD8 TM (na) | ATCTACATCTGGGCGCCCTTGGCCGGGACT |
| | | TGTGGGGTCCTTCTCCTGTCACTGGTTATC |
| | | ACCCTTTACTGC |
| 14 | 4-1BB | KRGRKKLLYIFKQPFMRPVQTTQEEDGCSC |
| | intracellular | RFPEEEEGGCEL |
| | domain (aa) | |
| 15 | 4-1BB | AAACGGGGCAGAAAGAAACTCCTGTATATA |
| | intracellular | TTCAAACAACCATTTATGAGACCAGTACAA |
| | domain (na) | ACTACTCAAGAGGAAGATGGCTGTAGCTGC |
| | | CGATTTCCAGAAGAAGAAGAAGGAGGATGT |
| | | GAACTG |
| 16 | CD27 (aa) | QRRKYRSNKGESPVEPAEPCRYSCPREEEG |
| | | STIPIQEDYRKPEPACSP |
| 17 | CD27 (na) | AGGAGTAAGAGGAGCAGGCTCCTGCACAGT |
| | | GACTACATGAACATGACTCCCCGCCGCCCC |
| | | GGGCCCACCCGCAAGCATTACCAGCCCTAT |
| | | GCCCCACCACGCGACTTCGCAGCCTATCGC |
| | | TCC |
| 18 | CD3-zeta (aa) | RVKFSRSADAPAYKQGQNQLYNELNLGRRE |
| | | EYDVLDKRRGRDPEMGGKPRRKNPQEGLYN |
| | | ELQKDKMAEAYSEIGMKGERRRGKGHDGLY |
| | | QGLSTATKDTYDALHMQALPPR |
| 19 | CD3-zeta (na) | AGAGTGAAGTTCAGCAGGAGCGCAGACGCC |
| | | CCCGCGTACAAGCAGGGCCAGAACCAGCTC |
| | | TATAACGAGCTCAATCTAGGACGAAGAGAG |
| | | GAGTACGATGTTTTGGACAAGAGACGTGGC |
| | | CGGGACCCTGAGATGGGGGGAAAGCCGAGA |
| | | AGGAAGAACCCTCAGGAAGGCCTGTACAAT |
| | | GAACTGCAGAAAGATAAGATGGCGGAGGCC |
| | | TACAGTGAGATTGGGATGAAAGGCGAGCGC |
| | | CGGAGGGGCAAGGGGCACGATGGCCTTTAC |
| | | CAGGGTCTCAGTACAGCCACCAAGGACACC |
| | | TACGACGCCCTTCACATGCAGGCCCTGCCC |
| | | CCTCGC |
| 20 | CD3-zeta (aa) | RVKFSRSADAPAYQQGQNQLYNELNLGRRE |
| | | EYDVLDKRRGRDPEMGGKPRRKNPQEGLYN |
| | | ELQKDKMAEAYSEIGMKGERRRGKGHDGLY |
| | | QGLSTATKDTYDALHMQALPPR |
| 21 | CD3-zeta (na) | AGAGTGAAGTTCAGCAGGAGCGCAGACGCC |
| | | CCCGCGTACCAGCAGGGCCAGAACCAGCTC |
| | | TATAACGAGCTCAATCTAGGACGAAGAGAG |
| | | GAGTACGATGTTTTGGACAAGAGACGTGGC |
| | | CGGGACCCTGAGATGGGGGGAAAGCCGAGA |
| | | AGGA |
| | | AGAACCCTCAGGAAGGCCTGTACAATGAAC |
| | | TGCAGAAAGATAAGATGGCGG |
| | | AGGCCTACAGTGAGATTGGGATGAAAGGCG |
| | | AGCGCCGGAGGGGCAAGGGGC |
| | | ACGATGGCCTTTACCAGGGTCTCAGTACAG |
| | | CCACCAAGGACACCTACGACGCCCTTCACA |
| | | TGCAGGCCCTGCCCCCTCGC |
| 22 | linker | GGGGS |
| 23 | linker | GGTGGCGGAGGTTCTGGAGGTGGAGGTTCC |
| 24 | PD-1 | Pgwfldspdrpwnpptfspallvvtegdna |
| | extracellular | tftcsfsntsesfvlnwyrmspsnqtdkla |
| | domain (aa) | afpedrsqpgqdcrfrvtqlpngrdfhmsv |
| | | vrarrndsgtylegaislapkaqikeslra |
| | | elrvterraevptahpspsprpagqfqtlv |
| 25 | PD-1 | Cccggatggtttctggactctccggatcgc |
| | extracellular | ccgtggaatcccccaaccttctcaccggca |
| | domain (na) | ctcttggttgtgactgagggcgataatgcg |
| | | accttcacgtgctcgttctccaacacctcc |
| | | gaatcattcgtgctgaactggtaccgcatg |
| | | agcccgtcaaaccagaccgacaagctcgcc |
| | | gcgtttccggaagatcggtcgcaaccggga |
| | | caggattgtcggttccgcgtgactcaactg |
| | | ccgaatggcagagacttccacatgagcgtg |
| | | gtccgcgctaggcgaaacgactccgggacc |
| | | tacctgtgcggagccatctcgctggcgcct |
| | | aaggcccaaatcaaagagagcttgagggcc |
| | | gaactgagagtgaccgagcgcagagctgag |
| | | gtgccaactgcacatccatccccatcgcct |
| | | cggcctgcggggcagtttcagaccctggtc |
| 26 | PD-1 CAR (aa) | Malpvtalllplalllhaarppgwfldspd |
| | with signal | rpwnpptfspallvvtegdnatftcsfsnt |
| | | sesfvlnwyrmspsnqtdklaafpedrsqp |
| | | gqdcrfrvtqlpngrdfhmsvvrarrndsg |
| | | tylcgaislapkaqikeslraelrvterra |
| | | evptahpspsprpagqfqtlvtttpaprpp |
| | | tpaptiasqplslrpeacrpaaggavhtrg |
| | | ldfacdiyiwaplagtcgvlllslvitlyc |
| | | krgrkkllyifkqpfmrpvqttqeedgcsc |
| | | rfpeeeeggcelrvkfsrsadapaykqgqn |
| | | qlynelnlgrreeydvldkrrgrdpemggk |
| | | prrknpqeglynelqkdkmaeayseigmkg |
| | | errrgkghdglyqglstatkdtydaIhmqa |
| | | lppr |
| 27 | PD-1 CAR (na) | Atggccctccctgtcactgccctgcttctc |
| | | cccctcgcactcctgctccacgccgctaga |
| | | ccacccggatggtttctggactctccggat |
| | | cgcccgtggaatcccccaaccttctcaccg |
| | | gcactcttggttgtgactgagggcgataat |
| | | gcgaccttcacgtgctcgttctccaacacc |
| | | tccgaatcattcgtgctgaactggtaccgc |
| | | atgagcccgtcaaaccagaccgacaagctc |
| | | gccgcgtttccggaagatcggtcgcaaccg |
| | | ggacaggattgtcggttccgcgtgactcaa |
| | | ctgccgaatggcagagacttccacatgagc |
| | | gtggtccgcgctaggcgaaacgactccggg |
| | | acctacctgtgcggagccatctcgctggcg |
| | | cctaaggcccaaatcaaagagagcttgagg |
| | | gccgaactgagagtgaccgagcgcagagct |
| | | gaggtgccaactgcacatccatccccatcg |
| | | cctcggcctgcggggcagtttcagaccctg |
| | | gtcacgaccactccggcgccgcgcccaccg |
| | | actccggccccaactatcgcgagccagccc |
| | | ctgtcgctgaggccggaagcatgccgccct |
| | | gccgccggaggtgctgtgcatacccgggga |
| | | ttggacttcgcatgcgacatctacatttgg |
| | | gctcctctcgccggaacttgtggcgtgctc |
| | | cttctgtccctggtcatcaccctgtactgc |
| | | aagcggggtcggaaaaagcttctgtacatt |
| | | ttcaagcagcccttcatgaggcccgtgcaa |
| | | accacccaggaggaggacggttgctcctgc |
| | | cggttccccgaagaggaagaaggaggttgc |
| | | gagctgcgcgtgaagttctcccggagcgcc |
| | | gacgcccccgcctataagcagggccagaac |
| | | cagctgtacaacgaactgaacctgggacgg |
| | | cgggaagagtacgatgtgctggacaagcgg |
| | | cgcggccgggaccccgaaatggggggaagc |
| | | ctagaagaaagaaccctcaggaaggcctgt |
| | | ataacgagctgcagaaggacaagatggccg |
| | | aggcctactccgaaattgggatgaagggag |
| | | agcggcggaggggaaaggggcacgacggcc |
| | | tgtaccaaggactgtccaccgccaccaagg |
| | | acacatacgatgccctgcacatgcaggccc |
| | | ttccccctcgc |
| 28 | linker | (Gly-Gly-Gly-Ser)n, |
| | | where n = 1-10 |
| 29 | linker | (Gly4 Ser)4 |
| 30 | linker | (Gly4 Ser)3 |
| 31 | linker | (Gly3Ser) |
| 39 | PD1 CAR (aa) | Pgwfldspdrpwnpptfspallvvtegdna |
| | | tftcsfsntsesfvlnwyrmspsnqtdkla |
| | | afpedrsqpgqderfrvtqlpngrdfhmsv |
| | | vrarrndsgtylegaislapkaqikeslra |
| | | elrvterraevptahpspsprpagqfqtlv |
| | | tttpaprpptpaptiasqplslrpeacrpa |
| | | aggavhtrgldfacdiyiwaplagtcgvll |
| | | lslvitlyckrgrkkllyifkqpfmrpvqt |
| | | tqeedgcscrfpeeeeggcelrvkfsrsad |
| | | apaykqgqnqlynelnlgrreeydvldkrr |
| | | grdpemggkprrknpqeglynelqkdkmae |
| | | ayseigmkgerrrgkghdglyqglstatkd |
| | | tydalhmqalppr |

### Methods of Manufacture

Lentiviral vectors described herein (e.g., those made using a method described herein), can be used, e.g., in the in vitro manufacture of CAR-T cells.

CARTs disclosed herein can be manufactured ex vivo by any known methods in the art. For example, methods described in WO2012/079000, or WO2020/047452 (both incorporated herein by reference) may be used. CARTs disclosed herein can also be manufactured in vivo by any known methods in the art. For example, methods described in WO2020/176397 (incorporated herein by reference). An immune effector cell (e.g., T cell or NK cell) may express one CAR, or two or more CARs.

In some embodiments, the methods disclosed herein may manufacture immune effector cells engineered to express one or more CARs in less than 24 hours. Without wishing to be bound by theory, the methods provided herein preserve the undifferentiated phenotype of T cells, such as naive T cells, during the manufacturing process. These CAR-expressing cells with an undifferentiated phenotype may persist longer and/or expand better in vivo after infusion. In some embodiments, CART cells produced by the manufacturing methods provided herein comprise a higher percentage of stem cell memory T cells, compared to CART cells produced by the traditional manufacturing process, e.g., as measured using scRNA-seq.

In some embodiments, CART cells produced by the manufacturing methods provided herein comprise a higher percentage of effector T cells, compared to CART cells produced by the traditional manufacturing process, e.g., as measured using scRNA-seq. In some embodiments, CART cells produced by the manufacturing methods provided herein better preserve the sternness of T cells, compared to CART cells produced by the traditional manufacturing process, e.g., as measured using scRNA-seq. In some embodiments, CART cells produced by the manufacturing methods provided herein show a lower level of hypoxia, compared to CART cells produced by the traditional manufacturing process, e.g., as measured using scRNA-seq. In some embodiments, CART cells produced by the manufacturing methods provided herein show a lower level of autophagy, compared to CART cells produced by the traditional manufacturing process, e.g., as measured using scRNA-seq. In some embodiments, the immune effector cells are engineered to comprise a nucleic acid molecule encoding one or more CARs disclosed herein.

In some embodiments, the methods disclosed herein do not involve using a bead, such as Dynabeads® (for example, CD3/CD28 Dynabeads®), and do not involve a de-beading step. In some embodiments, the CART cells manufactured by the methods disclosed herein may be administered to a subject with minimal ex vivo expansion, for example, less than 1 day, less than 12 hours, less than 8 hours, less than 6 hours, less than 4 hours, less than 3 hours, less than 2 hours, less than 1 hour, or no ex vivo expansion. Accordingly, the methods described herein provide a fast manufacturing process of making improved CAR-expressing cell products for use in treating a disease in a subject.

In some embodiments, the present disclosure provides methods of making a population of cells (for example, T cells) that express a chimeric antigen receptor (CAR) comprising: (i) contacting a population of cells (for example, T cells, for example, T cells isolated from a frozen or fresh leukapheresis product) with an agent that stimulates a CD3/TCR complex and/or an agent that stimulates a costimulatory molecule on the surface of the cells; (ii) contacting the population of cells (for example, T cells) with a nucleic acid molecule(s) (for example, a DNA or RNA molecule) encoding the CAR(s), thereby providing a population of cells (for example, T cells) comprising the nucleic acid molecule, and (iii) harvesting the population of cells (for example, T cells) for storage (for example, reformulating the population of cells in cryopreservation media) or administration, wherein: (a) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 26 hours after the beginning of step (i), for example, no later than 22, 23, or 24 hours after the beginning of step (i), for example, no later than 24 hours after the beginning of step (i); (b) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 30 hours after the beginning of step (ii), for example, no later than 22, 23, 24, 25, 26, 27, 28, 29, or 30 hours after the beginning of step (ii); or (c) the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i). In some embodiments, the nucleic acid molecule in step (ii) is a DNA molecule. In some embodiments, the nucleic acid molecule in step (ii) is an RNA molecule. In some embodiments, the nucleic acid molecule in step (ii) is on a viral vector, for example, a viral vector chosen from a lentivirus vector, an adenoviral vector, or a retrovirus vector. In some embodiments, the nucleic acid molecule in step (ii) is on a non-viral vector. In some embodiments, the nucleic acid molecule in step (ii) is on a plasmid. In some embodiments, the nucleic acid molecule in step (ii) is not on any vector. In some embodiments, step (ii) comprises transducing the population of cells (for example, T cells) a viral vector(s) comprising a nucleic acid molecule encoding the CAR(s).

In some embodiments, the population of cells (for example, T cells) is collected from an apheresis sample (for example, a leukapheresis sample) from a subject.

In some embodiments, the apheresis sample (for example, a leukapheresis sample) is collected from the subject and shipped as a frozen sample (for example, a cryopreserved sample) to a cell manufacturing facility. Then the frozen apheresis sample is thawed, and T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then seeded for CART manufacturing using the activation process described herein. In some embodiments, the selected T cells (for example, CD4+ T cells and/or CD8+ T cells) undergo one or more rounds of freeze-thaw before being seeded for CART manufacturing.

In some embodiments, the apheresis sample (for example, a leukapheresis sample) is collected from the subject and shipped as a fresh product (for example, a product that is not frozen) to a cell manufacturing facility. T cells (for example, CD4+ T cells and/or CD 8+ T cells) are selected from the apheresis sample, for example, using a cell sorting machine (for example, a CliniMACS® Prodigy® device). The selected T cells (for example, CD4+ T cells and/or CD8+ T cells) are then seeded for CART manufacturing using the activation process described herein. In some embodiments, the selected T cells (for example, CD4+ T cells and/or CD8+ T cells) undergo one or more rounds of freeze-thaw before being seeded for CART manufacturing.

In some embodiments, the apheresis sample (for example, a leukapheresis sample) is collected from the subject. T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then shipped as a frozen sample (for example, a cryopreserved sample) to a cell manufacturing facility. The selected T cells (for example, CD4+ T cells and/or CD8+ T cells) are later thawed and seeded for CART manufacturing using the activation process described herein.

In some embodiments, cells (for example, T cells) are contacted with anti-CD3 and anti-CD28 antibodies for, for example, 12 hours, followed by transduction with a vector (for example, a lentiviral vector) (e.g. one or more vectors) encoding a CAR (e.g. one or more CARs). 24 hours after culture initiation, the cells are washed and formulated for storage or administration.

Without wishing to be bound by theory, brief CD3 and CD28 stimulation may promote efficient transduction of self-renewing T cells. Compared to traditional CART manufacturing approaches, the activation process provided herein does not involve prolonged ex vivo expansion. Similar to the cytokine process, the activation process provided herein also preserves undifferentiated T cells during CART manufacturing.

In some embodiments, the population of cells is contacted with an agent that stimulates a CD3/TCR complex and/or an agent that stimulates a costimulatory molecule on the surface of the cells.

In some embodiments, the agent that stimulates a CD3/TCR complex is an agent that stimulates CD3. In some embodiments, the agent that stimulates a costimulatory molecule is an agent that stimulates CD28, ICOS, CD27, HVEM, LIGHT, CD40, 4-1BB, 0X40, DR3, GITR, CD30, TIMI, CD2, CD226, or any combination thereof. In some embodiments, the agent that stimulates a costimulatory molecule is an agent that stimulates CD28. In some embodiments, the agent that stimulates a CD3/TCR complex is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand). In some embodiments, the agent that stimulates a CD3/TCR complex is an antibody. In some embodiments, the agent that stimulates a CD3/TCR complex is an anti-CD3 antibody. In some embodiments, the agent that stimulates a costimulatory molecule is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand). In some embodiments, the agent that stimulates a costimulatory molecule is an antibody. In some embodiments, the agent that stimulates a costimulatory molecule is an anti-CD28 antibody. In some embodiments, the agent that stimulates a CD3/TCR complex or the agent that stimulates a costimulatory molecule does not comprise a bead. In some embodiments, the agent that stimulates a CD3/TCR complex comprises an anti-CD3 antibody covalently attached to a colloidal polymeric nanomatrix. In some embodiments, the agent that stimulates a costimulatory molecule comprises an anti-CD28 antibody covalently attached to a colloidal polymeric nanomatrix. In some embodiments, the agent that stimulates a CD3/TCR complex and the agent that stimulates a costimulatory molecule comprise T Cell TransAct™.

In some embodiments, the matrix comprises or consists of a polymeric, for example, biodegradable or biocompatible inert material, for example, which is non-toxic to cells. In some embodiments, the matrix is composed of hydrophilic polymer chains, which obtain maximal mobility in aqueous solution due to hydration of the chains. In some embodiments, the mobile matrix may be of collagen, purified proteins, purified peptides, polysaccharides, glycosaminoglycans, or extracellular matrix compositions. A polysaccharide may include for example, cellulose ethers, starch, gum arabic, agarose, dextran, chitosan, hyaluronic acid, pectins, xanthan, guar gum or alginate. Other polymers may include polyesters, polyethers, poly acrylates, polyacrylamides, polyamines, polyethylene imines, polyquaternium polymers, polyphosphazenes, polyvinylalcohols, poly vinylacetates, polyvinylpyrrolidones, block copolymers, or polyurethanes. In some embodiments, the mobile matrix is a polymer of dextran.

In some embodiments, the population of cells is contacted with a nucleic acid molecule (e.g. one or more nucleic acid molecules) encoding a CAR (e.g. one or more CARs). In some embodiments, the population of cells is transduced with a DNA molecule (e.g. one or more DNA molecules) encoding a CAR (e.g. one or more CARs).

In some embodiments, in the case of a co-transduction of two nucleic acid molecules (e.g., lentiviral vectors), each of which encodes a CAR disclosed herein, each of the vectors containing nucleic acid molecules encoding the CAR can be added to the reaction mixture (e.g., containing a cell population) at a different multiplicity of infection (MOI).

Without wishing to be bound by theory, it is believed that, in some embodiments, using different MOIs for the vectors containing nucleic acid molecules which encode distinct CAR molecules may affect the final composition of the cellular population. For example, in the case of a co transduction of a lentiviral vector encoding one CAR and a lentiviral vector encoding another CAR targeting a different target, different MOIs can be used to maximize the percent of preferred mono CART cells and dual CART cells, while resulting in fewer undesired mono CART cells and untransduced cells.

The precise MOI used for each vector can be adjusted or determined based on a number of factors, including, but not limited to, properties of the batch of viral vector, characteristics of the cells to be transduced, and transduction efficiency. In some embodiments, contacting the population of cells with the nucleic acid molecule(s) encoding the CAR(s) occurs simultaneously with contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0.5 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 20 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 19 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 18 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 17 hours after the beginning of contacting the population of cells with the agent that stimulates a CD3/TCR complex and/or the agent that stimulates a costimulatory molecule on the surface of the cells described above.

In some embodiments, contacting the population of cells with the nucleic acid molecule(s) encoding the CAR(s) occurs no later than 16 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 15 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 14 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 14 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 13 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 12 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 11 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 10 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 9 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 8 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 7 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 6 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 5 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 4 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 3 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR(s) occurs no later than 2 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 1 hour after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 30 minutes after the beginning of contacting the population of cells with the agent that stimulates a CD3/TCR complex and/or the agent that stimulates a costimulatory molecule on the surface of the cells described above.

In some embodiments, the population of cells is harvested for storage or administration.

In some embodiments, the population of cells is harvested for storage or administration no later than 72, 60, 48, 36, 32, 31, 30, 29, 28, 27, 26, 25, 24, 23, 22, 21, 20, 19, or 18 hours after the beginning of contacting the population of cells with the agent that stimulates a CD3/TCR complex and/or the agent that stimulates a costimulatory molecule on the surface of the cells described above. In some embodiments, the population of cells is harvested for storage or administration no later than 26 hours after the beginning of contacting the population of cells with the agent that stimulates a CD3/TCR complex and/or the agent that stimulates a costimulatory molecule on the surface of the cells described above. In some embodiments, the population of cells is harvested for storage or administration no later than 25 hours after the beginning of contacting the population of cells with the agent that stimulates a CD3/TCR complex and/or the agent that stimulates a costimulatory molecule on the surface of the cells described above. In some embodiments, the population of cells is harvested for storage or administration no later than 24 hours after the beginning of contacting the population of cells with the agent that stimulates a CD3/TCR complex and/or the agent that stimulates a costimulatory molecule on the surface of the cells described above. In some embodiments, the population of cells is harvested for storage or administration no later than 23 hours after the beginning of contacting the population of cells with the agent that stimulates a CD3/TCR complex and/or the agent that stimulates a costimulatory molecule on the surface of the cells described above. In some embodiments, the population of cells is harvested for storage or administration no later than 22 hours after the beginning of contacting the population of cells with the agent that stimulates a CD3/TCR complex and/or the agent that stimulates a costimulatory molecule on the surface of the cells described above.

In some embodiments, the population of cells is not expanded ex vivo.

In some embodiments, the population of cells is expanded by no more than 5, 6, 7, 8, 9, 10, 11, 12, 13, 14, 15, 16, 17, 18, 19, 20, 25, 30, 35, 40, 45, 50, 55, or 60%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 5%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 10%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 15%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 20%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 25%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 30%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 35%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 40%, for example, as assessed by the number of living cells, compared to the population of cells before it is contacted with the agent that stimulates a CD3/TCR complex and/or the agent that stimulates a costimulatory molecule on the surface of the cells described above.

In some embodiments, the population of cells is expanded by no more than 1, 1.5, 2, 2.5, 3, 3.5, 4, 4.5, 5, 6, 7, 8, 9, 10, 11, 12, 16, 20, 24, 36, or 48 hours, for example, as assessed by the number of living cells, compared to the population of cells before it is contacted with the one or more cytokines described above.

In some embodiments, the activation process is conducted in serum free cell media. In some embodiments, the activation process is conducted in cell media comprising one or more cytokines chosen from: IL-2, IL-15 (for example, hetIL-15 (IL15/sIL-15Ra)), or IL-6 (for example, IL-6/sIL-6Ra). In some embodiments, hetIL-15 comprises the amino acid sequence of NWVNVISDLKKIEDLIQSMHIDATLYTESDVHPSCKVTAMKCFLLELQVISLESGDASIHDTV ENLIILANNSLSSNGNVTESGCKECEELEEKNIKEFLQSFVHIVQMFINTSITCPPPMSVEHADI WVKSYSLYSRERYICNSGFKRKAGTSSLTECVLNKATNVAHWTTPSLKCIRDPALVHQRPAP PSTVTTAGVTPQPESL SPSGKEPAASSPSSNNTAATTAAIVPGSQLMPSKSPSTGTTEISSHESS HGTPSQTTAKNWELTASASHQPPGVYPQG (SEQ ID NO: 309). In some embodiments, hetIL-15 comprises an amino acid sequence having at least about 70, 75, 80, 85, 90, 95, or 99% identity to SEQ ID NO: 309.

In some embodiments, the activation process is conducted in cell media comprising a LSD1 inhibitor.

In some embodiments, the activation process is conducted in cell media comprising a MALT1 inhibitor. In some embodiments, the serum free cell media comprises a serum replacement. In some embodiments, the serum replacement is CTS™ Immune Cell Serum Replacement (ICSR). In some embodiments, the level of ICSR can be, for example, up to 5%, for example, about 1%, 2%, 3%, 4%, or 5%. Without wishing to be bound by theory, using cell media, for example, Rapid Media shown in Table 21 or Table 25, comprising ICSR, for example, 2% ICSR, may improve cell viability during a manufacture process described herein.

In some embodiments, the present disclosure provides methods of making a population of cells (for example, T cells) that express a chimeric antigen receptor (CAR) comprising: (a) providing an apheresis sample (for example, a fresh or cryopreserved leukapheresis sample) collected from a subject; (b) selecting T cells from the apheresis sample (for example, using negative selection, positive selection, or selection without beads); (c) seeding isolated T cells at, for example, 1×10⁶ to 1×10⁷ cells/mL; (d) contacting T cells with an agent that stimulates T cells, for example, an agent that stimulates a CD3/TCR complex and/or an agent that stimulates a costimulatory molecule on the surface of the cells (for example, contacting T cells with anti-CD3 and/or anti-CD28 antibody, for example, contacting T cells with TransAct); (e) contacting T cells with a nucleic acid molecule(s) (for example, a DNA or RNA molecule) encoding the CAR(s) (for example, contacting T cells with a virus comprising a nucleic acid molecule(s) encoding the CAR(s)) for, for example, 6-48 hours, for example, 20-28 hours; and (f) washing and harvesting T cells for storage (for example, reformulating T cells in cryopreservation media) or administration. In some embodiments, step (f) is performed no later than 30 hours after the beginning of step (d) or (e), for example, no later than 22, 23, 24, 25, 26, 27, 28, 29, or 30 hours after the beginning of step (d) or (e).

In some embodiments, provided herein is a population of cells (for example, immune effector cells, for example, T cells or NK cells) made by any of the manufacturing processes described herein.

In some embodiments, the percentage of naive cells, for example, naive T cells, for example, CD45RA+CD45RO− CCR7+ T cells, in the population of cells at the end of the manufacturing process (for example, at the end of the cytokine process or the activation process described herein) (1) is the same as, (2) differs, for example, by no more than 4, 5, 6, 7, 8, 9, 10, 11, 12, 13, 14, or 15%, from, or (3) is increased, for example, by at least 5, 6, 7, 8, 9, 10, 11, 12, 13, 14, 15, 16, 17, 18, 19, 20, 21, 22, 23, 24, or 25%, as compared to, the percentage of naive cells, for example, naive T cells, for example, CD45RA+CD45RO− CCR7+ cells, in the population of cells at the beginning of the manufacturing process (for example, at the beginning of the cytokine process or the activation process described herein). In some embodiments, the population of cells at the end of the manufacturing process (for example, at the end of the cytokine process or the activation process described herein) shows a higher percentage of naive cells, for example, naive T cells, for example, CD45RA+CD45RO− CCR7+ T cells (for example, at least 5, 6, 7, 8, 9, 10, 11, 12, 13, 14, 15, 16, 17, 18, 19, 20, 25, 30, 35, 40, 45, or 50% higher), compared with cells made by an otherwise similar method which lasts, for example, more than 26 hours (for example, which lasts more than 5, 6, 7, 8, 9, 10, 11, or 12 days) or which involves expanding the population of cells in vitro for, for example, more than 3 days (for example, expanding the population of cells in vitro for 3, 4, 5, 6, 7, 8, 9, 10, 11, 12, 13, 14, or 15 days).

In some embodiments, the percentage of naive cells, for example, naive T cells, for example, CD45RA+CD45RO− CCR7+ T cells, in the population of cells at the end of the manufacturing process (for example, at the end of the cytokine process or the activation process described herein) is not less than 20, 25, 30, 35, 40, 45, 50, 55, or 60%.

In some embodiments, the percentage of central memory cells, for example, central memory T cells, for example, CD95+ central memory T cells, in the population of cells at the end of the manufacturing process (for example, at the end of the cytokine process or the activation process described herein) (1) is the same as, (2) differs, for example, by no more than 4, 5, 6, 7, 8, 9, 10, 11, 12, 13, 14, or 15% from, or (3) is decreased, for example, by at least 5, 6, 7, 8, 9, 10, 11, 12, 13, 14, 15, 16, 17, 18, 19, 20, 21, 22, 23, 24, or 25%, as compared to, the percentage of central memory cells, for example, central memory T cells, for example, CD95+ central memory T cells, in the population of cells at the beginning of the manufacturing process (for example, at the beginning of the cytokine process or the activation process described herein). In some embodiments, the population of cells at the end of the manufacturing process (for example, at the end of the cytokine process or the activation process described herein) shows a lower percentage of central memory cells, for example, central memory T cells, for example, CD95+ central memory T cells (for example, at least 5, 6, 7, 8, 9, 10, 11, 12, 13, 14, 15, 16, 17, 18, 19, 20, 25, 30, 35, 40, 45, or 50% lower), compared with cells made by an otherwise similar method which lasts, for example, more than 26 hours (for example, which lasts more than 5, 6, 7, 8, 9, 10, 11, or 12 days) or which involves expanding the population of cells in vitro for, for example, more than 3 days (for example, expanding the population of cells in vitro for 3, 4, 5, 6, 7, 8, 9, 10, 11, 12, 13, 14, or 15 days).

In some embodiments, the percentage of central memory cells, for example, central memory T cells, for example, CD95+ central memory T cells, in the population of cells at the end of the manufacturing process (for example, at the end of the cytokine process or the activation process described herein) is no more than 40, 45, 50, 55, 60, 65, 70, 75, or 80%.

In some embodiments, the population of cells at the end of the manufacturing process (for example, at the end of the cytokine process or the activation process described herein) after being administered in vivo, persists longer or expands at a higher level (for example, at least 20, 25, 30, 35, 40, 45, 50, 55, 60, 65, 70, 75, 80, 85, or 90% higher), compared with cells made by an otherwise similar method which lasts, for example, more than 26 hours (for example, which lasts more than 5, 6, 7, 8, 9, 10, 11, or 12 days) or which involves expanding the population of cells in vitro for, for example, more than 3 days (for example, expanding the population of cells in vitro for 3, 4, 5, 6, 7, 8, 9, 10, 11, 12, 13, 14, or 15 days).

In some embodiments, the population of cells has been enriched for IL6R-expressing cells (for example, cells that are positive for IL6Ra and/or I L6 Kb) prior to the beginning of the manufacturing process (for example, prior to the beginning of the cytokine process or the activation process described herein). In some embodiments, the population of cells comprises, for example, no less than 30, 35, 40, 45, 50, 55, 60, 65, 70, 75, or 80% of IL6R-expressing cells (for example, cells that are positive for IL6Ra and/or I L6 Kb) at the beginning of the manufacturing process (for example, at the beginning of the cytokine process or the activation process described herein).

### In Vitro CAR-T Manufacture

Lentiviral vectors described herein (e.g., those made using a method described herein), can be used, e.g., in the in vitro manufacture of CAR-T cells.

In some embodiments, cells transduced with the viral vector as described herein, are expanded, e.g., by a method described herein. In some embodiments, the cells are expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In some embodiments, the cells are expanded for a period of 4 to 9 days. In some embodiments, the cells are expanded for a period of 8 days or less, e.g., 7, 6 or 5 days. In some embodiments, the cells are expanded in culture for 5 days, and the resulting cells are more potent than the same cells expanded in culture for 9 days under the same culture conditions. Potency can be defined, e.g., by various T cell functions, e.g. proliferation, target cell killing, cytokine production, activation, migration, or combinations thereof. In some embodiments, the cells are expanded for 5 days show at least a one, two, three, or four-fold increase in cells doublings upon antigen stimulation as compared to the same cells expanded in culture for 9 days under the same culture conditions. In some embodiments, the cells are expanded in culture for 5 days, and the resulting cells exhibit higher proinflammatory cytokine production, e.g., IFN-γ and/or GM-CSF levels, as compared to the same cells expanded in culture for 9 days under the same culture conditions. In some embodiments, the cells expanded for 5 days show at least a one, two, three, four, five, ten-fold or more increase in pg/ml of proinflammatory cytokine production, e.g., IFN-γ and/or GM-CSF levels, as compared to the same cells expanded in culture for 9 days under the same culture conditions.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed, and the cells directly resuspended in culture media.

It is recognized that the in vitro methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS Immune Cell Serum Replacement” Clinical & Translational Immunology (2015) 4, e31; doi:10.1038/cti.2014.31.

In some aspects, T cells are isolated from peripheral blood lymphocytes by lysing the red blood cells and depleting the monocytes, for example, by centrifugation through a PERCOLL™ gradient or by counterflow centrifugal elutriation. The isolated T cells may be further used in the methods described herein.

The methods described herein can include, e.g., selection of a specific subpopulation of immune effector cells, e.g., T cells, that are a T regulatory cell-depleted population, CD25+ depleted cells, using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells.

In some embodiments, T regulatory cells, e.g., CD25+ T cells, are removed from the population using an anti-CD25 antibody, or fragment thereof, or a CD25-binding ligand, IL-2. In some embodiments, the anti-CD25 antibody, or fragment thereof, or CD25-binding ligand is conjugated to a substrate, e.g., a bead, or is otherwise coated on a substrate, e.g., a bead. In some embodiments, the anti-CD25 antibody, or fragment thereof, is conjugated to a substrate as described herein.

In some embodiments, the T regulatory cells, e.g., CD25+ T cells, are removed from the population using CD25 depletion reagent from Miltenyi™. In some embodiments, the ratio of cells to CD25 depletion reagent is 1×10⁷ cells to 20 μL, or 1×10⁷ cells to 15 μL, or 1×10⁷ cells to 10 μL, or 1×10⁷ cells to 5 μL, or 1×10⁷ cells to 2.5 μL, or 1×10⁷ cells to 1.25 μL. In some embodiments, e.g., for T regulatory cells, e.g., CD25+ depletion, greater than 500 million cells/ml is used. In a further aspect, a concentration of cells of 600, 700, 800, or 900 million cells/ml is used.

In some embodiments, the population of immune effector cells to be depleted includes about 6×10⁹ CD25+ T cells. In other aspects, the population of immune effector cells to be depleted include about 1×10⁹ to 1×10¹⁰ CD25+ T cell, and any integer value in between. In some embodiments, the resulting population T regulatory depleted cells has 2×10⁹ T regulatory cells, e.g., CD25+ cells, or less (e.g., 1×10⁹, 5×10⁸, 1×10⁸, 5×10⁷, 1×10⁷, or less CD25+ cells).

In some embodiments, the T regulatory cells, e.g., CD25+ cells, are removed from the population using the CliniMAC system with a depletion tubing set, such as, e.g., tubing 162-01. In some embodiments, the CliniMAC system is run on a depletion setting such as, e.g., DEPLETION2.1.

Without wishing to be bound by a particular theory, decreasing the level of negative regulators of immune cells (e.g., decreasing the number of unwanted immune cells, e.g., T_{REG} cells), in a subject prior to apheresis or during manufacturing of a CAR-expressing cell product can reduce the risk of subject relapse. For example, methods of depleting T_{REG} cells are known in the art. Methods of decreasing T_{REG} cells include, but are not limited to, cyclophosphamide, anti-GITR antibody (an anti-GITR antibody described herein), CD25-depletion, and combinations thereof.

In some embodiments, the manufacturing methods comprise reducing the number of (e.g., depleting) T_{REG} cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete T_{REG} cells prior to manufacturing of the CAR-expressing cell (e.g., T cell, NK cell) product.

In some embodiments, a subject is pre-treated with one or more therapies that reduce T_{REG} cells prior to collection of cells for CAR-expressing cell product manufacturing, thereby reducing the risk of subject relapse to CAR-expressing cell treatment. In some embodiments, methods of decreasing T_{REG} cells include, but are not limited to, administration to the subject of one or more of cyclophosphamide, anti-GITR antibody, CD25-depletion, or a combination thereof. Administration of one or more of cyclophosphamide, anti-GITR antibody, CD25-depletion, or a combination thereof, can occur before, during or after an infusion of the CAR-expressing cell product.

In some embodiments, a subject is pre-treated with cyclophosphamide prior to collection of cells for CAR-expressing cell product manufacturing, thereby reducing the risk of subject relapse to CAR-expressing cell treatment. In some embodiments, a subject is pre-treated with an anti-GITR antibody prior to collection of cells for CAR-expressing cell product manufacturing, thereby reducing the risk of subject relapse to CAR-expressing cell treatment.

In some embodiments, the population of cells to be removed are neither the regulatory T cells or tumor cells, but cells that otherwise negatively affect the expansion and/or function of CART cells, e.g. cells expressing CD14, CD11b, CD33, CD15, or other markers expressed by potentially immune suppressive cells. In some embodiments, such cells are envisioned to be removed concurrently with regulatory T cells and/or tumor cells, or following said depletion, or in another order.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, e.g., a tumor antigen that does not comprise CD25, e.g., CD19, CD30, CD38, CD123, CD20, CD14 or CD11b, to thereby provide a population of T regulatory depleted, e.g., CD25+ depleted, and tumor antigen depleted cells that are suitable for expression of a CAR, e.g., a CAR described herein. In some embodiments, tumor antigen expressing cells are removed simultaneously with the T regulatory, e.g., CD25+ cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, e.g., CD25+ cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory depleted, e.g., CD25+ depleted cells, and check point inhibitor depleted cells, e.g., PD1+, LAG3+ and/or TIM3+ depleted cells. Exemplary check point inhibitors include B7-H1, 37-1, CD160, P1H, 2B4, PD1, TIM3, CEACAM (e.g., CEACAM-1, CEACAM-3 and/or CEACAM-5), LAG3, TIGIT, CTLA-4, BTLA, and LAIR1. In some embodiments, check point inhibitor expressing cells are removed simultaneously with the T regulatory, e.g., CD25+ cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, e.g., CD25+ cells, and the removal of the check point inhibitor expressing cells is sequential, and can occur, e.g., in either order.

Methods described herein can include a positive selection step. For example, T cells can isolated by incubation with anti-CD3/anti-CD28 (e.g., 3×28)-conjugated beads, such as DYNABEADS® M-450 CD3/CD28 T, for a time period sufficient for positive selection of the desired T cells. In some embodiments, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours, e.g., 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. In some embodiments, a T cell population can be selected that expresses one or more of IFN-γ, TNFα, IL-17A, IL-2, IL-3, IL-4, GM-CSF, IL-10, IL-13, granzyme B, and perform, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In some aspects, it may be desirable to significantly decrease the volume in which beads and cells are mixed together (e.g., increase the concentration of cells), to ensure maximum contact of cells and beads. For example, in some aspects, a concentration of 10 billion cells/ml, 9 billion/ml, 8 billion/ml, 7 billion/ml, 6 billion/ml, or 5 billion/ml is used. In some aspects, a concentration of 1 billion cells/ml is used. In yet some aspects, a concentration of cells from 75, 80, 85, 90, 95, or 100 million cells/ml is used. In further aspects, concentrations of 125 or 150 million cells/ml can be used.

Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In some embodiments, it may be desirable to use lower concentrations of cells. By significantly diluting the mixture of T cells and surface (e.g., particles such as beads), interactions between the particles and cells are minimized. This selects for cells that express high amounts of desired antigens to be bound to the particles. For example, CD4+ T cells express higher levels of CD28 and are more efficiently captured than CD8+ T cells in dilute concentrations. In some aspects, the concentration of cells used is 5×10⁶/ml. In other aspects, the concentration used can be from about 1×10⁵/ml to 1×10⁶/ml, and any integer value in between.

In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provide a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some aspects, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, can be isolated and frozen for later use in immune effector cell therapy for any number of diseases or conditions that would benefit from immune effector cell therapy, such as those described herein. In some aspects a blood sample or an apheresis is taken from a generally healthy subject. In some aspects, a blood sample or an apheresis is taken from a generally healthy subject who is at risk of developing a disease, but who has not yet developed a disease, and the cells of interest are isolated and frozen for later use. In some aspects, the T cells may be expanded, frozen, and used at a later time. In some aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present invention, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in some aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In some embodiments, a T cell population is diaglycerol kinase (DGK)-deficient. DGK-deficient cells include cells that do not express DGK RNA or protein or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In some embodiments, a T cell population is Ikaros-deficient. Ikaros-deficient cells include cells that do not express Ikaros RNA or protein, or have reduced or inhibited Ikaros activity, Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

In some embodiments, the NK cells are obtained from the subject. In another embodiment, the NK cells are an NK cell line, e.g., NK-92 cell line (Conkwest).

In a particular exemplary aspect, subjects may undergo leukapheresis, wherein leukocytes are collected, enriched, or depleted ex vivo to select and/or isolate the cells of interest, e.g., T cells.

These T cell isolates may be expanded by methods described herein. Subjects in need thereof may subsequently undergo standard treatment with high dose chemotherapy followed by peripheral blood stem cell transplantation. In some aspects, following or concurrent with the transplant, subjects receive an infusion of the expanded CAR T cells as prepared by the methods of the present invention. In an additional aspect, expanded cells are administered before or following surgery.

### Additional Expressed Agents

Co-Expression of an Agent that Enhances CAR Activity

In the embodiments contemplated herein, it is appreciated that additional agents may be encoded in the vectors described herein above. Accordingly, these agents are described below in relation to the CAR-expressing cell.

In another embodiment, a CAR-expressing immune effector cell described herein can further express another agent, e.g., an agent which enhances the activity of a CAR-expressing cell. For example, in some embodiments, the agent can be an agent which inhibits an inhibitory molecule. Examples of inhibitory molecules include PD-1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4 and TGFR beta, e.g., as described herein. In some embodiments, the agent that inhibits an inhibitory molecule comprises a first polypeptide, e.g., an inhibitory molecule, associated with a second polypeptide that provides a positive signal to the cell, e.g., an intracellular signaling domain described herein. In some embodiments, the agent comprises a first polypeptide, e.g., of an inhibitory molecule such as PD-1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4, or TGFR beta, or a fragment of any of these, and a second polypeptide which is an intracellular signaling domain described herein (e.g., comprising a costimulatory domain (e.g., 41BB, CD27 or CD28, e.g., as described herein) and/or a primary signaling domain (e.g., a CD3 zeta signaling domain described herein). In some embodiments, the agent comprises a first polypeptide of PD-1 or a fragment thereof, and a second polypeptide of an intracellular signaling domain described herein (e.g., a CD28, CD27, OX40 or 4-IBB signaling domain described herein and/or a CD3 zeta signaling domain described herein).

### Co-Expression of a Second CAR

In some embodiments, the CAR-expressing cell described herein can further comprise a second CAR, for example, a second CAR that includes a different antigen binding domain, for example, to the same target (for example, CD19) or a different target (for example, a target other than CD19, for example, a target described herein).

In some embodiments, the CAR-expressing cell described herein, e.g., the CAR-expressing cell manufactured using a method described herein, comprises (i) a first nucleic acid molecule encoding a first CAR that binds to BCMA and (ii) a second nucleic acid molecule encoding a second CAR that binds to CD19. In some embodiments, the first CAR comprises an anti-BCMA binding domain, a first transmembrane domain, and a first intracellular signaling domain, wherein the anti-BCMA binding domain comprises a heavy chain variable region (VH) comprising a heavy chain complementary determining region 1 (HC CDR1), a heavy chain complementary determining region 2 (HC CDR2), and a heavy chain complementary determining region 3 (HC CDR3), and a light chain variable region (VL) comprising a light chain complementary determining region 1 (LC CDR1), a light chain complementary determining region 2 (LC CDR2), and a light chain complementary determining region 3 (LC CDR3), wherein the HC CDR1, HC CDR2, HC CDR3, LC CDR1, LC CDR2, and LC CDR3 comprise the amino acid sequences of SEQ ID NOs: 86, 87, 88, 95, 96, and 97, respectively. In some embodiments, the second CAR comprises an anti-CD19 binding domain, a second transmembrane domain, and a second intracellular signaling domain, wherein the anti-CD19 binding domain comprises a VH comprising a HC CDR1, a HC CDR2, and a HC CDR3, and a VL comprising a LC CDR1, a LC CDR2, and a LC CDR3, wherein the HC CDR1, HC CDR2, HC CDR3, LC CDR1, LC CDR2, and LC CDR3 comprise the amino acid sequences of SEQ ID NOs: 760, 687, 762, 763, 764, and 765, respectively. In some embodiments, (i) the VH and VL of the anti-BCMA binding domain comprise the amino acid sequences of SEQ ID NOs: 93 and 102, respectively. In some embodiments, the VH and VL of the anti-CD19 binding domain comprise the amino acid sequences of SEQ ID NOs: 250A and 251A, respectively. In some embodiments, the anti-BCMA binding domain comprises the amino acid sequence of SEQ ID NO: 105. In some embodiments, the anti-CD19 binding domain comprises the amino acid sequence of SEQ ID NO: 758. In some embodiments, the first CAR comprises the amino acid sequence of SEQ ID NO: 107. In some embodiments, the second CAR comprise the amino acid sequence of SEQ ID NO: 225.

In some embodiments, the CAR-expressing cell described herein, e.g., the CAR-expressing cell manufactured using a method described herein, comprises (i) a first nucleic acid molecule encoding a first CAR that binds to CD22 and (ii) a second nucleic acid molecule encoding a second CAR that binds to CD19. In some embodiments, the CD22 CAR comprises a CD22 antigen binding domain, and a first transmembrane domain; a first co-stimulatory signaling domain; and/or a first primary signaling domain. In some embodiments, the CD19 CAR comprises a CD19 antigen binding domain, and a second transmembrane domain; a second co-stimulatory signaling domain; and/or a second primary signaling domain.

In some embodiments, the CD22 antigen binding domain comprises one or more (e.g., all three) light chain complementarity determining region 1 (LC CDR1), light chain complementarity determining region 2 (LC CDR2), and light chain complementarity determining region 3 (LC CDR3) of a CD22 binding domain described herein, e.g., in Tables 15, 16, 30, 31, or 32; and/or one or more (e.g., all three) heavy chain complementarity determining region 1 (HC CDR1), heavy chain complementarity determining region 2 (HC CDR2), and heavy chain complementarity determining region 3 (HC CDR3) of a CD22 binding domain described herein, e.g., in Tables 15, 16, 30, 31 or 32. In an embodiment, the CD22 antigen binding domain comprises a LC CDR1, LC CDR2 and LC CDR3 of a CD22 binding domain described herein, e.g., in Table 15, 16, 30, 31 or 32; and/or a HC CDR1, HC CDR2 and HC CDR3 of a CD22 binding domain described herein, e.g., in Tables 15, 16, 30, 31 or 32. In some embodiments, the CD19 antigen binding domain comprises: one or more (e.g., all three) LC CDR1, LC CDR2, and LC CDR3 of a CD19 binding domain described herein, e.g., in Tables 1, 30, 31, or 32; and/or one or more (e.g., all three) HC CDR1, HC CDR2, and HC CDR3 of a CD19 binding domain described herein, e.g., in Tables 1, 30, 31, and 32. In some embodiments, the CD19 antigen binding domain comprises a LC CDR1, LC CDR2 and LC CDR3 of a CD19 binding domain described herein, e.g., in Tables 1, 30, 31, and 32; and/or a HC CDR1, HC CDR2 and HC CDR3 of a CD19 binding domain described herein, e.g., in Tables 1, 30, 31, and 32.

In some embodiment, the CD22 antigen binding domain (e.g., an scFv) comprises a light chain variable (VL) region of a CD22 binding domain described herein, e.g., in Tables 30 or 32; and/or a heavy chain variable (VH) region of a CD22 binding domain described herein, e.g., in Tables 30 or 32. In some embodiments, the CD22 antigen binding domain comprises a VL region comprising an amino acid sequence having at least one, two or three modifications (e.g., substitutions) but not more than 30, 20 or 10 modifications (e.g., substitutions) of a CD22 VL region sequence provided in Table 30 or 32. In some embodiments, the CD22 antigen binding domain comprises a VL region comprising an amino acid sequence provided in Table 30 or 32, or a sequence having at least about 80%, 85%, 90%, 92%, 95%, 97%, 98%, or 99% sequence identity to any of the aforesaid sequences. In some embodiments, the CD22 antigen binding domain comprises a VH region comprising an amino acid sequence having at least one, two or three modifications (e.g., substitutions) but not more than 30, 20 or 10 modifications (e.g., substitutions) of a CD22 VH region sequence provided in Table 30 or 32. In some embodiments, the CD22 antigen binding domain comprises a VH region comprising the amino acid sequence of a CD22 VH region sequence provided in Table 30 or 32, or a sequence having at least about 80%, 85%, 90%, 92%, 95%, 97%, 98%, or 99% sequence identity to any of the aforesaid sequences. In some embodiments, the CD19 antigen binding domain (e.g., an scFv) comprises a VL region of a CD19 binding domain described herein, e.g., in Tables 1, 30, or 32; and/or a VH region of a CD19 binding domain described herein, e.g., in Tables 1, 30, or 32. In some embodiments, the CD19 antigen binding domain comprises a VL region comprising an amino acid sequence having at least one, two or three modifications (e.g., substitutions) but not more than 30, 20 or 10 modifications (e.g., substitutions) of a CD19 VL region sequence provided in Tables 1, 30, or 32. In some embodiments, the CD19 antigen binding domain comprises a VL region comprising the amino acid sequence of a CD19 VL region sequence provided in Tables 1, 30, or 32, or a sequence having at least about 80%, 85%, 90%, 92%, 95%, 97%, 98%, or 99% sequence identity to any of the aforesaid sequences. In some embodiments, the CD19 antigen binding domain comprises a VH region comprising an amino acid sequence having at least one, two or three modifications (e.g., substitutions) but not more than 30, 20 or 10 modifications (e.g., substitutions) of a CD19 VH region sequence provided in Tables 1, 30, or 32. In some embodiments, the CD19 antigen binding domain comprises a VH region comprising the amino acid sequence of a CD19 VH region sequence provided in Tables 1, 30, or 32, or a sequence having at least about 80%, 85%, 90%, 92%, 95%, 97%, 98%, or 99% sequence identity to any of the aforesaid sequences.

In some embodiments, the CD22 antigen binding comprises an scFv comprising an amino acid sequence having at least one, two or three modifications (e.g., substitutions) but not more than 30, 20 or 10 modifications (e.g., substitutions) of a CD22 scFv sequence provided in Table 30 or 32. In some embodiments, the CD22 antigen binding comprises an scFv comprising an amino acid sequence of a CD22 scFv sequence provided in Table 30 or 32, or a sequence having at least about 80%, 85%, 90%, 92%, 95%, 97%, 98%, or 99% sequence identity to any of the aforesaid sequences. In some embodiments, the CD19 antigen binding domain comprises an scFv comprising an amino acid sequence having at least one, two or three modifications (e.g., substitutions) but not more than 30, 20 or 10 modifications (e.g., substitutions) of a CD19 scFv sequence provided in Tables 1, 30, or 32. In some embodiments, the CD19 antigen binding domain comprises an scFv comprising the amino acid sequence of a CD19 scFv sequence provided in Tables 1, 30, or 32, or a sequence having at least about 80%, 85%, 90%, 92%, 95%, 97%, 98%, or 99% sequence identity to any of the aforesaid sequences.

In some embodiments, the CD22 CAR molecule and/or the CD19 CAR molecule comprises an additional component, e.g., a signal peptide, a hinge, a transmembrane domain, a co-stimulatory signaling domain and/or a first primary signaling domain, a P2A site, and/or a linker, comprising an amino acid sequence provided in Table 33, or a sequence having at least about 70%, 75%, 80%, 85%, 90%, 92%, 95%, 97%, 98%, or 99% sequence identity to any of the aforesaid sequences; or is encoded by a nucleotide sequence provided in Table 33, or a sequence having at least about 70%, 75%, 80%, 85%, 90%, 92%, 95%, 97%, 98%, or 99% sequence identity to any of the aforesaid sequences.

Exemplary nucleotide and amino acid sequences of a CAR molecule, e.g., a dual CAR molecule comprising (i) a first CAR that binds to CD22 and (ii) a second CAR that binds to CD19 disclosed herein, is provided in Table 30.

TABLE 30
Dual and tandem CD19-CD22 CAR sequences

| | | |
|---|---|---|
| | SEQ | |
| | ID | |
| Identifier | NO | Sequence |

Tandem CD19-CD22 CARs

| | | |
|---|---|---|
| CG#c171 | 813 | atggccctccctgtcaccgccctgctgctt |
| | | ccgctggctcttctgctccacgccgctcgg |
| | | cccgaaattgtgatgacccagtcacccgcc |
| | | actcttagcctttcacccggtgagcgcgca |
| | | accctgtcttgcagagcctcccaagacatc |
| | | tcaaaataccttaattggtatcaacagaag |
| | | cccggacaggctcctcgccttctgatctac |
| | | cacaccagccggctccattctggaatccct |
| | | gccaggttcagcggtagcggatctgggacc |
| | | gactacaccctcactatcagctcactgcag |
| | | ccagaggacttcgctgtctatttctgtcag |
| | | caagggaacaccctgccctacacctttgga |
| | | cagggcaccaagctcgagattaaaggtgga |
| | | ggtggcagcggaggaggtgggtccggcggt |
| | | ggaggaagccaggtccaactccaagaaagc |
| | | ggaccgggtcttgtgaagccatcagaaact |
| | | ctttcactgacttgtactgtgagcggagtg |
| | | tctctccccgattacggggtgtcttggatc |
| | | agacagccaccggggaagggtctggaatgg |
| | | attggagtgatttggggctctgagactact |
| | | tactaccaatcatccctcaagtcacgcgtc |
| | | accatctcaaaggacaactctaagaatcag |
| | | gtgtcactgaaactgtcatctgtgaccgca |
| | | gccgacaccgccgtgtactattgcgctaag |
| | | cattactattatggcgggagctacgcaatg |
| | | gattactggggacagggtactctggtcacc |
| | | gtgtccagcttggcagaagccgccgcgaaa |
| | | gaagtgcagcttcaacaatcaggaccagga |
| | | ctcgtcaaaccatcacagaccctctccctc |
| | | acatgtgccatctccggggactccatgttg |
| | | agcaattccgacacttggaattggattaga |
| | | caaagcccgtcccggggtctggaatggttg |
| | | ggacgcacctaccaccggtctacttggtac |
| | | gacgactacgcgtcatccgtgcggggaaga |
| | | gtgtccatcaacgtggacacctccaagaac |
| | | cagtacagcctgcagcttaatgccgtgact |
| | | cctgaggatacgggcgtctactactgcgcc |
| | | cgcgtccgcctgcaagacgggaacagctgg |
| | | agcgatgcattcgatgtctggggccaggga |
| | | actatggtcaccgtgtcgtctgggggcggt |
| | | ggatcgggtggcgggggttcggggggcggc |
| | | ggctctcagtccgctcttacccaaccggcc |
| | | tcagcctcggggagccccggccagagcgtg |
| | | accatttcctgcaccggcacttcatccgac |
| | | gtgggcggctacaactacgtgtcctggtac |
| | | caacagcacccgggaaaggcccccaagctc |
| | | atgatctacgacgtgtccaacaggccctcg |
| | | ggagtgtccaaccggttctcgggttcgaaa |
| | | tcgggaaacacagccagcctgaccatcagc |
| | | ggactgcaggctgaagatgaagccgactac |
| | | tactgctcctcctacacctcgtcatccacg |
| | | ctctacgtgttcggcactggaactcagctg |
| | | actgtgctgaccactaccccagcaccgagg |
| | | ccacccaccccggctcctaccatcgcctcc |
| | | cagcctctgtccctgcgtccggaggcatgt |
| | | agacccgcagctggtggggccgtgcatacc |
| | | cggggtcttgacttcgcctgcgatatctac |
| | | atttgggcccctctggctggtacttgcggg |
| | | gtcctgctgctttcactcgtgatcactctt |
| | | tactgtaagcgcggtcggaagaagctgctg |
| | | tacatctttaagcaacccttcatgaggcct |
| | | gtgcagactactcaagaggaggacggctgt |
| | | tcatgccggttcccagaggaggaggaaggc |
| | | ggctgcgaactgcgcgtgaaattcagccgc |
| | | agcgcagatgctccagcctaccagcagggg |
| | | cagaaccagctctacaacgaactcaatctt |
| | | ggtcggagagaggagtacgacgtgctggac |
| | | aagcggagaggacgggacccagaaatgggg |
| | | ggaagccgcgcagaaagaatccccaagagg |
| | | gcctgtacaacgagctccaaaaggataaga |
| | | tggcagaagcctatagcgagattggtatga |
| | | aaggggaacgcagaagaggcaaaggccacg |
| | | acggactgtaccagggactcagcaccgcca |
| | | ccaaggacacctatgacgctcttcacatgc |
| | | aggccctgccgcctcgg |
| | 814 | MALPVTALLLPLALLLHAARPEIVMTQSPA |
| | | TLSLSPGERATLSCRASQDISKYLNWYQQK |
| | | PGQAPRLLIYHTSRLHSGIPARFSGSGSGT |
| | | DYTLTISSLQPEDFAVYFCQQGNTLPYTFG |
| | | QGTKLEIKGGGGSGGGGSGGGGSQVQLQES |
| | | GPGLVKPSETLSLTCTVSGVSLPDYGVSWI |
| | | RQPPGKGLEWIGVIWGSETTYYQSSLKSRV |
| | | TISKDNSKNQVSLKLSSVTAADTAVYYCAK |
| | | HYYYGGSYAMDYWGQGTLVTVSSLAEAAAK |
| | | EVQLQQSGPGLVKPSQTLSLTCAISGDSML |
| | | SNSDTWNWIRQSPSRGLEWLGRTYHRSTWY |
| | | DDYASSVRGRVSINVDTSKNQYSLQLNAVT |
| | | PEDTGVYYCARVRLQDGNSWSDAFDVWGQG |
| | | TMVTVSSGGGGSGGGGSGGGGSQSALTQPA |
| | | SASGSPGQSVTISCTGTSSDVGGYNYVSWY |
| | | QQHPGKAPKLMIYDVSNRPSGVSNRFSGSK |
| | | SGNTASLTISGLQAEDEADYYCSSYTSSST |
| | | LYVFGTGTQLTVLTTTPAPRPPTPAPTIAS |
| | | QPLSLRPEACRPAAGGAVHTRGLDFACDIY |
| | | IWAPLAGTCGVLLLSLVITLYCKRGRKKLL |
| | | YIFKQPFMRPVQTTQEEDGCSCRFPEEEEG |
| | | GCELRVKFSRSADAPAYQQGQNQLYNELNL |
| | | GRREEYDVLDKRRGRDPEMGGKPRRKNPQE |
| | | GLYNELQKDKMAEAYSEIGMKGERRRGKGH |
| | | DGLYQGLSTATKDTYDALHMQALPPR |
| CG#c182 | 815 | atggccctccctgtcaccgccctgctgctt |
| | | ccgctggctcttctgctccacgccgctcgg |
| | | cccgaagtgcagcttcaacaatcaggacca |
| | | ggactcgtcaaaccatcacagaccctctcc |
| | | ctcacatgtgccatctccggggactccatg |
| | | ttgagcaattccgacacttggaattggatt |
| | | agacaaagcccgtcccggggtctggaatgg |
| | | ttgggacgcacctaccaccggtctacttgg |
| | | tacgacgactacgcgtcatccgtgcgggga |
| | | agagtgtccatcaacgtggacacctccaag |
| | | aaccagtacagcctgcagcttaatgccgtg |
| | | actcctgaggatacgggcgtctactactgc |
| | | gcccgcgtccgcctgcaagacgggaacagc |
| | | tggagcgatgcattcgatgtctggggccag |
| | | ggaactatggtcaccgtgtcgtctgggggc |
| | | ggtggatcgggtggcgggggttcggggggg |
| | | gcggctctcagtccgctcttacccaaccgg |
| | | cctcagcctcggggagccccggccagagcg |
| | | tgaccatttcctgcaccggcacttcatccg |
| | | acgtgggcggctacaactacgtgtcctggt |
| | | accaacagcacccgggaaaggcccccaagc |
| | | tcatgatctacgacgtgtccaacaggccct |
| | | cgggagtgtccaaccggttctcgggttcga |
| | | aatcgggaaacacagccagcctgaccatca |
| | | gcggactgcaggctgaagatgaagccgact |
| | | actactgctcctcctacacctcgtcatcca |
| | | cgctctacgtgttcggcactggaactcagc |
| | | tgactgtgctggggggggagggagtgaaat |
| | | tgtgatgacccagtcacccgccactcttag |
| | | cctttcacccggtgagcgcgcaaccctgtc |
| | | ttgcagagcctcccaagacatctcaaaata |
| | | ccttaattggtatcaacagaagcccggaca |
| | | ggctcctcgccttctgatctaccacaccag |
| | | ccggctccattctggaatccctgccaggtt |
| | | cagcggtagcggatctgggaccgactacac |
| | | cctcactatcagctcactgcagccagagga |
| | | cttcgctgtctatttctgtcagcaagggaa |
| | | caccctgccctacacctttggacagggcac |
| | | caagctcgagattaaaggtggaggtggcag |
| | | cggaggaggtgggtccggcggtggaggaag |
| | | ccaggtccaactccaagaaagcggaccggg |
| | | tcttgtgaagccatcagaaactctttcact |
| | | gacttgtactgtgagcggagtgtctctccc |
| | | cgattacggggtgtcttggatcagacagcc |
| | | accggggaagggtctggaatggattggagt |
| | | gatttggggctctgagactacttactacca |
| | | atcatccctcaagtcacgcgtcaccatctc |
| | | aaaggacaactctaagaatcaggtgtcact |
| | | gaaactgtcatctgtgaccgcagccgacac |
| | | cgccgtgtactattgcgctaagcattacta |
| | | ttatggcgggagctacgcaatggattactg |
| | | gggacagggtactctggtcaccgtgtccag |
| | | caccactaccccagcaccgaggccacccac |
| | | cccggctcctaccatcgcctcccagcctct |
| | | gtccctgcgtccggaggcatgtagacccgc |
| | | agctggtggggccgtgcatacccggggtct |
| | | tgacttcgcctgcgatatctacatttgggc |
| | | ccctctggctggtacttgcggggtcctgct |
| | | gctttcactcgtgatcactctttactgtaa |
| | | gcgcggtcggaagaagctgctgtacatctt |
| | | taagcaacccttcatgaggcctgtgcagac |
| | | tactcaagaggaggacggctgttcatgccg |
| | | gttcccagaggaggaggaaggcggctgcga |
| | | actgcgcgtgaaattcagccgcagcgcaga |
| | | tgctccagcctaccagcaggggcagaacca |
| | | gctctacaacgaactcaatcttggtcggag |
| | | agaggagtacgacgtg |
| | 816 | ctggacaagcggagaggacgggacccagaa |
| | | atggggggaagccgcgcagaaagaatcccc |
| | | aagagggcctgtacaacgagctccaaaagg |
| | | ataagatggcagaagcctatagcgagattg |
| | | gtatgaaaggggaacgcagaagaggcaaag |
| | | gccacgacggactgtaccagggactcagca |
| | | ccgccaccaaggacacctatgacgctcttc |
| | | acatgcaggccctgccgcctcggMALPVTA |
| | | LLLPLALLLHAARPEVQLQQSGPGLVKPSQ |
| | | TLSLTCAISGDSMLSNSDTWNWIRQSPSRG |
| | | LEWLGRTYHRSTWYDDYASSVRGRVSINVD |
| | | TSKNQYSLQLNAVTPEDTGVYYCARVRLQD |
| | | GNSWSDAFDVWGQGTMVTVSSGGGGSGGGG |
| | | SGGGGSQSALTQPASASGSPGQSVTISCTG |
| | | TSSDVGGYNYVSWYQQHPGKAPKLMIYDVS |
| | | NRPSGVSNRFSGSKSGNTASLTISGLQAED |
| | | EADYYCSSYTSSSTLYVFGTGTQLTVLGGG |
| | | GSEIVMTQSPATLSLSPGERATLSCRASQD |
| | | ISKYLNWYQQKPGQAPRLLIYHTSRLHSGI |
| | | PARFSGSGSGTDYTLTISSLQPEDFAVYFC |
| | | QQGNTLPYTFGQGTKLEIKGGGGSGGGGSG |
| | | GGGSQVQLQESGPGLVKPSETLSLTCTVSG |
| | | VSLPDYGVSWIRQPPGKGLEWIGVIWGSET |
| | | TYYQSSLKSRVTISKDNSKNQVSLKLSSVT |
| | | AADTAVYYCAKHYYYGGSYAMDYWGQGTLV |
| | | TVSSTTTPAPRPPTPAPTIASQPLSLRPEA |
| | | CRPAAGGAVHTRGLDFACDIYIWAPLAGTC |
| | | GVLLLSLVITLYCKRGRKKLLYIFKQPFMR |
| | | PVQTTQEEDGCSCRFPEEEEGGCELRVKFS |
| | | RSADAPAYQQGQNQLYNELNLGRREEYDVL |
| | | DKRRGRDPEMGGKPRRKNPQEGLYNELQKD |
| | | KMAEAYSEIGMKGERRRGKGHDGLYQGLST |
| | | ATKDTYDALHMQALPPR |
| CG#c188 | 817 | atggccctccctgtcaccgccctgctgctt |
| | | ccgctggctcttctgctccacgccgctcgg |
| | | ccccagtccgctcttacccaaccggcctca |
| | | gcctcggggagccccggccagagcgtgacc |
| | | atttcctgcaccggcacttcatccgacgtg |
| | | ggcggctacaactacgtgtcctggtaccaa |
| | | cagcacccgggaaaggcccccaagctcatg |
| | | atctacgacgtgtccaacaggccctcggga |
| | | gtgtccaaccggttctcgggttcgaaatcg |
| | | ggaaacacagccagcctgaccatcagcgga |
| | | ctgcaggctgaagatgaagccgactactac |
| | | tgctcctcctacacctcgtcatccacgctc |
| | | tacgtgttcggcactggaactcagctgact |
| | | gtgctgggcggaggaggctccgaagtgcag |
| | | cttcaacaatcaggaccaggactcgtcaaa |
| | | ccatcacagaccctctccctcacatgtgcc |
| | | atctccggggactccatgttgagcaattcc |
| | | gacacttggaattggattagacaaagcccg |
| | | tcccggggtctggaatggttgggacgcacc |
| | | taccaccggtctacttggtacgacgactac |
| | | gcgtcatccgtgcggggaagagtgtccatc |
| | | aacgtggacacctccaagaaccagtacagc |
| | | ctgcagcttaatgccgtgactcctgaggat |
| | | acgggcgtctactactgcgcccgcgtccgc |
| | | ctgcaagacgggaacagctggagcgatgca |
| | | ttcgatgtctggggccagggaactatggtc |
| | | accgtgtcgtctggagggggagggagtgaa |
| | | attgtgatgacccagtcacccgccactctt |
| | | agcctttcacccggtgagcgcgcaaccctg |
| | | tcttgcagagcctcccaagacatctcaaaa |
| | | taccttaattggtatcaacagaagcccgga |
| | | caggctcctcgccttctgatctaccacacc |
| | | agccggctccattctggaatccctgccagg |
| | | ttcagcggtagcggatctgggaccgactac |
| | | accctcactatcagctcactgcagccagag |
| | | gacttcgctgtctatttctgtcagcaaggg |
| | | aacaccctgccctacacctttggacagggc |
| | | accaagctcgagattaaaggtggaggtggc |
| | | agcggaggaggtgggtccggcggtggagga |
| | | agccaggtccaactccaagaaagcggaccg |
| | | ggtcttgtgaagccatcagaaactctttca |
| | | ctgacttgtactgtgagcggagtgtctctc |
| | | cccgattacggggtgtcttggatcagacag |
| | | ccaccggggaagggtctggaatggattgga |
| | | gtgatttggggctctgagactacttactac |
| | | caatcatccctcaagtcacgcgtcaccatc |
| | | tcaaaggacaactctaagaatcaggtgtca |
| | | ctgaaactgtcatctgtgaccgcagccgac |
| | | accgccgtgtactattgcgctaagcattac |
| | | tattatggcgggagctacgcaatggattac |
| | | tggggacagggtactctggtcaccgtgtcc |
| | | agcaccactaccccagcaccgaggccaccc |
| | | accccggctcctaccatcgcctcccagcct |
| | | ctgtccctgcgtccggaggcatgtagaccc |
| | | gcagctggtggggccgtgcatacccggggt |
| | | cttgacttcgcctgcgatatctacatttgg |
| | | gcccctctggctggtacttgcggggtcctg |
| | | ctgctttcactcgtgatcactctttactgt |
| | | aagcgcggtcggaagaagctgctgtacatc |
| | | tttaagcaacccttcatgaggcctgtgcag |
| | | actactcaagaggaggacggctgttcatgc |
| | | cggttcccagaggaggaggaaggcggctgc |
| | | gaactgcgcgtgaaattcagccgcagcgca |
| | | gatgctccagcctaccagcaggggcagaac |
| | | cagctctacaacgaactcaatcttggtcgg |
| | | agagaggagtacgacgtgctggacaagcgg |
| | | agaggacgggacccagaaatgggcgggaag |
| | | ccgcgcagaaagaatccccaagagggcctg |
| | | tacaacgagctccaaaaggataagatggca |
| | | gaagcctatagcgagattggtatgaaaggg |
| | | gaacgcagaagaggcaaaggccacgacgga |
| | | ctgtaccagggactcagcaccgccaccaag |
| | | gacacctatgacgctcttcacatgcaggcc |
| | | ctgccgcctcgg |
| | 818 | MALPVTALLLPLALLLHAARPQSALTQPAS |
| | | ASGSPGQSVTISCTGTSSDVGGYNYVSWYQ |
| | | QHPGKAPKLMIYDVSNRPSGVSNRFSGSKS |
| | | GNTASLTISGLQAEDEADYYCSSYTSSSTL |
| | | YVFGTGTQLTVLGGGGSEVQLQQSGPGLVK |
| | | PSQTLSLTCAISGDSMLSNSDTWNWIRQSP |
| | | SRGLEWLGRTYHRSTWYDDYASSVRGRVSI |
| | | NVDTSKNQYSLQLNAVTPEDTGVYYCARVR |
| | | LQDGNSWSDAFDVWGQGTMVTVSSGGGGSE |
| | | IVMTQSPATLSLSPGERATLSCRASQDISK |
| | | YLNWYQQKPGQAPRLLIYHTSRLHSGIPAR |
| | | FSGSGSGTDYTLTISSLQPEDFAVYFCQQG |
| | | NTLPYTFGQGTKLEIKGGGGSGGGGGGGGS |
| | | QVQLQESGPGLVKPSETLSLTCTVSGVSLP |
| | | DYGVSWIRQPPGKGLEWIGVIWGSETTYYQ |
| | | SSLKSRVTISKDNSKNQVSLKLSSVTAADT |
| | | AVYYCAKHYYYGGSYAMDYWGQGTLVTVSS |
| | | TTTPAPRPPTPAPTIASQPLSLRPEACRPA |
| | | AGGAVHTRGLDFACDIYIWAPLAGTCGVLL |
| | | LSLVITLYCKRGRKKLLYIFKQPFMRPVQT |
| | | TQEEDGCSCRFPEEEEGGCELRVKFSRSAD |
| | | APAYQQGQNQLYNELNLGRREEYDVLDKRR |
| | | GRDPEMGGKPRRKNPQEGLYNELQKDKMAE |
| | | AYSEIGMKGERRRGKGHDGLYQGLSTATKD |
| | | TYDALHMQALPPR |
| CG#c224 | 819 | atggccctgcccgtgactgcgctcctgctt |
| | | ccgttggccctgctcctgcatgccgccaga |
| | | cctcagtccgctctgactcagccggcctca |
| | | gcttcggggtcccctggtcaaagcgtcact |
| | | atttcctgtaccggaacctcatcagacgtg |
| | | ggcggctacaattacgtgtcctggtaccaa |
| | | cagcaccccggaaaggctcctaagcttatg |
| | | atctacgacgtgtccaaccggccgtcagga |
| | | gtgtccaacagattctccggctccaagagc |
| | | ggaaacactgccagcttgaccattagcggc |
| | | ttgcaggccgaggacgaagccgactactac |
| | | tgctctagctacacatcctcgtctaccctc |
| | | tacgtgtttggaacggggacccagctgact |
| | | gtgctcgggggtggaggatcagaggtgcaa |
| | | ctccagcagtccggtcctggcctcgtgaaa |
| | | ccgtcccaaaccctgtccctgacttgcgcc |
| | | atctcgggcgactccatgtgtccaattccg |
| | | acacctggaactggattagacaatcgccta |
| | | gccggggactcgaatggctgggccggacct |
| | | accaccggtccacgtggtatgacgactacg |
| | | caagctccgtccggggaagggtgtccatta |
| | | acgtcgatacctccaagaaccagtacagcc |
| | | ttcagctgaacgctgtgacccccgaggata |
| | | ccggcgtctactactgtgcaagagtgcgat |
| | | tgcaggatggaaactcgtggtcggacgcat |
| | | tcgatgtctggggacagggaactatggtga |
| | | ccgtgtcctcgggcggaggcgggagcggag |
| | | gaggaggctctggcggaggaggaagcgaga |
| | | ttgtcatgactcagtccccggccacactct |
| | | ccctgtcacccggagaaagagcaaccctga |
| | | gctgcagggcgtcccaggacatctcgaagt |
| | | acctgaactggtaccagcagaagcctggac |
| | | aagcaccccgcctcctgatctaccacacct |
| | | cgcggctgcattcgggaatccccgccagat |
| | | tctcagggagcggatcaggaaccgactaca |
| | | ccctgactatctcgagcctgcaaccagagg |
| | | atttcgccgtgtacttctgccagcaaggaa |
| | | acaccctgccctacacctttggacagggaa |
| | | ccaagctcgagattaaggggggggtggatc |
| | | gggagggggtggatcaggaggaggggctca |
| | | caagtccagctgcaagaatccggtccggga |
| | | cttgtgaagccgtccgaaaccctgtcactg |
| | | acttgcactgtgtccggggtgtcattgccc |
| | | gactacggcgtgagctggattcggcagccc |
| | | cctggaaagggattggaatggatcggcgtg |
| | | atctggggttcggaaactacctactatcag |
| | | tcctcactgaagtcccgcgtgaccatcagc |
| | | aaggataattccaaaaaccaagtgtctctg |
| | | aagctctccagcgtcactgccgccgatact |
| | | gccgtgtactactgcgccaagcactactat |
| | | tacggcggttcgtacgccatggactactgg |
| | | ggcaagggacactcgtgaccgtgtcatcca |
| | | ccactaccccagcaccgaggccacccaccc |
| | | cggctcctaccatcgcctcccagcctctgt |
| | | ccctgcgtccggaggcatgtagacccgcag |
| | | ctggtggggccgtgcatacccggggtcttg |
| | | acttcgcctgcgatatctacatttgggccc |
| | | ctctggctggtacttgcggggtcctgctgc |
| | | tttcactcgtgatcactctttactgtaagc |
| | | gcggtcggaagaagctgctgtacatcttta |
| | | agcaacccttcatgaggcctgtgcagacta |
| | | ctcaagaggaggacggctgttcatgccggt |
| | | tcccagaggaggaggaaggcggctgcgaac |
| | | tgcgcgtgaaattcagccgcagcgcagatg |
| | | ctccagcctaccagcaggggcagaaccagc |
| | | tctacaacgaactcaatcttggtcggagag |
| | | aggagtacgacgtgctggacaagcggagag |
| | | gacgggacccagaaatgggcgggaagccgc |
| | | gcagaaagaatccccaagagggcctgtaca |
| | | acgagctccaaaaggataagatggcagaag |
| | | cctatagcgagattggtatgaaaggggaac |
| | | gcagaagaggcaaaggccacgacggactgt |
| | | accagggactcagcaccgccaccaaggaca |
| | | cctatgacgctcttcacatgcaggccctgc |
| | | cgcctcgg |
| | 820 | MALPVTALLLPLALLLHAARPQSALTQPAS |
| | | ASGSPGQSVTISCTGTSSDVGGYNYVSWYQ |
| | | QHPGKAPKLMIYDVSNRPSGVSNRFSGSKS |
| | | GNTASLTISGLQAEDEADYYCSSYTSSSTL |
| | | YVFGTGTQLTVLGGGGSEVQLQQSGPGLVK |
| | | PSQTLSLTCAISGDSMLSNSDTWNWIRQSP |
| | | SRGLEWLGRTYHRSTWYDDYASSVRGRVSI |
| | | NVDTSKNQYSLQLNAVTPEDTGVYYCARVR |
| | | LQDGNSWSDAFDVWGQGTMVTVSSGGGGSG |
| | | GGGSGGGGSEIVMTQSPATLSLSPGERATL |
| | | SCRASQDISKYLNWYQQKPGQAPRLLIYHT |
| | | SRLHSGIPARFSGSGSGTDYTLTISSLQPE |
| | | DFAVYFCQQGNTLPYTFGQGTKLEIKGGGG |
| | | SGGGGSGGGGSQVQLQESGPGLVKPSETLS |
| | | LTCTVSGVSLPDYGVSWIRQPPGKGLEWIG |
| | | VIWGSETTYYQSSLKSRVTISKDNSKNQVS |
| | | LKLSSVTAADTAVYYCAKHYYYGGSYAMDY |
| | | WGQGTLVTVSSTTTPAPRPPTPAPTIASQP |
| | | LSLRPEACRPAAGGAVHTRGLDFACDIYIW |
| | | APLAGTCGVLLLSLVITLYCKRGRKKLLYI |
| | | FKQPFMRPVQTTQEEDGCSCRFPEEEEGGC |
| | | ELRVKFSRSADAPAYQQGQNQLYNELNLGR |
| | | REEYDVLDKRRGRDPEMGGKPRRKNPQEGL |
| | | YNELQKDKMAEAYSEIGMKGERRRGKGHDG |
| | | LYQGLSTATKDTYDALHMQALPPR |
| CG#c227 | 821 | atggccctgcccgtgactgcgctcctgctt |
| | | ccgttggccctgctcctgcatgccgccaga |
| | | cctcagtccgctctgactcagccggcctca |
| | | gcttcggggtcccctggtcaaagcgtcact |
| | | atttcctgtaccggaacctcatcagacgtg |
| | | ggcggctacaattacgtgtcctggtaccaa |
| | | cagcaccccggaaaggctcctaagcttatg |
| | | atctacgacgtgtccaaccggccgtcagga |
| | | gtgtccaacagattctccggctccaagagc |
| | | ggaaacactgccagcttgaccattagcggc |
| | | ttgcaggccgaggacgaagccgactactac |
| | | tgctctagctacacatcctcgtctaccctc |
| | | tacgtgtttggaacggggacccagctgact |
| | | gtgctcgggggtggaggatcagaggtgcaa |
| | | ctccagcagtccggtcctggcctcgtgaaa |
| | | ccgtcccaaaccctgtccctgacttgcgcc |
| | | atctcgggcgactccatgctgtccaattcc |
| | | gacacctggaactggattagacaatcgcct |
| | | agccggggactcgaatggctgggccggacc |
| | | taccaccggtccacgtggtatgacgactac |
| | | gcaagctccgtccggggaagggtgtccatt |
| | | aacgtcgatacctccaagaaccagtacagc |
| | | cttcagctgaacgctgtgacccccgaggat |
| | | accggcgtctactactgtgcaagagtgcga |
| | | ttgcaggatggaaactcgtggtcggacgca |
| | | ttcgatgtctggggacagggaactatggtc |
| | | actgtgtcctccggcggtggaggctcgggg |
| | | gggggcggctcaggaggaggcggctcacaa |
| | | gtccagctgcaagaatccggtccgggactt |
| | | gtgaagccgtccgaaaccctgtcactgact |
| | | tgcactgtgtccggggtgtcattgcccgac |
| | | tacggcgtgagctggattcggcagccccct |
| | | ggaaagggattggaatggatcggcgtgatc |
| | | tggggttcggaaactacctactatcagtcc |
| | | tcactgaagtcccgcgtgaccatcagcaag |
| | | gataattccaaaaaccaagtgtctctgaag |
| | | ctctccagcgtcactgccgccgatactgcc |
| | | gtgtactactgcgccaagcactactattac |
| | | ggcggttcgtacgccatggactactgggga |
| | | caaggcactcttgtgactgtgtcaagcggc |
| | | ggtggagggagcggtgggggcggttcagga |
| | | ggaggcggatcagagatcgtgatgacccaa |
| | | tccccagccaccctgtccctcagccctgga |
| | | gaaagagccaccctgagctgccgggcctcc |
| | | caggatatcagcaagtacttgaactggtac |
| | | caacaaaagccggggcaggcgccccggctc |
| | | ctgatctaccacacctcgcgcctccactca |
| | | ggtatccccgccagattctcagggagcggc |
| | | tccggtactgactacaccctgactatttcc |
| | | tcactgcagccagaggactttgccgtgtac |
| | | ttctgccagcagggaaacactctgccgtac |
| | | accttcgggcagggaacgaagcttgaaatt |
| | | aagaccactaccccagcaccgaggccaccc |
| | | accccggctcctaccatcgcctcccagcct |
| | | ctgtccctgcgtccggaggcatgtagaccc |
| | | gcagctggtggggccgtgcatacccggggt |
| | | cttgacttcgcctgcgatatctacatttgg |
| | | gcccctctggctggtacttgcggggtcctg |
| | | ctgctttcactcgtgatcactctttactgt |
| | | aagcgcggtcggaagaagctgctgtacatc |
| | | tttaagcaacccttcatgaggcctgtgcag |
| | | actactcaagaggaggacggctgttcatgc |
| | | cggttcccagaggaggaggaaggcggctgc |
| | | gaactgcgcgtgaaattcagccgcagcgca |
| | | gatgctccagcctaccagcaggggcagaac |
| | | cagctctacaacgaactcaatcttggtcgg |
| | | agagaggagtacgacgtgctggacaagcgg |
| | | agaggacgggacccagaaatgggcgggaag |
| | | ccgcgcagaaagaatccccaagagggcctg |
| | | tacaacgagctccaaaaggataagatggca |
| | | gaagcctatagcgagattggtatgaaaggg |
| | | gaacgcagaagaggcaaaggccacgacgga |
| | | ctgtaccagggactcagcaccgccaccaag |
| | | gacacctatgacgctcttcacatgcaggcc |
| | | ctgccgcctcgg |
| | 822 | MALPVTALLLPLALLLHAARPQSALTQPAS |
| | | ASGSPGQSVTISCTGTSSDVGGYNYVSWYQ |
| | | QHPGKAPKLMIYDVSNRPSGVSNRFSGSKS |
| | | GNTASLTISGLQAEDEADYYCSSYTSSSTL |
| | | YVFGTGTQLTVLGGGGSEVQLQQSGPGLVK |
| | | PSQTLSLTCAISGDSMLSNSDTWNWIRQSP |
| | | SRGLEWLGRTYHRSTWYDDYASSVRGRVSI |
| | | NVDTSKNQYSLQLNAVTPEDTGVYYCARVR |
| | | LQDGNSWSDAFDVWGQGTMVTVSSGGGGSG |
| | | GGGSGGGGSQVQLQESGPGLVKPSETLSLT |
| | | CTVSGVSLPDYGVSWIRQPPGKGLEWIGVI |
| | | WGSETTYYQSSLKSRVTISKDNSKNQVSLK |
| | | LSSVTAADTAVYYCAKHYYYGGSYAMDYWG |
| | | QGTLVTVSSGGGGSGGGGSGGGGSEIVMTQ |
| | | SPATLSLSPGERATLSCRASQDISKYLNWY |
| | | QQKPGQAPRLLIYHTSRLHSGIPARFSGSG |
| | | SGTDYTLTISSLQPEDFAVYFCQQGNTLPY |
| | | TFGQGTKLEIKTTTPAPRPPTPAPTIASQP |
| | | LSLRPEACRPAAGGAVHTRGLDFACDIYIW |
| | | APLAGTCGVLLLSLVITLYCKRGRKKLLYI |
| | | FKQPFMRPVQTTQEEDGCSCRFPEEEEGGC |
| | | ELRVKFSRSADAPAYQQGQNQLYNELNLGR |
| | | REEYDVLDKRRGRDPEMGGKPRRKNPQEGL |
| | | YNELQKDKMAEAYSEIGMKGERRRGKGHDG |
| | | LYQGLSTATKDTYDALHMQALPPR |

Dual CD19-CD22 CARs
CG#c201

| | | |
|---|---|---|
| Full length | 823 | atggccctccctgtcaccgccctgctgctt |
| CD19-CD22 | | ccgctggctcttctgctccacgccgctcgg |
| Dual CAR | | cccgaagtgcagctgcagcagtcagggcct |
| nucleic | | ggcctggtcaagccgtcgcagaccctctcc |
| acid | | ctgacatgcgccattagcggggactccatg |
| | | ctgagcaactcggacacctggaactggatt |
| | | cggcagtccccttcccggggactcgagtgg |
| | | ctcggacgcacctaccatcggagcacttgg |
| | | tacgacgactacgcctcctccgtgagaggt |
| | | cgcgtgtcgatcaacgtggatacctcgaag |
| | | aaccagtatagcttgcaactgaacgccgtg |
| | | acccctgaggataccggagtgtactattgt |
| | | gcgagagtcaggctgcaagacggaaactcc |
| | | tggtccgacgcatttgatgtctggggacag |
| | | ggtactatggtcacggtgtcatctggaggc |
| | | ggaggatcgcaaagcgccctgactcagccg |
| | | gcttcggctagcggttcaccggggcagtcc |
| | | gtgactatctcctgcaccgggacttcctcc |
| | | gacgtgggaggctacaattacgtgtcctgg |
| | | taccagcaacaccccggcaaagccccaaag |
| | | ctgatgatctacgacgtcagcaacagaccc |
| | | agcggagtgtccaaccggttcagcggctcc |
| | | aagtccggcaacaccgcctccctgaccatc |
| | | agcgggcttcaggccgaagatgaggcggat |
| | | tactactgctcctcgtacacctcaagctca |
| | | actctgtacgtgttoggcaccggtactcag |
| | | ctcaccgtgctgaccactaccccagcaccg |
| | | aggccacccaccccggctcctaccatcgcc |
| | | tcccagcctctgtccctgcgtccggaggca |
| | | tgtagacccgcagctggtggggccgtgcat |
| | | acccggggtcttgacttcgcctgcgatatc |
| | | tacatttgggcccctctggctggtacttgc |
| | | ggggtcctgctgctttcactcgtgatcact |
| | | ctttactgtaagcgcggtcggaagaagctg |
| | | ctgtacatctttaagcaacccttcatgagg |
| | | cctgtgcagactactcaagaggaggacggc |
| | | tgttcatgccggttcccagaggaggaggaa |
| | | ggcggctgcgaactgcgcgtgaaattcagc |
| | | cgcagcgcagatgctccagcctaccagcag |
| | | gggcagaaccagctctacaacgaactcaat |
| | | cttggtcggagagaggagtacgacgtgctg |
| | | gacaagcggagaggacgggacccagaaatg |
| | | ggcgggaagccgcgcagaaagaatccccaa |
| | | gagggcctgtacaacgagctccaaaaggat |
| | | aagatggcagaagcctatagcgagattggt |
| | | atgaaaggggaacgcagaagaggcaaaggc |
| | | cacgacggactgtaccagggactcagcacc |
| | | gccaccaaggacacctatgacgctcttcac |
| | | atgcaggccctgccgcctcggggaagcgga |
| | | gctactaacttcagcctgctgaagcaggct |
| | | ggagacgtggaggagaaccctggacctatg |
| | | gccttaccagtgaccgccttgctcctgccg |
| | | ctggccttgctgctccacgccgccaggccg |
| | | gaaattgtgatgacccagtcacccgccact |
| | | cttagcctttcacccggtgagcgcgcaacc |
| | | ctgtcttgcagagcctcccaagacatctca |
| | | aaataccttaattggtatcaacagaagccc |
| | | ggacaggctcctcgccttctgatctaccac |
| | | accagccggctccattctggaatccctgcc |
| | | aggttcagcggtagcggatctgggaccgac |
| | | tacaccctcactatcagctcactgcagcca |
| | | gaggacttcgctgtctatttctgtcagcaa |
| | | gggaacaccctgccctacacctttggacag |
| | | ggcaccaagctcgagattaaaggtggaggt |
| | | ggcagcggaggaggtgggtccggcggtgga |
| | | ggaagccaggtccaactccaagaaagcgga |
| | | ccgggtcttgtgaagccatcagaaactctt |
| | | tcactgacttgtactgtgagcggagtgtct |
| | | ctccccgattacggggtgtcttggatcaga |
| | | cagccaccggggaagggtctggaatggatt |
| | | ggagtgatttggggctctgagactacttac |
| | | taccaatcatccctcaagtcacgcgtcacc |
| | | atctcaaaggacaactctaagaatcaggtg |
| | | tcactgaaactgtcatctgtgaccgcagcc |
| | | gacaccgccgtgtactattgcgctaagcat |
| | | tactattatgggggagctacgcaatggatt |
| | | actggggacagggtactctggtcaccgtgt |
| | | ccagcaccacgacgccagcgccgcgaccac |
| | | caacaccggcgcccaccatcgcgtcgcagc |
| | | ccctgtccctgcgcccagaggcgtgccggc |
| | | cagcggggggggcgcagtgcacacgagggg |
| | | gctggacttcgcctgtgatatctacatctg |
| | | ggcgcccttggccgggacttgtggggtcct |
| | | tctcctgtcactggttatcaccctttactg |
| | | caaacggggcagaaagaaactcctgtatat |
| | | attcaaacaaccatttatgagaccagtaca |
| | | aactactcaagaggaagatggctgtagctg |
| | | ccgatttccagaagaagaagaaggaggatg |
| | | tgaactgagagtgaagttcagcaggagcgc |
| | | agacgcccccgcgtaccagcagggccagaa |
| | | ccagctctataacgagctcaatctaggacg |
| | | aagagaggagtacgatgttttggacaagag |
| | | acgtggccgggaccctgagatggggggaaa |
| | | gccgagaaggaagaaccctcaggaaggcct |
| | | gtacaatgaactgcagaaagataagatggc |
| | | ggaggcctacagtgagattgggatgaaagg |
| | | cgagcgccggaggggcaaggggcacgatgg |
| | | cctttaccagggtctcagtacagccaccaa |
| | | ggacacctacgacgcccttcacatgcaggc |
| | | cctgccccctcgc |
| Full length | 824 | MALPVTALLLPLALLLHAARPEVQLQQSGP |
| CD19-CD22 | | GLVKPSQTLSLTCAISGDSMLSNSDTWNWI |
| Dual CAR | | RQSPSRGLEWLGRTYHRSTWYDDYASSVRG |
| amino acid | | RVSINVDTSKNQYSLQLNAVTPEDTGVYYC |
| | | ARVRLQDGNSWSDAFDVWGQGTMVTVSSGG |
| | | GGSQSALTQPASASGSPGQSVTISCTGTSS |
| | | DVGGYNYVSWYQQHPGKAPKLMIYDVSNRP |
| | | SGVSNRFSGSKSGNTASLTISGLQAEDEAD |
| | | YYCSSYTSSSTLYVFGTGTQLTVLTTTPAP |
| | | RPPTPAPTIASQPLSLRPEACRPAAGGAVH |
| | | TRGLDFACDIYIWAPLAGTCGVLLLSLVIT |
| | | LYCKRGRKKLLYIFKQPFMRPVQTTQEEDG |
| | | CSCRFPEEEEGGCELRVKFSRSADAPAYQQ |
| | | GQNQLYNELNLGRREEYDVLDKRRGRDPEM |
| | | GGKPRRKNPQEGLYNELQKDKMAEAYSEIG |
| | | MKGERRRGKGHDGLYQGLSTATKDTYDALH |
| | | MQALPPRGSGATNFSLLKQAGDVEENPGPM |
| | | ALPVTALLLPLALLLHAARPEIVMTQSPAT |
| | | LSLSPGERATLSCRASQDISKYLNWYQQKP |
| | | GQAPRLLIYHTSRLHSGIPARFSGSGSGTD |
| | | YTLTISSLQPEDFAVYFCQQGNTLPYTFGQ |
| | | GTKLEIKGGGGSGGGGSGGGGSQVQLQESG |
| | | PGLVKPSETLSLTCTVSGVSLPDYGVSWIR |
| | | QPPGKGLEWIGVIWGSETTYYQSSLKSRVT |
| | | ISKDNSKNQVSLKLSSVTAADTAVYYCAKH |
| | | YYYGGSYAMDYWGQGTLVTVSSTTTPAPRP |
| | | PTPAPTIASQPLSLRPEACRPAAGGAVHTR |
| | | GLDFACDIYIWAPLAGTCGVLLLSLVITLY |
| | | CKRGRKKLLYIFKQPFMRPVQTTQEEDGCS |
| | | CRFPEEEEGGCELRVKFSRSADAPAYQQGQ |
| | | NQLYNELNLGRREEYDVLDKRRGRDPEMGG |
| | | KPRRKNPQEGLYNELQKDKMAEAYSEIGMK |
| | | GERRRGKGHDGLYQGLSTATKDTYDALHMQ |
| | | ALPPR |
| CD22 CAR | 825 | MALPVTALLLPLALLLHAARPEVQLQQSGP |
| (with P2A | | GLVKPSQTLSLTCAISGDSMLSNSDTWNWI |
| site) | | RQSPSRGLEWLGRTYHRSTWYDDYASSVRG |
| | | RVSINVDTSKNQYSLQLNAVTPEDTGVYYC |
| | | ARVRLQDGNSWSDAFDVWGQGTMVTVSSGG |
| | | GGSQSALTQPASASGSPGQSVTISCTGTSS |
| | | DVGGYNYVSWYQQHPGKAPKLMIYDVSNRP |
| | | SGVSNRFSGSKSGNTASLTISGLQAEDEAD |
| | | YYCSSYTSSSTLYVFGTGTQLTVLTTTPAP |
| | | RPPTPAPTIASQPLSLRPEACRPAAGGAVH |
| | | TRGLDFACDIYIWAPLAGTCGVLLLSLVIT |
| | | LYCKRGRKKLLYIFKQPFMRPVQTTQEEDG |
| | | CSCRFPEEEEGGCELRVKFSRSADAPAYQQ |
| | | GQNQLYNELNLGRREEYDVLDKRRGRDPEM |
| | | GGKPRRKNPQEGLYNELQKDKMAEAYSEIG |
| | | MKGERRRGKGHDGLYQGLSTATKDTYDALH |
| | | MQALPPRGSGATNFSLLKQAGDVEENPG |
| CD19 CAR | 826 | PMALPVTALLLPLALLLHAARPEIVMTQSP |
| | | ATLSLSPGERATLSCRASQDISKYLNWYQQ |
| | | KPGQAPRLLIYHTSRLHSGIPARFSGSGSG |
| | | TDYTLTISSLQPEDFAVYFCQQGNTLPYTF |
| | | GQGTKLEIKGGGGSGGGGSGGGGSQVQLQE |
| | | SGPGLVKPSETLSLTCTVSGVSLPDYGVSW |
| | | IRQPPGKGLEWIGVIWGSETTYYQSSLKSR |
| | | VTISKDNSKNQVSLKLSSVTAADTAVYYCA |
| | | KHYYYGGSYAMDYWGQGTLVTVSSTTTPAP |
| | | RPPTPAPTIASQPLSLRPEACRPAAGGAVH |
| | | TRGLDFACDIYIWAPLAGTCGVLLLSLVIT |
| | | LYCKRGRKKLLYIFKQPFMRPVQTTQEEDG |
| | | CSCRFPEEEEGGCELRVKFSRSADAPAYQQ |
| | | GQNQLYNELNLGRREEYDVLDKRRGRDPEM |
| | | GGKPRRKNPQEGLYNELQKDKMAEAYSEIG |
| | | MKGERRRGKGHDGLYQGLSTATKDTYDALH |
| | | MQALPPR |

CG#c203

| | | |
|---|---|---|
| Full length | 827 | atggccttaccagtgaccgccttgctcctg |
| CD19-CD22 | | ccgctggccttgctgctccacgccgccagg |
| Dual CAR | | ccggaaattgtgatgacccagtcacccgcc |
| nucleic | | actcttagcctttcacccggtgagcgcgca |
| acid | | accctgtcttgcagagcctoccaagacatc |
| | | tcaaaataccttaattggtatcaacagaag |
| | | cccggacaggctcctcgccttctgatctac |
| | | cacaccagccggctccattctggaatccct |
| | | gccaggttcagcggtagcggatctgggacc |
| | | gactacaccctcactatcagctcactgcag |
| | | ccagaggacttcgctgtctatttctgtcag |
| | | caagggaacaccctgccctacacctttgga |
| | | cagggcaccaagctcgagattaaaggtgga |
| | | ggtggcagcggaggaggtgggtccggcggt |
| | | ggaggaagccaggtccaactccaagaaagc |
| | | ggaccgggtcttgtgaagccatcagaaact |
| | | ctttcactgacttgtactgtgagcggagtg |
| | | tctctccccgattacggggtgtcttggatc |
| | | agacagccaccggggaagggtctggaatgg |
| | | attggagtgatttggggctctgagactact |
| | | tactaccaatcatccctcaagtcacgcgtc |
| | | accatctcaaaggacaactctaagaatcag |
| | | gtgtcactgaaactgtcatctgtgaccgca |
| | | gccgacaccgccgtgtactattgcgctaag |
| | | cattactattatggcgggagctacgcaatg |
| | | gattactggggacagggtactctggtcacc |
| | | gtgtccagcaccacgacgccagcgccgcga |
| | | ccaccaacaccggcgcccaccatcgcgtcg |
| | | cagcccctgtccctgcgcccagaggcgtgc |
| | | cggccagcggggggggcgcagtgcacacga |
| | | gggggctggacttcgcctgtgatatctaca |
| | | tctgggcgcccttggccgggacttgtgggg |
| | | tccttctcctgtcactggttatcacccttt |
| | | actgcaaacggggcagaaagaaactcctgt |
| | | atatattcaaacaaccatttatgagaccag |
| | | tacaaactactcaagaggaagatggctgta |
| | | gctgccgatttccagaagaagaagaaggag |
| | | gatgtgaactgagagtgaagttcagcagga |
| | | gcgcagacgcccccgcgtaccagcagggcc |
| | | agaaccagctctataacgagctcaatctag |
| | | gacgaagagaggagtacgatgttttggaca |
| | | agagacgtggccgggaccctgagatggggg |
| | | gaaagccgagaaggaagaaccctcaggaag |
| | | gcctgtacaatgaactgcagaaagataaga |
| | | tggcggaggcctacagtgagattgggatga |
| | | aaggcgagcgccggaggggcaaggggcacg |
| | | atggcctttaccagggtctcagtacagcca |
| | | ccaaggacacctacgacgcccttcacatgc |
| | | aggccctgccccctcgcggaagcggagcta |
| | | ctaacttcagcctgctgaagcaggctggag |
| | | acgtggaggagaaccctggacctatggccc |
| | | tccctgtcaccgccctgctgcttccgctgg |
| | | ctcttctgctccacgccgctcggcccgaag |
| | | tgcagctgcagcagtcagggcctggcctgg |
| | | tcaagccgtcgcagaccctctccctgacat |
| | | gcgccattagcggggactccatgctgagca |
| | | actcggacacctggaactggattcggcagt |
| | | ccccttcccggggactcgagtggctcggac |
| | | gcacctaccatcggagcacttggtacgacg |
| | | actacgcctcctccgtgagaggtcgcgtgt |
| | | cgatcaacgtggatacctcgaagaaccagt |
| | | atagcttgcaactgaacgccgtgacccctg |
| | | aggataccggagtgtactattgtgcgagag |
| | | tcaggctgcaagacggaaactcctggtccg |
| | | acgcatttgatgtctggggacagggtacta |
| | | tggtcacggtgtcatctggaggcggaggat |
| | | cgcaaagcgccctgactcagccggcttcgg |
| | | ctagcggttcaccggggcagtccgtgacta |
| | | tctcctgcaccgggacttcctccgacgtgg |
| | | gaggctacaattacgtgtcctggtaccagc |
| | | aacaccccggcaaagccccaaagctgatga |
| | | tctacgacgtcagcaacagacccagcggag |
| | | tgtccaaccggttcagcggctccaagtccg |
| | | gcaacaccgcctccctgaccatcagcgggc |
| | | ttcaggccgaagatgaggcggattactact |
| | | gctcctcgtacacctcaagctcaactctgt |
| | | acgtgttcggcaccggtactcagctcaccg |
| | | tgctgaccactaccccagcaccgaggccac |
| | | ccaccccggctcctaccatcgcctcccagc |
| | | ctctgtccctgcgtccggaggcatgtagac |
| | | ccgcagctggtggggccgtgcatacccggg |
| | | gtcttgacttcgcctgcgatatctacattt |
| | | gggcccctctggctggtacttgcggggtcc |
| | | tgctgctttcactcgtgatcactctttact |
| | | gtaagcgcggtcggaagaagctgctgtaca |
| | | tctttaagcaacccttcatgaggcctgtgc |
| | | agactactcaagaggaggacggctgttcat |
| | | gccggttcccagaggaggaggaaggcggct |
| | | gcgaactgcgcgtgaaattcagccgcagcg |
| | | cagatgctccagcctaccagcaggggcaga |
| | | accagctctacaacgaactcaatcttggtc |
| | | ggagagaggagtacgacgtgctggacaagc |
| | | ggagaggacgggacccagaaatggggggaa |
| | | gccgcgcagaaagaatccccaagagggcct |
| | | gtacaacgagctccaaaaggataagatggc |
| | | agaagcctatagcgagattggtatgaaagg |
| | | ggaacgcagaagaggcaaaggccacgacgg |
| | | actgtaccagggactcagcaccgccaccaa |
| | | ggacacctatgacgctcttcacatgcaggc |
| | | cctgccgcctcgg |
| Full length | 828 | MALPVTALLLPLALLLHAARPEIVMTQSPA |
| CD19-CD22 | | TLSLSPGERATLSCRASQDISKYLNWYQQK |
| Dual CAR | | PGQAPRLLIYHTSRLHSGIPARFSGSGSGT |
| amino acid | | DYTLTISSLQPEDFAVYFCQQGNTLPYTFG |
| | | QGTKLEIKGGGGSGGGGSGGGGSQVQLQES |
| | | GPGLVKPSETLSLTCTVSGVSLPDYGVSWI |
| | | RQPPGKGLEWIGVIWGSETTYYQSSLKSRV |
| | | TISKDNSKNQVSLKLSSVTAADTAVYYCAK |
| | | HYYYGGSYAMDYWGQGTLVTVSSTTTPAPR |
| | | PPTPAPTIASQPLSLRPEACRPAAGGAVHT |
| | | RGLDFACDIYIWAPLAGTCGVLLLSLVITL |
| | | YCKRGRKKLLYIFKQPFMRPVQTTQEEDGC |
| | | SCRFPEEEEGGCELRVKFSRSADAPAYQQG |
| | | QNQLYNELNLGRREEYDVLDKRRGRDPEMG |
| | | GKPRRKNPQEGLYNELQKDKMAEAYSEIGM |
| | | KGERRRGKGHDGLYQGLSTATKDTYDALHM |
| | | QALPPRGSGATNFSLLKQAGDVEENPGPMA |
| | | LPVTALLLPLALLLHAARPEVQLQQSGPGL |
| | | VKPSQTLSLTCAISGDSMLSNSDTWNWIRQ |
| | | SPSRGLEWLGRTYHRSTWYDDYASSVRGRV |
| | | SINVDTSKNQYSLQLNAVTPEDTGVYYCAR |
| | | VRLQDGNSWSDAFDVWGQGTMVTVSSGGGG |
| | | SQSALTQPASASGSPGQSVTISCTGTSSDV |
| | | GGYNYVSWYQQHPGKAPKLMIYDVSNRPSG |
| | | VSNRFSGSKSGNTASLTISGLQAEDEADYY |
| | | CSSYTSSSTLYVFGTGTQLTVLTTTPAPRP |
| | | PTPAPTIASQPLSLRPEACRPAAGGAVHTR |
| | | GLDFACDIYIWAPLAGTCGVLLLSLVITLY |
| | | CKRGRKKLLYIFKQPFMRPVQTTQEEDGCS |
| | | CRFPEEEEGGCELRVKFSRSADAPAYQQGQ |
| | | NQLYNELNLGRREEYDVLDKRRGRDPEMGG |
| | | KPRRKNPQEGLYNELQKDKMAEAYSEIGMK |
| | | GERRRGKGHDGLYQGLSTATKDTYDALHMQ |
| | | ALPPR |
| CD19 CAR | 829 | MALPVTALLLPLALLLHAARPEIVMTQSPA |
| (with P2A | | TLSLSPGERATLSCRASQDISKYLNWYQQK |
| site) | | PGQAPRLLIYHTSRLHSGIPARFSGSGSGT |
| | | DYTLTISSLQPEDFAVYFCQQGNTLPYTFG |
| | | QGTKLEIKGGGGSGGGGSGGGGSQVQLQES |
| | | GPGLVKPSETLSLTCTVSGVSLPDYGVSWI |
| | | RQPPGKGLEWIGVIWGSETTYYQSSLKSRV |
| | | TISKDNSKNQVSLKLSSVTAADTAVYYCAK |
| | | HYYYGGSYAMDYWGQGTLVTVSSTTTPAPR |
| | | PPTPAPTIASQPLSLRPEACRPAAGGAVHT |
| | | RGLDFACDIYIWAPLAGTCGVLLLSLVITL |
| | | YCKRGRKKLLYIFKQPFMRPVQTTQEEDGC |
| | | SCRFPEEEEGGCELRVKFSRSADAPAYQQG |
| | | QNQLYNELNLGRREEYDVLDKRRGRDPEMG |
| | | GKPRRKNPQEGLYNELQKDKMAEAYSEIGM |
| | | KGERRRGKGHDGLYQGLSTATKDTYDALHM |
| | | QALPPRGSGATNFSLLKQAGDVEENPG |
| CD22 CAR | 830 | PMALPVTALLLPLALLLHAARPEVQLQQSG |
| | | PGLVKPSQTLSLTCAISGDSMLSNSDTWNW |
| | | IRQSPSRGLEWLGRTYHRSTWYDDYASSVR |
| | | GRVSINVDTSKNQYSLQLNAVTPEDTGVYY |
| | | CARVRLQDGNSWSDAFDVWGQGTMVTVSSG |
| | | GGGSQSALTQPASASGSPGQSVTISCTGTS |
| | | SDVGGYNYVSWYQQHPGKAPKLMIYDVSNR |
| | | PSGVSNRFSGSKSGNTASLTISGLQAEDEA |
| | | DYYCSSYTSSSTLYVFGTGTQLTVLTTTPA |
| | | PRPPTPAPTIASQPLSLRPEACRPAAGGAV |
| | | HTRGLDFACDIYIWAPLAGTCGVLLLSLVI |
| | | TLYCKRGRKKLLYIFKQPFMRPVQTTQEED |
| | | GCSCRFPEEEEGGCELRVKFSRSADAPAYQ |
| | | QGQNQLYNELNLGRREEYDVLDKRRGRDPE |
| | | MGGKPRRKNPQEGLYNELQKDKMAEAYSEI |
| | | GMKGERRRGKGHDGLYQGLSTATKDTYDAL |
| | | HMQALPPR |

CG#c230

| | | |
|---|---|---|
| Full length | 831 | atggcacttcccgtcaccgccctgctgctc |
| CD19-CD22 | | ccactcgccctccttctgcacgccgcccgc |
| Dual CAR | | cccgaagtgcagctgcagcagtcaggaccg |
| nucleic | | ggcctggtcaaaccttcgcagactctgtcc |
| acid | | ctgacttgcgctataagcggggactccatg |
| | | ctgagcaattcggacacttggaactggatt |
| | | cgccaaagccccagccggggtctggaatgg |
| | | ctgggaaggacctaccatcgctctacttgg |
| | | tacgacgactacgccagctccgtgcgagga |
| | | cgcgtgtccatcaacgtggacacctccaag |
| | | aaccagtactcgcttcaactcaacgcagtg |
| | | acccctgaagataccggagtctactattgc |
| | | gcccgcgtgcggctccaggacgggaactcc |
| | | tggtcggacgctttcgatgtctggggacag |
| | | ggcactatggtcaccgtcagctccggcggc |
| | | ggcggtagccaatcggcgctgacacagccg |
| | | gcttccgcctcgggatcgcctggacagtcg |
| | | gtgaccatctcgtgcactggaacctcctcc |
| | | gacgtgggggctacaattatgtgtcatggt |
| | | accagcagcacccgggaaaggcccctaagc |
| | | tgatgatctacgacgtgtccaatagaccta |
| | | gcggggtgtcaaacagattctcoggatcca |
| | | aatccggaaacactgcctccctgaccattt |
| | | ccggactgcaggccgaggacgaagccgatt |
| | | actactgctcctcttacacctcctcatcca |
| | | ccctctacgtgtttgggactgggacccagc |
| | | tgaccgtcctcactaccaccccggccccgc |
| | | ggccccctacaccggcaccgactattgcca |
| | | gccagcctctctcgctgcggccggaggcct |
| | | gccgcccagccgccggcggagccgtgcaca |
| | | cccgcggtctggacttcgcgtgcgatatct |
| | | acatctgggctccgctggccgggacttgtg |
| | | gcgtgctgctgctgtctctggtcatcacac |
| | | tgtactgcaagcgcggaagaaagaagctgc |
| | | tctacatcttcaagcaacccttcatgcggc |
| | | ctgtgcagaccacccaggaagaggatggct |
| | | gctcctgccggttcccggaggaagaagagg |
| | | gcggatgcgaactgcgcgtgaagttcagcc |
| | | gaagcgccgacgccccggcctaccagcagg |
| | | gccagaaccaactgtacaacgaactcaacc |
| | | tgggtcggagagaagagtacgacgtgctgg |
| | | acaaaagacgcggcagggaccccgagatgg |
| | | gcggaaagcctcgccgcaagaacccgcagg |
| | | agggcctctacaacgagctgcagaaggaca |
| | | agatggccgaagcctactcagagatcggca |
| | | tgaagggggagcggaggcgcgggaagggcc |
| | | acgacggtttgtaccaaggactttccactg |
| | | cgaccaaggacacctacgatgccctccata |
| | | tgcaagccctgccgccccggggttccggag |
| | | ctaccaacttctcgctgttgaagcaggccg |
| | | gagatgtcgaggaaaacccgggacctatgg |
| | | ccctgccagtgaccgcgctcctgctgcccc |
| | | tggctctgctgcttcacgcggcccggcctg |
| | | agattgtgatgactcagagcccggcgaccc |
| | | tgtccctgtcccccggggagagagcaaccc |
| | | tgtcgtgccgggcctoccaagacatctcaa |
| | | agtacctcaattggtatcagcagaagccag |
| | | gacaggctccacggttgctgatctaccaca |
| | | cttcgagactgcactcaggaatccccgcgc |
| | | ggttttccggttccggctccgggaccgact |
| | | acaccctgaccatcagctcgctccagcctg |
| | | aggatttcgcagtgtacttctgtcagcaag |
| | | gaaacacccttccatacaccttcggacagg |
| | | gtaccaagctggaaatcaagggaggaggag |
| | | gatctgggggcggtggttccggaggcggtg |
| | | gaagccaagtgcagctccaggaaagcggac |
| | | ccgggctggtcaagccgagcgaaaccctct |
| | | cactgacttgtactgtgtccggagtgtccc |
| | | tgcctgactatggagtgtcctggatccgac |
| | | agccccccggaaagggtctggagtggattg |
| | | gggtcatctggggctccgaaactacctact |
| | | accagagcagcctcaagagccgggtcacca |
| | | tttcaaaggataactccaagaatcaagtgt |
| | | ccctgaagctgtcctcagtgacagccgcag |
| | | acaccgccgtgtactactgcgccaagcact |
| | | actactacggaggctcctacgcaatggact |
| | | actggggacaaggcactttggtcactgtgt |
| | | caagcaccaccacccctgcgcctcggcctc |
| | | ctaccccggctcccactatcgcgagccagc |
| | | cgctgagcctgcggcctgaggcttgccgac |
| | | cggccgctggcggcgccgtgcatactcggg |
| | | gcctcgactttgcctgtgacatctacatct |
| | | gggcccccctggccggaacgtgcggagtgc |
| | | tgctgctgtcgctggtcattaccctgtatt |
| | | gcaaacgcggaaggaagaagctgttgtaca |
| | | ttttcaagcagcccttcatgcgcccggtgc |
| | | aaactactcaggaggaagatggctgttcct |
| | | gtcggttccccgaagaggaagaaggcggct |
| | | gcgagttgagggtcaagttctcccggtccg |
| | | ccgatgctcccgcctaccaacaggggcaga |
| | | accagctttataacgaactgaacctgggca |
| | | ggagggaggaatatgatgtgttggataagc |
| | | gccggggccgggacccagaaatggggggaa |
| | | agcccagaagaaagaaccctcaagagggac |
| | | tttacaacgaattgcagaaagacaaaatgg |
| | | ccgaggcctactccgagattgggatgaagg |
| | | gcgaaagacggagaggaaaggggcacgacg |
| | | ggctctaccagggactcagcaccgccacca |
| | | aagatacctacgacgccctgcatatgcagg |
| | | cgctgccgccgcgc |
| Full length | 824 | MALPVTALLLPLALLLHAARPEVQLQQSGP |
| CD19-CD22 | | GLVKPSQTLSLTCAISGDSMLSNSDTWNWI |
| Dual CAR | | RQSPSRGLEWLGRTYHRSTWYDDYASSVRG |
| amino acid | | RVSINVDTSKNQYSLQLNAVTPEDTGVYYC |
| | | ARVRLQDGNSWSDAFDVWGQGTMVTVSSGG |
| | | GGSQSALTQPASASGSPGQSVTISCTGTSS |
| | | DVGGYNYVSWYQQHPGKAPKLMIYDVSNRP |
| | | SGVSNRFSGSKSGNTASLTISGLQAEDEAD |
| | | YYCSSYTSSSTLYVFGTGTQLTVLTTTPAP |
| | | RPPTPAPTIASQPLSLRPEACRPAAGGAVH |
| | | TRGLDFACDIYIWAPLAGTCGVLLLSLVIT |
| | | LYCKRGRKKLLYIFKQPFMRPVQTTQEEDG |
| | | CSCRFPEEEEGGCELRVKFSRSADAPAYQQ |
| | | GQNQLYNELNLGRREEYDVLDKRRGRDPEM |
| | | GGKPRRKNPQEGLYNELQKDKMAEAYSEIG |
| | | MKGERRRGKGHDGLYQGLSTATKDTYDALH |
| | | MQALPPRGSGATNFSLLKQAGDVEENPGPM |
| | | ALPVTALLLPLALLLHAARPEIVMTQSPAT |
| | | LSLSPGERATLSCRASQDISKYLNWYQQKP |
| | | GQAPRLLIYHTSRLHSGIPARFSGSGSGTD |
| | | YTLTISSLQPEDFAVYFCQQGNTLPYTFGQ |
| | | GTKLEIKGGGGSGGGGSGGGGSQVQLQESG |
| | | PGLVKPSETLSLTCTVSGVSLPDYGVSWIR |
| | | QPPGKGLEWIGVIWGSETTYYQSSLKSRVT |
| | | ISKDNSKNQVSLKLSSVTAADTAVYYCAKH |
| | | YYYGGSYAMDYWGQGTLVTVSSTTTPAPRP |
| | | PTPAPTIASQPLSLRPEACRPAAGGAVHTR |
| | | GLDFACDIYIWAPLAGTCGVLLLSLVITLY |
| | | CKRGRKKLLYIFKQPFMRPVQTTQEEDGCS |
| | | CRFPEEEEGGCELRVKFSRSADAPAYQQGQ |
| | | NQLYNELNLGRREEYDVLDKRRGRDPEMGG |
| | | KPRRKNPQEGLYNELQKDKMAEAYSEIGMK |
| | | GERRRGKGHDGLYQGLSTATKDTYDALHMQ |
| | | ALPPR |
| CD22 CAR | 825 | MALPVTALLLPLALLLHAARPEVQLQQSGP |
| (with P2A | | GLVKPSQTLSLTCAISGDSMLSNSDTWNWI |
| site) | | RQSPSRGLEWLGRTYHRSTWYDDYASSVRG |
| | | RVSINVDTSKNQYSLQLNAVTPEDTGVYYC |
| | | ARVRLQDGNSWSDAFDVWGQGTMVTVSSGG |
| | | GGSQSALTQPASASGSPGQSVTISCTGTSS |
| | | DVGGYNYVSWYQQHPGKAPKLMIYDVSNRP |
| | | SGVSNRFSGSKSGNTASLTISGLQAEDEAD |
| | | YYCSSYTSSSTLYVFGTGTQLTVLTTTPAP |
| | | RPPTPAPTIASQPLSLRPEACRPAAGGAVH |
| | | TRGLDFACDIYIWAPLAGTCGVLLLSLVIT |
| | | LYCKRGRKKLLYIFKQPFMRPVQTTQEEDG |
| | | CSCRFPEEEEGGCELRVKFSRSADAPAYQQ |
| | | GQNQLYNELNLGRREEYDVLDKRRGRDPEM |
| | | GGKPRRKNPQEGLYNELQKDKMAEAYSEIG |
| | | MKGERRRGKGHDGLYQGLSTATKDTYDALH |
| | | MQALPPRGSGATNFSLLKQAGDVEENPG |
| CD19 CAR | 826 | PMALPVTALLLPLALLLHAARPEIVMTQSP |
| | | ATLSLSPGERATLSCRASQDISKYLNWYQQ |
| | | KPGQAPRLLIYHTSRLHSGIPARFSGSGSG |
| | | TDYTLTISSLQPEDFAVYFCQQGNTLPYTF |
| | | GQGTKLEIKGGGGSGGGGSGGGGSQVQLQE |
| | | SGPGLVKPSETLSLTCTVSGVSLPDYGVSW |
| | | IRQPPGKGLEWIGVIWGSETTYYQSSLKSR |
| | | VTISKDNSKNQVSLKLSSVTAADTAVYYCA |
| | | KHYYYGGSYAMDYWGQGTLVTVSSTTTPAP |
| | | RPPTPAPTIASQPLSLRPEACRPAAGGAVH |
| | | TRGLDFACDIYIWAPLAGTCGVLLLSLVIT |
| | | LYCKRGRKKLLYIFKQPFMRPVQTTQEEDG |
| | | CSCRFPEEEEGGCELRVKFSRSADAPAYQQ |
| | | GQNQLYNELNLGRREEYDVLDKRRGRDPEM |
| | | GGKPRRKNPQEGLYNELQKDKMAEAYSEIG |
| | | MKGERRRGKGHDGLYQGLSTATKDTYDALH |
| | | MQALPPR |

CD22 and CD19 CDRs of a dual CAR of the disclosure (e.g., a dual CAR molecule comprising (i) a first CAR that binds to CD22 and (ii) a second CAR that binds to CD19) are provided in Table 31.

TABLE 31
CD22 and CD19 CDR sequences

| | | |
|---|---|---|
| | SEQ ID | |
| Identifier | NO | Sequence |

CD22 CDRs

| | | |
|---|---|---|
| HCDR1 | 745 | SNSDTWN |
| (Kabat) | | |
| HCDR2 | 746 | RTYHRSTWYDDYASSVRG |
| (Kabat) | | |
| HCDR3 | 748 | VRLQDGNSWSDAFDV |
| (Kabat) | | |
| HCDR1 | 832 | GDSMLSNSD |
| (Chothia) | | |
| HCDR2 | 833 | YHRSTWY |
| (Chothia) | | |
| HCDR3 | 748 | VRLQDGNSWSDAFDV |
| (Chothia) | | |
| HCDR1 | 834 | GDSMLSNSDT |
| (IMGT) | | |
| HCDR2 | 835 | TYHRSTWYD |
| (IMGT) | | |
| HCDR3 | 836 | ARVRLQDGNSWSDAFDV |
| (IMGT) | | |
| LCDR1 (Kabat) | 95 | TGTSSDVGGYNYVS |
| LCDR2 (Kabat) | 96 | DVSNRPS |
| LCDR3 (Kabat) | 97 | SSYTSSSTLYV |
| LCDR1 | 98 | TSSDVGGYNY |
| (Chothia) | | |
| LCDR2 | 99 | DVS |
| (Chothia) | | |
| LCDR3 | 100 | YTSSSTLY |
| (Chothia) | | |
| LCDR1 | 101 | SSDVGGYNY |
| (IMGT) | | |
| LCDR2 | 99 | DVS |
| (IMGT) | | |
| LCDR3 | 97 | SSYTSSSTLYV |
| (IMGT) | | |

CD19 CDRs

| | | |
|---|---|---|
| HCDR1 | 837 | GVSLPDYGVS |
| (Kabat) | | |
| HCDR2 | 838 | VIWGSETTYYSSSLKS |
| (Kabat) | 687 | VIWGSETTYYQSSLKS |
| | 839 | VIWGSETTYYNSSLKS |
| HCDR3 | 762 | HYYYGGSYAMDY |
| (Kabat) | | |
| LCDR1 | 763 | RASQDISKYLN |
| LCDR2 | 764 | HTSRLHS |
| LCDR3 | 300 | QQGNTLPYT |

Table 32 provides nucleotide and amino acid sequence for CD19 and CD22 binding domains of a dual CAR or a tandem CAR disclosed herein, e.g., a dual CAR or a tandem CAR comprising (i) a first CAR that binds to CD22 and (ii) a second CAR that binds to CD19.

TABLE 32
CD19 and CD22 binding domains

| | SEQ ID | |
|---|---|---|
| Identifier | NO | Sequence |
| scFv CAR19 | 840 | gaaattgtgatgacccagtcacccgccactcttagcctttcacccggtgagcgcgcaaccctgtctt |
| in c201, c203 | | gcagagcctcccaagacatctcaaaataccttaattggtatcaacagaagcccggacaggctcctc |
| and tandem | | gccttctgatctaccacaccagccggctccattctggaatccctgccaggttcagcggtagcggat |
| CARs c171, | | ctgggaccgactacaccctcactatcagctcactgcagccagaggacttcgctgtctatttctgtca |
| c182, c188 | | gcaagggaacaccctgccctacacctttggacagggcaccaagctcgagattaaaggtggaggt |
| | | ggcagcggaggaggtgggtccggcggtggaggaagccaggtccaactccaagaaagcggac |
| | | cgggtcttgtgaagccatcagaaactctttcactgacttgtactgtgagcggagtgtctctccccgat |
| | | tacggggtgtcttggatcagacagccaccggggaagggtctggaatggattggagtgatttgggg |
| | | ctctgagactacttactaccaatcatccctcaagtcacgcgtcaccatctcaaaggacaactctaag |
| | | aatcaggtgtcactgaaactgtcatctgtgaccgcagccgacaccgccgtgtactattgcgctaag |
| | | cattactattatggcgggagctacgcaatggattactggggacagggtactctggtcaccgtgtcca |
| | | gc |
| | 758 | EIVMTQSPATLSLSPGERATLSCRASQDISKYLNWYQQKPGQA |
| | | PRLLIYHTSRLHSGIPARFSGSGSGTDYTLTISSLQPEDFAVYFC |
| | | QQGNTLPYTFGQGTKLEIKGGGGSGGGGSGGGGSQVQLQESG |
| | | PGLVKPSETLSLTCTVSGVSLPDYGVSWIRQPPGKGLEWIGVI |
| | | WGSETTYYQSSLKSRVTISKDNSKNQVSLKLSSVTAADTAVY |
| | | YCAKHYYYGGSYAMDYWGQGTLVTVSS |
| scFv CAR19 | 841 | gagattgtcatgactcagtccccggccacactctccctgtcacccggagaaagagcaaccctgag |
| in c224 | | ctgcagggcgtcccaggacatctcgaagtacctgaactggtaccagcagaagcctggacaagca |
| | | ccccgcctcctgatctaccacacctcgcggctgcattcgggaatccccgccagattctcagggag |
| | | cggatcaggaaccgactacaccctgactatctcgagcctgcaaccagaggatttcgccgtgtactt |
| | | ctgccagcaaggaaacaccctgccctacacctttggacagggaaccaagctcgagattaagggg |
| | | ggtggtggatcgggagggggtggatcaggaggaggcggctcacaagtccagctgcaagaatcc |
| | | ggtccgggacttgtgaagccgtccgaaaccctgtcactgacttgcactgtgtccggggtgtcattg |
| | | cccgactacggcgtgagctggattcggcagccccctggaaagggattggaatggatcggcgtga |
| | | tctggggttcggaaactacctactatcagtcctcactgaagtcccgcgtgaccatcagcaaggata |
| | | attccaaaaaccaagtgtctctgaagctctccagcgtcactgccgccgatactgccgtgtactactg |
| | | cgccaagcactactattacggggttcgtacgccatggactactggggccaagggacactcgtga |
| | | ccgtgtcatcc |
| | 758 | EIVMTQSPATLSLSPGERATLSCRASQDISKYLNWYQQKPGQA |
| | | PRLLIYHTSRLHSGIPARFSGSGSGTDYTLTISSLQPEDFAVYFC |
| | | QQGNTLPYTFGQGTKLEIKGGGGSGGGGSGGGGSQVQLQESG |
| | | PGLVKPSETLSLTCTVSGVSLPDYGVSWIRQPPGKGLEWIGVI |
| | | WGSETTYYQSSLKSRVTISKDNSKNQVSLKLSSVTAADTAVY |
| | | YCAKHYYYGGSYAMDYWGQGTLVTVSS |
| scFv CAR19 | 842 | caagtccagctgcaagaatccggtccgggacttgtgaagccgtccgaaaccctgtcactgacttg |
| in c227 | | cactgtgtccggggtgtcattgcccgactacggcgtgagctggattcggcagccccctggaaagg |
| | | gattggaatggatcggcgtgatctggggttcggaaactacctactatcagtcctcactgaagtcccg |
| | | cgtgaccatcagcaaggataattccaaaaaccaagtgtctctgaagctctccagcgtcactgccgc |
| | | cgatactgccgtgtactactgcgccaagcactactattacggggttcgtacgccatggactactgg |
| | | ggacaaggcactcttgtgactgtgtcaagcggcggtggagggagcggtgggggcggttcagga |
| | | ggaggcggatcagagatcgtgatgacccaatccccagccaccctgtccctcagccctggagaaa |
| | | gagccaccctgagctgccgggcctcccaggatatcagcaagtacttgaactggtaccaacaaaa |
| | | gccggggcaggcgccccggctcctgatctaccacacctcgcgcctccactcaggtatccccgcc |
| | | agattctcagggagcggctccggtactgactacaccctgactatttcctcactgcagccagaggac |
| | | tttgccgtgtacttctgccagcagggaaacactctgccgtacaccttcgggcagggaacgaagctt |
| | | gaaattaag |
| | 843 | QVQLQESGPGLVKPSETLSLTCTVSGVSLPDYGVSWIRQPPGK |
| | | GLEWIGVIWGSETTYYQSSLKSRVTISKDNSKNQVSLKLSSVT |
| | | AADTAVYYCAKHYYYGGSYAMDYWGQGTLVTVSSGGGGS |
| | | GGGGSGGGGSEIVMTQSPATLSLSPGERATLSCRASQDISKYL |
| | | NWYQQKPGQAPRLLIYHTSRLHSGIPARFSGSGSGTDYTLTISS |
| | | LQPEDFAVYFCQQGNTLPYTFGQGTKLEIK |
| scFv CAR19 | 844 | gagattgtgatgactcagagcccggcgaccctgtccctgtcccccggggagagagcaaccctgt |
| in c230 | | cgtgccgggcctcccaagacatctcaaagtacctcaattggtatcagcagaagccaggacaggct |
| | | ccacggttgctgatctaccacacttcgagactgcactcaggaatccccgcgcggttttccggttccg |
| | | gctccgggaccgactacaccctgaccatcagctcgctccagcctgaggatttcgcagtgtacttct |
| | | gtcagcaaggaaacacccttccatacaccttcggacagggtaccaagctggaaatcaagggagg |
| | | aggaggatctgggggcggtggttccggaggcggtggaagccaagtgcagctccaggaaagcg |
| | | gacccgggctggtcaagccgagcgaaaccctctcactgacttgtactgtgtccggagtgtccctgc |
| | | ctgactatggagtgtcctggatccgacagccccccggaaagggtctggagtggattggggtcatc |
| | | tggggctccgaaactacctactaccagagcagcctcaagagccgggtcaccatttcaaaggataa |
| | | ctccaagaatcaagtgtccctgaagctgtcctcagtgacagccgcagacaccgccgtgtactactg |
| | | cgccaagcactactactacggaggctcctacgcaatggactactggggacaaggcactttggtca |
| | | ctgtgtcaagc |
| | 758 | EIVMTQSPATLSLSPGERATLSCRASQDISKYLNWYQQKPGQA |
| | | PRLLIYHTSRLHSGIPARFSGSGSGTDYTLTISSLQPEDFAVYFC |
| | | QQGNTLPYTFGQGTKLEIKGGGGSGGGGSGGGGSQVQLQESG |
| | | PGLVKPSETLSLTCTVSGVSLPDYGVSWIRQPPGKGLEWIGVI |
| | | WGSETTYYQSSLKSRVTISKDNSKNQVSLKLSSVTAADTAVY |
| | | YCAKHYYYGGSYAMDYWGQGTLVTVSS |
| ScFVCAR22 | 845 | gaagtgcagctgcagcagtcagggcctggcctggtcaagccgtcgcagaccctctccctgacat |
| in c201 and | | gcgccattagcggggactccatgctgagcaactcggacacctggaactggattcggcagtcccct |
| c203 | | tcccggggactcgagtggctcggacgcacctaccatcggagcacttggtacgacgactacgcct |
| | | cctccgtgagaggtcgcgtgtcgatcaacgtggatacctcgaagaaccagtatagcttgcaactga |
| | | acgccgtgacccctgaggataccggagtgtactattgtgcgagagtcaggctgcaagacggaaa |
| | | ctcctggtccgacgcatttgatgtctggggacagggtactatggtcacggtgtcatctggaggcgg |
| | | aggatcgcaaagcgccctgactcagccggcttcggctagcggttcaccggggcagtccgtgact |
| | | atctcctgcaccgggacttcctccgacgtgggaggctacaattacgtgtcctggtaccagcaacac |
| | | cccggcaaagccccaaagctgatgatctacgacgtcagcaacagacccagcggagtgtccaac |
| | | cggttcagcggctccaagtccggcaacaccgcctccctgaccatcagcgggcttcaggccgaag |
| | | atgaggcggattactactgctcctcgtacacctcaagctcaactctgtacgtgttcggcaccggtact |
| | | cagctcaccgtgctg |
| | 846 | EVQLQQSGPGLVKPSQTLSLTCAISGDSMLSNSDTWNWIRQSP |
| | | SRGLEWLGRTYHRSTWYDDYASSVRGRVSINVDTSKNQYSL |
| | | QLNAVTPEDTGVYYCARVRLQDGNSWSDAFDVWGQGTMVT |
| | | VSSGGGGSQSALTQPASASGSPGQSVTISCTGTSSDVGGYNYV |
| | | SWYQQHPGKAPKLMIYDVSNRPSGVSNRFSGSKSGNTASLTIS |
| | | GLQAEDEADYYCSSYTSSSTLYVFGTGTQLTVL |
| ScFVCAR22 | 847 | gaagtgcagctgcagcagtcaggaccgggcctggtcaaaccttcgcagactctgtccctgacttg |
| in 230 | | cgctataagcggggactccatgctgagcaattcggacacttggaactggattcgccaaagcccca |
| | | gccggggtctggaatggctgggaaggacctaccatcgctctacttggtacgacgactacgccagc |
| | | tccgtgcgaggacgcgtgtccatcaacgtggacacctccaagaaccagtactcgcttcaactcaa |
| | | cgcagtgacccctgaagataccggagtctactattgcgcccgcgtgcggctccaggacgggaac |
| | | tcctggtcggacgctttcgatgtctggggacagggcactatggtcaccgtcagctccggcggcgg |
| | | cggtagccaatcggcgctgacacagccggcttccgcctcgggatcgcctggacagtcggtgacc |
| | | atctcgtgcactggaacctcctccgacgtgggcggctacaattatgtgtcatggtaccagcagcac |
| | | ccgggaaaggcccctaagctgatgatctacgacgtgtccaatagacctagcggggtgtcaaaca |
| | | gattctccggatccaaatccggaaacactgcctccctgaccatttccggactgcaggccgaggac |
| | | gaagccgattactactgctcctcttacacctcctcatccaccctctacgtgtttgggactgggaccca |
| | | gctgaccgtcctc |
| | 846 | EVQLQQSGPGLVKPSQTLSLTCAISGDSMLSNSDTWNWIRQSP |
| | | SRGLEWLGRTYHRSTWYDDYASSVRGRVSINVDTSKNQYSL |
| | | QLNAVTPEDTGVYYCARVRLQDGNSWSDAFDVWGQGTMVT |
| | | VSSGGGGSQSALTQPASASGSPGQSVTISCTGTSSDVGGYNYV |
| | | SWYQQHPGKAPKLMIYDVSNRPSGVSNRFSGSKSGNTASLTIS |
| | | GLQAEDEADYYCSSYTSSSTLYVFGTGTQLTVL |
| ScFVCAR22 | 848 | gaagtgcagcttcaacaatcaggaccaggactcgtcaaaccatcacagaccctctccctcacatgt |
| in 171, c182 | | gccatctccggggactccatgttgagcaattccgacacttggaattggattagacaaagcccgtcc |
| | | cggggtctggaatggttgggacgcacctaccaccggtctacttggtacgacgactacgcgtcatc |
| | | cgtgcggggaagagtgtccatcaacgtggacacctccaagaaccagtacagcctgcagcttaatg |
| | | ccgtgactcctgaggatacgggcgtctactactgcgcccgcgtccgcctgcaagacgggaacag |
| | | ctggagcgatgcattcgatgtctggggccagggaactatggtcaccgtgtcgtctgggggcggtg |
| | | gatcgggtggcgggggttcggggggcggcggctctcagtccgctcttacccaaccggcctcagc |
| | | ctcggggagccccggccagagcgtgaccatttcctgcaccggcacttcatccgacgtgggcggc |
| | | tacaactacgtgtcctggtaccaacagcacccgggaaaggcccccaagctcatgatctacgacgt |
| | | gtccaacaggccctcgggagtgtccaaccggttctcgggttcgaaatcgggaaacacagccagc |
| | | ctgaccatcagcggactgcaggctgaagatgaagccgactactactgctcctcctacacctcgtca |
| | | tccacgctctacgtgttcggcactggaactcagctgactgtgctg |
| | 753 | EVQLQQSGPGLVKPSQTLSLTCAISGDSMLSNSDTWNWIRQSP |
| | | SRGLEWLGRTYHRSTWYDDYASSVRGRVSINVDTSKNQYSL |
| | | QLNAVTPEDTGVYYCARVRLQDGNSWSDAFDVWGQGTMVT |
| | | VSSGGGGSGGGGSGGGGSQSALTQPASASGSPGQSVTISCTGT |
| | | SSDVGGYNYVSWYQQHPGKAPKLMIYDVSNRPSGVSNRFSG |
| | | SKSGNTASLTISGLQAEDEADYYCSSYTSSSTLYVFGTGTQLT |
| | | VL |
| ScFVCAR22 | 849 | cagtccgctcttacccaaccggcctcagcctcggggagccccggccagagcgtgaccatttcctg |
| in c188 | | caccggcacttcatccgacgtgggcggctacaactacgtgtcctggtaccaacagcacccggga |
| | | aaggcccccaagctcatgatctacgacgtgtccaacaggccctcgggagtgtccaaccggttctc |
| | | gggttcgaaatcgggaaacacagccagcctgaccatcagcggactgcaggctgaagatgaagc |
| | | cgactactactgctcctcctacacctcgtcatccacgctctacgtgttcggcactggaactcagctga |
| | | ctgtgctgggcggaggaggctccgaagtgcagcttcaacaatcaggaccaggactcgtcaaacc |
| | | atcacagaccctctccctcacatgtgccatctccggggactccatgttgagcaattccgacacttgg |
| | | aattggattagacaaagcccgtcccggggtctggaatggttgggacgcacctaccaccggtctact |
| | | tggtacgacgactacgcgtcatccgtgcggggaagagtgtccatcaacgtggacacctccaaga |
| | | accagtacagcctgcagcttaatgccgtgactcctgaggatacgggcgtctactactgcgcccgc |
| | | gtccgcctgcaagacgggaacagctggagcgatgcattcgatgtctggggccagggaactatgg |
| | | tcaccgtgtcgtct |
| | 850 | QSALTQPASASGSPGQSVTISCTGTSSDVGGYNYVSWYQQHP |
| | | GKAPKLMIYDVSNRPSGVSNRFSGSKSGNTASLTISGLQAEDE |
| | | ADYYCSSYTSSSTLYVFGTGTQLTVLGGGGSEVQLQQSGPGL |
| | | VKPSQTLSLTCAISGDSMLSNSDTWNWIRQSPSRGLEWLGRT |
| | | YHRSTWYDDYASSVRGRVSINVDTSKNQYSLQLNAVTPEDTG |
| | | VYYCARVRLQDGNSWSDAFDVWGQGTMVTVSS |
| ScFVCAR22 | 851 | cagtccgctctgactcagccggcctcagcttcggggtcccctggtcaaagcgtcactatttcctgta |
| in c224 | | ccggaacctcatcagacgtgggggctacaattacgtgtcctggtaccaacagcaccccggaaa |
| | | ggctcctaagcttatgatctacgacgtgtccaaccggccgtcaggagtgtccaacagattctccgg |
| | | ctccaagagcggaaacactgccagcttgaccattagcggcttgcaggccgaggacgaagccga |
| | | ctactactgctctagctacacatcctcgtctaccctctacgtgtttggaacggggacccagctgactg |
| | | tgctcgggggtggaggatcagaggtgcaactccagcagtccggtcctggcctcgtgaaaccgtc |
| | | ccaaaccctgtccctgacttgcgccatctcgggcgactccatgctgtccaattccgacacctggaa |
| | | ctggattagacaatcgcctagccggggactcgaatggctgggccggacctaccaccggtccacg |
| | | tggtatgacgactacgcaagctccgtccggggaagggtgtccattaacgtcgatacctccaagaa |
| | | ccagtacagccttcagctgaacgctgtgacccccgaggataccggcgtctactactgtgcaagag |
| | | tgcgattgcaggatggaaactcgtggtcggacgcattcgatgtctggggacagggaactatggtg |
| | | accgtgtcctcg |
| | 850 | QSALTQPASASGSPGQSVTISCTGTSSDVGGYNYVSWYQQHP |
| | | GKAPKLMIYDVSNRPSGVSNRFSGSKSGNTASLTISGLQAEDE |
| | | ADYYCSSYTSSSTLYVFGTGTQLTVLGGGGSEVQLQQSGPGL |
| | | VKPSQTLSLTCAISGDSMLSNSDTWNWIRQSPSRGLEWLGRT |
| | | YHRSTWYDDYASSVRGRVSINVDTSKNQYSLQLNAVTPEDTG |
| | | VYYCARVRLQDGNSWSDAFDVWGQGTMVTVSS |
| ScFVCAR22 | 852 | cagtccgctctgactcagccggcctcagcttcggggtcccctggtcaaagcgtcactatttcctgta |
| in c227 | | ccggaacctcatcagacgtgggcggctacaattacgtgtcctggtaccaacagcaccccggaaa |
| | | ggctcctaagcttatgatctacgacgtgtccaaccggccgtcaggagtgtccaacagattctccgg |
| | | ctccaagagcggaaacactgccagcttgaccattagcggcttgcaggccgaggacgaagccga |
| | | ctactactgctctagctacacatcctcgtctaccctctacgtgtttggaacggggacccagctgactg |
| | | tgctcgggggtggaggatcagaggtgcaactccagcagtccggtcctggcctcgtgaaaccgtc |
| | | ccaaaccctgtccctgacttgcgccatctcgggcgactccatgctgtccaattccgacacctggaa |
| | | ctggattagacaatcgcctagccggggactcgaatggctgggccggacctaccaccggtccacg |
| | | tggtatgacgactacgcaagctccgtccggggaagggtgtccattaacgtcgatacctccaagaa |
| | | ccagtacagccttcagctgaacgctgtgacccccgaggataccggcgtctactactgtgcaagag |
| | | tgcgattgcaggatggaaactcgtggtcggacgcattcgatgtctggggacagggaactatggtc |
| | | actgtgtcctcc |
| | 850 | QSALTQPASASGSPGQSVTISCTGTSSDVGGYNYVSWYQQHP |
| | | GKAPKLMIYDVSNRPSGVSNRFSGSKSGNTASLTISGLQAEDE |
| | | ADYYCSSYTSSSTLYVFGTGTQLTVLGGGGSEVQLQQSGPGL |
| | | VKPSQTLSLTCAISGDSMLSNSDTWNWIRQSPSRGLEWLGRT |
| | | YHRSTWYDDYASSVRGRVSINVDTSKNQYSLQLNAVTPEDTG |
| | | VYYCARVRLQDGNSWSDAFDVWGQGTMVTVSS |

Table 33 provides nucleotide and amino acid sequences for additional CAR components, e.g., signal peptide, linkers and P2A sites, that can be used in a CAR molecule, e.g., a dual CAR molecule described herein (for example, a dual CAR molecule comprising (i) a first CAR that binds to CD22 and (ii) a second CAR that binds to CD19).

TABLE 33
Additional CAR components

| | SEQ ID | |
|---|---|---|
| Identifier | NO | Sequence |
| Signal peptide for | 853 | atggccctccctgtcaccgccctgctgcttccgctggctcttctgctccacgccgctcggcc |
| CAR22 in c201, | | c |
| c203, and tandem | 2 | MALPVTALLLPLALLLHAARP |
| CARs c171, c182, | | |
| c188 | | |
| Signal peptide | 854 | atggccctgcccgtgactgcgctcctgcttccgttggccctgctcctgcatgccgccagac |
| in tandem CARs | | ct |
| c224, c227 | 2 | MALPVTALLLPLALLLHAARP |
| Signal peptide | 855 | atggccttaccagtgaccgccttgctcctgccgctggccttgctgctccacgccgccaggc |
| CAR19 in c201 and | | cg |
| c203 | 2 | MALPVTALLLPLALLLHAARP |
| Signal peptide | 856 | atggcacttcccgtcaccgccctgctgctcccactcgccctccttctgcacgccgcccgcc |
| CAR22 in c230 | | cc |
| | 2 | MALPVTALLLPLALLLHAARP |
| Signal peptide | 857 | atggccctgccagtgaccgcgctcctgctgcccctggctctgctgcttcacgcggcccgg |
| CAR19 in c230 | | cct |
| | 2 | MALPVTALLLPLALLLHAARP |
| CD8 hinge and | 858 | accactaccccagcaccgaggccacccaccccggctcctaccatcgcctcccagcctctg |
| transmembrane | | tccctgcgtccggaggcatgtagacccgcagctggtggggccgtgcatacccggggtctt |
| CAR22 in c201 and | | gacttcgcctgcgatatctacatttgggcccctctggctggtacttgcggggtcctgctgcttt |
| c203, and in | | cactcgtgatcactctttactgt |
| tandem CARs | 202 | TTTPAPRPPTPAPTIASQPLSLRPEACRPAAGGAVHTRGLD |
| c171, c182, c188, | | FACDIYIWAPLAGTCGVLLLSLVITLYC |
| c224, c227 | | |
| CD8 hinge and | 859 | actaccaccccggccccgcggccccctacaccggcaccgactattgccagccagcctct |
| transmembrane | | ctcgctgcggccggaggcctgccgcccagccgccggcggagccgtgcacacccgcgg |
| CAR22 | | tctggacttcgcgtgcgatatctacatctgggctccgctggccgggacttgtggcgtgctgc |
| In c230 | | tgctgtctctggtcatcacactgtactgc |
| | 202 | TTTPAPRPPTPAPTIASQPLSLRPEACRPAAGGAVHTRGLD |
| | | FACDIYIWAPLAGTCGVLLLSLVITLYC |
| CD8 hinge and | 860 | accacgacgccagcgccgcgaccaccaacaccggcgcccaccatcgcgtcgcagccc |
| transmembrane | | ctgtccctgcgcccagaggcgtgccggccagcggggggggcgcagtgcacacgagg |
| CAR19 in c201 and | | gggctggacttcgcctgtgatatctacatctgggcgcccttggccgggacttgtggggtcct |
| c203 | | tctcctgtcactggttatcaccctttactgc |
| | 202 | TTTPAPRPPTPAPTIASQPLSLRPEACRPAAGGAVHTRGLD |
| | | FACDIYIWAPLAGTCGVLLLSLVITLYC |
| CD8 hinge and | 861 | accaccacccctgcgcctcggcctcctaccccggctcccactatcgcgagccagccgctg |
| transmembrane | | agcctgcggcctgaggcttgccgaccggccgctggcggcgccgtgcatactcggggcct |
| CAR19 | | cgactttgcctgtgacatctacatctgggcccccctggccggaacgtgcggagtgctgctg |
| In c230 | | ctgtcgctggtcattaccctgtattgc |
| | 202 | TTTPAPRPPTPAPTIASQPLSLRPEACRPAAGGAVHTRGLD |
| | | FACDIYIWAPLAGTCGVLLLSLVITLYC |
| 4-1BB | 862 | aagcgcggtcggaagaagctgctgtacatctttaagcaacccttcatgaggcctgtgcaga |
| CAR22 in c201 and | | ctactcaagaggaggacggctgttcatgccggttcccagaggaggaggaaggcggctgc |
| c203, and tandem | | gaactg |
| CARs c171, c182, | 14 | KRGRKKLLYIFKQPFMRPVQTTQEEDGCSCRFPEEEEGGC |
| c188, c224, c227 | | EL |
| 4-1BB | 15 | aaacggggcagaaagaaactcctgtatatattcaaacaaccatttatgagaccagtacaaa |
| CAR19 in c201 and | | ctactcaagaggaagatggctgtagctgccgatttccagaagaagaagaaggaggatgtg |
| c203 | | aactg |
| | 14 | KRGRKKLLYIFKQPFMRPVQTTQEEDGCSCRFPEEEEGGC |
| | | EL |
| 4-1BB | 863 | aagcgcggaagaaagaagctgctctacatcttcaagcaacccttcatgcggcctgtgcag |
| CAR22 in c230 | | accacccaggaagaggatggctgctcctgccggttcccggaggaagaagagggcggat |
| | | gcgaactg |
| | 14 | KRGRKKLLYIFKQPFMRPVQTTQEEDGCSCRFPEEEEGGC |
| | | EL |
| 4-1BB | 864 | aaacgcggaaggaagaagctgttgtacattttcaagcagcccttcatgcgcccggtgcaaa |
| CAR19 in c230 | | ctactcaggaggaagatggctgttcctgtcggttccccgaagaggaagaaggcggctgc |
| | | gagttg |
| | 14 | KRGRKKLLYIFKQPFMRPVQTTQEEDGCSCRFPEEEEGGC |
| | | EL |
| CD3zeta | 865 | cgcgtgaaattcagccgcagcgcagatgctccagcctaccagcaggggcagaaccagct |
| CAR22 in c201 and | | ctacaacgaactcaatcttggtcggagagaggagtacgacgtgctggacaagcggagag |
| c203, and tandem | | gacgggacccagaaatgggcgggaagccgcgcagaaagaatccccaagagggcctgt |
| CARs 171, c182, | | acaacgagctccaaaaggataagatggcagaagcctatagcgagattggtatgaaaggg |
| c188, c224, c227 | | gaacgcagaagaggcaaaggccacgacggactgtaccagggactcagcaccgccacc |
| | | aaggacacctatgacgctcttcacatgcaggccctgccgcctcgg |
| | 20 | RVKFSRSADAPAYQQGQNQLYNELNLGRREEYDVLDKR |
| | | RGRDPEMGGKPRRKNPQEGLYNELQKDKMAEAYSEIGM |
| | | KGERRRGKGHDGLYQGLSTATKDTYDALHMQALPPR |
| CD3zeta | 21 | agagtgaagttcagcaggagcgcagacgcccccgcgtaccagcagggccagaaccag |
| CAR19 in c201 and | | ctctataacgagctcaatctaggacgaagagaggagtacgatgttttggacaagagacgtg |
| c203 | | gccgggaccctgagatggggggaaagccgagaaggaagaaccctcaggaaggcctgt |
| | | acaatgaactgcagaaagataagatggcggaggcctacagtgagattgggatgaaaggc |
| | | gagcgccggaggggcaaggggcacgatggcctttaccagggtctcagtacagccacca |
| | | aggacacctacgacgcccttcacatgcaggccctgccccctcgc |
| | 20 | RVKFSRSADAPAYQQGQNQLYNELNLGRREEYDVLDKR |
| | | RGRDPEMGGKPRRKNPQEGLYNELQKDKMAEAYSEIGM |
| | | KGERRRGKGHDGLYQGLSTATKDTYDALHMQALPPR |
| CD3zeta | 866 | cgcgtgaagttcagccgaagcgccgacgccccggcctaccagcagggccagaaccaa |
| CAR22 in c230 | | ctgtacaacgaactcaacctgggtcggagagaagagtacgacgtgctggacaaaagacg |
| | | cggcagggaccccgagatgggcggaaagcctcgccgcaagaacccgcaggagggcc |
| | | tctacaacgagctgcagaaggacaagatggccgaagcctactcagagatcggcatgaag |
| | | ggggagcggaggcgcgggaagggccacgacggtttgtaccaaggactttccactgcga |
| | | ccaaggacacctacgatgccctccatatgcaagccctgccgccccgg |
| | 20 | RVKFSRSADAPAYQQGQNQLYNELNLGRREEYDVLDKR |
| | | RGRDPEMGGKPRRKNPQEGLYNELQKDKMAEAYSEIGM |
| | | KGERRRGKGHDGLYQGLSTATKDTYDALHMQALPPR |
| CD3zeta | 867 | agggtcaagttctcccggtccgccgatgctcccgcctaccaacaggggcagaaccagctt |
| CAR19 in c230 | | tataacgaactgaacctgggcaggagggaggaatatgatgtgttggataagcgccgggg |
| | | ccgggacccagaaatggggggaaagcccagaagaaagaaccctcaagagggactttac |
| | | aacgaattgcagaaagacaaaatggccgaggcctactccgagattgggatgaagggcga |
| | | aagacggagaggaaaggggcacgacgggctctaccagggactcagcaccgccaccaa |
| | | agatacctacgacgccctgcatatgcaggcgctgccgccgcgc |
| | 20 | RVKFSRSADAPAYQQGQNQLYNELNLGRREEYDVLDKR |
| | | RGRDPEMGGKPRRKNPQEGLYNELQKDKMAEAYSEIGM |
| | | KGERRRGKGHDGLYQGLSTATKDTYDALHMQALPPR |
| Linker between | 868 | ttggcagaagccgccgcgaaa |
| scFVs in c171 | 869 | LAEAAAK |
| Linker between | 870 | ggtggaggtggcagcggaggaggtgggtccggcggtggaggaagc |
| scFVs in | 104 | GGGGSGGGGSGGGGS |
| c182, c188 | | |
| Linker between | 871 | ggcggaggcgggagcggaggaggaggctctggcggaggaggaagc |
| scFVs in c224 | 104 | GGGGSGGGGSGGGGS |
| Linker between | 872 | ggcggtggaggctcgggggggggcggctcaggaggaggcggctca |
| scFVs in c227 | 104 | GGGGSGGGGSGGGGS |
| P2A in c201, c203 | 873 | ggaagcggagctactaacttcagcctgctgaagcaggctggagacgtggaggagaacc |
| | | ctggacct |
| | 874 | GSGATNFSLLKQAGDVEENPGP |
| P2A in c230 | 875 | ggttccggagctaccaacttctcgctgttgaagcaggccggagatgtcgaggaaaacccg |
| | | ggacct |
| | 874 | GSGATNFSLLKQAGDVEENPGP |
| Gly4Ser linker | 876 | Ggtggaggtggcagc |
| | 877 | GGGGS |

In some embodiments, the CAR-expressing immune effector cell described herein can further comprise a second CAR, e.g., a second CAR that includes a different antigen binding domain, e.g., to the same target (e.g., a target described above) or a different target. In some embodiments, the second CAR includes an antigen binding domain to a target expressed on the same cancer cell type as the target of the first CAR. In some embodiments, the CAR-expressing immune effector cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. While not wishing to be bound by theory, placement of a costimulatory signaling domain, e.g., 4-1BB, CD28, CD27, or OX-40, onto the first CAR, and the primary signaling domain, e.g., CD3 zeta, on the second CAR can limit the CAR activity to cells where both targets are expressed. In some embodiments, the CAR expressing immune effector cell comprises a first CAR that includes an antigen binding domain that targets, e.g., a target described above, a transmembrane domain and a costimulatory domain and a second CAR that targets an antigen other than antigen targeted by the first CAR (e.g., an antigen expressed on the same cancer cell type as the first target) and includes an antigen binding domain, a transmembrane domain and a primary signaling domain. In another embodiment, the CAR expressing immune effector cell comprises a first CAR that includes an antigen binding domain that targets, e.g., a target described above, a transmembrane domain and a primary signaling domain and a second CAR that targets an antigen other than antigen targeted by the first CAR (e.g., an antigen expressed on the same cancer cell type as the first target) and includes an antigen binding domain to the antigen, a transmembrane domain and a costimulatory signaling domain.

In some embodiments, the CAR-expressing immune effector cell comprises a CAR described herein, e.g., a CAR to a target described above, and an inhibitory CAR. In some embodiments, the inhibitory CAR comprises an antigen binding domain that binds an antigen found on normal cells but not cancer cells, e.g., normal cells that also express the target. In some embodiments, the inhibitory CAR comprises the antigen binding domain, a transmembrane domain and an intracellular domain of an inhibitory molecule. For example, the intracellular domain of the inhibitory CAR can be an intracellular domain of PD1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4, or TGFR beta.

In some embodiments, an immune effector cell (e.g., T cell, NK cell) comprises a first CAR comprising an antigen binding domain that binds to a tumor antigen as described herein, and a second CAR comprising a PD1 extracellular domain or a fragment thereof.

In some embodiments, the cell further comprises an inhibitory molecule as described above.

In some embodiments, the second CAR in the cell is an inhibitory CAR, wherein the inhibitory CAR comprises an antigen binding domain, a transmembrane domain, and an intracellular domain of an inhibitory molecule. The inhibitory molecule can be chosen from one or more of: PD1, PD-L1, CTLA-4, TIM-3, LAG-3, VISTA, BTLA, TIGIT, LAIR1, CD160, 2B4, TGFR beta, CEACAM-1, CEACAM-3, and CEACAM-5. In some embodiments, the second CAR molecule comprises the extracellular domain of PD1 or a fragment thereof.

In embodiments, the second CAR molecule in the cell further comprises an intracellular signaling domain comprising a primary signaling domain and/or an intracellular signaling domain.

In other embodiments, the intracellular signaling domain in the cell comprises a primary signaling domain comprising the functional domain of CD3 zeta and a costimulatory signaling domain comprising the functional domain of 4-1BB.

In some embodiments, the antigen binding domain of the first CAR molecule comprises a scFv and the antigen binding domain of the second CAR molecule does not comprise a scFv. For example, the antigen binding domain of the first CAR molecule comprises a scFv and the antigen binding domain of the second CAR molecule comprises a camelid VHH domain.

### Conformation of CARs

In the embodiments contemplated herein, it is appreciated that the conformation of one or more CARs could be modulated by the vectors described herein above. Accordingly, these conformations are described below in relation to the CAR-expressing cell.

### Split CAR

In some embodiments, the CAR-expressing cell uses a split CAR. The split CAR approach is described in more detail in publications WO2014/055442 and WO2014/055657. Briefly, a split CAR system comprises a cell expressing a first CAR having a first antigen binding domain and a costimulatory domain (e.g., 41BB), and the cell also expresses a second CAR having a second antigen binding domain and an intracellular signaling domain (e.g., CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens.

### Multiple CAR

In some aspects, the CAR-expressing cell described herein can further comprise a second CAR, e.g., a second CAR that includes a different antigen binding domain, e.g., to the same target or a different target (e.g., a target other than a cancer associated antigen described herein or a different cancer associated antigen described herein). In some embodiments, the second CAR includes an antigen binding domain to a target expressed the same cancer cell type as the cancer associated antigen. In some embodiments, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. While not wishing to be bound by theory, placement of a costimulatory signaling domain, e.g., 4-1BB, CD28, CD27 or OX-40, onto the first CAR, and the primary signaling domain, e.g., CD3 zeta, on the second CAR can limit the CAR activity to cells where both targets are expressed. In some embodiments, the CAR expressing cell comprises a first cancer associated antigen CAR that includes an antigen binding domain that binds a target antigen described herein, a transmembrane domain and a costimulatory domain and a second CAR that targets a different target antigen (e.g., an antigen expressed on that same cancer cell type as the first target antigen) and includes an antigen binding domain, a transmembrane domain and a primary signaling domain. In another embodiment, the CAR expressing cell comprises a first CAR that includes an antigen binding domain that binds a target antigen described herein, a transmembrane domain and a primary signaling domain and a second CAR that targets an antigen other than the first target antigen (e.g., an antigen expressed on the same cancer cell type as the first target antigen) and includes an antigen binding domain to the antigen, a transmembrane domain and a costimulatory signaling domain.

In some embodiments, the disclosure provides a first and second CAR, wherein the antigen binding domain of one of said first CAR said second CAR does not comprise a variable light domain and a variable heavy domain. In some embodiments, the antigen binding domain of one of said first CAR said second CAR is an scFv, and the other is not an scFv. In some embodiments, the antigen binding domain of one of said first CAR said second CAR comprises a single VH domain, e.g., a camelid, shark, or lamprey single VH domain, or a single VH domain derived from a human or mouse sequence. In some embodiments, the antigen binding domain of one of said first CAR said second CAR comprises a nanobody. In some embodiments, the antigen binding domain of one of said first CAR said second CAR comprises a camelid VHH domain.

Once the methods described herein are performed, various assays can be used to evaluate the activity of, for e.g., the ability to expand T cells following antigen stimulation, sustain T cell expansion in the absence of re-stimulation, and anti-cancer activities in appropriate in vitro and animal models. Assays to evaluate the effects of a CAR of the present invention are known to those of skill in the art and generally described below.

Western blot analysis of CAR expression in primary T cells can be used to detect the presence of monomers and dimers. See, e.g., Milone et al., Molecular Therapy 17(8): 1453-1464 (2009). Very briefly, T cells (1:1 mixture of CD4⁺ and CD8⁺ T cells) expressing the CARs are expanded in vitro for more than 10 days followed by lysis and SDS-PAGE under reducing conditions. CARs containing the full length TCR-ζ cytoplasmic domain and the endogenous TCR-ζ chain are detected by western blotting using an antibody to the TCR-ζ chain. The same T cell subsets are used for SDS-PAGE analysis under non-reducing conditions to permit evaluation of covalent dimer formation.

In vitro expansion of CAR⁺ T cells following antigen stimulation can be measured by flow cytometry.

Sustained CAR⁺ T cell expansion in the absence of re-stimulation can also be measured. See, e.g., Milone et al., Molecular Therapy 17(8): 1453-1464 (2009). Briefly, mean T cell volume (fl) is measured on day 8 of culture using a Coulter Multisizer III particle counter, a Nexcelom Cellometer Vision or Millipore Scepter, following stimulation with αCD3/αCD28 coated magnetic beads on day 0, and transduction with the indicated CAR on day 1.

Animal models can also be used to measure a CART activity. For example, xenograft model using human a cancer associated antigen described herein-specific CAR⁺ T cells to treat a primary human pre-B ALL in immunodeficient mice can be used. See, e.g., Milone et al., Molecular Therapy 17(8): 1453-1464 (2009).

Dose dependent CAR treatment response can be evaluated. See, e.g., Milone et al., Molecular Therapy 17(8): 1453-1464 (2009). For example, peripheral blood is obtained 35-70 days after establishing leukemia in mice injected on day 21 with CAR T cells, an equivalent number of mock-transduced T cells, or no T cells. Mice from each group are randomly bled for determination of peripheral blood a cancer associate antigen as described herein⁺ ALL blast counts and then killed on days 35 and 49. The remaining animals are evaluated on days 57 and 70.

Assessment of cell proliferation and cytokine production has been previously described, e.g., at Milone et al., Molecular Therapy 17(8): 1453-1464 (2009).

Cytotoxicity can be assessed by a standard 51Cr-release assay. See, e.g., Milone et al., Molecular Therapy 17(8): 1453-1464 (2009

Imaging technologies can be used to evaluate specific trafficking and proliferation of CARs in tumor-bearing animal models. Such assays have been described, for example, in Barrett et al., Human Gene Therapy 22:1575-1586 (2011).

Other assays, including those described herein as well as those that are known in the art can also be used to evaluate the CARs described herein.

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of the current disclosure.

### Example 1: Expi293F Cells as Supportive LV Production System

Most current LV vector production methods involve HEK293T cells, which comprise SV40 T-antigen. In the field, the presence of the SV40 T-antigen in the producer cells is generally thought to be beneficial for vector production. In addition, HEK293T cells show increased cell growth and transfection efficiency in comparison to HEK293 cells that lack SV40 T-antigen. This experiment evaluates the Expi293F cells, a cell line lacking SV40 T-antigen, as supportive LV production system to minimize safety concerns. As shown in this Example, cells lacking the large T-antigen are shown to give satisfactory yield and purity of lentiviral vectors. The cells tested herein are also beneficial because they reduce the potential for recombination events that might result in replication competent lentiviruses (RCLs), thereby reducing the risk of viral replication and insertion into the host DNA at an undesired locus. Commercial Expi293F™ cells (ThermoFisher—Catalog #A14527—Lot #1994635) were obtained. Lentiviral productivity of Expi293F cell lines were compared against the productivity of lentiviral vectors in HEK293T cell line. Both Expi293F™ cells and HEK293T suspension cells were seeded at a concentration of 0.15E6 cells/mL in FreeStyle™ Medium in a SF 250 mL flask and was and cultured for 3 days at 150 rpm. After three days of cell growth and amplification, the cells were transfected with model GOI plasmid encoding a humanized CD19 CAR (C1). 0.4 μg DNA/E6 cells were mixed with PEIpro® (0.4 μL/1E6 cells) and were allowed to form a transfection complex. The transfection complex was directly added to the cell culture. 24 hours post transfection, sodium butyrate and 25 U/mL benzonase with MgCl₂ (2 mM final concentration) were added to the culture media. Cells were harvested 48 hours post-infection for analytical purpose. Lentiviral productivity for each cell line was determined and compared using a TU assay and ELISA.

Analytical methods: The samples were analyzed by two different methods, TU assay and p24 ELISA, in order to provide a functional titer (measurement of the total of virions capable of integrating into cells) and to assess the quality of production through the ratio PP/IP (physical particles/infectious particles), an important parameter relating to reducing cytotoxicity and increasing the efficiency of cell transduction. This ratio indicated the percentage of viral particles that were infectious as compared to the overall viral particles (physical titer).

Functional titer: TU assay: The Transduction Units assay was based on transduction of HEK293-T cells followed by extraction of genomic DNA and quantification of the viral copies by amplifying a vector specific sequence—lentiviral WPRE element—and a house-keeping gene—albumin sequence known to be present in two copies per human cell—in a duplex qPCR. After normalization and correlation to the number of cells seeded, the concentration of transducing units, i.e., infectious viral particles that were able to deliver their genome into a target cell followed by integration in the host cell genome, was calculated.

p24 enzyme-linked immunosorbent assay (Elisa): This method provided an approximation of effective LVV concentration by detecting all the physical particles, whether functional or not (i.e., immature forms, empty particles) as well as free p24 proteins in the supernatant. The physical titer (Lentivirus Particle (LP)/mL) was quantified by a p24 Elisa measuring the lentiviral capsid protein p24. The p24 core antigen was detected directly in the lentiviral supernatant with a HIV-1 p24 ELISA kit. This Elisa measured the concentration of p24 (pg/mL) which was proportional to the amount of lentiviral particles (LP/mL).

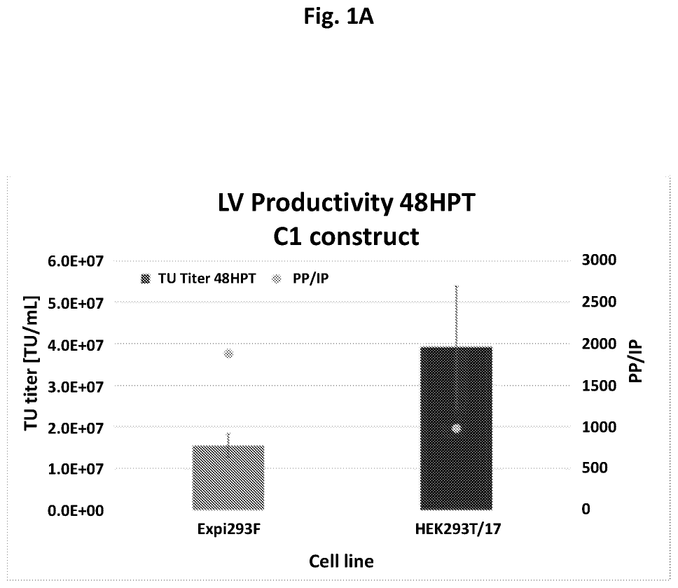

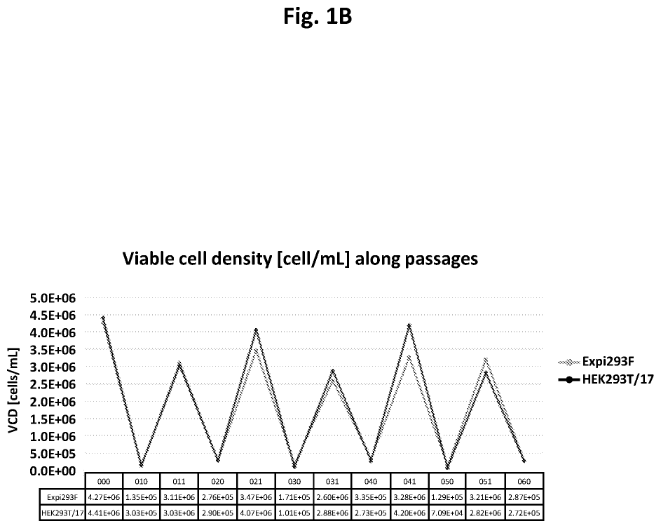

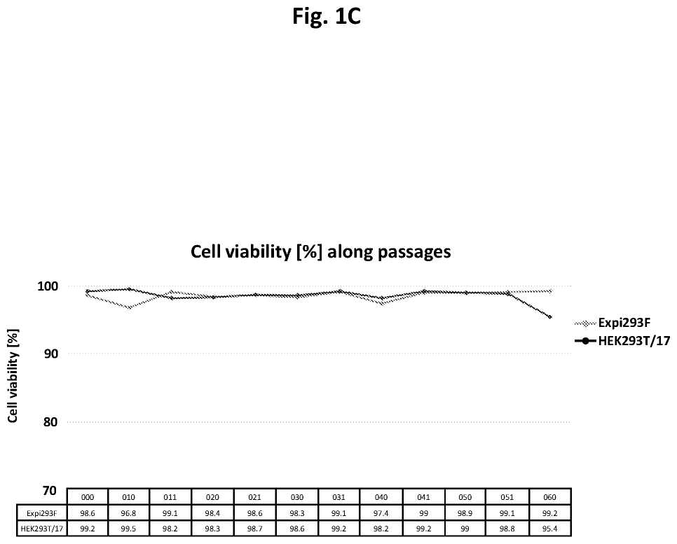

This experiment showed that HEK293T/17 cells show a LV productivity of ˜3.9E7 TU/mL. Expi293F cells generate a satisfactory LV productivity (˜1.5E7 TU/mL) (FIG. 1A). A higher ratio PP/IP is observed (˜1882) which might highlight a reduced assembly efficiency of Expi293F cells. The cell densities observed at each passage were comparable between both cell lines (˜3×10⁶ cells/mL) (FIG. 1B). The population doubling time for the two cell lines were found to be slightly different: around 19 h for HEK293T/17 cells and 21 h for Expi293F (FIG. 1B). Both cell lines showed >90% viability in culture (FIG. 1C). Further development was launched to optimize Expi293F performance.

### Example 2: Evaluation of the Effects of Various Transfection Reagents in Increasing Lentiviral Productivity

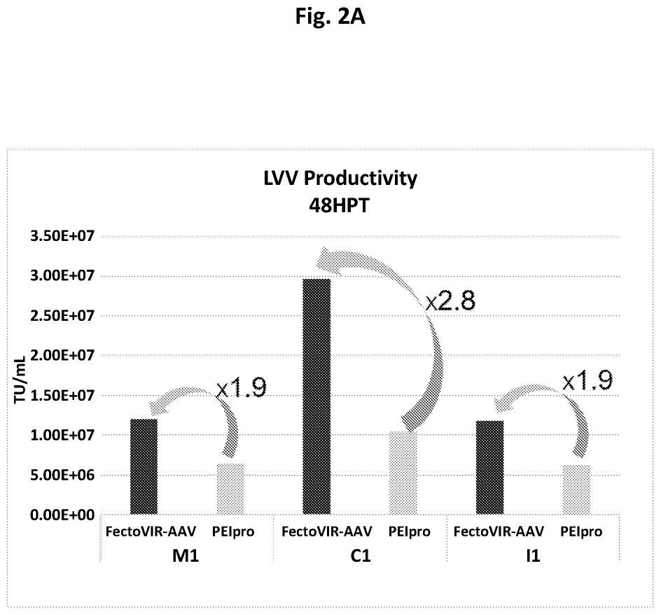

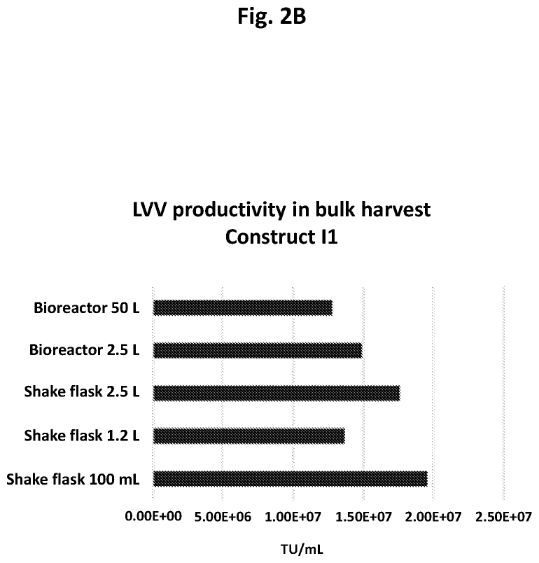

Expi293F™ cells were seeded in one SF 250 mL flask to reach a final density superior to 0.15E6 cells/mL. Cells were seeded at a concentration of 0.15E6 cells/mL in FreeStyle™ Medium in a SF 250 mL flask and was cultured for 3 days at 150 rpm. After three days of cell growth and amplification, the cells were transfected with 3 different model CAR constructs (comprising a humanized CD19 CAR (C1), a CD19-CD22 CAR dual Car (I1), or comprising a humanized CAR and a Tet2 shRNA (M1). PEIpro® or FectoVIR®-AAV was used as transfection reagent. 0.4 μg DNA of each construct/E6 cells were mixed with either PEIpro® (0.4 μL/1E6 cells) or FectoVIR®-AAV (0.4 μL/1E6 cells) and were allowed to form a transfection complex. The transfection complexes were added to the cell culture directly. 24 hours post transfection, 25 U/mL benzonase with MgCl₂ (2 mM final concentration) were added to each culture media. Cells were harvested 48 hours post-infection for analytical purpose. Lentiviral productivity for each cell line was determined using TU assay and Elisa and compared. The transfection reagent FectoVIR®-AAV increased the LV productivity of Expi293F cells significantly, from 1.9 fold to 2.8 fold depending on the gene of interest compared to PEIpro® reagent (FIG. 2A). The gain of productivity was retained in large-scale production (FIG. 2B and Table 1).

TABLE 1
Raw data of lentiviral productivity in bulk harvest

| | Transfection | TU titer | Ratio |
|---|---|---|---|
| GOI | Reagent | (TU/mL) | PP/IP |
| M1 | FectoVIR ®-AAV | 1.20E+07 | 1188 |
| | PEIpro ® | 6.38E+06 | 1955 |
| C1 | FectoVIR ®-AAV | 2.97E+07 | 953 |
| | PEIpro ® | 1.05E+07 | 1157 |
| I1 | FectoVIR ®-AAV | 1.18E+07 | 1800 |
| | PEIpro ® | 6.29E+06 | 1583 |

Furthermore, a reduced ratio PP/IP was observed with when constructs were transfected with FectoVIR ®-AAV highlighting a better transfection efficiency.

### Example 3: Determination of Suitable DNA Quantity for Higher Viral Production

This example describes determining the amount of DNA used for transfection to increase viral yield.

Expi293F™ cells were thawed and seeded in one SF 250 mL flask to reach a final density superior to 0.15E6 cells/mL. Cells were seeded at a concentration of 0.15E6 cells/mL in FreeStyle™ 293 Expression Medium in a SF 250 mL flask and cultured for 3 days at 150 rpm. Cells were cultured routinely to reach a suitable amount for the seeding of a 50 L culture. Expi293F cells were inoculated in the single use stirred tank bioreactor in FreeStyle™ culture medium, and a suitable cell density was reached (1.50×10⁶ cells/mL-2.50×10⁶ cells/mL). The viability of the cells was assessed to be ≥90%. Transfection was performed 72 hours after seeding. 0.3 μg DNA/E6 cells, 0.4 μg DNA/E6 cells, 0.5 μg DNA/E6, or 0.6 μg DNA/E6 cells were mixed with FectoVIR-AAV® in 1:1 ratio in Opti-MEM™ I Reduced Serum Medium (5% wv) and was incubated for 30 minutes to allow for formation of a transfection complex. The transfection complexes were directly added to the cell culture. 20 hours post transfection, 25 U/mL benzonase with MgCl₂ (2 mM final concentration) were added to the culture media. Conditioned media containing the lentiviral vector particles were harvested 48 hours after transfection.

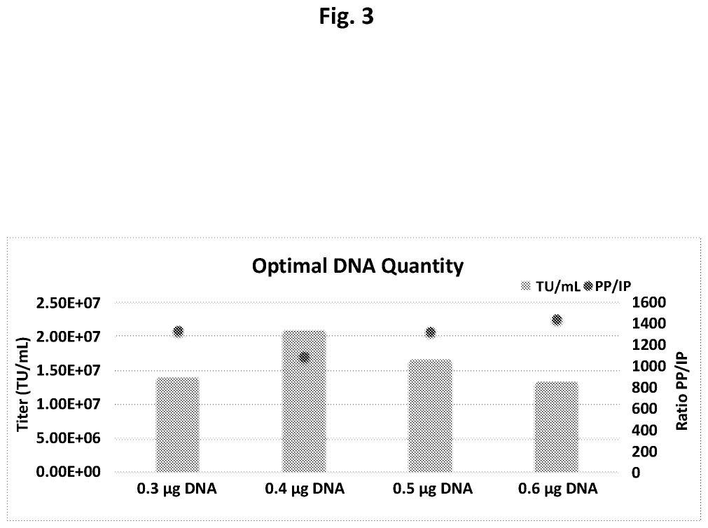

The data showed that the highest viral production was obtained with 0.4 μg DNA/1E6 cells (FIG. 3). Furthermore, viral production obtained with 0.4 μg DNA/1E6 cells was higher than the viral production obtained with 1p g DNA/1E6 cells recommended by the provider (data not shown).

### Example 4: Determination of Modification of Culture Media pH Before Transfection on Lentiviral Productivity

This example demonstrates that lowering the pH of the culture media from 7.1 to 6.7±0.05 increases lentiviral productivity.

Expi293F™ cells were thawed and seeded in one SF250 mL to reach a final density superior to 0.15E6 cells/mL. Cells were cultured for four days under standard culture conditions and scaled up to reach a suitable amount for the seeding of a 2.5 L culture. FreeStyle™ Medium, a chemically defined, serum-free and protein-free medium was used as a culture medium.

After three days of cell growth and amplification, a suitable cell density at transfection defined during the development was reached (1.50×10⁶ cells/mL-2.50×10⁶ cells/mL). The viability of the cells was assessed to be ≥90%. Transfection was performed 72 hours after seeding. After three days of cell growth and amplification, the cells were transfected with 3 model constructs (C1, I1, and M1). Before transfection, the pH setpoint was modified from 7.1 to 6.7±0.05. The transfection was performed after reaching this new setpoint. The cells were transfected with transfection complexes obtained by mixing and incubating 0.4 μg DNA of each construct/E6 cells with FectoVIR®-AAV reagent (0.4 μL/1E6 cells). 24 hours post transfection, 25 U/mL benzonase with MgCl₂ (2 mM final concentration) were added to each culture media. Cells were harvested 48 hours post-infection for analytical purpose. Lentiviral productivity for each cell line was determined using TU assay and Elisa and compared.

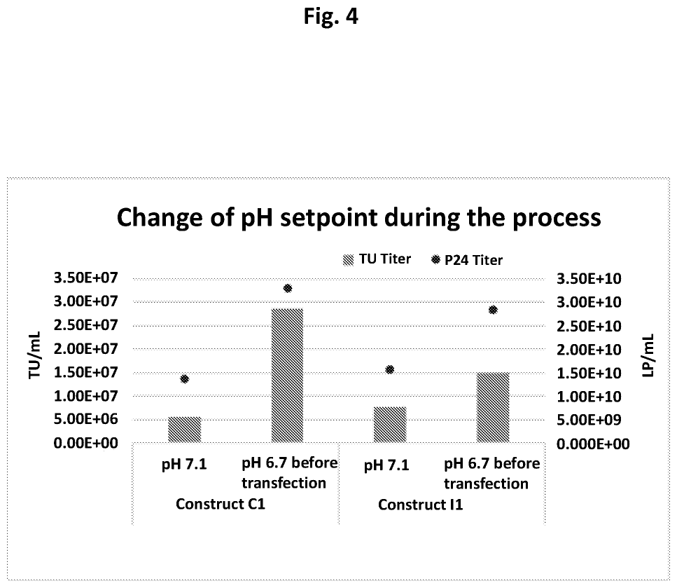

The data showed lentiviral productivity increased about 3-fold for both constructs when the pH was shifted to 6.7 before transfection (FIG. 4). In contrast another study showed that lowering the pH to 6.7 earlier in the process (at inoculation) negatively impacted the cell growth and slowed down the production process (data not shown). Thus, this experiment shows that a carefully timed lowering of the pH yields both high cell growth and higher LV productivity.

### Example 5: Lentivirus Production

All vector production and cell culture were done using Expi293F™ human embryonic kidney (HEK) cells are derived from the 293F cell line and also using HEK293T cells.

One vial of the Expi293F™ cells was thawed and seeded in one SF250 mL flask to reach a final density superior to 0.15E6 cells/mL. Cells were cultured for four days under standard culture conditions using FreeStyle™ medium and scaled up to reach the needed cells amount for the seeding of the stirred tank bioreactor 50 L.

Expi293F cells were inoculated in the single use stirred tank bioreactor at 0.2×10⁶ cells/mL+0.025 in FreeStyle™ medium in a final volume of 47 L. After three days of cell growth and amplification, a suitable cell density at transfection defined during the development was reached (1.50×10⁶ cells/mL-2.50×10⁶ cells/mL). The viability of the cells was assessed to be ≥90%. Transfection was performed 72 hours after seeding. Before transfection, the pH setpoint was modified from 7.1 to 6.7±0.05. The transfection was performed after reaching this new setpoint.

Transient transfection of Expi293F cells was achieved with four different plasmids consisting of three helper plasmids and one transfer (GOI) plasmid:
(i) a plasmid encoding for the glycoprotein of the vesicular stomatitis virus (VSV-g) envelope,
(ii) a plasmid encoding for the structural proteins and viral enzymes,
(iii) a plasmid encoding a post-transcriptional regulator (Rev), and
(iv) the transfer plasmid containing the transgene and the minimal cis-acting sequences that are required for viral RNA production, processing and packaging.

FectoVIR®-AAV (Ref 120-100, Polyplus) was used as a transfection reagent. 0.4 μg DNA/E6 cells were mixed with FectorVIR-AAV® (0.4 μL/1E6 cells) in Opti-MEM™ I Reduced Serum Medium (5% wv) and incubated for 30 minutes to allow for formation of a transfection complex. The transfection complex was directly added to the cell culture. 20 hours post transfection, 25 U/mL benzonase with MgCl₂ (2 mM final concentration) were added to the culture media. Conditioned media containing the lentiviral vector particles were harvested 48 hours after transfection. TU assay and p24 ELISA were performed as described in Example 1.

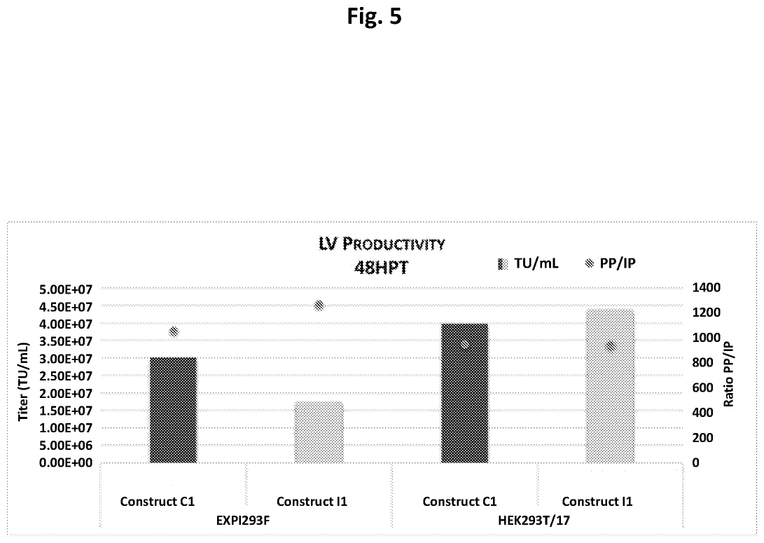

The data showed, application of conditions as described in Examples 2 to 4 increased the lentiviral productivity in Expi293F cells to 3E7 TU/mL for C1 compared to ˜1.5E7 TU/mL obtained in control conditions according to Example 1. The lentiviral productivity was found to be 1.5E7 TU/mL in bulk harvest for I1. FIG. 5 shows the comparative lentiviral productivity using C1 and I1 construct in two production systems: (i) Expi293F cells using FectoVIR®-AAV as a transfection reagent and (ii) HEK293T cells using PEIpro® as a transfection reagent.

### Example 6: Addition of Arginine Improves Filtration Process Time in Various Experimental Setup

Filtration was performed using C1 as a model vector to obtain the clarified harvest. To test the effect of arginine as a stabilizing agent, the starting material containing lentiviral vector was spiked with 1 M arginine-HCl to achieve a final arginine concentration of 50 mM. The clarified harvest was used as untreated control.

Clarified harvest samples were subjected to filtration using PIPES formulation buffer (20 mM PIPES 75 mM NaCl 73 mM Sucrose pH 6.5), to concentrate and re-buffer the lentiviral vector solution. The concentrated and re-buffered lentiviral vector solution was recovered from the filtration skid and the system were flushed hold-up volumes of PIPES filtration buffer.

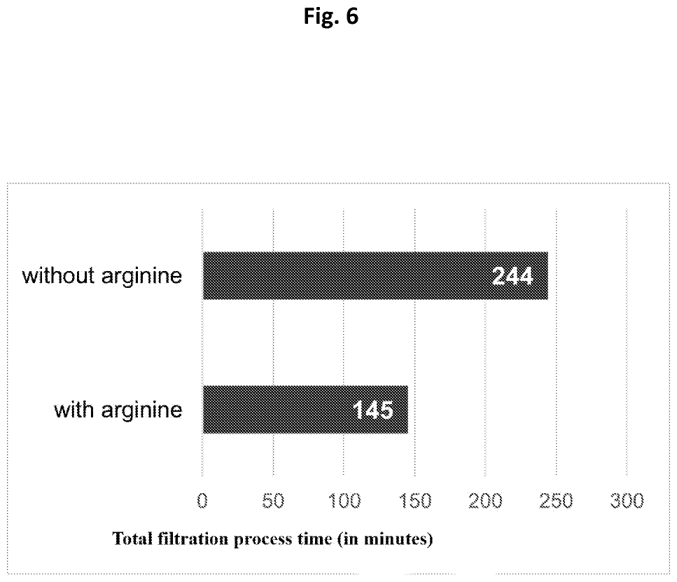

Determination of transducing unit (TU) titer on HEK-293T cells in the first filtrate was performed according to the bioanalytical test method described in Example 1. Determination of p24 by enzyme-linked immunosorbent assay (ELISA) was performed as described in Example 1. The data showed in presence of arginine, the process time was reduced from 244 min to 145 min compared to the control run when samples were subjected to ultrafiltration (FIG. 6). In conclusion, the addition of arginine (50 mM final concentration) improved the process time by about 40%.

### Example 7: Presence of Arginine Improved Vector Recovery During Filtration

This experiment describes the impact of the addition of arginine on the vector recovery of the subsequent filtration steps.

Clarified harvest was prepared and was spiked with 50 mM arginine. Untreated clarified harvest was used as control. For both samples 200 mL clarified harvest were used. The vector concentration in the clarified harvest containing arginine was 4.8E+06 TU/mL, whereas the starting concentration in the control clarified harvest sample was 9.5E+06 TU/mL. The clarified harvest was filtrated and concentrated according to viral purification procedures known to a person skilled in the arts.

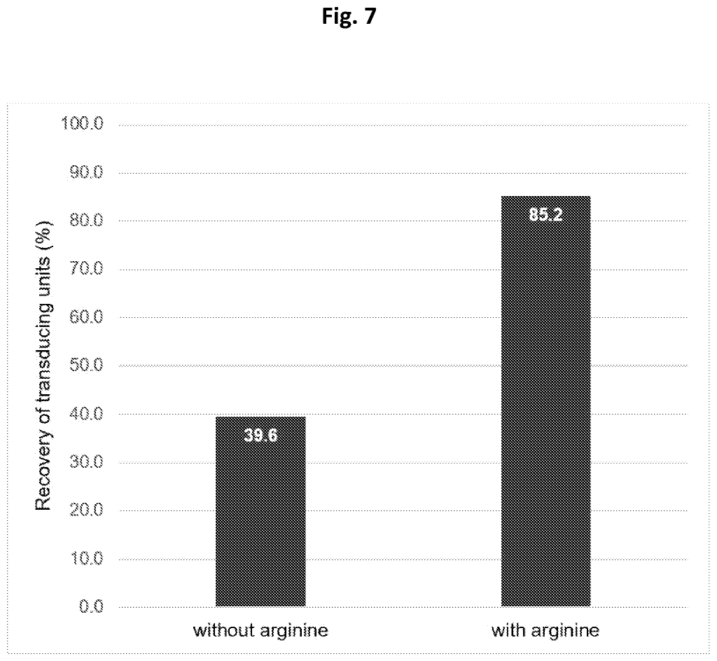

The sample containing arginine had an end volume of 22.3 mL and a vector concentration of 3.7E+07 TU/mL resulting in a vector recovery of 85%, whereas the control sample without arginine had an end volume of 20.5 mL and a vector concentration of 3.7E+07 TU/mL resulting in a vector recovery of only 40% (FIG. 7).

### Examples 8: Further Purification of Clarified Harvest after Subjecting the Samples to Filtration Trials

This experiment described further purification procedure of the clarified harvest. Clarified harvest was spiked with 1 M arginine-HCl to achieve a final arginine concentration of 50 mM. The samples were subjected to filtration using as described in Example 6 to obtain first purification intermediate. Benzonase treatment using 50 U/mL was performed on the samples. Chromatography was performed using PIPES exchange buffer to equilibrate and wash the column.

The purification intermediate (first filtrate) obtained after chromatography was spiked with 1 M arginine-HCl to a final concentration of 75 mM arginine and subjected to filtration using PIPES formulation buffer. The second filtrate was collected for further analysis.

### Example 9: Robust High Vector Recoveries can be Achieved for Both Filtration Steps in the Lentiviral Downstream Process in the Presence of Arginine

This experiment described the vector recovery increased further when arginine spike was implemented prior to filtration steps.

This downstream process was used for the purification of M1 constructs and RCV construct, which were produced using an Expi293F cell-line. Using different vector constructs of product M1, the addition of 75 mM arginine prior to filtrate 1 and filtrate 2 was tested for a range of vector concentrations and also on multiple constructs.

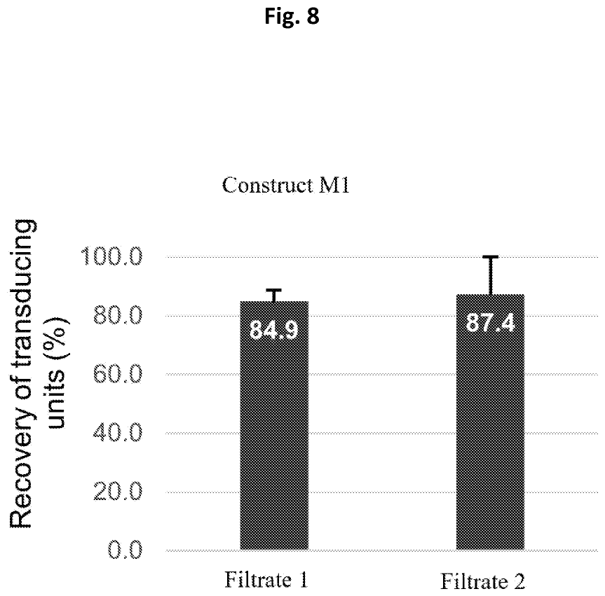

For filtrate 1, an average starting volume of 3168 mL (range: 3031-3224 mL) with an average TU titer of 7.5E+06 TU/mL (range: 4.3E+06-1.2E+07 TU/mL) was used. After concentration and filtration, the recovered filtrate 1 retentate had an average volume of 564 mL (range: 526-611 mL) and TU titer of 3.6E+07 TU/mL (range: 1.9E+07-5.7E+07 TU/mL). Accordingly, the overall concentration factor was about 5.6 and vector recovery in terms of transducing units was on average 85% (FIG. 8).

For filtrate 2, an average starting volume of 559 mL (range: 522-592 mL) with an average TU titer of 2.6E+07 TU/mL (range: 1.5E+07-4.1E+07 TU/mL) was used. After concentration and filtration, the recovered filtrate 2 retentate had an average volume of 46.3 mL (range: 24.3-74.4 ML) and TU titer of 3.4E+08 TU/mL (range: 1.3E+08-1.0E+09 TU/mL). Accordingly, the overall concentration factor was about 13.5 (range: 7.5-22.6) and vector recovery in terms of transducing units was on average 87% (FIG. 8).

### Example 10: Arginine Reduces the Presence of Aggregates in a Concentration Dependent Manner

The example describes the impact of arginine on the presence of aggregates.

Filtrate 2 samples were taken after completion of purification according to Example 8 and were treated as follows: (1) One sample was mixed with 0.825 M arginine to achieve a final arginine concentration of 150 mM. (2) Another sample was mixed with an equal amount 2.475 mM arginine to achieve a final concentration of 300 mM. (3) A control sample was treated with an equal amount of PIPES formulation buffer to keep the final arginine concentration at 75 mM (similar to filtrate 2 filtrate obtained in Example 9) and to account for the dilution caused by the addition of arginine to the other two samples. The samples were analyzed for sub visible particles by micro-flow imaging (MFI).

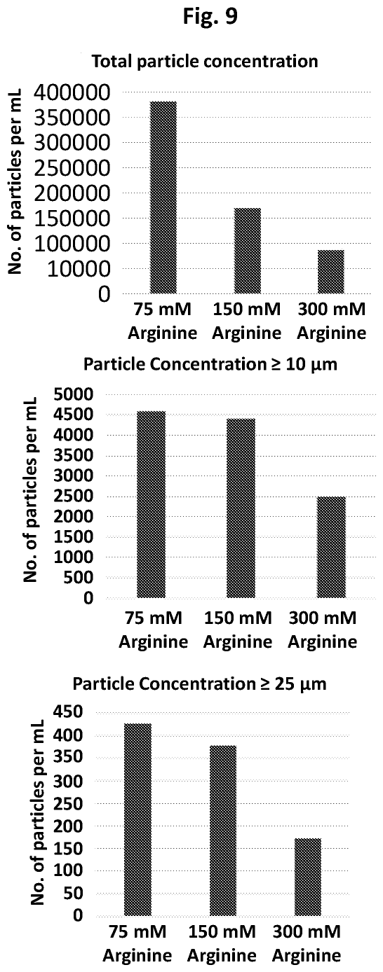

Presence of arginine was found to reduce the particle count and size in a concentration dependent manner (FIG. 9). The addition of arginine seemed to provide the lentiviral particles a resistance to aggregation. In conclusion, arginine was found to be a suitable stabilizing agent for the purification of lentiviral vectors to improve lentiviral vector yields in purification processes.

### Example 11: Presence of Benzonase Decreased DNA Impurities

This example describes the impact of benzonase on DNA impurities.

The lentivirus production was performed as described in Example 5 at 250 mL SF, except for a) a change in the time of benzonase addition; and/or b) a change in the amount of benzonase. In Example 5, benzonase was added at a concentration of 24 U/mL at 24 HPT. With a first batch of LVV (C1), the experiment included varying the addition of benzonase at 5 U/mL, 15 U/mL, 25 U/mL, and 50 U/mL at both 3 HPT and 24 HPT, as seen in Table 12a).

TABLE 12a
Benzonase Addition (bold: control condition)

| Concentration | Process Time |
|---|---|
| / | / |

| | | |
|---|---|---|
| 5 | U/mL | 3 HPT |
| 15 | U/mL | |
| 25 | U/mL | |
| 50 | U/mL | |
| 5 | U/mL | 24 HPT |
| 15 | U/mL | |
| 25 | U/mL | |
| 50 | U/mL | |

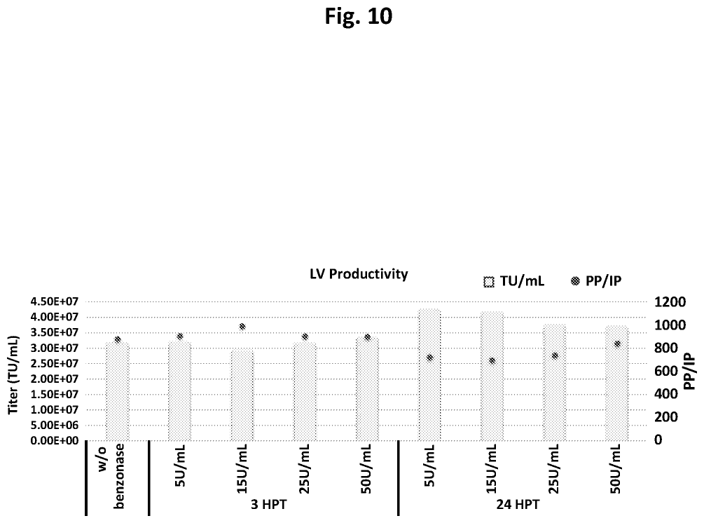

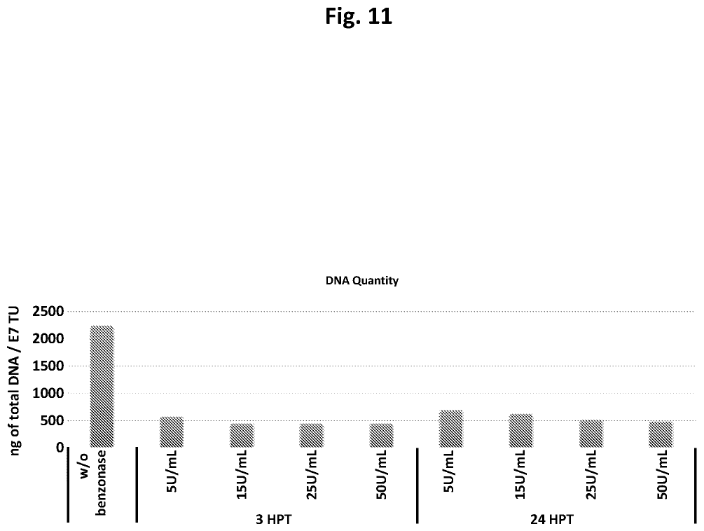

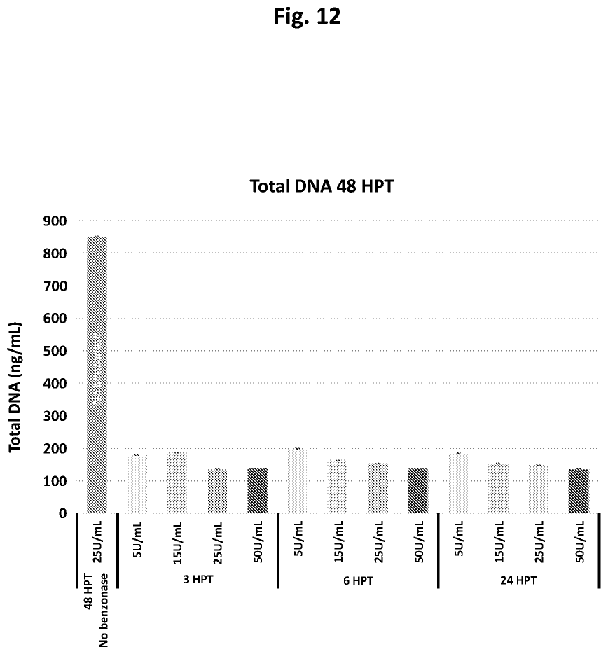

As seen in FIG. 10, there was no impact of benzonase on productivity of infections LVV (TU/mL—TU Assay), showing between 2.9E+7-4.3E+7 TU/mL at harvest, or the ratio of PP/IP (Physical Particles/Infectious Particles) at harvest with varying concentrations of benzonase and different times of addition. However, there was a significant decrease in the total DNA quantity after the addition of benzonase after transfection (FIG. 11). These results were further confirmed with a second batch of LVV production (C1), additionally adding benzonase at 6 HPT (Table 12b; FIG. 12).

TABLE 12b
Benzonase Addition (bold: control condition)

| | |
|---|---|
| Concentration | Process Time |

| | | | |
|---|---|---|---|
| 25 | U/mL | 48 | HPT |
| 5 | U/mL | 3 | HPT |
| 15 | U/mL | | |
| 25 | U/mL | | |
| 50 | U/mL | | |
| 5 | U/mL | 6 | HPT |
| 15 | U/mL | | |
| 25 | U/mL | | |
| 50 | U/mL | | |
| 5 | U/mL | 24 | HPT |
| 15 | U/mL | | |
| 25 | U/mL | | |
| 50 | U/mL | | |

These data show that the addition of benzonase at varying concentrations and time does not affect the productivity of infectious LVV but significantly decreased the concentration of DNA impurities that may affect production.

### Example 12: Incubation Time and Volume of Complexation

This example describes the impact of incubation time and volume of complexation.

The lentivirus production was performed as described in Example 5 at 250 mL SF, except for a) a change in the time of incubation; and/or b) a change in the volume of complexation. In Example 5, FectoVIR®-AAV (Ref 120-100, Polyplus) was used as a transfection reagent. 0.4 μg DNA/E6 cells were mixed with FectorVIR-AAV® (0.4 μL/1E6 cells) in Opti-MEM™ I Reduced Serum Medium (5% wv) and incubated for 30 minutes to allow for formation of a transfection complex (C1). In this example, the complexation volume was varied from 5% wv, 7.5% wv, and 10% wv at 15, 30, 45, and 60 minute incubations (Table 13a)

TABLE 13a
Complexation variables experiment design (bold: control)

| Volume (%) | Time (min) |
|---|---|
| 5 | 15 |
| 7.5 | |
| 10 | |
| 5 | 30 |
| 7.5 | |
| 10 | |
| 5 | 45 |
| 7.5 | |
| 10 | |
| 5 | 60 |
| 7.5 | |
| 10 | |

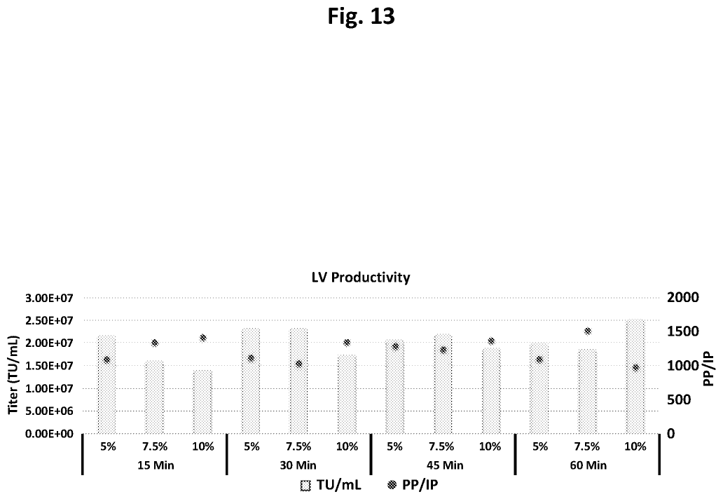

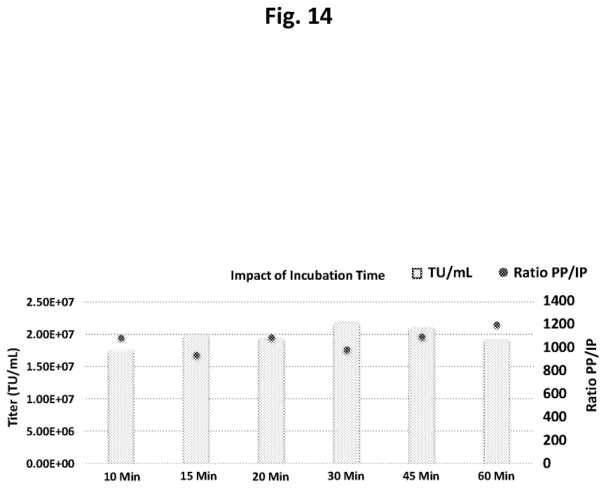

As seen in FIG. 13, there was no positive impact on transfection efficiency observed with increased complexation volume. Furthermore, similar LLV productivity (˜2E+7 TU/mL at harvest), was observed with 5% complexation volume with incubations ranging from 15-60 minutes. These results for a 5% complexation volume were further confirmed with a second batch LVV production using a dual CAR containing LVV (I1) at 10, 15, 20, 30, 45, and 60 minutes (FIG. 14).

These data show that varying incubation time and complexation volume does not affect the productivity of infectious LVV but show a stability of the transfection complex over the incubation period.

### Example 13: Robustness of Process

This example demonstrates the robustness of the production process at varying scales with two different constructs (C1 and I1).

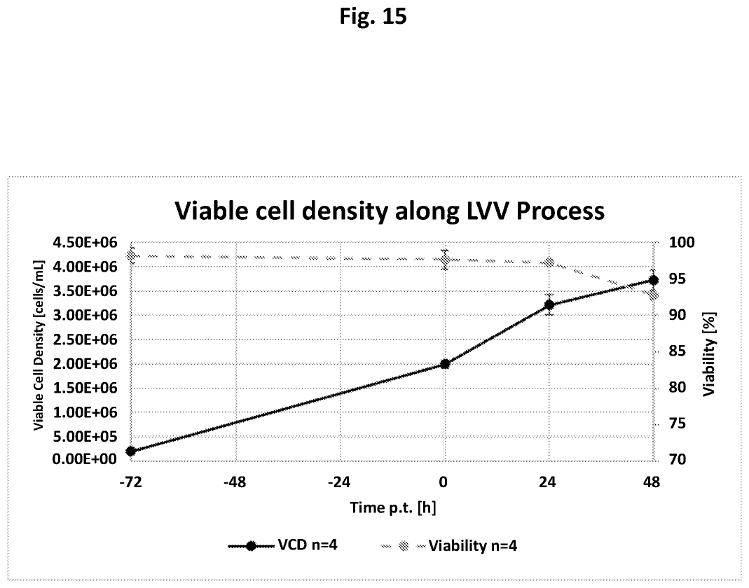

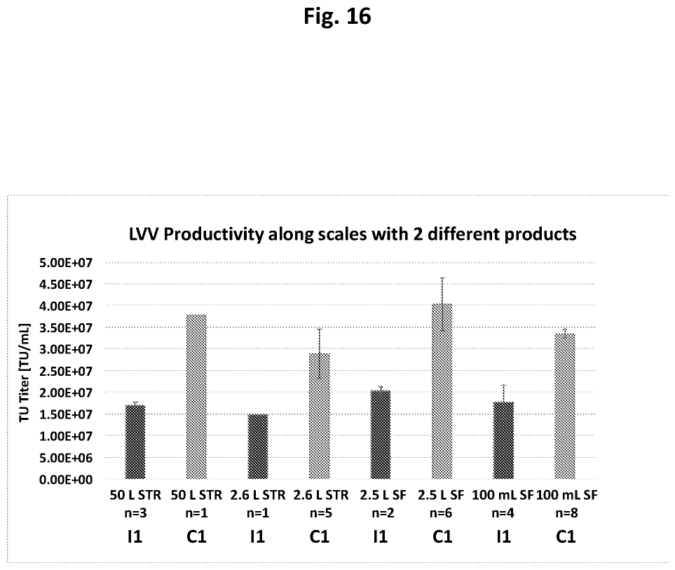

The lentivirus production was performed as described in Example 5, except for a change in the scale of production. Firstly, FIG. 15 shows the cell growth in a stir tank bioreactor at 50 L scale for construct I1. To further test the robustness with varying constructs, FIG. 16 shows the robustness of the process using two different constructs (C1 and I1) at four different scales (50 L, 2.6 L, 2.5 L shake flask (SF), and 100 mL SF).

These data show, for example, that the process is reproducible and robust across multiple scales with varying constructs and can be used for the production of many different LVV.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

What is claimed is:

1. A method of manufacturing a lentiviral vector, comprising:
a) providing a plurality of mammalian (e.g., human) cells,
b) contacting the plurality of mammalian cells with:
i) FectoVIR®-AAV transfection reagent, and
ii) nucleic acid encoding a therapeutic effector, e.g., a therapeutic protein (e.g., a CAR) and sufficient LTR sequence for packaging into a viral particle, and optionally nucleic acid encoding a lentiviral packaging protein, a lentiviral envelope protein, and,
under conditions that allow the nucleic acid to be introduced into at least a subset of the cells; and
c) culturing the cell under conditions suitable for production of the lentiviral vector.

2. The method of claim 1, which when the plurality of mammalian cells is in a 50 L culture yields a number of transducing units per ml culture that is no less than 50%, 60%, 70%, or 80% the number of transducing units per ml culture in an otherwise similar 100 ml culture.

3. The method of claim 1 or 2, which yields at least 1×10⁷ or 3×10⁷ or at least 1×10⁸ transducing units when used under conditions described in Example 5.

4. The method of any of claims 1-3, which yields a ratio of equal to or less than 1188:1, 953:1, and 1800:1 PP (physical particles): IP (infectious particles).

5. The method of claim 1 or 2, wherein the mammalian cells are 293 cells, e.g., Expi293F cells.

6. The method of any of claims 1-5, wherein the FectoVIR®-AAV is used at a concentration of 0.3-0.6 μl FectoVIR®-AAV/million cells, e.g., about 0.4 μl/million cells.

7. The method of any of claims 1-6, wherein the nucleic acid is used at a concentration of 0.3-0.6 μg of nucleic acid/million cells, e.g., about 0.4 μg/million cells.

8. The method of any of claims 1-7 wherein the ratio of FectoVIR®-AAV: DNA for transfection 1:0.5 to 1:2, e.g., about 1:1 (wherein optionally the DNA for transfection comprises DNA encoding the therapeutic effector, DNA encoding one or more retroviral packaging protein and DNA encoding a retroviral envelope protein).

9. The method of any of claims 1-8, wherein the FectoVIR®-AAV transfection reagent is complexed with the nucleic acid.

10. The method of any of claims 1-9, which further comprises admixing the FectoVIR®-AAV transfection reagent with the nucleic acid before step b).

11. The method of claim 9 or 10, wherein complexation volume of the transfection reagent and the nucleic acid is between about 1% and about 15%, e.g., about 1% and about 10% (e.g., about 5-7.5% or 7.5-10%).

12. The method of claim 11, wherein the complexation volume is 3-7%, 4-6%, or about 5%.

13. The method of any one of claims 10-12, wherein the FectoVIR®-AAV transfection reagent and the nucleic acid are incubated for sufficient time to allow complexation to occur, e.g., about 10-90 minutes, e.g., 15-60, e.g., 15-30, 30-45, or 45-60 minutes.

14. A method of manufacturing a lentiviral vector, comprising:
a) culturing a plurality of mammalian (e.g., human) cells at a pH of above about 6.9 or about 6.9-7.3, e.g., about 7.0-7.1;
b) subsequently to step a), adjusting the pH of the culture to about 6.0-6.8, e.g., 6.6-6.8, e.g., about 6.7;
c) subsequently to step b), contacting the culture with a transfection reagent and DNA.

15. The method of claim 14, wherein the transfection reagent comprises FectoVIR®-AAV transfection reagent.

16. The method of claim 14 or 15, wherein the DNA encodes one or more retroviral packaging protein, a retroviral envelope protein, and a therapeutic effector, e.g., a therapeutic protein (e.g., a CAR).

17. The method of any of claims 14-16, wherein a) comprises culturing the cells for about 2-4 days, e.g., about 3 days.

18. The method of any of claims 14-17, which further comprises an additional step of culturing the cells between steps b) and c).

19. The method of any of claims 14-18, which further comprises an additional step of culturing the cells after step c).

20. The method of any of claims 14-19, wherein step b) comprises lowering the pH by about 0.1, 0.2, 0.3, 0.4, 0.5, 0.6, 0.7, 0.8, 0.9, or 1.

21. The method of any one of claims 1-20, wherein prior to step a), the plurality of mammalian cells are inoculated at between 0.1×10⁶ cells/mL—and 0.3×10⁶ cells/mL (e.g., about 0.15×10⁶ cells/mL or about 0.2×10⁶ cells/mL) in culture medium (e.g., FreeStyle™ medium) at a final volume.

22. The method of claim 21, wherein the plurality of mammalian cells are inoculated between 50 and 80 hours (e.g., about 55 hours, about 60 hours, about 65 hours, about 70 hours, about 72 hours, about 75 hours, or about 80 hours) prior to step a).

23. The method of step 21 or 22, wherein the plurality of mammalian cells are cultured under conditions suitable to allow for cell growth and amplification to a suitable cell density at transfection (e.g., between about 1.0×10⁶ cells/mL and about 3.0×10⁶ cells/mL (e.g., between 1.5×10⁶ cells/mL and 2.5×10⁶ cells/mL).

24. A method of manufacturing a lentiviral vector, comprising:
a) providing a composition comprising the lentiviral vector and at least one impurity (e.g., wherein the composition comprises a clarified cell harvest or a filtrate), and
b) contacting the composition with arginine or a salt thereof.

25. The method of claim 24, wherein one or more of:
i) the arginine is at a concentration of about 25-50 mM (about 50 mM), 50-100 mM (e.g., about 75 mM), 100-200 mM (e.g., about 150 mM), or 200-400 (e.g., about 300) mM arginine); or
ii) the arginine is at a concentration sufficient to increase level of transducing units of the lentiviral vector by about 10%-300%, 20%-180%, 30%-160%, 50%-150%, 75%-125% or about 100% compared to an otherwise similar composition, e.g., in an assay according to Example 7;
iii) after step b) the composition shows a total particle concentration per ml of less than 400,000, 300,000, 200,000, or 100,000, as measured by micro-flow imaging, e.g., in an assay described in Example 10, wherein optionally the particles comprise aggregated lentivirus;
iv) after step b) the composition shows a concentration of particles that are ≥10 μm per ml of less than about 5,000, 4,500, 4,000, 3,500, 3,000, or 2,500, as measured by micro-flow imaging, e.g., in an assay described in Example 10 wherein optionally the particles comprise aggregated lentivirus;
v) after step b) the composition shows a concentration of particles that are ≥25 μm per ml of less than about 500, 400, 300, or 200, as measured by micro-flow imaging, e.g., in an assay described in Example 10 wherein optionally the particles comprise aggregated lentivirus;
vi) after step b), the composition shows reduced aggregation of the lentiviral vector compared to an otherwise similar filtrate without addition of the arginine or salt thereof;
vii) recovery of transducing units of the lentiviral vector is greater than an otherwise similar control without arginine added, e.g., by at least about 10%, 20%, 50%, 100%, or 200%, e.g., as measured in an assay according to Example 7.

26. The method of claim 24 or 25, wherein b) comprises contacting the composition with a solution comprising the arginine and a buffer, wherein optionally the buffer is PIPES, wherein optionally the PIPES is at a concentration of from about 10 mM to about 50 mM, e.g., about of 20 mM in the solution.

27. The method of claim 26, wherein the solution has a pH of about 6.0 to about 7.0, e.g., about 6.5.

28. The method of claim 26 or 27, wherein the solution further comprises a salt, wherein optionally the salt is selected from the group consisting of sodium chloride, magnesium chloride, and calcium chloride, e.g., sodium chloride.

29. The method of claim 28, wherein the salt is present in the solution at a concentration of from about 25-150 mM, e.g., 50-100 mM, e.g., about 75 mM.

30. The method of claim 28 or 29, wherein the concentration of the salt in the solution has a pH of about 6.5.

31. The method of any of claims 26-30, wherein the solution further comprises a carbohydrate, e.g., a non-reducing carbohydrate, e.g., sucrose or trehalose.

32. The method of claim 31, wherein the carbohydrate is present in the solution at a concentration of from about 1% to about 10% by weight per volume of said solution, e.g., about 2% to about 5% by weight per volume of the solution, about 2.5% by weight per volume of the solution.

33. The method of claim 31 or 32, wherein the carbohydrate is present in the solution at a concentration of about 30-150 mM (about 73 mM), or 150-300 (e.g., about 220) mM.

34. The method of any of claims 26-33, wherein the solution further comprises one or both of NaCl (e.g., about 25-150 mM, e.g., 50-100 mM, e.g., about 75 mM), and sucrose (e.g., about 30-150 mM, e.g., about 73 mM, or e.g., about 150-300 mM, e.g., about 220 mM, or about 2.5% by weight per volume) of the solution.

35. The method of claim 26, wherein the solution comprises 20 mM PIPES, 75 mM sodium chloride, and 2.5% sucrose by weight per volume of the solution, and wherein the solution has a pH of about 6.5.

36. The method of claim 26, wherein the solution comprises 20 mM PIPES, 75 mM sodium chloride, and 73 mM sucrose and wherein the solution has a pH of about 6.5.

37. The method of claim 26, wherein the solution comprises 20 mM PIPES, 75 mM sodium chloride, and 220 mM sucrose and wherein the solution has a pH of about 6.5.

38. The method of claim 26, wherein the solution further comprises 20 mM PIPES, 75 mM arginine, e.g., arginine-HCl, and wherein the solution has a pH of about 6.5.

39. The method of any of claims 26-38, wherein the osmolality of said solution is from about 270 mOsm/kg to about 330 mOsm/kg, e.g., about 275 mOsm/kg to about 300 mOsm/k, e.g., about 285 mOsm/kg.

40. The method of any of claims 26-39, which further comprises: c) performing a purification step, e.g., a filtration step, on the composition of b), thereby producing a semi-purified composition comprising the lentiviral vector.

41. The method of claim 40, which further comprises, after step c), contacting the semi-purified composition with arginine or a salt thereof.

42. The method of any of claims 24-41, wherein the arginine encapsulates the lentiviral vector.

43. The method of any of claims 24-42, wherein the arginine stabilizes the lentiviral vector.

44. The method of any of claims 24-43, wherein the impurity comprises a protein (e.g., a host cell protein), a nucleic acid (e.g., a host cell nucleic acid), a carbohydrate (e.g., a host cell carbohydrate), a lipid, an enzyme, a salt, a buffer, or any combination thereof.

45. The method of any one of claims 1-44, wherein the cell density at transfection is between about 1.0×10⁶ cells/mL and about 3.0×10⁶ cells/mL (e.g., between 1.5×10⁶ cells/mL and 2.5×10⁶ cells/mL).

46. The method of any one of claims 1-45, wherein the viability of the cells is, or is assessed to be, at least 90% (e.g., 91%, 92%, 93%, 94%, 95%, 96%, 97%, 98%, 99%, or 100%) at the time of transfection.

47. The method of claim 46, wherein the viability of the cells is measured at or around the time of transfection (e.g., within 30 minutes prior to transfection).

48. The method of any one of claims 1-47, wherein the method is used for a process with two or more nucleic acids (e.g., two or more plasmids, e.g., two plasmids, three plasmids, four plasmids, or five plasmids).

49. An aqueous composition comprising a lentiviral vector, arginine, a 1,4-piperazinediethanesulfonic acid (PIPES) buffer, and a salt.

50. The aqueous composition of claim 49, wherein the arginine in the aqueous composition is at a concentration of about 25-50 mM (about 50 mM), 50-100 mM (e.g., about 75 mM), 100-200 mM (e.g., about 150 mM), or 200-400 (e.g., about 300) mM arginine), wherein optionally the PIPES aqueous composition is at a concentration of from about 10 mM to about 50 mM, e.g., about, e.g., 20 mM.

51. The aqueous composition of claim 49 or 50, wherein the aqueous composition has a pH of about 6.0 to about 7.0, e.g., about 6.5.

52. The aqueous composition of any one of claims 49-51, wherein the aqueous composition further comprises a salt, wherein optionally the salt is selected from the group consisting of sodium chloride, magnesium chloride, and calcium chloride.

53. The aqueous composition of any one of claims 49-52, wherein the salt is sodium chloride (NaCl).

54. The aqueous composition of any one of claims 49-53, wherein the salt in the aqueous composition is from about 25 mM to about 150 mM, e.g., about 50 mM to about 75 mM.

55. The aqueous composition of any one of claims 49-54, wherein the aqueous composition comprises 20 mM PIPES and 75 mM sodium chloride, and wherein the aqueous composition has a pH of about 6.5.

56. The aqueous composition of any one of claims 49-55, wherein the aqueous composition further comprises a carbohydrate, e.g., a non-reducing carbohydrate, e.g., sucrose or trehalose.

57. The aqueous composition of any one of claims 49-56, wherein the carbohydrate is present in the aqueous composition at a concentration of from about 1% to about 10% by weight per volume of said solution, e.g., about 2% to about 5% by weight per volume of the aqueous composition, about 2.5% by weight per volume of the aqueous composition.

58. The aqueous composition of any one of claims 49-57. wherein the carbohydrate is present in the aqueous composition at a concentration of from about 30-150 mM (about 73 mM), or 150-300 (e.g., about 220) mM.

59. The aqueous composition of any one of claims 49-58, wherein the aqueous composition comprises one or both of NaCl (e.g., about 25-150 mM, e.g., 50-100 mM, e.g., about 75 mM), and sucrose (e.g., about 30-150 mM, e.g., about 73 mM, or e.g., about 150-300 mM, e.g., about 220 mM, or about 2.5% by weight per volume) of the aqueous composition.

60. The aqueous composition of any one of claims 49-59, wherein the aqueous composition comprises 20 mM PIPES, 75 mM sodium chloride, and 2.5% sucrose by weight per volume of the aqueous composition and wherein the aqueous composition has a pH of about 6.5.

61. The aqueous composition of any one of claims 49-60, wherein the aqueous composition comprises 20 mM PIPES, 75 mM sodium chloride and 73 mM sucrose and wherein the aqueous composition has a pH of about 6.5.

62. The aqueous composition of any one of claims 49-61, wherein the aqueous composition comprises 20 mM PIPES, 75 mM sodium chloride and 220 mM sucrose and wherein the aqueous composition has a pH of about 6.5.

63. The aqueous composition of any one of claims 49-62, wherein the osmolality of said aqueous composition is from about 270 mOsm/kg to about 330 mOsm/kg, e.g., about 275 mOsm/kg to about 300 mOsm/k, e.g., about 285 mOsm/kg.

64. The aqueous composition of any one of claims 49-63, wherein the lentiviral vector of any preceding claims is present at a concentration of from about 3×10⁸ TU/mL to about 5×10⁸ TU/mL.

65. The aqueous composition of any of claims 49-64 which is free of one or more proteins selected from the group consisting of human serum albumin (HSA), recombinant human serum albumin (rHSA), bovine serum albumin (BSA), and a lipoprotein.

66. The lentiviral vector of any of the preceding claims, wherein lentiviral vector comprises a transgene, e.g., a transgene encoding a protein, e.g., a protein comprising a chimeric antigen receptor (CAR).

67. The lentiviral vector of any of the preceding claims, wherein said CAR comprises, in an N-terminal to C-terminal direction, an antigen binding domain, a transmembrane domain, and one or more signaling domains.

68. The lentiviral vector of any of the preceding claims, wherein said signaling domain comprises one or more primary signaling domains and/or one or more costimulatory signaling domains.

69. The lentiviral vector of any of the preceding claims, wherein one of said one or more primary signaling domains comprises a CD3-zeta stimulatory domain.

70. The lentiviral vector of any of the preceding claims, wherein one or more of said costimulatory signaling domains comprises an intracellular domain selected from a costimulatory protein selected from the group consisting of OX40, CD27, CD28, CDS, ICAM-1, LFA-1 (CD11a/CD18), ICOS(CD278), 4-1BB (CD137), CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, e.g., a 4-1 BB (CD137) costimulatory domain or a CD28 costimulatory domain.

71. The lentiviral vector of any of the preceding claims, wherein said antigen binding domain is an scFv.

72. The lentiviral vector of any of the preceding claims, wherein said antigen binding domain binds to an antigen selected from the group consisting of CD19; CD123; CD22; CD30; CD171; CS-1; C-type lectin-like molecule-1, CD33; epidermal growth factor receptor variant III (EGFRvlll); ganglioside G2 (GD2); ganglioside GD3; TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fins-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD1 17); lnterleukin-13 receptor subunit alpha-2; mesothelin; Interleukin 1 1 receptor alpha (IL-1 1 Ra); prostate stem cell antigen (PSCA); Protease Serine 21; vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3; transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51 E2 (OR51 E2); TCR Gamma Alternate Reading Frame Protein (TARP); Wilms tumor protein (WT1); Cancer/testis antigen 1 (NY-ESO-1); Cancer/testis antigen 2 (LAGE-1 a); Melanoma-associated antigen 1 (MAGE-A1); ETS translocation-variant gene 6, located on chromosome 12p (ETV6-AML); sperm protein 17 (SPA17); X Antigen Family, Member 1 A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1, melanoma antigen recognized by T cells 1; Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1 B1 (CYP1 B1); CCCTC-Binding Factor (Zinc Finger Protein)-Like, Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLECI2A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1), e.g., to CD19, CD22, mesothelin, or CD123.

73. The lentiviral vector of any of the preceding claims, wherein said CAR comprises an anti-CD19 antibody or a fragment thereof, a 4-1 BB (CD137) transmembrane domain, and a CD3-zeta signaling domain.

74. The lentiviral vector of any of the preceding claims, comprises a second transgene, e.g., a second transgene encoding a second protein, e.g., a second protein comprising a second chimeric antigen receptor (CAR).

75. A method of manufacturing a lentiviral vector, comprising:
a) providing a population of human cells (e.g., 293 cells);
b) introducing into the cells nucleic acid encoding a retroviral packaging protein, a retroviral envelope protein, and a therapeutic effector, e.g., a therapeutic protein (e.g., a CAR),
c) contacting the cells with benzonase at a time about 2-6 (e.g., about 3), 4-10 (e.g., about 6), 6-40, 10-40, 10-30 (e.g., about 24), or about 20 hours after step b); and
d) culturing the cells under conditions suitable for production of the lentiviral vector.

76. The method of claim 75, wherein Benzonase is added 6-10 hours, 10-20 hours, 20-30 hours, 30-40 hours, or 40-50 hours, before harvest of lentiviral vector from the cells.

77. A method of manufacturing a lentiviral vector, comprising:
a) providing a population of human cells (e.g., 293 cells);
b) introducing into the cells nucleic acid encoding a retroviral packaging protein, a retroviral envelope protein, and a therapeutic effector, e.g., a therapeutic protein (e.g., a CAR),
c) contacting the cells with benzonase (e.g., 3-24 hours after step b);
d) culturing the cells under conditions suitable for production of the lentiviral vector;
e) harvesting the lentiviral vectors from cells 6-10 hours, 10-20 hours, 20-30 hours, 30-40 hours, or 40-50 hours after step c).

78. The method of any of claims 75-77, wherein benzonase is at a concentration of about 10-40 U/mL, e.g., 20-30 U/mL, e.g., about 25 U/mL.

79. The method of any of claims 75-78, wherein benzonase is at a concentration of about 3-60 U/mL, 3-10 U/mL, 3-7 U/mL, 4-6 U/mL, or about 5 U/mL.

80. The method of any of claims 75-79, wherein the benzonase is at a concentration of 5-50, 5-15, 15-25, or 25-50 U/mL.

81. The method of any of claims 75-80, which further comprises, before step c), contacting the benzonase with MgCl₂, e.g., at about 1-5 mM, 1-3 mM, or about 2 mM.

82. A method of manufacturing a lentiviral vector, comprising:
a) providing a plurality of mammalian (e.g., human) cells, wherein the plurality of mammalian cells do not comprise SV40 large T antigen (e.g., wherein the cell is a fibroblast cell, e.g., an embryonic kidney fibroblast cell, e.g., an Expi293F cell), wherein the plurality of mammalian cells comprise a nucleic acid (e.g., DNA) encoding one or more retroviral packaging protein, a retroviral envelope protein, and a therapeutic effector, e.g., a therapeutic protein (e.g., a CAR),
b) culturing the cell under conditions suitable for production of the lentiviral vector.

83. The method of claim 82, wherein a) comprises introducing the nucleic acid into the plurality of mammalian cells.

84. The method of any of claims 75-83, which further comprises at least partially separating the lentiviral vector from the plurality of mammalian cells.

85. The method of claim 83 or 84, wherein the one or more retroviral packaging proteins comprises a lentiviral gag, a lentiviral pol, or a lentiviral rev, or any combination thereof.

86. The method of any of claims 83-85, wherein the retroviral envelope protein comprises a VSV-G.

87. A preparation of lentiviral vector, comprising:
a plurality of lentiviral vectors that comprise:
a) a lentivirus genome encoding a therapeutic effector, e.g., a therapeutic protein (e.g., a CAR), and
b) an envelope enclosing the lentivirus genome (wherein optionally the envelope comprises VSV-G);
wherein the preparation comprises at least 5×10⁷, 1×10⁸, 1×10⁹, or 1×10¹⁰, transducing units;
wherein the preparation comprises less than 10 μg/ml or less than 1 μg/ml of nucleic acid (e.g., DNA) encoding SV40 large T antigen.

88. The method of any of claims 83-87, wherein the plurality of lentiviral vectors comprises at least 1×10⁹, 2×10⁹, 5×10⁹, or 1×10¹⁰, 2×10¹⁰, 5×10¹⁰, 1×10¹¹, 2×10¹¹, 5×10¹, or 1×10¹² of the cells.

89. The method of any of claims 83-88, wherein the plurality of mammalian cells are in a culture volume of at least 5, 10, 20, 50, 100, 200, or 500 L.

90. The method of any of claim 83-89, which comprises culturing the plurality of mammalian cells in serum-free medium.

91. The method of any of claim 83-90, wherein the plurality of mammalian cells are grown in suspension.

92. The method of any of claims 83-91, wherein the CAR comprises a CD19 CAR (e.g., a humanized CD19 CAR, e.g., as described in WO2014153270A1.

93. The method of any of claims 83-91, wherein the CAR comprises a dual CAR (e.g., a humanized CD19-CD22 CAR, e.g., as described in WO2016164731A2.

94. The method of any of claims 83-91, wherein the nucleic acid encoding a CAR further encodes a shRNA, e.g., as described in WO2017049166A.

95. The method of any of claims 83-94, wherein the lentiviral vector is produced in cells cultured in the absence of serum.

96. The method or composition of any of the preceding claims, wherein the lentiviral vector is characterized by a hydrodynamic radius of 100±25 nm as measured by dynamic light scattering (DLS).

97. The method or composition of any of the preceding claims, wherein the lentiviral vector maintains said hydrodynamic radius of 100±25 nm within a temperature range of from 25° C. to 55° C.

98. The method or composition of any of the preceding claims, wherein the lentiviral vector is characterized by a polydispersity of from 10% to 25%.

99. The method or composition of any of the preceding claims, wherein the lentiviral vector maintains said polydispersity of from 10% to 25% within a temperature range of from 25° C. to 55° C.

100. The method or composition of any of the preceding claims, wherein the lentiviral vector maintains a concentration after 3 freeze/thaw cycles of from about 70% to about 100% relative to the concentration of said lentiviral vector in said aqueous composition prior to said freeze/thaw cycles, wherein each of said freeze/thaw cycles comprises freezing said aqueous composition and subsequently allowing said aqueous composition to thaw at room temperature.

101. The method or composition of any of the preceding claims, wherein the lentiviral vector maintains said concentration of from about 70% to about 100% after 6-10 of said freeze/thaw cycles, e.g., after 6-9 of said freeze/thaw cycles.

102. An aqueous composition comprising a lentiviral vector, a buffer selected from the group consisting of a phosphate buffer, a sodium citrate buffer, a 2-(N-morpholino) ethanesulfonic acid (MES) buffer, a 3-morpholinopropane-1-sulfonic acid (MOPS) buffer, and a salt.

103. The aqueous composition of claim 102, wherein said salt is selected from the group consisting of sodium chloride, magnesium chloride, and calcium chloride.

104. The aqueous composition of claim 101 or 103, wherein said aqueous composition further comprises a non-reducing carbohydrate selected from the group consisting of sucrose and trehalose.
